SEQUENCE LISTING (2 sequences)

SEQ ID NO: 1 - DNA, 32 bp, Artificial Sequence
  Description of Artificial Sequence Synthetic primer
gcgcctcgagccgcttcaatggggtctacgct

SEQ ID NO: 2 - DNA, 32 bp, Artificial Sequence
  Description of Artificial Sequence Synthetic primer
ggccggatcctcggagacatcgttgcgggtat

(19) United States
(12) Patent Application Publication
(10) Pub. No.: US 2015/0071904 A1
(43) Pub. Date: Mar. 12, 2015

(54) COMPOSITIONS AND METHODS TO BOOST ENDOGENOUS ROS PRODUCTION FROM BACTERIA

(71) Applicant: Trustees of Boston University, Boston, MA (US)

(72) Inventors: James J. Collins, Newton, MA (US); Mark P. Brynildsen, Pennington, NJ (US); Jonathan A. Winkler, Brighton, MA (US); Catherine Spina, Boston, MA (US)

(21) Appl. No.: 14/370,558

(22) PCT Filed: Jan. 4, 2013
(86) PCT No.: PCT/US13/20239

§ 371 Date: Jul. 3, 2014

Related U.S. Application Data
(60) Provisional application No. 61/583,662, filed on Jan. 6, 2012.

(51) Int. Cl. A61K 45/06

(52) U.S. Cl. CPC A61K 45/06
USPC 424/94.4

(57) ABSTRACT

Provided herein are compositions and methods comprising ROS target modulators that increase ROS flux and endogenus ROS production, thereby potentiating oxidative attack by antibiotics and biocides.

### CROSS-REFERENCE TO RELATED APPLICATIONS

This application claims benefit under 35 U.S.C. §119(e) of U.S. Provisional Patent Application Ser. No. 61/583,662 filed on 6 Jan. 2012, the contents of which are incorporated herein by reference in its entirety.

### GOVERNMENT SUPPORT

This invention was made with Government Support under Contract No. 0D003644 awarded by the National Institutes of Health. The Government has certain rights in the invention.

### FIELD OF THE INVENTION

The field of the invention relates to potentiating and boosting endogenous ROS production in bacteria.

### BACKGROUND OF THE INVENTION

The ever-increasing incidence of antibiotic-resistant infections combined with a weak pipeline of new antibiotics has created a global public health crisis^{1,2}. Reactive oxygen species (ROS) are produced by the immune system as a defense against microbes⁶ and can be induced by bactericidal antibiotics' to kill bacteria.

### SUMMARY OF THE INVENTION

As demonstrated herein, the inventors have discovered that ROS production can be predictably enhanced in bacteria, such as aerobic and facultative anaerobic bacteria (e.g., E. coli), thereby increasing the bacteria's susceptibility to oxidative attack. To do so, an ensemble of genome-scale metabolic models was created capable of predicting ROS production in E. coli and other bacteria. The metabolic network models were systematically perturbed and flux distributions analyzed to identify targets predicted to increase ROS production. In silico predictions were experimentally validated and shown to confer increased susceptibility to oxidants (O2−, H2O2, NaOCl). The validated targets also increased susceptibility to killing by bactericidal antibiotics. Accordingly, the work described herein establishes a systems-based method to rationally tune ROS production in bacteria, and demonstrates that increased microbial ROS production can potentiate killing by oxidants and antibiotic treatment. Thus, provided herein are compositions comprising ROS target modulators, such as inhibitors of: ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, and/or fumarase B, for increasing endogenous ROS production and potentiating antibiotics and biocides, and methods thereof.

Accordingly, provided herein, in some aspects, are methods for inhibiting a bacterial infection by increasing ROS (reactive oxygen species) production in a bacteria, the methods comprising administering to a subject having or at risk for a bacterial infection an effective amount of one or more ROS target modulator compounds and an effective amount of an antibiotic agent.

Also provided herein, in some aspects, are methods for inhibiting a bacterial infection by increasing ROS (reactive oxygen species) production in a bacteria, the methods comprising administering to a subject having or at risk for a bacterial infection an effective amount of a pharmaceutical composition comprising one or more ROS target modulator compounds and an antibiotic agent.

In some aspects provided herein are methods for treating a bacterial infection by increasing ROS (reactive oxygen species) production in a bacteria, comprising administering to a patient having a bacterial infection and undergoing treatment with an antibiotic agent, an effective amount of one or more ROS target modulator compounds.

In some embodiments of these methods and all such methods described herein, the ROS target modulator is an inhibitor of an enzyme involved in bacterial glycolysis, pentose-phosphate pathway, EntnerDoudoroff pathway, TCA cycle, glyoxylate shunt, aerobic respiration, or acetate metabolism.

In some embodiments of these methods and all such methods described herein, the ROS target modulator is an inhibitor of: ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, or fumarase B.

In some embodiments of these methods and all such methods described herein, the inhibitor of ATP synthase is selected from IF1, an efrapeptin, aurovertin B, citreoviridin, α-zearalenol, and any analogs thereof.

In some embodiments of these methods and all such methods described herein, the inhibitor of succinate dehydrogenase is selected from carboxin, thenoyltrifluoroacetone, malonate, malate, oxaloacetate, and any analogs thereof.

In some embodiments of these methods and all such methods described herein, the inhibitor of glutamate dehydrogenase is selected from bromofuroate; 3-carboxy-5-bromofuroic acid; Palmitoyl-Coenzyme-A; orthovanadate; vanadyl sulphate, vanadyl acetylacetonate, glutarate; 2-oxoglutarate; estrogen; pyridine-2,6-dicarboxylic acid; and (−)-epigallocatechin gailate (EGCG).

In some embodiments of these methods and all such methods described herein, the inhibitor of NADH dehydrogenase is selected from Amytal; Amytal Sodium; Annonin VI; Aurachin A; Aurachin B; Aureothin; Benzimidazole; Bullactin; calnexin; Capsaicin; Ethoxyformic anhydride; Ethoxyquin; Fenpyroximate; Mofarotene; mofarotene 2-oxoglutarate dehydrogenase; Molvizarin; Myxalamide PI; M2-type pyruvate kinase; Otivarin; Pethidine; rhein; Phenalamid A2; Phenoxan; Piericidin A; p-chloromercuribenzoate; Ranolazine; Rolliniasatin-1; Rolliniasatin-2; Rotenone; Squamocin; Thiangazole rotenoids; thiol reagents; Demerol; iron chelators; NAD+(nicotinamide adenine dinucleotide; oxidized form); AMP (adenosine monophosphate); ADP (adenosine diphosphate); ADP-ribosylation factor 3; ATP (adenosine triphosphate); guanidinium salts; NADH; barbituates; gossypol; polyphenol; dihydroxynaphthoic acids; acetogenin; adenosine diphosphate ribose; rotenoid; acetogenin; nitrosothiols; peroxynitrite; carvedilol; arylazido-beta-alanyl NAD+; adriamycin; 4-hydroxy-2-nonenal; pyridine derivatives; 2-heptyl-4-hydroxyquinoline N-oxide; dicumarol; o-phenanthroline; and 2;2′-dipyridyl.

In some embodiments of these methods and all such methods described herein, the inhibitor of pyruvate dehydrogenase is selected from

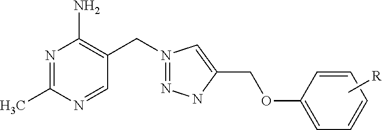

where R is 2-Cl-4-NO₂, 4-NO₂, 4-COOH, or H; secondary amides of (R)-3;3;3-Trifluoro-2-hydroxy-2-methylpropionic acid glyoxylate; (R)-3;3;3-Trifluoro-2-hydroxy-2-methylpropionamides; anilides of (R)-Trifluoro-2-hydroxy-2-methylpropionic acidhydroxypyruvate; kynurenate; xanthurenate; α-cyano-4-hydroxycinnamic acid; bromopyruvic acid; fluropyruvic acid; AZD-7545; phosphonate and phosphinate analogs of pyruvate; mono- and bifunctional arsenoxides; branched-chain 2-oxo acids; 2-oxo-3-alkynoic acids; tetrahydrothiamin diphosphate (ThDP); and 2-thiazolone and 2-thiothiazolone analogs of ThDP.

In some embodiments of these methods and all such methods described herein, the inhibitor of cytochrome oxidase is selected from azide; nitric oxide; cytochrome P450 oxidase inhibitors; aurachin A; Aurachin C; aurachin D; tridecylstigmatelli; stigmatellin; nigericin; hydroxylamine; heptylhydroxyquinoline N-oxide (HQNO); nonylhydroxyquinoline N-oxide (NQNO); dibromothymoquinone (DBMIB); piericidin A; and undecylhydroxydioxobenzo-thiazole (UHDBT).

In some embodiments of these methods and all such methods described herein, the inhibitor of glucose-6-phosphate dehydrogenase is selected from dehydroepiandrosterone (DHEA), DHEA-sulfate; 2-deoxyglucose; halogenated DHEA; epiandrosterone; isoflurane; sevoflurane; diazepam; CBF-BS2; cystamine; 16α-bromoepiandrosterone; 16α-hydroxy-5-androsten-17-one; 16α-fluoro-5-androsten-17-one; 16α-fluoro-16β-methyl-5-androsten-17-one; 16α-methyl-5-androsten-17-one; 16β-methyl-5-androsten-17-one; 16α-hydroxy-5α-androstan-17-one; 16α-fluoro-5α-androstan-17-one; 16α-fluoro-160-methyl-5α-androstan-17-one; 16α-methyl-5α-androstan-17-one; 16β-methyl-5α-androstan-17-one; and 2-amino-2-deoxy-D-glucose-6-phosphate.

In some embodiments of these methods and all such methods described herein, the inhibitor of 6-phosphogluconate dehydrogenase is selected from 6-aminonicotinamide; aldonate 4-phospho-d-erythronate; 5,6-Dideoxy-6-phosphono-d-arabino-hexonate; and 5-deoxy-5-phosphono-d-arabinonate.

In some embodiments of these methods and all such methods described herein, the inhibitor of succinyl-CoA synthetase is selected from LY266500 and vanadium sulphate.

In some embodiments of these methods and all such methods described herein, the inhibitor of triose phosphate isomerase is selected from 3-haloacetol phosphate; glycidol phosphate; phosphoenol pyruvate; DHAP; GAP; 2-phosphoglycollate; phosphoglycolohydroxamate; 3-phosphoglycerate; glycerol phosphate; phosphoenol pyruvate; 2;9-Dimethyl-β-carbolines and derivatives thereof; 3-(2-benzothiazolylthio)-1-propanesulfonic acid; 2-carboxyethylphosphonic acid; 2-phosphoglyceric acid; N-hydroxy-4-phosphono-butanamide; and [2(formyl-hydroxy-amino)-ethyl]-phosphonic acid.

In some embodiments of these methods and all such methods described herein, the inhibitor of phosphofructokinase is selected from aurintricarboxylic acid; pyruvate; 2-deoxy-2-fluoro-D-glucose; citrate and halogenated derivatives of citrate; fructose 2,6-bisphosphate; N-(2-methoxyethyl)-bromoacetamide; N-(2-ethoxyethyl)-bromoacetamide; N-(3-methoxypropyl)-bromoacetamide); phosphoglycerate; taxodone; taxodione; euparotin acetate eupacunin; vernolepin; argaric acid, quinaldic acid; and 5′-p-flurosuflonylbenzoyl adenosine.

In some embodiments of these methods and all such methods described herein, the inhibitor of the fumarase B is selected from trans-aconitate; bromomesaconate; citrate; meso-tartaric acid; bismuth; DL-fluoromalic acid; and S-2,3-Dicarboxyaziridine.

In some embodiments of these methods and all such methods described herein, the ROS target modulator is an inhibitor of E. coli cyoA, nuoG, or sdhC, or an ortholog thereof.

In some embodiments of these methods and all such methods described herein, the ROS target modulator is selected for its ability to boost ROS production or increase susceptibility to oxidative stress.

In some embodiments of these methods and all such methods described herein, the ROS is O₂⁻, H₂O₂, or O₂⁻ and H₂O₂.

In some embodiments of these methods and all such methods described herein, the antibiotic is bactericidal or bacteriostatic.

In some embodiments of these methods and all such methods described herein, the antibiotic agent is a β-lactam, fluoroquinoline, macrolide, nitroimidazole compound, tetracycline, vancomycin, bacitracin, macrolide; lincosamide, chloramphenicol, amphotericin, cefazolins, clindamycins, mupirocins, sulfonamides, trimethoprim, rifampicin, metronidazole, quinolone, novobiocin; polymixin; gramicidin, aminoglycoside, or any salts or variants thereof.

In some embodiments of these methods and all such methods described herein, the antibiotic agent is not an aminoglycoside.

In some embodiments of these methods and all such methods described herein, the β-lactam antibiotic agent is a penam antibiotic or a penicillin antibiotic. In some embodiments of these methods, the penicillin antibiotic is selected from amoxicillin, ampicillin, methicillin, oxacillin, nafcillin, cloxacillin, dicloxacillin, flucloxacillin, azlocillin, carbenicillin, ticarcillin, mezlocillin, piperacillin, penicillin, benzathine penicillin, benzylpenicillin, phenoxymethylpenicillin, procaine penicillin; temocillin; co-amoxiclav; and mecillinam.

In some embodiments of these methods and all such methods described herein, the β-lactam antibiotic agent is a cephalosporin or cephamycin. In some embodiments of these methods, the cephalosporin or cephamycin is selected from cefazolin, cefalexin, cefalotin, cefdinir, cefepime, cefotaxime, cefpodoxime proxetil, ceftobiprole, ceftaroline fosamil, cephalosporin C, cephalothin, cefaclor, cefamandole, cefuroxime, cefotetan, cefoxitin, cefixime, ceftazidime, ceftriaxone, and cefpirome.

In some embodiments of these methods and all such methods described herein, the β-lactam antibiotic agent is a carbapenem. In some embodiments of these methods, the carbapenem is selected from ertapenem, meropenem, imipenem, doripenem, panipenem/betamipron, biapenem, razupenem, and tebipenem.

In some embodiments of these methods and all such methods described herein, the β-lactam antibiotic agent is a penem. In some embodiments of these methods, the penem is selected from thiopenems, oxypenems, aminopenems, alkylpenems, and arylpenems.

In some embodiments of these methods and all such methods described herein, the β-lactam antibiotic agent is a monobactam. In some embodiments of these methods, the monobactam is selected from aztreonam, tigemonam, nocardicin A, and tabtoxinine β-lactam.

In some embodiments of these methods and all such methods described herein, when the antibiotic agent is a β-lactam antibiotic agent, the one or more ROS target modulators is selected from a cytochrome oxidase inhibitor, an NADH dehydrogenase inhibitor, a succinate dehydrogenase inhibitor, or any combination thereof.

In some embodiments of these methods and all such methods described herein, the fluorquinolone antibiotic agent is selected from ciprofloxacin, moxifloxacin, ofloxacin, balofloxacin, grepafloxacin, levofloxacin, pazufloxacin, sparfloxacin, temafloxacin, and tosufloxacin.

In some embodiments of these methods and all such methods described herein, when the antibiotic agent is a fluorquinolone antibiotic agent, the one or more ROS target modulators is selected from a cytochrome oxidase inhibitor, an NADH dehydrogenase inhibitor, a succinate dehydrogenase inhibitor, a phospho acetyl transferase inhibitor, or any combination thereof.

In some embodiments of these methods and all such methods described herein, the nitroimidazole compound antibiotic is selected from metronidazole, tinidazole, and nimorazole.

In some embodiments of these methods and all such methods described herein, the tetracycline antibiotic agent is selected from tetracycline, chlortetracycline, oxytetracycline, demeclocycline, doxycycline, lymecycline, meclocycline, methacycline, minocycline, and rolitetracycline.

In some embodiments of these methods and all such methods described herein, the bacterial infection involves a gram positive or gram negative bacteria.

In some embodiments of these methods and all such methods described herein, the bacterial infection is of an aerobic bacteria or facultative anaerobic bacteria.

In some embodiments of these methods and all such methods described herein, the bacterial infection is caused by a bacterial pathogen having an active metabolic system comprising glycolysis, pentose-phosphate pathway, and/or EntnerDoudoroff pathway.

In some embodiments of these methods and all such methods described herein, the bacterial infection is caused by a bacterial pathogen having an active metabolic system comprising the TCA cycle, glyoxylate shunt, and/or acetate metabolism.

In some embodiments of these methods and all such methods described herein, the bacterial infection is of an enteric or respiratory pathogen.

In some embodiments of these methods and all such methods described herein, the bacterial infection is pneumonia, strep throat, bacteremia, sepsis, toxic shock syndrome, endocarditis, abscess, an infection of skin or soft tissue, or is an infected wound or burn.

In some embodiments of these methods and all such methods described herein, the bacterial infection is necrotizing fasciitis, osteomyelitis, peritonitis, infected surgical wound, or diabetic ulcer.

In some embodiments of these methods and all such methods described herein, the bacterial infection is a chronic or persistent bacterial infection.

In some embodiments of these methods and all such methods described herein, the bacterial infection is an acute or non-latent bacterial infection.

In some embodiments of these methods and all such methods described herein, the infection is a surface wound, burn, or infection; infection of a mucosal surface; respiratory infection; infections of the eyes, ears, nose, or throat; or infection of an intestinal pathogen.

In some embodiments of these methods and all such methods described herein, the bacterial infection is resistant to one or more anti-microbial agents.

In some embodiments of these methods and all such methods described herein, the bacterial infection involves one or more of E. coli, Mycobacterium sp., Staphylococcus sp., Haemophilus sp., Salmonella sp., Streptococcus sp., Neisseria sp., Pseudomonas sp., Klebsiella sp., Enterobacter sp., Acinetobacter sp., Listeria sp., Campylobacter sp., Enterococcus sp., Bacillus sp., Corynebacterium sp., Clostridium sp., Bacteroides sp., Treponema sp., Lactobacillus sp., Nocardia sp.; Actinomyces sp., Mobiluncus sp., Peptostreptococcus sp., Brucella sp., Campylobacter sp., Proteus sp.; Shigella sp.; Yersinia sp., Aeromonas sp., Vibrio sp., Acinetobacter sp., Flavobacterium sp.; Burkholderia sp., Bacteroides sp., Prevotella sp., Fusobacterium sp., Borrelia sp., Chlamydia sp., Legionella sp., and Leptospira sp.

In some embodiments of these methods and all such methods described herein, the bacterial infection involves one or more of E. coli, Klebsiella pneumoniae, Acinetobacter baumanii, Pseudomonas aeruginosa, Streptococcus pneumoniae, Mycobacterium tuberculosis, Staphylococcus aureus, Haemophilus influenzae, and Salmonella typhimurium.

In some embodiments of these methods and all such methods described herein, the ROS target modulator and the antibiotic agent are co-formulated.

In some embodiments of these methods and all such methods described herein, the ROS target modulator and the antibiotic agent are administered separately.

In some embodiments of these methods and all such methods described herein, the ROS target modulator is administered systemically or locally.

In some embodiments of these methods and all such methods described herein, the ROS target modulator is administered intravenously, orally, or topically.

In some embodiments of these methods and all such methods described herein, the bacterial infection occurs at or in a surface wound or burn, and the ROS target modulator is administered topically to the affected area.

In some embodiments of these methods and all such methods described herein, the ROS target modulator is formulated as a cream, gel, foam, spray, or as a tablet or capsule for oral delivery.

In some aspects, provided herein are ROS target modulator for use in inhibiting or treating a bacterial infection by increasing ROS (reactive oxygen species) production in a bacteria.

In some embodiments of these uses and all such uses described herein, the ROS target modulator is an inhibitor of an enzyme involved in bacterial glycolysis, pentose-phosphate pathway, EntnerDoudoroff pathway, TCA cycle, glyoxylate shunt, aerobic respiration, or acetate metabolism.

In some embodiments of these uses and all such uses described herein, the ROS target modulator is an inhibitor of: ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, or fumarase B.

In some embodiments of these uses and all such uses described herein, the inhibitor of ATP synthase is selected from IF1, an efrapeptin, aurovertin B, citreoviridin, α-zearalenol, and any analogs thereof.

In some embodiments of these uses and all such uses described herein, the inhibitor of succinate dehydrogenase is selected from carboxin, thenoyltrifluoroacetone, malonate, malate, oxaloacetate, and any analogs thereof.

In some embodiments of these uses and all such uses described herein, the inhibitor of glutamate dehydrogenase is selected from bromofuroate; 3-carboxy-5-bromofuroic acid; Palmitoyl-Coenzyme-A; orthovanadate; vanadyl sulphate, vanadyl acetylacetonate, glutarate; 2-oxoglutarate; estrogen; pyridine-2,6-dicarboxylic acid; and (−)-epigallocatechin gailate (EGCG).

In some embodiments of these uses and all such uses described herein, the inhibitor of NADH dehydrogenase is selected from Amytal; Amytal Sodium; Annonin VI; Aurachin A; Aurachin B; Aureothin; Benzimidazole; Bullactin; calnexin; Capsaicin; Ethoxyformic anhydride; Ethoxyquin; Fenpyroximate; Mofarotene; mofarotene 2-oxoglutarate dehydrogenase; Molvizarin; Myxalamide PI; M2-type pyruvate kinase; Otivarin; Pethidine; rhein; Phenalamid A2; Phenoxan; Piericidin A; p-chloromercuribenzoate; Ranolazine; Rolliniasatin-1; Rolliniasatin-2; Rotenone; Squamocin; Thiangazole rotenoids; thiol reagents; Demerol; iron chelators; NAD+(nicotinamide adenine dinucleotide; oxidized form); AMP (adenosine monophosphate); ADP (adenosine diphosphate); ADP-ribosylation factor 3; ATP (adenosine triphosphate); guanidinium salts; NADH; barbituates; gossypol; polyphenol; dihydroxynaphthoic acids; acetogenin; adenosine diphosphate ribose; rotenoid; acetogenin; nitrosothiols; peroxynitrite; carvedilol; arylazido-beta-alanyl NAD+; adriamycin; 4-hydroxy-2-nonenal; pyridine derivatives; 2-heptyl-4-hydroxyquinoline N-oxide; dicumarol; o-phenanthroline; and 2;2′-dipyridyl.

In some embodiments of these uses and all such uses described herein, the inhibitor of pyruvate dehydrogenase is selected from

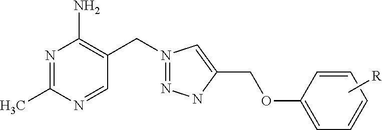

where R is 2-Cl-4-NO₂, 4-NO₂, 4-COOH, or H; secondary amides of (R)-3;3;3-Trifluoro-2-hydroxy-2-methylpropionic acid glyoxylate; (R)-3;3;3-Trifluoro-2-hydroxy-2-methylpropionamides; anilides of (R)-Trifluoro-2-hydroxy-2-methylpropionic acidhydroxypyruvate; kynurenate; xanthurenate; α-cyano-4-hydroxycinnamic acid; bromopyruvic acid; fluropyruvic acid; AZD-7545; phosphonate and phosphinate analogs of pyruvate; mono- and bifunctional arsenoxides; branched-chain 2-oxo acids; 2-oxo-3-alkynoic acids; tetrahydrothiamin diphosphate (ThDP); and 2-thiazolone and 2-thiothiazolone analogs of ThDP.

In some embodiments of these uses and all such uses described herein, the inhibitor of cytochrome oxidase is selected from azide; nitric oxide; cytochrome P450 oxidase inhibitors; aurachin A; Aurachin C; aurachin D; tridecylstigmatelli; stigmatellin; nigericin; hydroxylamine; heptylhydroxyquinoline N-oxide (HQNO); nonylhydroxyquinoline N-oxide (NQNO); dibromothymoquinone (DBMIB); piericidin A; and undecylhydroxydioxobenzo-thiazole (UHDBT).

In some embodiments of these uses and all such uses described herein, the inhibitor of glucose-6-phosphate dehydrogenase is selected from dehydroepiandrosterone (DHEA), DHEA-sulfate; 2-deoxyglucose; halogenated DHEA; epiandrosterone; isoflurane; sevoflurane; diazepam; CBF-BS2; cystamine; 16α-bromoepiandrosterone; 16α-hydroxy-5-androsten-17-one; 16α-fluoro-5-androsten-17-one; 16α-fluoro-16β-methyl-5-androsten-17-one; 16α-methyl-5-androsten-17-one; 16β-methyl-5-androsten-17-one; 16α-hydroxy-5α-androstan-17-one; 16α-fluoro-5α-androstan-17-one; 16α-fluoro-160-methyl-5α-androstan-17-one; 16α-methyl-5α-androstan-17-one; 16β-methyl-5α-androstan-17-one; and 2-amino-2-deoxy-D-glucose-6-phosphate.

In some embodiments of these uses and all such uses described herein, the inhibitor of 6-phosphogluconate dehydrogenase is selected from 6-aminonicotinamide; aldonate 4-phospho-d-erythronate; 5,6-Dideoxy-6-phosphono-d-arabino-hexonate; and 5-deoxy-5-phosphono-d-arabinonate.

In some embodiments of these uses and all such uses described herein, the inhibitor of succinyl-CoA synthetase is selected from LY266500 and vanadium sulphate.

In some embodiments of these uses and all such uses described herein, the inhibitor of triose phosphate isomerase is selected from 3-haloacetol phosphate; glycidol phosphate; phosphoenol pyruvate; DHAP; GAP; 2-phosphoglycollate; phosphoglycolohydroxamate; 3-phosphoglycerate; glycerol phosphate; phosphoenol pyruvate; 2;9-Dimethyl-β-carbolines and derivatives thereof; 3-(2-benzothiazolylthio)-1-propanesulfonic acid; 2-carboxyethylphosphonic acid; 2-phosphoglyceric acid; N-hydroxy-4-phosphono-butanamide; and [2(formyl-hydroxy-amino)-ethyl]-phosphonic acid.

In some embodiments of these uses and all such uses described herein, the inhibitor of phosphofructokinase is selected from aurintricarboxylic acid; pyruvate; 2-deoxy-2-fluoro-D-glucose; citrate and halogenated derivatives of citrate; fructose 2,6-bisphosphate; N-(2-methoxyethyl)-bromoacetamide; N-(2-ethoxyethyl)-bromoacetamide; N-(3-methoxypropyl)-bromoacetamide); phosphoglycerate; taxodone; taxodione; euparotin acetate eupacunin; vernolepin; argaric acid, quinaldic acid; and 5′-p-flurosuflonylbenzoyl adenosine.

In some embodiments of these uses and all such uses described herein, the inhibitor of the fumarase B is selected from trans-aconitate; bromomesaconate; citrate; meso-tartaric acid; bismuth; DL-fluoromalic acid; and S-2,3-Dicarboxyaziridine.

TIn some embodiments of these uses and all such uses described herein, the ROS target modulator is an inhibitor of E. coli cyoA, nuoG, or sdhC, or an ortholog thereof.

In some embodiments of these uses and all such uses described herein, the ROS target modulator is selected for its ability to boost ROS production or increase susceptibility to oxidative stress.

In some embodiments of these uses and all such uses described herein, the ROS is O₂⁻, H₂O₂, or O₂⁻ and H₂O₂.

In some embodiments of these uses and all such uses described herein, the bacterial infection involves a gram positive or gram negative bacteria.

In some embodiments of these uses and all such uses described herein, the bacterial infection is of an aerobic bacteria or facultative anaerobic bacteria.

In some embodiments of these uses and all such uses described herein, the bacterial infection is caused by a bacterial pathogen having an active metabolic system comprising glycolysis, pentose-phosphate pathway, and/or EntnerDoudoroff pathway.

In some embodiments of these uses and all such uses described herein, the bacterial infection is caused by a bacterial pathogen having an active metabolic system comprising the TCA cycle, glyoxylate shunt, and/or acetate metabolism.

In some embodiments of these uses and all such uses described herein, the bacterial infection is of an enteric or respiratory pathogen.

In some embodiments of these uses and all such uses described herein, the bacterial infection is pneumonia, strep throat, bacteremia, sepsis, toxic shock syndrome, endocarditis, abscess, an infection of skin or soft tissue, or is an infected wound or burn.

In some embodiments of these uses and all such uses described herein, the bacterial infection is necrotizing fasciitis, osteomyelitis, peritonitis, infected surgical wound, or diabetic ulcer.

In some embodiments of these uses and all such uses described herein, the bacterial infection is a chronic or persistent bacterial infection.

In some embodiments of these uses and all such uses described herein, the bacterial infection is an acute or non-latent bacterial infection.

In some embodiments of these uses and all such uses described herein, the infection is a surface wound, burn, or infection; infection of a mucosal surface; respiratory infection; infections of the eyes, ears, nose, or throat; or infection of an intestinal pathogen.

In some embodiments of these uses and all such uses described herein, the bacterial infection is resistant to one or more anti-microbial agents.

In some embodiments of these uses and all such uses described herein, the bacterial infection involves one or more of E. coli, Mycobacterium sp., Staphylococcus sp., Haemophilus sp., Salmonella sp., Streptococcus sp., Neisseria sp., Pseudomonas sp., Klebsiella sp., Enterobacter sp., Acinetobacter sp., Listeria sp., Campylobacter sp., Enterococcus sp., Bacillus sp., Corynebacterium sp., Clostridium sp., Bacteroides sp., Treponema sp., Lactobacillus sp., Nocardia sp.; Actinomyces sp., Mobiluncus sp., Peptostreptococcus sp., Brucella sp., Campylobacter sp., Proteus sp.; Shigella sp.; Yersinia sp., Aeromonas sp., Vibrio sp., Acinetobacter sp., Flavobacterium sp.; Burkholderia sp., Bacteroides sp., Prevotella sp., Fusobacterium sp., Borrelia sp., Chlamydia sp., Legionella sp., and Leptospira sp.

In some embodiments of these uses and all such uses described herein, the bacterial infection involves one or more of E. coli, Klebsiella pneumoniae, Acinetobacter baumanii, Pseudomonas aeruginosa, Streptococcus pneumoniae, Mycobacterium tuberculosis, Staphylococcus aureus, Haemophilus influenzae, and Salmonella typhimurium.

In some embodiments of these uses and all such uses described herein, the ROS target modulator is co-formulated with an antibiotic agent. In some such embodiments, the antibiotic is bactericidal. In some such embodiments, the antibiotic is a β-lactam or fluoroquinolone antibiotic.

Also provided herein in some aspects are methods for inhibiting a bacterial infection by increasing ROS (reactive oxygen species) production in a bacteria, the methods comprising administering to a subject having or at risk for a bacterial infection an effective amount of one or more ROS target modulator compounds selected and an effective amount of an antibiotic agent, wherein the ROS target modulator is an inhibitor of ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, or fumarase B, and wherein the antibiotic agent is a β-lactam, fluoroquinoline, macrolide, nitroimidazole compound, tetracycline, vancomycin, bacitracin, macrolide; lincosamide, chloramphenicol, amphotericin, cefazolins, clindamycins, mupirocins, sulfonamides, trimethoprim, rifampicin, metronidazole, quinolone, novobiocin; polymixin; gramicidin, aminoglycoside, or any salts or variants thereof.

In some embodiments of these methods and all such methods described herein, the ROS target modulator and the antibiotic agent are co-formulated.

In some embodiments of these methods and all such methods described herein, the ROS target modulator and the antibiotic agent are administered separately.

In some embodiments of these methods and all such methods described herein, the bacterial infection involves a gram positive or gram negative bacteria.

In some embodiments of these methods and all such methods described herein, the bacterial infection is of an aerobic bacteria or facultative anaerobic bacteria.

In some embodiments of these methods and all such methods described herein, the bacterial infection is one or more of sepsis, bacteremia, pneumonia, endocarditis, skin or soft tissue infection, or an infected wound or burn.

In some embodiments of these methods and all such methods described herein, the bacterial infection comprises E. coli, P. aeroginusa, K pneumoniae, or A. Baumanii.

In some embodiments of these methods and all such methods described herein, the bacterial infection is an acute or non-latent infection.

In some embodiments of these methods and all such methods described herein, the bacterial infection is a chronic or persistent bacterial infection.

In some embodiments of these methods and all such methods described herein, the antibiotic is bactericidal.

In some embodiments of these methods and all such methods described herein, the antibiotic is a β-lactam or fluoroquinolone antibiotic.

Also provided herein in some aspects are methods for making an antimicrobial composition, comprising: selecting a gene whose deletion increases ROS production or sensitivity to oxidative stress in a bacteria, selecting an inhibitor of said gene, and formulating said inhibitor for administration.

In some embodiments of these methods and all such methods described herein, the gene is an enzyme that loses electrons to a flavin, quinone, and/or transition metal center during catalysis, said transition metal center optionally being an iron sulfur protein, aconitase, fumarase, or dihydroxy acid dehydratase.

In some embodiments of these methods and all such methods described herein, the gene is a bacterial: ATP synthase, succinate dehydrogenase, glutamate dehydrogenase, NADH dehydrogenase, pyruvate dehydrogenase, cytochrome oxidase, glucose-6-phosphate dehydrogenase, 6-phosphogluconate dehydrogenase, succinyl-CoA ligase, triose phosphate isomerase, phosphate acetyltransferase, phosphofructokinase, or fumerase B.

In some embodiments of these methods and all such methods described herein, the inhibitor is co-formulated with a bactericidal antibiotic.

In some embodiments of these methods and all such methods described herein, the bactericidal antibiotic is a β-lactam or fluoroquinolone antibiotic.

In some embodiments of these methods and all such methods described herein, the bacteria is an aerobe or facultative anaerobe.

In some embodiments of these methods and all such methods described herein, the bacteria is a causative agent of sepsis, pneumonia, skin or soft tissue infection, or infected burn or wound.

In some embodiments of these methods and all such methods described herein, the bacteria is E. coli, K pneumoniae, A. baumanii, or P. aeruginosa.

In some embodiments of these methods and all such methods described herein, the inhibitor is formulated for intravenous, topical, or oral delivery.

In some aspects, provided herein are methods for identifying metabolic perturbations that increase sensitivity towards oxidative stress in a microorganism, the methods comprising the steps of:
a. generating a genome-scale metabolic model of systems-level ROS production in the microorganism;
b. systematically deleting genes from the genome-scale metabolic model to identify genes that alter basal ROS production in the microorganism, wherein an increase in the basal ROS production in the microorganism is indicative that deletion of the gene(s) increases sensitivity towards oxidative stress in the microorganism; and
c. measuring ROS production in a variant of the microorganism genetically modified to lack the genes that alter basal ROS production identified in step (b).

In some embodiments of these methods and all such methods described herein, the microorganism is Escherichia coli, Mycobaterium tuberculosis, Staphylococcus aureus, Haemophilus influenzae, Klebsiella pneumoniae, Pseudomonas aeruginosa, Acintebacter baumanii, or Salmonella typhimurium.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” In various embodiments, the term “about” when used in connection with percentages means±10, ±5, or, ±1%.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

It is understood that the following detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### BRIEF DESCRIPTION OF THE DRAWINGS

FIG. 1 depicts a systems approach to enhance microbial ROS production. Methodology for the development and validation of an ensemble of systems-level models of E. coli metabolism for estimation of basal ROS production. ROS-generating reactions were incorporated into a metabolic reconstruction and FBA framework′. Network perturbations via single-gene knockouts were performed in silico using FBA to identify alterations that affect ROS production. In silico predictions were evaluated experimentally for ROS production and susceptibility to killing by oxidants and antibiotics.

FIGS. 2A-2D show in silico predictions and experimental measures of H₂O₂ and O₂⁻ levels. FIG. 2A shows predicted relative H₂O₂ levels of various strains compared to wildtype. Darker shading designates strains whose mean H₂O₂ production levels were simulated to be >5% higher than wildtype over both ensembles, whereas lighter shading designates strains whose mean H₂O₂ production levels were simulated to be <5% higher than wildtype over both ensembles. FIG. 2B shows experimentally measured relative fluorescence/OD600 of strains with the H₂O₂-sensitive reporter (dps promoter-gfp). Darker shading designates strains that were experimentally measured to have increased levels of H₂O₂ compared to wildtype (p-value<0.05), whereas lighter shading designates strains that were experimentally measured to have levels of H₂O₂ that do not exceed those of wildtype. FIG. 2C shows predicted O₂⁻ levels of various mutants compared to wildtype. Darker shading designates strains whose mean O₂⁻ production levels were simulated to be >5% higher than wildtype over both ensembles, whereas lighter shading designates strains whose mean O₂⁻ production levels were simulated to be <5% higher than wildtype over both ensembles. FIG. 2D shows experimentally measured relative fluorescence/OD600 of strains with the O₂⁻-sensitive reporter (soxS promoter-gfp). Darker shading designates strains that were experimentally measured to have increased levels of O₂⁻ compared to wildtype (p-value<0.05), whereas lighter shading designates strains that were experimentally measured to have levels of O₂⁻ that do not exceed those of wildtype. * denotes genes that are essential in the described media conditions, grey denotes genes that were not experimentally examined (for consistency between diagrams, these genes were also denoted by grey in 2A and 2C, though in silico predictions were computed).

FIGS. 3A-3F demonstrate evaluation of susceptibility to killing by oxidants. FIG. 3A shows a time course of predicted target strains and wildtype treated with H₂O₂. FIG. 3B shows a time course of negative control strains and wildtype treated with H₂O₂. FIG. 3C shows a time course of predicted target strains and wildtype treated with menadione. FIG. 3D shows a time course of negative control strains and wildtype treated with menadione. FIG. 3E shows a time course of predicted target strains and wildtype treated with NaOCl. FIG. 3F shows a time course of negative control strains and wildtype treated with NaOCl. Mean±SEM are shown for all plots.

FIGS. 4A-4F show an evaluation of susceptibility to killing by bactericidal antibiotics, and combination treatments with a chemical inhibitor. FIG. 4A shows a time course of predicted target strains and wildtype treated with ampicillin FIG. 4B shows a time course of negative control strains and wildtype treated with ampicillin FIG. 4C shows a time course of predicted target strains and wildtype treated with ofloxacin. FIG. 4D shows a time course of negative control strains and wildtype treated with ofloxacin. FIG. 4E shows a time course of wildtype cells treated with carboxin alone, H₂O₂ alone, a combination of carboxin and H₂O₂, or no treatment. FIG. 4F shows a time course of wildtype cells treated with carboxin alone, ampicillin alone, a combination of carboxin and ampicillin, or no treatment. Mean±SEM are shown for FIGS. 4A-4F.

FIGS. 5A-5B depict in silico predictions and experimental measures of H₂O₂ levels in genetic mutants using the HyPer protein system. FIG. 5A shows predicted relative H₂O₂ levels of various strains compared to wildtype. Darker shading designates strains whose mean H₂O₂ production levels were simulated to be >5% higher than wildtype over both ensembles, whereas lighter shading designates strains whose mean H₂O₂ production levels were simulated to be <5% higher than wildtype over both ensembles. FIG. 5B shows experimentally measured relative 500/420 fluorescene ratios of strains with the highly specific, H₂O₂-sensitive HyPer protein. Darker shading designates strains that were experimentally measured to have increased levels of H₂O₂ compared to wildtype (p-value<0.05), whereas lighter shading designates strains that were experimentally measured to have levels of H₂O₂ that do not exceed those of wildtype. * denotes genes that are essential in the described media conditions, grey denotes genes that were not experimentally examined (for consistency between diagrams, these genes were also denoted by grey in 5A, though in silico predictions were computed).

FIGS. 6A-6D demonstrate evaluation of susceptibility to killing by ciprofloxacin and gentamicin. FIG. 6A shows a time course of predicted target strains and wildtype treated with 15 ng/mL ciprofloxacin. FIG. 6B shows a time course of negative control strains and wildtype treated with 15 ng/mL ciprofloxacin. FIG. 6C shows a time of predicted target strains and wildtype treated with 500 ng/mL gentamicin. FIG. 6D shows a time course of negative control strains and wildtype treated with 500 ng/mL gentamicin. Mean±SEM are shown for FIGS. 6A-6D. We note that ΔatpC demonstrated increased sensitivity toward gentamicin, which, without wishing to be limited or bound by theory, may be the result of its positive impact on proton motive force²⁰ as well as its effect on basal ROS production.

FIGS. 7A-7D demonstrate evaluation of susceptibility to killing by the bacteriostatic drugs tetracycline and chloramphenicol. FIG. 7A shows a time course of predicted target strains and wildtype treated with 10 μg/mL tetracycline. FIG. 7B shows a time course of negative control strains and wildtype treated with 10 μg/mL tetracycline. FIG. 7C shows a time course of predicted target strains and wildtype treated with 15 μg/mL chloramphenicol. FIG. 7D shows a time course of negative control strains and wildtype treated with 15 μg/mL chloramphenicol. Mean±SEM are shown for FIGS. 7A-7D.

FIG. 8 depicts ampicillin and carboxin dose responses. Wild-type dose response of ampicillin and carboxin after 4 hours of treatment is shown. Each point shows the percent survival of wildtype treated with the respective levels of ampicillin and carboxin relative to the no-carboxin control.

### DETAILED DESCRIPTION

Provided herein are compositions and methods comprising ROS target modulators that increase ROS flux and endogenous ROS production, thereby potentiating oxidative attack by antibiotics and biocide. These ROS targets were identified, in part, using the systems-based, genome-scale ROS metabolic models and experimental validation, as described herein. The compositions, methods, and approaches described herein comprising ROS target modulators provide efficient means of improving treatment of bacterial infections and inhibiting bacterial replication and growth, by providing novel means of increasing efficacy and potency of known antibiotic agents, such as, for example, β-lactams and fluoroquinolones. By increasing efficacy and potency of known antibiotic agents, the compositions and methods comprising ROS target modulators also permit lower dosages of antibiotic agents to be used with increased efficacy.

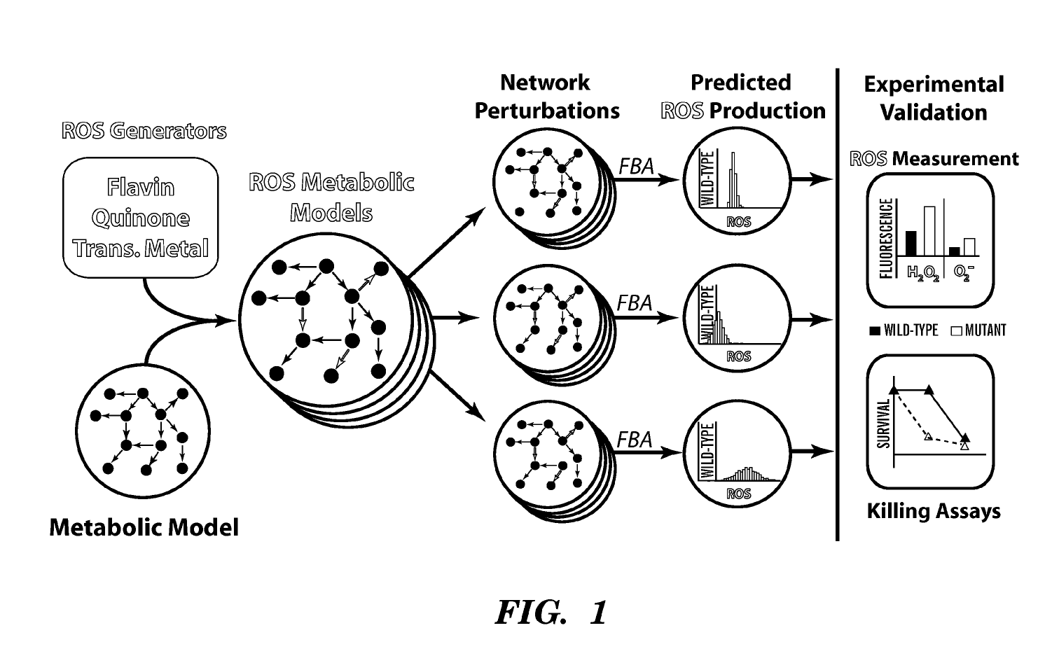

Reactive oxygen species (ROS) can damage DNA, RNA, proteins, and lipids, and cell death occurs when the level of ROS exceeds an organism's detoxification and repair capabilities^{7,8}. Despite this danger, aerobically growing bacteria endogenously generate ROS as a metabolic by-product, a risk balanced by an increased efficiency and yield of energy from growth substrates. Without wishing to be bound or limited by theory, at least two possible mechanisms exist to manipulate bacterial ROS metabolism and achieve increased sensitivity to oxidative attack: (1) amplification of endogenous ROS production, and (2) impairment of detoxification and repair systems. Whereas removal of detoxification and repair systems have been shown to increase susceptibility to oxidants^{8,9}, antibiotics^{10,11} and immunityl²⁻¹⁴, manipulation of endogenous ROS production remains largely unexplored. Endogenous ROS production has long been appreciated as a factor influencing the ability of an organism to survive oxidative stress¹⁵⁻¹⁷, but an inability to predict the outcome of genetic and environmental perturbations on ROS production¹⁸ has prevented exploration and exploitation of this phenomenon as an antimicrobial adjuvant. This inability derives from a limited systems-level understanding of a potentially, expansive and highly-integrated biochemical reaction network. Accordingly, in the studies described herein, we rationally tuned E. coli metabolism for increased ROS production (specifically, O₂⁻ and H₂O₂) to determine whether these effects can potentiate oxidative stress and antibacterial activity. Importantly, as described herein, the goals were to not overwhelm the oxidative detoxification and repair capabilities of E. coli with endogenously generated ROS, but rather to increase endogenous production such that the ability of E. coli to cope with exogenous oxidative stress would be compromised. Such a strategy would broadly potentiate antimicrobials that harness oxidative stress and provide a general approach for the discovery of antimicrobial adjuvants. To achieve this goal, in part, we developed an ensemble, genome-scale modeling approach that can quantitatively estimate ROS production from E. coli metabolism as described herein (FIG. 1).

Prior to the discoveries described herein, the sources for the majority of endogenous ROS produced by E. coli remain undefined¹⁸. The removal of enzymes that generate ROS in vitro has had seemingly little effect on whole-cell ROS production¹⁸. Previous studies have demonstrated that O₂⁻ and H₂O₂ can be produced when O₂ abstracts electrons from reduced flavin, quinol, and transition metal functional groups^{7, 17, 19-21}. Inspection of E. coli metabolism for enzymes that use these electron carriers identified 133 reactions, spanning many metabolic pathways, with the potential to generate ROS in the presence of O₂ (Table 1). The number of potential ROS generating reactions determined using the methods described herein is of comparable size to the number of reactions that generate ATP/ADP, NAD/H, and NADP/H, indicating that ROS could play a crucial, highly integrated role in bacterial metabolism. To rationally modify the production of such highly connected metabolites, a quantitative systems-level approach was required, as even removal of enzymes that endogenously produce ROS can increase or decrease production, depending on the redistribution of metabolic flux on the remaining ROS-generating enzymes¹⁸.

Systems-level metabolic modeling has been used extensively to optimize the production of desirable metabolites, and has significantly impacted the fields of biotechnology, metabolic discovery, and microbiology²²⁻²⁶. As demonstrated in the studies described herein, we employed flux balance analysis (FBA) with genome-scale metabolic models (GSMM) to simulate systems-level ROS production. In FBA, reaction stoichiometries are used to define a metabolic solution space, and linear programming identifies a flux distribution within that space that optimizes an objective function, such as biomass generation^{27,28}. Accuracy within the stoichiometric reaction network is critical to the performance of constraint-based techniques^{29,30}. Current metabolic reconstructions include consumption reactions, such as superoxide dismutase and catalase, and generation reactions involved in cofactor biosynthesis and alternate carbon metabolism, but are devoid of generation reactions that account for the majority of ROS produced³¹. To construct a metabolic model capable of estimating ROS production, we added 266 additional ROS production reactions to the E. coli GSMM³², and one O₂⁻ and one H₂O₂ producing reaction for each of the 133 potential sources (S Table 1). These potential ROS sources included all enzymes known to generate H₂O₂ and O₂⁻ in E. coli^{17,18, 21, 31, 33-35}, and this framework allowed separate (independent species balances) but simultaneous modeling of H₂O₂ and O₂⁻ production in E. coli, as described herein.

Optimization of an objective function is a critical feature of constraint-based techniques, and maximizing for biomass generation has proven to be effective in predicting redistribution of metabolic flux³⁶. However, when presented with competing pathways, constraint-based methods can be used to identify the most efficient pathway in terms of cellular resources as the one that carries flux. ROS-generating reactions are less efficient competing pathways where reducing equivalents are lost to O₂ instead of being transferred to the intended acceptor. Therefore, addition of ROS-generating reactions to the GSMM is necessary to model ROS metabolism, but insufficient since the reactions will not carry flux.

To address these constraints, we recognized that ROS-generating reactions are coupled to their more efficient counterpart, in the sense that initial electron transfer from reactant to electron carrier proceeds normally and is dictated by requirements for the intended products, and that it is the promiscuity of the reduced electron carrier with O₂ that generates ROS. Thus, ROS flux is a function of the number of electrons transferred to the electron carrier, and consequently dependent on the reaction flux of the intended reaction. Accordingly, in the studies described herein, the flux of O₂⁻ and H₂O₂ from ROS-generating enzyme, was assumed to be proportional to the reaction flux, vᵢ. This results in proportionality between ROS flux from enzyme, and the number of electrons transferred by enzymeᵢ, and is accomplished by coupling the intended enzyme reaction to both its O₂⁻ and H₂O₂ side-reactions. This coupling requires specification of the proportion of electrons that flow to O₂ to form O₂⁻ and H₂O₂ for each of the 133 potential ROS sources. These values vary significantly from enzyme to enzyme^{7,20}, and are largely undefined due to the absence of in vivo measurements. Keeping this indeterminacy in mind, we employed ensemble approaches, as described herein.

Two ensembles of ROS-GSMMs were constructed, each with 1,000 different models. The proportions of electron flow from reaction, to generate O₂⁻ and H₂O₂ were captured by the constants c_{i,O2}⁻ and c_{i, H2O2}. One ensemble derived these constants from a Gaussian distribution in order to model a distributed ROS production network (many significant generators), while the other ensemble derived these constants from an exponential distribution to model a centralized ROS production network (few significant generators). Further, it was specified that ROS could only be produced from these reactions and not consumed, with the exception of O₂⁻ attack of Fe—S centers, and that the in silico O₂⁻ and H₂O₂ production rates of the wildtype GSMM had to match the best available experimental estimates^{16,37}. Thus, every stoichiometric reaction network within the ensembles had the exact same production rate of O₂⁻ and H₂O₂ for its wildtype GSMM. Also, as described herein, the existence of alternative optimal solutions for ROS production of each wildtype network was examined using flux varability analysis (FVA)³⁸. At a biomass production rate of 100%, all wildtype networks generate a unique solution for the flux of H₂O₂ and O₂⁻

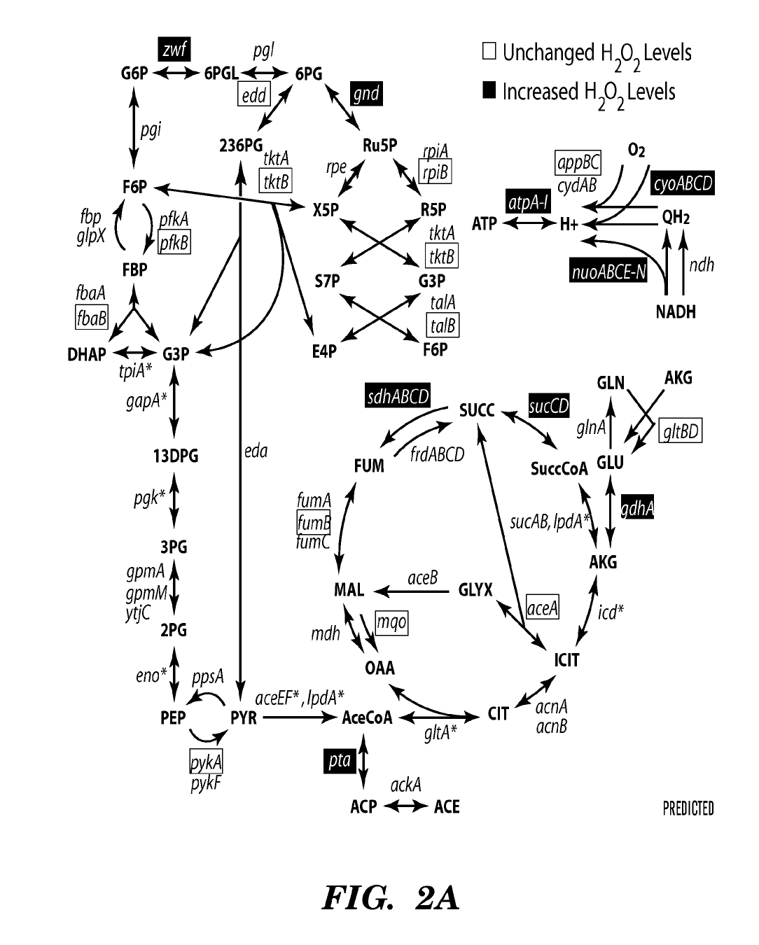

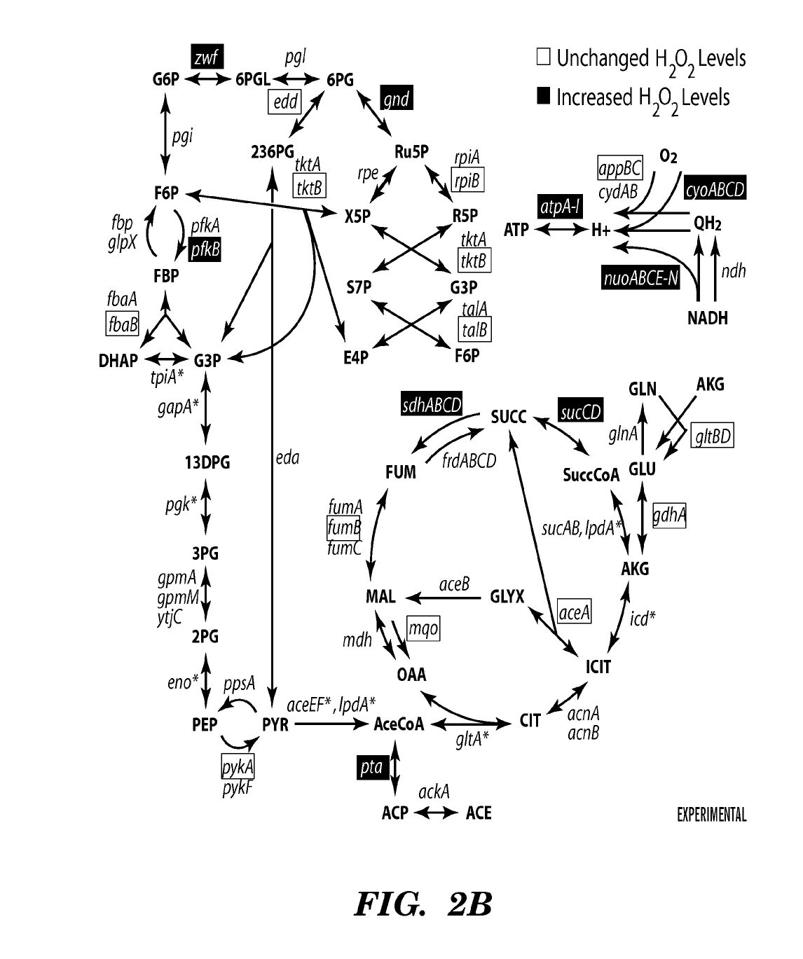

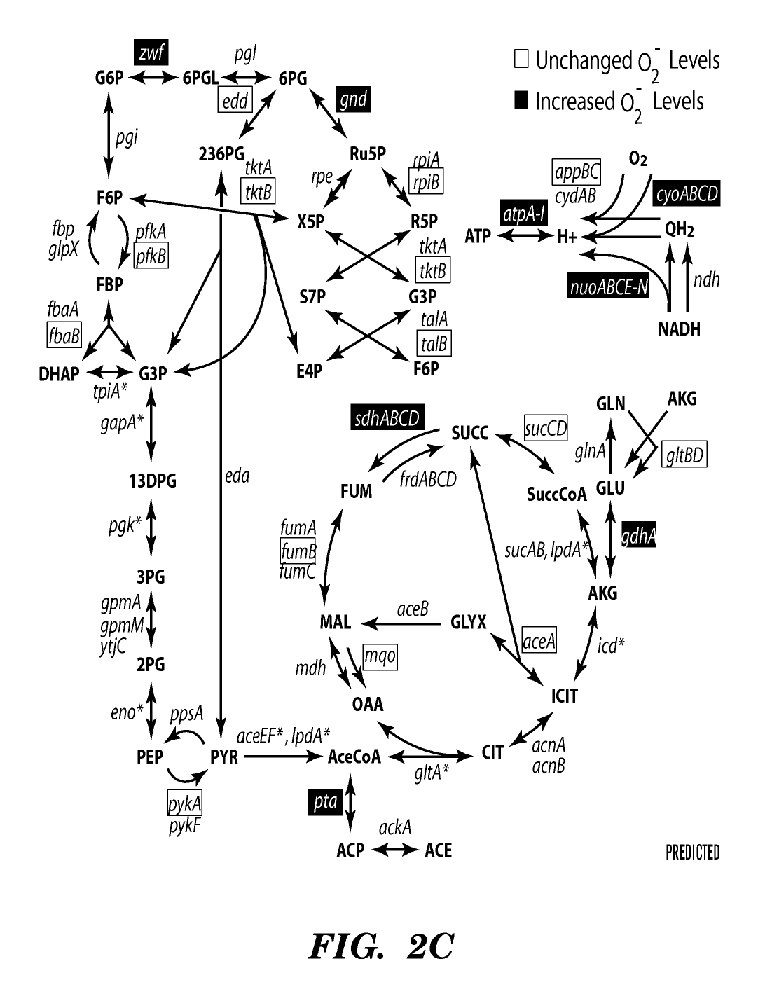

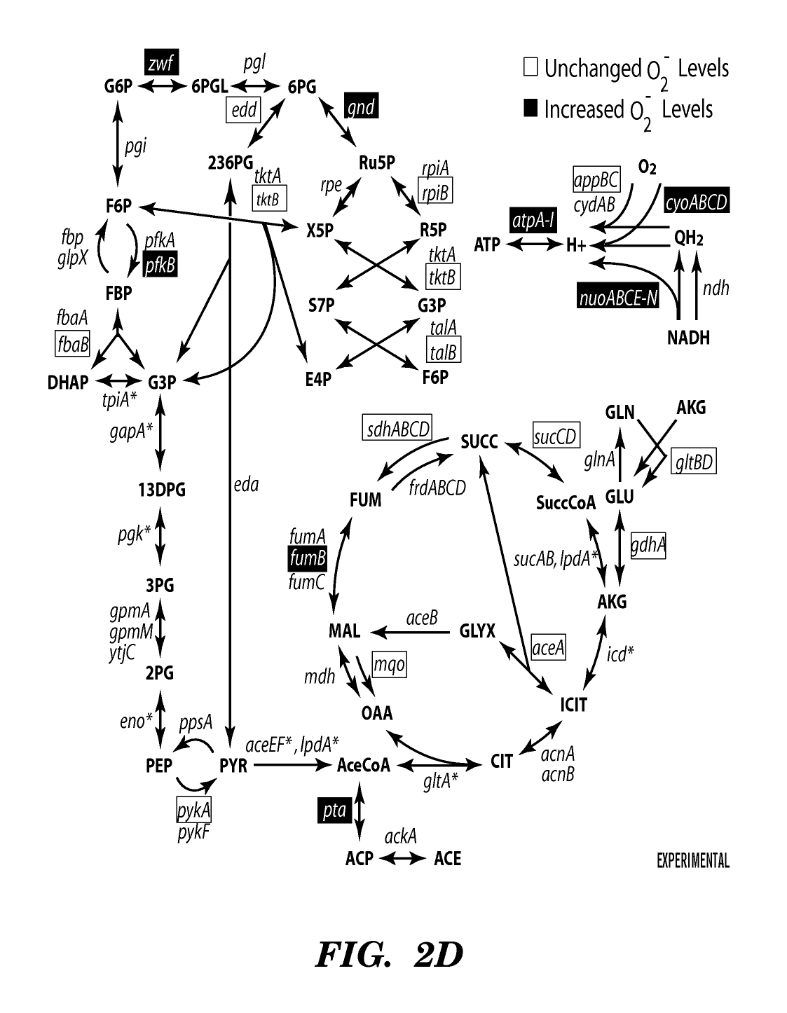

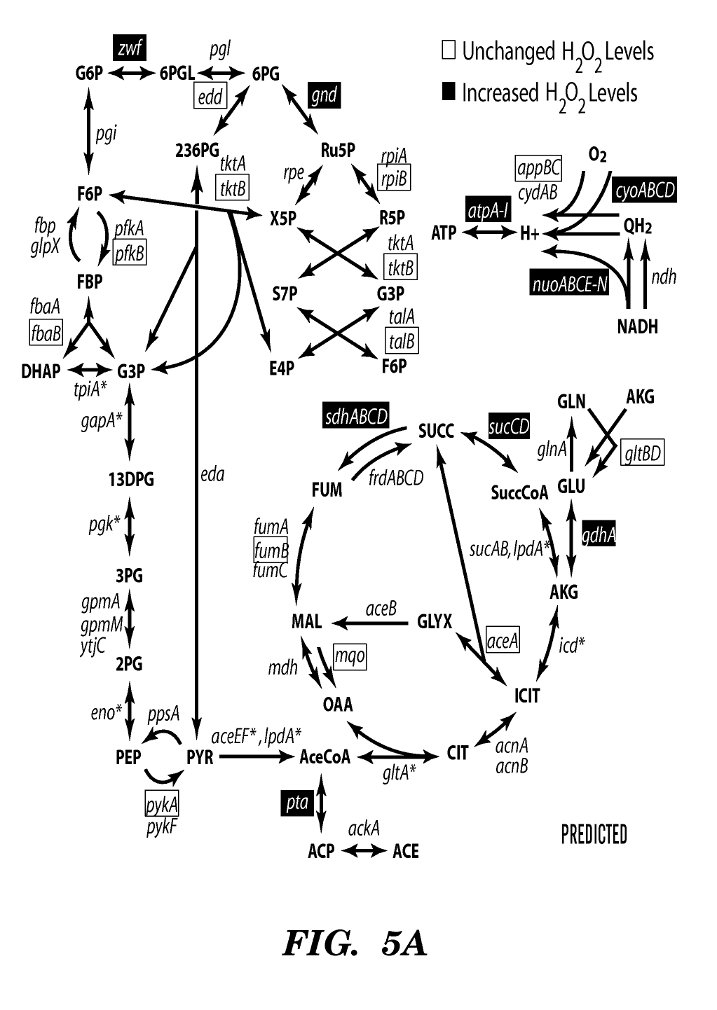

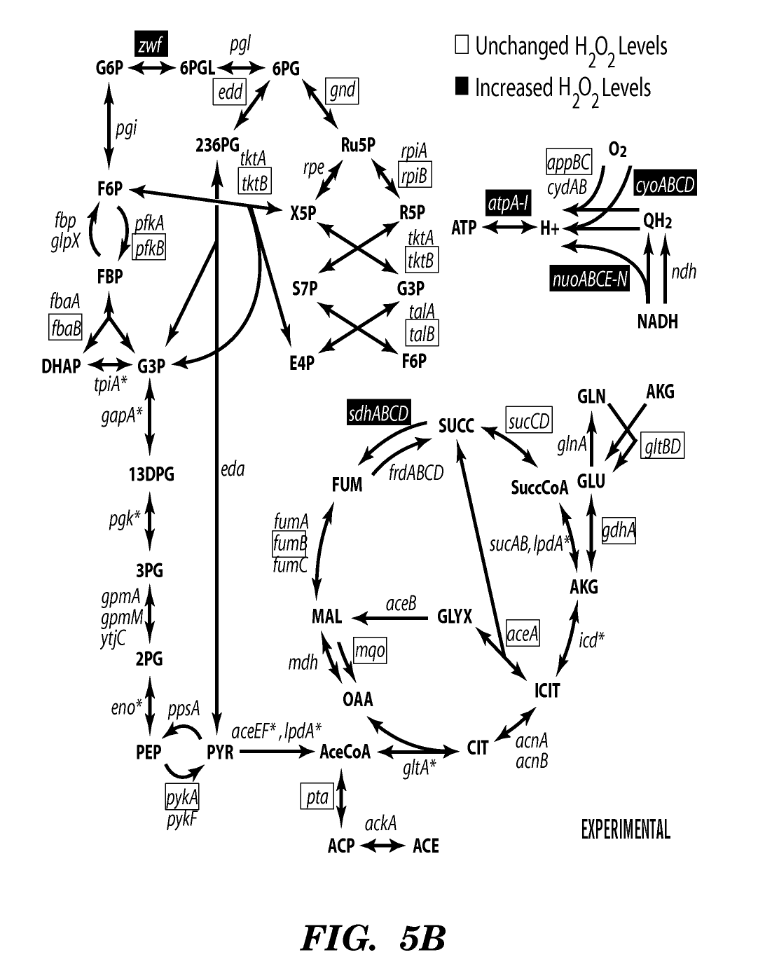

Using these ensembles, perturbations to the metabolic network alter basal ROS production were explored in silico. As described herein, we performed a systematic gene deletion analysis in which genes were removed one at a time and reaction fluxes recalculated, while optimizing for biomass. These analyses provided quantitative distributions of ROS production (O₂⁻ and H₂O₂) from mutant E. coli (FIG. 1), and allowed identification of deletions likely to alter basal ROS production (FIGS. 2A, 2C, 5A). To account for variable growth rates of mutant strains, ROS flux was normalized by biomass production (BM), and calculations are therefore H₂O₂/BM and O₂⁻/BM (mmol/gDW produced). From analyses of aerobic glucose minimal media, the gene deletions identified as being most likely to increase ROS production encoded for ATP synthase (atpA-I), pyruvate dehydrogenase (aceEF, lpd), NADH dehydrogenase complex I (nuoABCE-N), glutamate dehydrogenase (gdhA), cytochrome bo (cyoABCD), and triose phosphate isomerase (tpiA). Investigation of the flux distributions for these mutants identified a general trend for ROS production where predicted increases correlated with inefficiencies in the production or usage of ATP.

Accordingly, provided herein in some aspects, are ROS targets and inhibitors of such targets, termed herein as “ROS target modulators” or “ROS target inhibitors” that impact basal ROS production, and compositions and methods of their use thereof, identified, in part, by the in silico perturbation results and experimental validation studies described herein.

To validate the approaches described herein and in silico analyses, a series of gene deletions that encode enzymes within glycolysis, the pentose-phosphate pathway, EntnerDoudoroff pathway, TCA cycle, glyoxylate shunt, aerobic respiration, acetate metabolism, and glutamate metabolism were experimentally tested (FIGS. 2B, 2D, 5B). This collection of enzymes included those predicted, using the methods described herein, to increase ROS (i.e., ROS targets) as well as those predicted, using the methods described herein, to leave ROS production unchanged (negative controls). The predictions included all twenty-one genetic mutants tested, both targets and negative controls, and that only those enzymes that were experimentally tested are shaded in FIGS. 2A-2D, 5A-5B. Isozymes selected for testing were based on literature evidence that indicated their removal would most closely reflect model assumptions³⁹, and deletions of pyruvate dehydrogenase and triose phosphate isomerase were not tested due to an inability to grow in minimal glucose media⁴⁰⁻⁴⁵.

To measure O₂⁻, a SoxR-controlled GFP-reporter system was employed, whereas to measure H₂O₂, an OxyR-controlled GFP-reporter system and the direct-sensing HyPer protein were both used⁴⁷. The experimental results provided herein show between 80-90% qualitative agreement with the in silico predictions of H₂O₂ and O₂⁻ production (FIGS. 2A-2D, 5A-5B, Correct predictions: dps-GFP: 19/21, soxS-GFP: 17/21, HyPer: 17/21). The probabilities that these levels of agreement would have occurred by chance, using the null hypothesis that random segregation of 21 genes into targets and negative controls would match experimental results as well as predictions from our modeling approach, are 3.7×10⁻⁴ (dpsGFP), 1.0×10⁻² (soxS-GFP), and 6.2×10⁻³ (HyPer). Accordingly, the experimental results described herein indicate that our systems-level, ensemble approach enables predictable tuning of ROS production in E. coli.

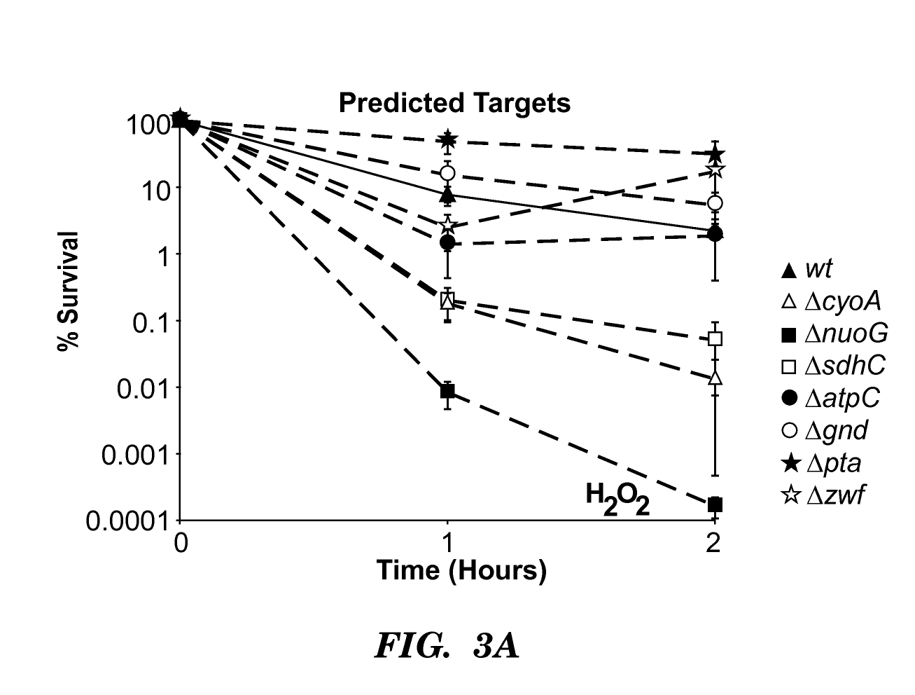

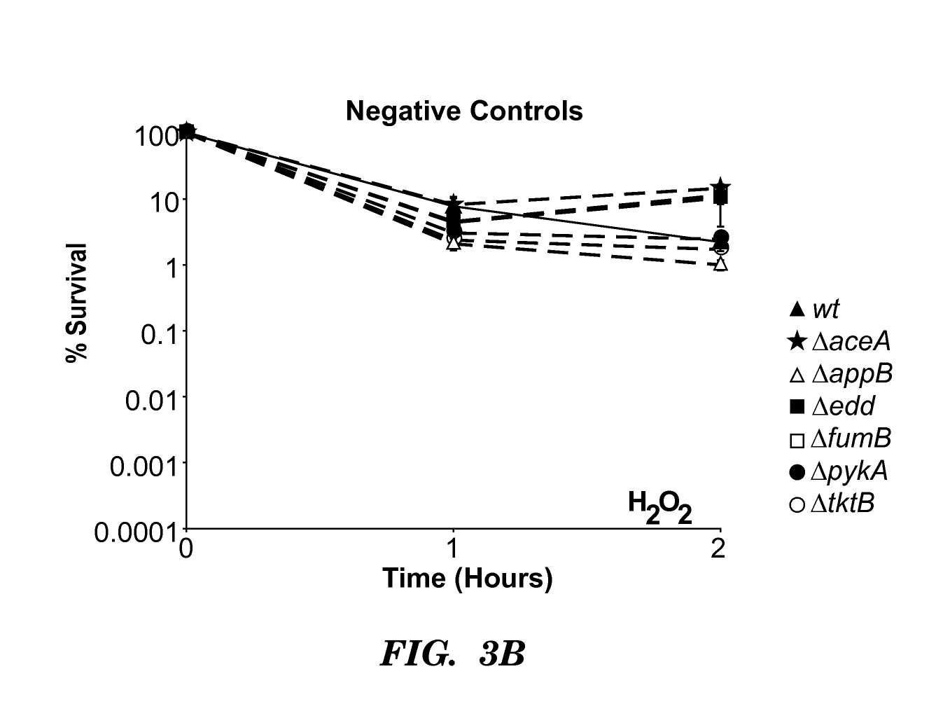

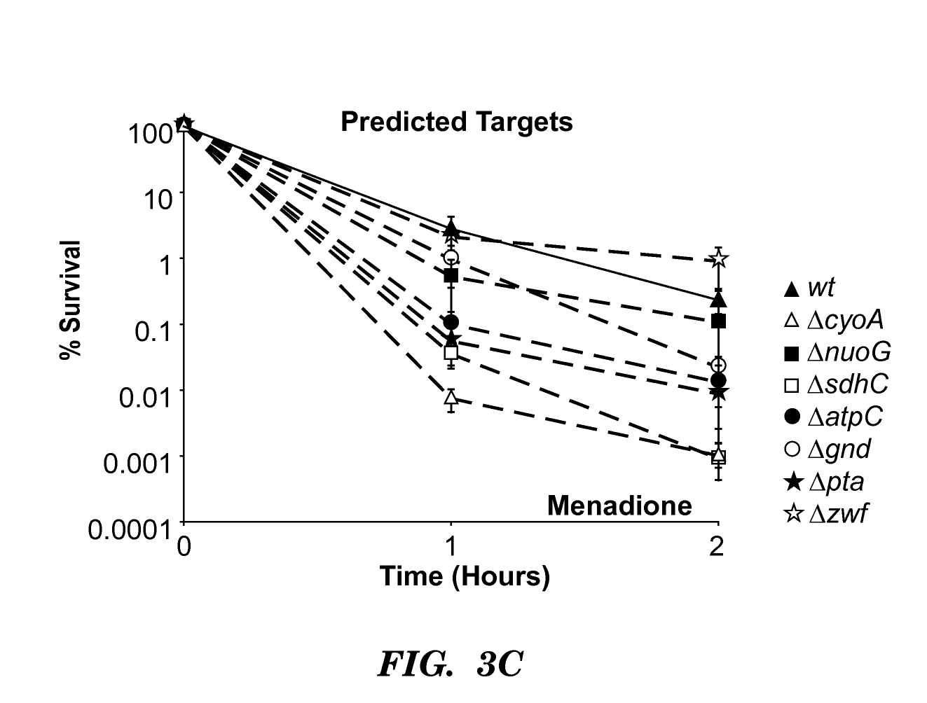

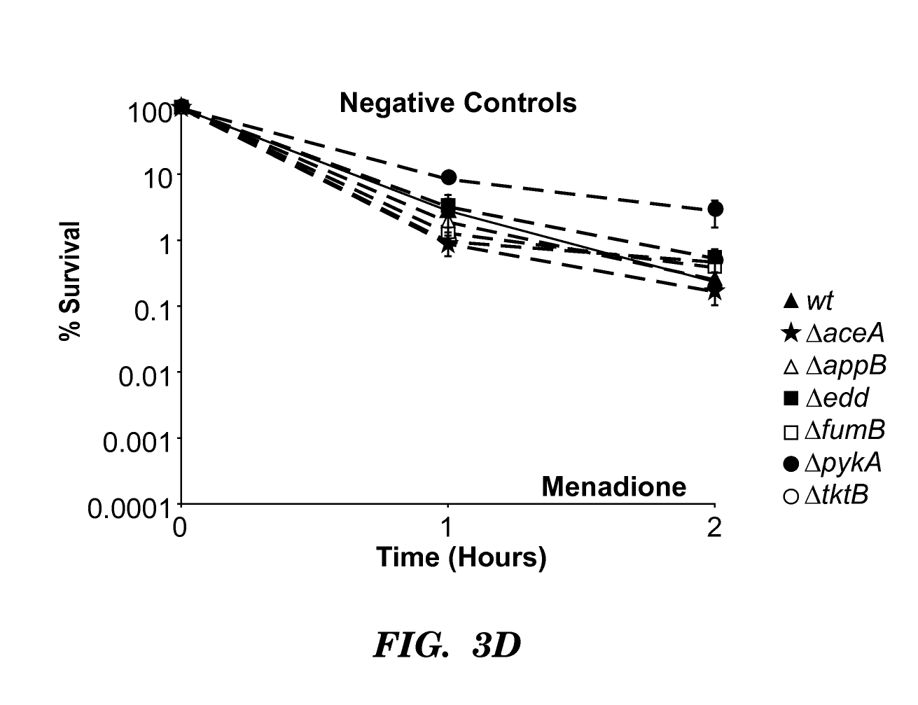

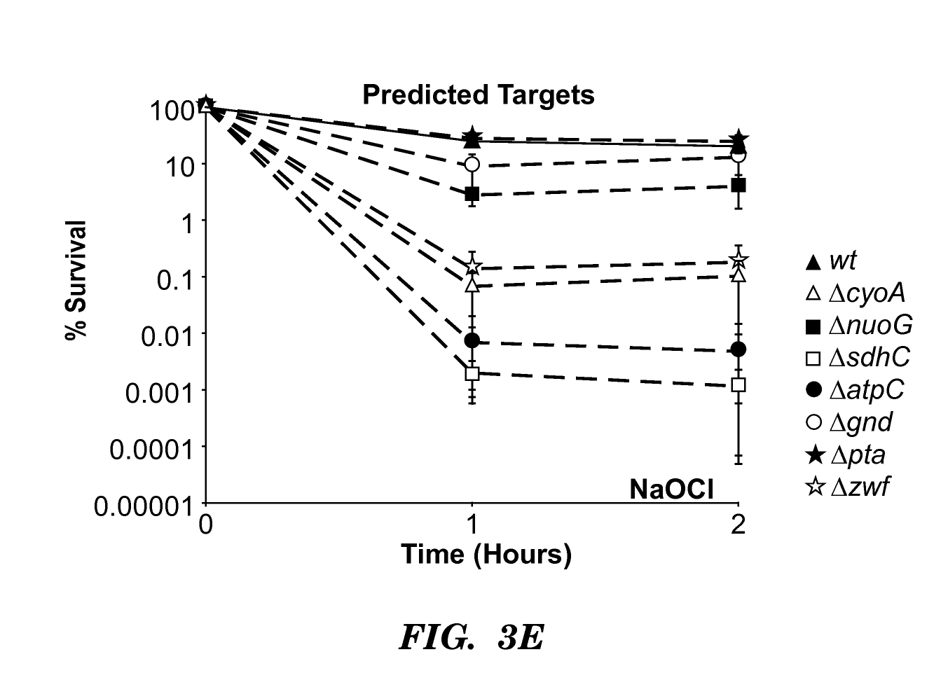

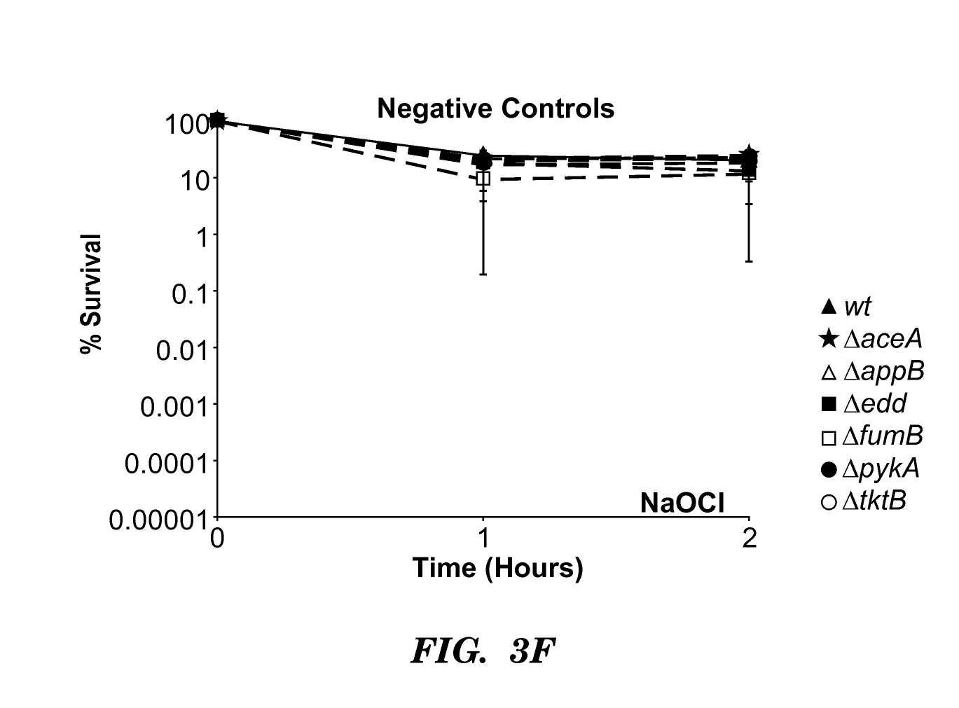

It was next determined whether increased basal production of O₂⁻ and/or H₂O₂ can translate into increased killing by oxidants. The oxidants tested were O₂⁻ (generated via menadione), H₂O₂, and NaOCl (bleach). O₂⁻ and H₂O₂ were chosen due to their inclusion in the model and importance for antibiotic action⁶, and NaOCl was chosen due to its use as a biocide. Strains chosen for testing of oxidant sensitivity were those with in silico predictions that were confirmed by experimental results (FIGS. 2A-2D). The experimental results indicate that increased basal production of O₂⁻ or H₂O₂ generally increases microbial susceptibility to oxidative attack (FIGS. 3A-3F). Genetic deletions that increase ROS production exhibited increased susceptibility to oxidants, while negative control strains that exhibited wildtype production levels of ROS did not. The probability this enrichment would have been observed by random selection is 2.5×10⁻⁵, and demonstrates that increased production of O₂⁻ and H₂O₂ can increase killing by oxidants. Some predictions conferred increased susceptibility to all oxidants tested (ΔcyoA and ΔsdhC), whereas others exhibited selective increases in sensitivity (e.g., Δzwf), indicating that sensitivity to one oxidant does not always translate to other oxidants. This, without wishing to be bound or limited by theory, likely derives from the differences in biochemical activity of the oxidants and the distinct cellular death pathways they induce^{7, 48, 49}. Accordingly, the results described herein clearly demonstrate that increasing endogenous production is a robust strategy to enhance the susceptibility of microbes to oxidative stress, and provide novel targets and inhibitors thereof for increasing susceptibility to oxidants.

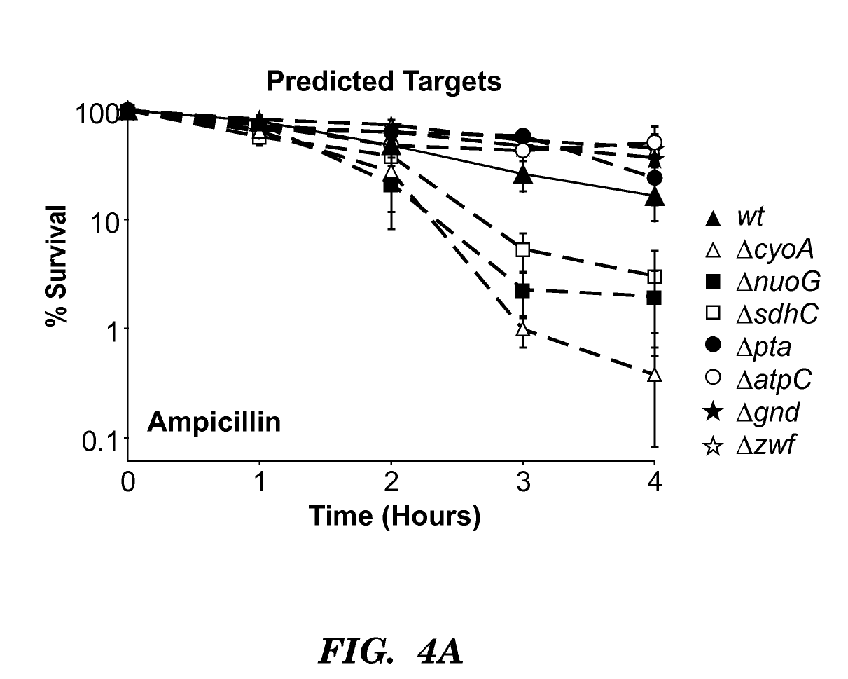

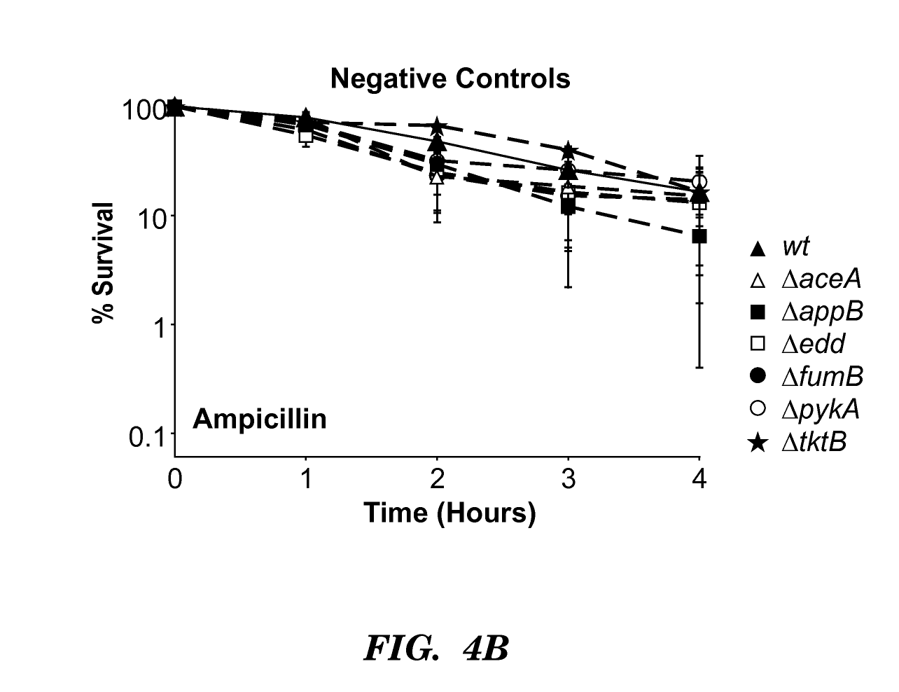

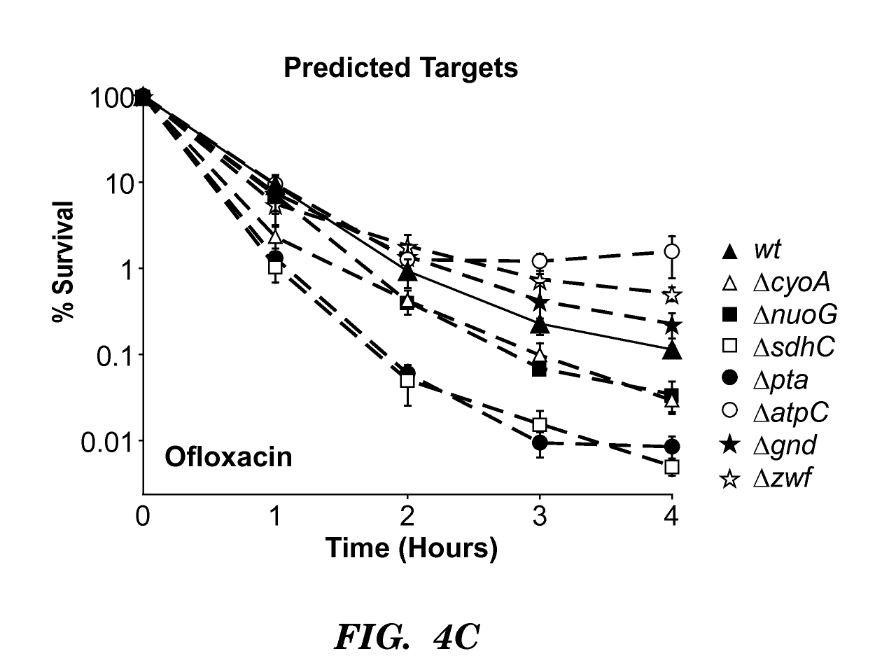

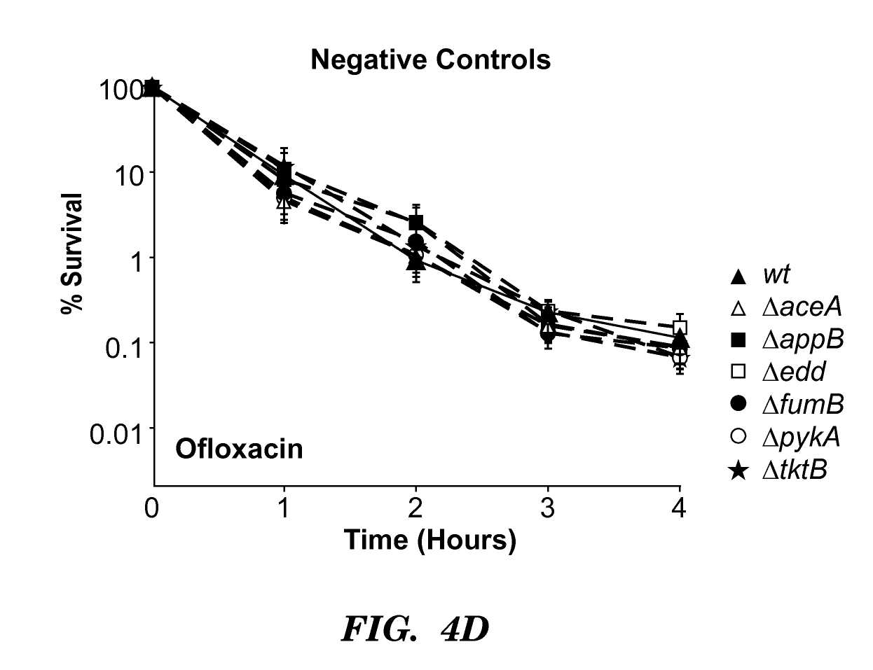

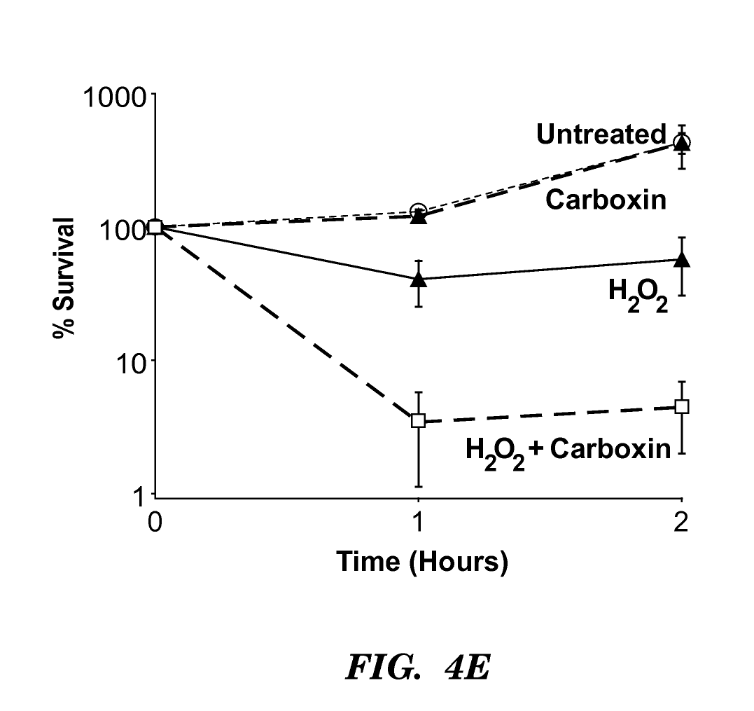

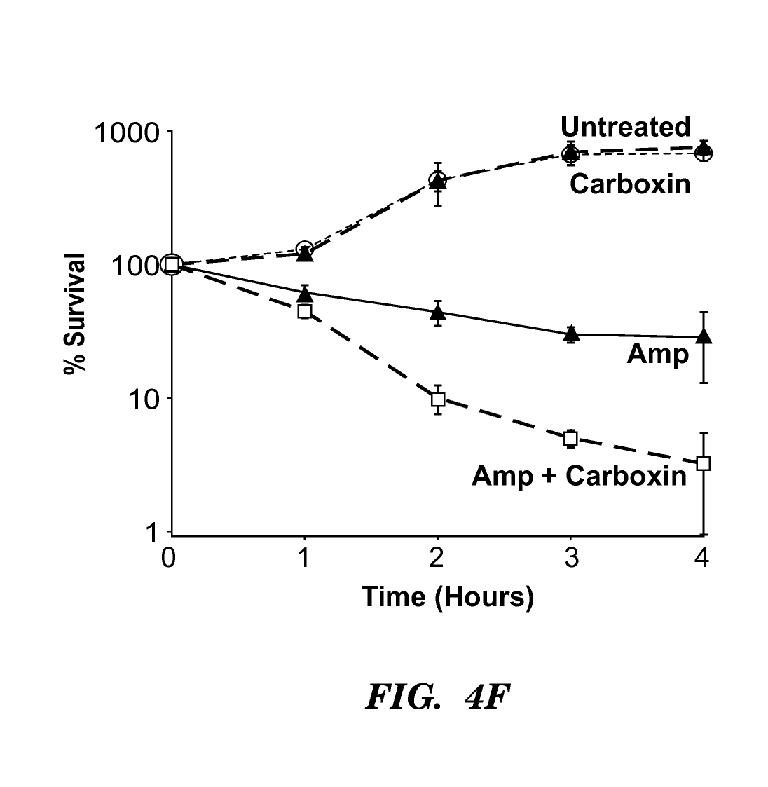

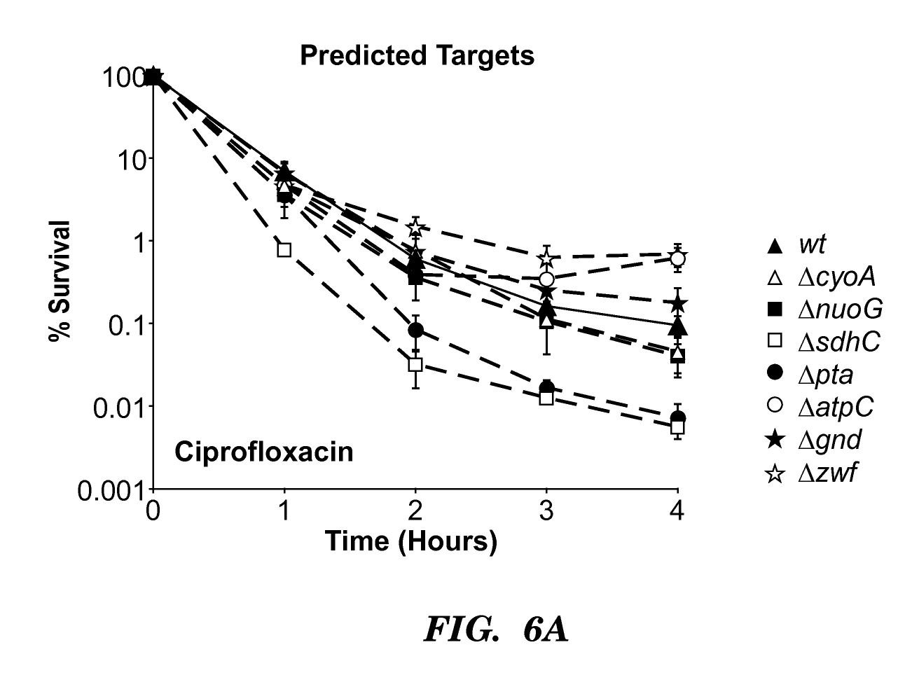

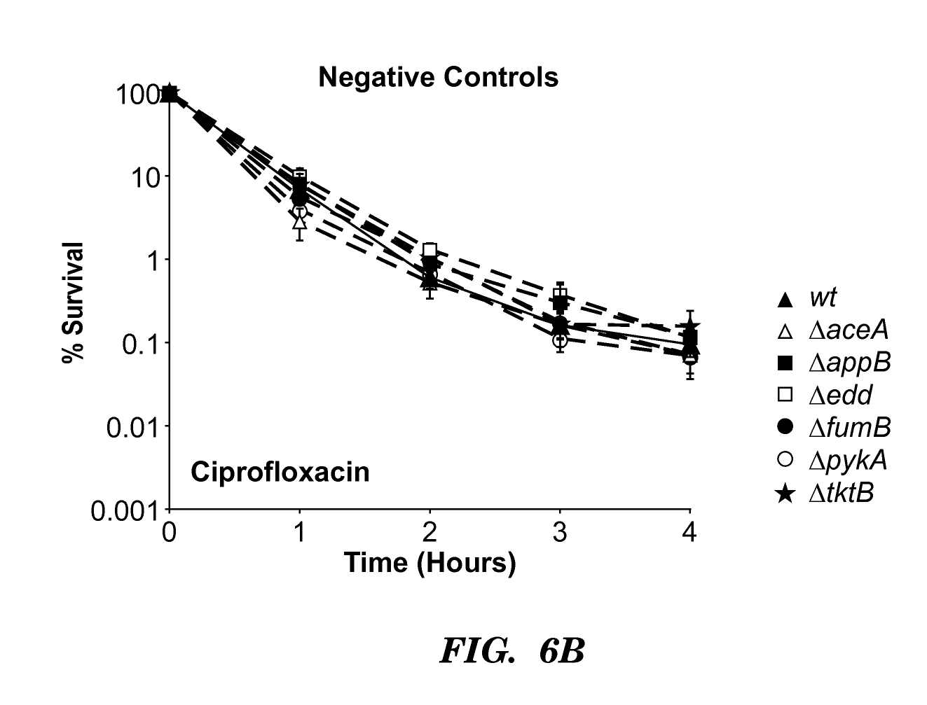

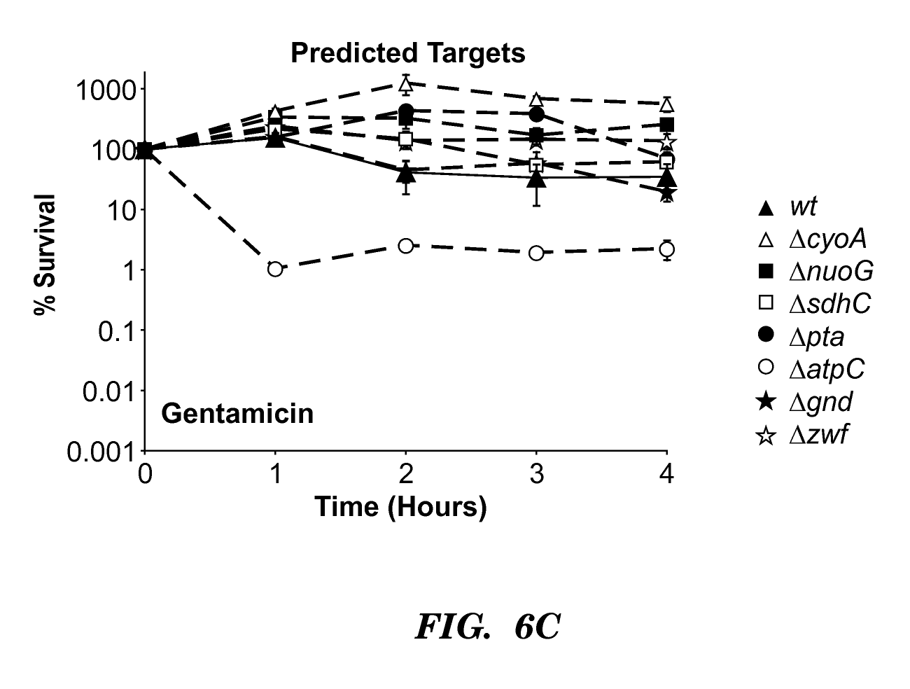

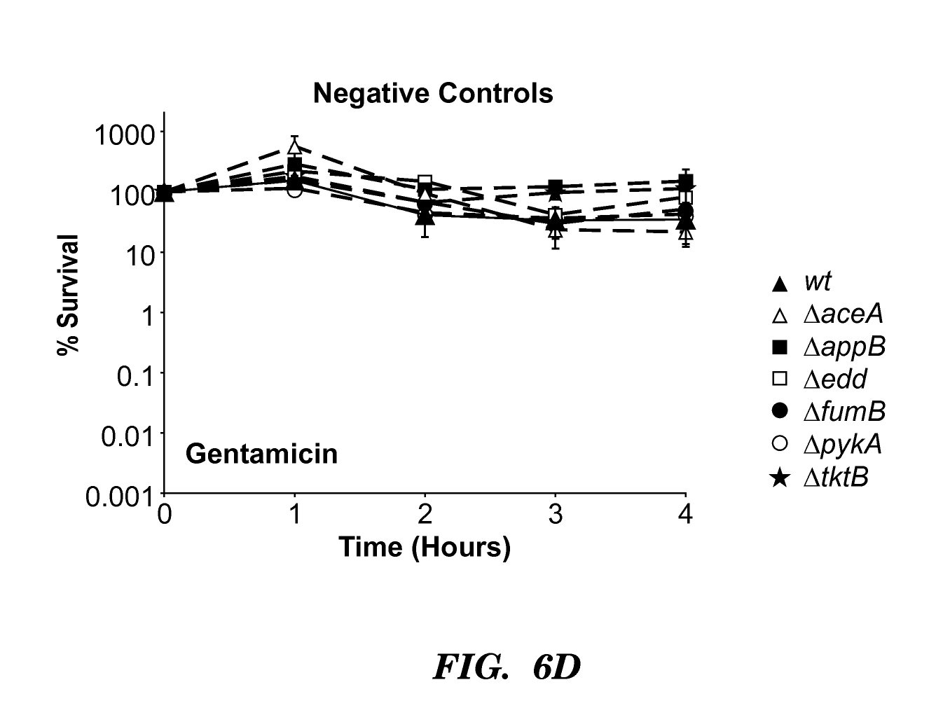

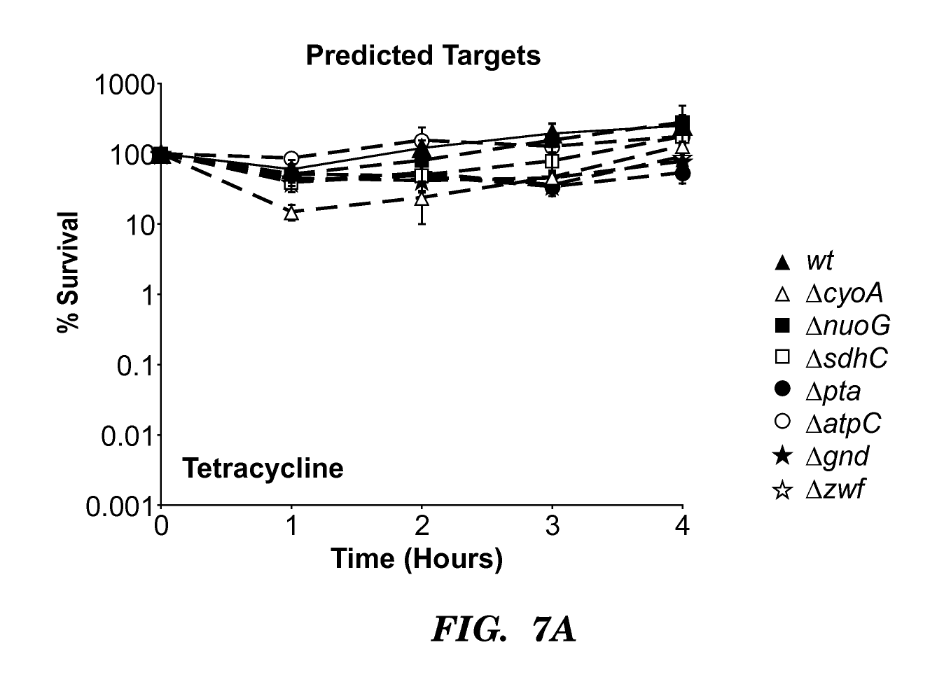

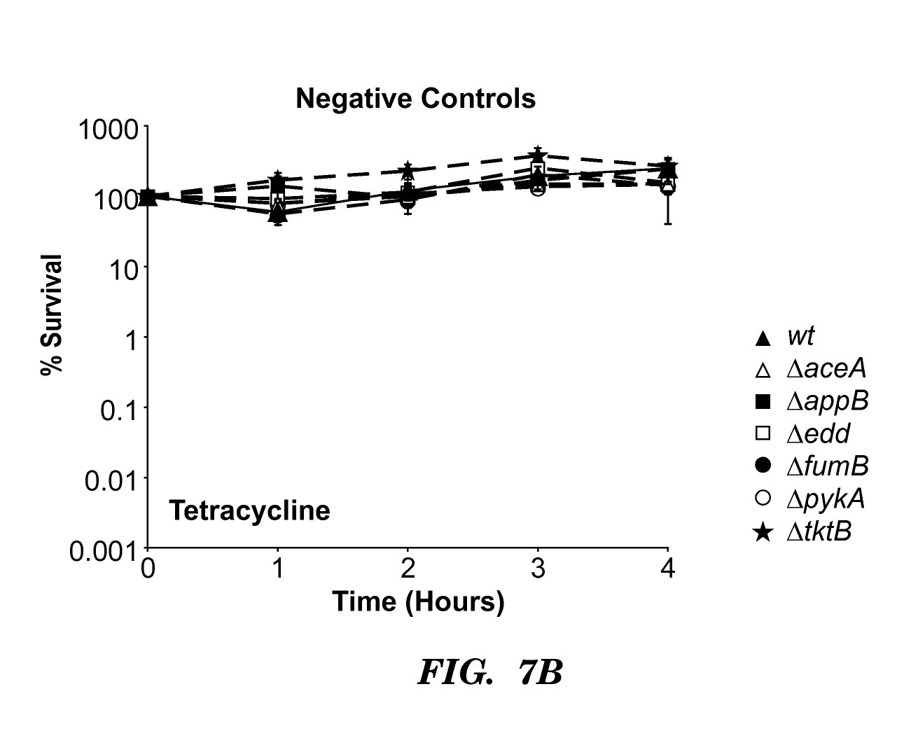

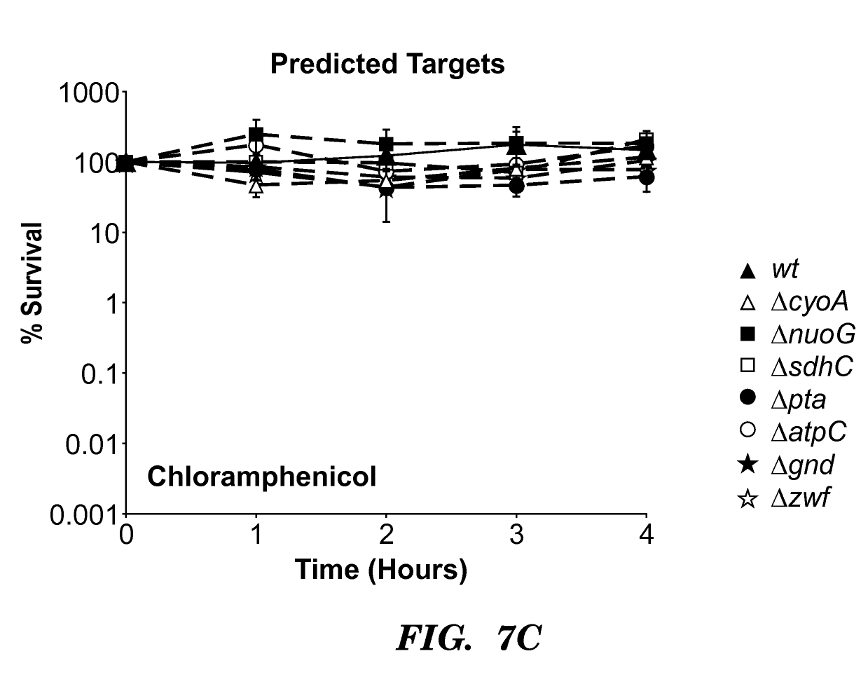

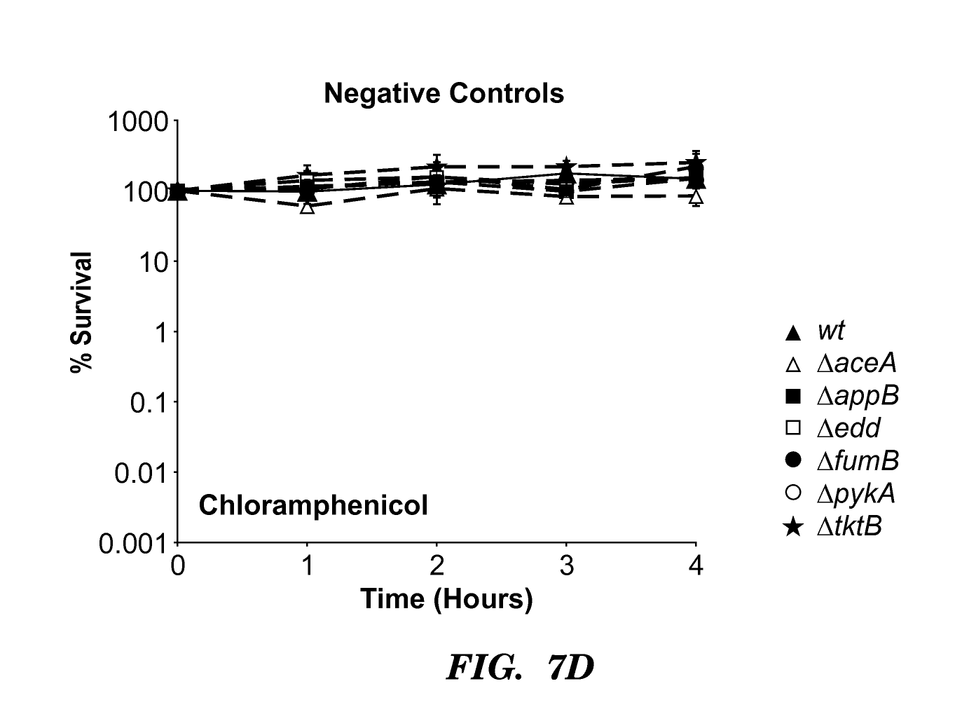

Bactericidal antibiotics have been shown to share a common mechanism of cell death that involves the production of ROS^{6,11}. It was next determined whether increased basal production of ROS could potentiate the action of bactericidal antibiotics (β-lactams: e.g., ampicillin, fluoroquinolones: e.g., ofloxacin, ciprofloxacin, aminoglycosides: gentamicin). Three of the validated targets identified herein (ΔcyoA, ΔnuoG, ΔsdhC) exhibited increased sensitivity to both β-lactam and fluoroquinolone antibiotics (FIGS. 4A, 4C 6A) and one of the targets (Δpta) exhibited increased sensitivity to only fluoroquinolones (FIGS. 4A, 6A), whereas all of the negative control strains displayed wildtype sensitivity to both antibiotic classes (FIGS. 4B, 4D, 6B). Accordingly, these results demonstrate a correct prediction rate for our approach of greater than 70% for both β-lactams and fluoroquinolone antibiotics. Sensitivity to aminoglycosides was also tested, though increased killing, in general, was, without wising to be bound or limited by theory, expected not to be observed. This expectation was, without wising to be bound or limited by theory, based on the fact that many of the gene deletions that increase basal ROS production negatively impact proton motive force, which is important for aminoglycoside uptake⁵⁰. As expected, the negative controls had similar sensitivity to gentamicin as wildtype, whereas many targets exhibited decreased sensitivity (FIGS. 6C, 6D). Further, ΔatpC demonstrated increased sensitivity toward gentamicin, which can be the result of its positive impact on proton motive force⁵¹ as well as its effect on basal ROS production. Accordingly, the data described herein indicate that bactericidal antibiotic primary target interactions must be enabled (e.g., via antibiotic uptake) in order to leverage ROS production as an adjuvant therapy. As also demonstrated herein, the activities of bacteriostatic antibiotics that do not produce ROS⁶, are unaffected by increases in basal ROS production (FIGS. 7A-7D).

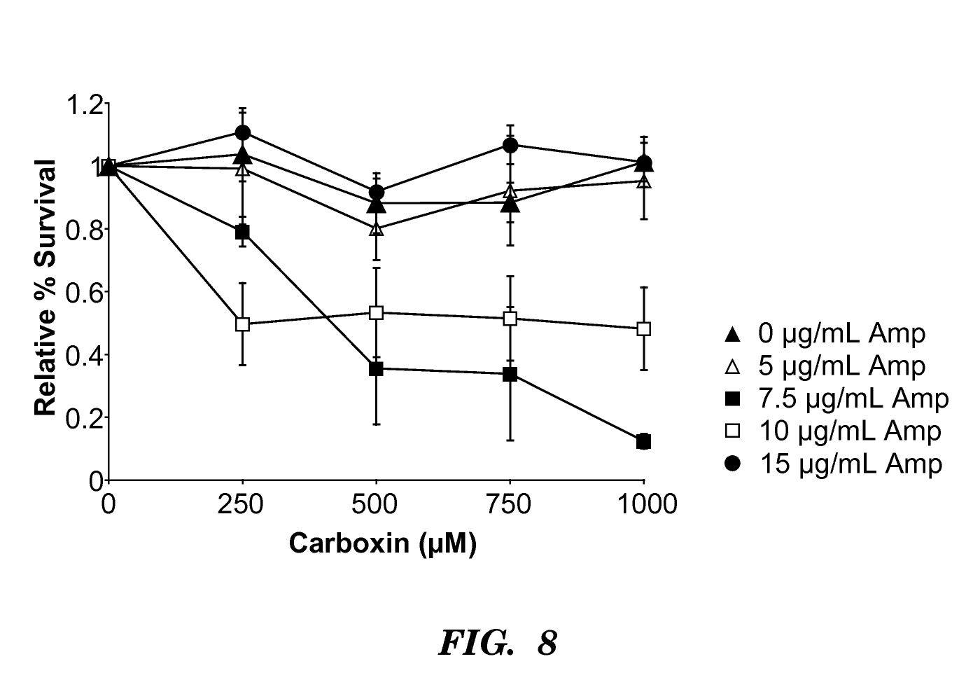

It was next demonstrated that chemical inhibition of one of the targets predicted using the methods described herein was sufficient to increase sensitivity to oxidants and bactericidal antibiotic treatment. To do so, wildtype cells were treated with carboxin, an inhibitor of succinate dehydrogenase³⁹, and susceptibility measured toward H₂O₂ and ampicillin, respectively. Addition of carboxin alone had no effect on the growth of wildtype cells (FIGS. 4E, 4F). However, wildtype cells treated with H₂O₂ and carboxin demonstrated increased sensitivity compared to wildtype cells treated with H₂O₂ alone (FIG. 4E). Similarly, wildtype cells treated with ampicillin and carboxin were significantly more sensitive to the antibiotic than cells treated with ampicillin alone (FIG. 4F). To more fully examine this synergy, a systematic drug screen was conducted spanning five concentrations for each compound (carboxin and ampicillin), including the untreated sample. This allowed us to calculate that carboxin concentrations of 250 μM or greater are synergistic with ampicillin concentrations between 7.5-10 μg/mL, using the Bliss Independence and Highest Single Agent models of drug synergism^{52, 53} (FIG. 8). Accordingly, these results demonstrate that chemical inhibition of a predicted and validated target (e.g., succinate dehydrogenase) is sufficient to increase sensitivity to oxidative attack and antibiotic treatment. While carboxin itself is not suitable as an antibiotic adjuvant due to human toxicity issues⁵⁴, validation that chemical inhibition of succinate dehydrogenase confers similar sensitivity as genetic perturbation allows translation of this finding to chemical library screening where non-toxic inhibitors of bacterial succinate dehydrogenase and other predicted targets may be found. Multiple studies have successfully screened chemical libraries for compounds with antimicrobial properties against pathogenic bacteria^{55, 56}, and the methods described herein can be used to complement this work by identifying novel enzyme targets for compounds that may have no antimicrobial properties alone, but which enhance the killing efficacy of current antibacterial agents.

Accordingly, provided herein, in some aspects, are systems-based methods to predictably tune microbial ROS production. By developing genome-scale ROS metabolic models, redistribution of ROS flux resulting from network perturbations was able to be predicted, and it was demonstrated experimentally that increased ROS flux can potentiate oxidative attack from antibiotic and biocide treatment. Accordingly, the approaches described herein allow rapid identification of antibacterial adjuvant targets, and are translatable to other pathogens of interest, such as, for example, Mycobacterium tuberculosis, Staphylococcus aureus, Haemophilus influenzae, and Salmonella typhimurium, where metabolic reconstructions are available⁵⁷⁻⁶¹.

### ROS Target Modulator Compositions and Methods Thereof

As described herein, using the systems-based, genome-scale ROS metabolic models described herein and experimental validation, novel biochemical targets have been identified that potentiate oxidative attack by antibiotics and biocide by increasing ROS flux and endogenous ROS production. Accordingly, provided herein, in some aspects, are compositions, including therapeutic compositions and combinations, comprising an effective amount of a ROS target modulator, and methods of preventing or treating bacterial infection with the same.

The terms “ROS target modulator,” or “modulator of a ROS target,” as used herein, refer to an agent or compound that causes or facilitates a qualitative or quantitative increase in or stimulates increases in production of basal reactive oxygen species (ROS) in cells. By increasing endogenous ROS production in bacterial cells, as described herein, the inventors have discovered that this increases or potentiates the activity or action of bactericidal antibiotics and increases sensitivity to oxidants, and consequent killing of bacteria. Accordingly, a ROS target modulator agent can, in some embodiments, be considered an adjuvant of an antibiotic for which it acts to potentiate its activity. Thus, in some aspects, provided herein are therapeutic compositions comprising a ROS target modulator and an antibiotic.

A ROS target modulator compound or agent described herein can increase or stimulate endogenous ROS production in a cell, such as a bacterial cell, by about at least 10% or more, at least 20% or more, at least 30% or more, at least 40% or more, at least 50% or more, at least 60% or more, at least 70% or more, at least 80% or more, at least 90% or more, at least 95% or more, at least 100%, at least 2-fold greater, at least 5-fold greater, at least 10-fold greater, at least 25-fold greater, at least 50-fold greater, at least 100-fold greater, at least 1000-fold greater, and all amounts in-between, in comparison to a reference or control level of ROS production in the absence of the ROS target modulator compound, or in the presence of the antibiotic alone. Methods and assays to identify such ROS stimulating compounds can be based on any method known to one of skill in the art, are found throughout the specification, in the drawings, and in the Examples section, such as the H₂O₂ and O₂⁻ productions assays described at FIGS. 2A-2D, and the time-course experiments described at FIGS. 3A-4F, for example.

As used herein, the term “adjuvant” can also be used to refer to an agent, such as the ROS target modulators described herein, which enhances or potentiates the pharmaceutical effect of another agent, such as an antibiotic, e.g., a β-lactam or fluoroquinolone antibiotic. In this sense, the ROS target modulator compounds, as disclosed herein, function as adjuvants to those bactericidal antibiotics that cause or act, in part, via ROS production, by further increasing basal ROS production in a cell, and thereby potentiating the activity of the antibiotics by about at least 10% or more, at least 20% or more, at least 30% or more, at least 40% or more, at least 50% or more, at least 60% or more, at least 70% or more, at least 80% or more, at least 90% or more, at least 95% or more, at least 100%, at least 2-fold greater, at least 5-fold greater, at least 10-fold greater, at least 25-fold greater, at least 50-fold greater, at least 100-fold greater, at least 1000-fold greater, and all amounts in-between, as compared to use of the antibiotic alone.

The term “agent” as used herein in reference to a ROS target modulator means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments of the aspects described herein, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, antisense RNAs, siRNAs, lipoproteins, aptamers, and modifications and combinations thereof etc. Compounds for use in the therapeutic compositions and methods described herein can be known to have a desired activity and/or property, e.g., increase endogenous ROS production, or can be selected from a library of diverse compounds, using screening methods known to one of ordinary skill in the art.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The following classes of inhibitors, as exemplified in E coli, can be effective for increasing ROS production in bacteria, including, but not limited to, E. coli, according to the compositions and methods described herein. Such bacteria include others with similar metabolic systems to E. coli, or those in which metabolic constructions are available, such as Mycobacterium tuberculosis, Staphylococcus aureus, Haemophilus influenzae, and Salmonella typhimurium⁵⁷⁻⁶¹, and other species determined to have similar metabolic systems using the systems-based, genome-scale ROS metabolic models described herein and consequent experimental validation. Accordingly, the bacteria being inhibited by the ROS target modulators and methods thereof described herein can therefore be an aerobic bacteria or a facultative anaerobe, such as one using mixed-acid fermentation in anaerobic conditions and producing lactate, succinate, ethanol, acetate and/or carbon dioxide, like E. coli; and/or the metabolic system can comprise glycolysis, pentose-phosphate pathway shunt, and/or the EntnerDoudoroff pathway. The bacteria's metabolic system can further comprise the TCA cycle and/or glyoxylate shunt. In these or other embodiments, the metabolic system can further comprise acetate metabolism.

ATP synthase Inhibitors

As demonstrated herein, deletion of ATP synthase in bacteria resulted in increased endogenous ROS production. Accordingly, in some aspects, provided herein are inhibitors of ATP synthase as ROS target modulators. The terms “ATP synthase inhibitors” or “inhibitors of ATP synthase,” as used herein, refer to an agent, molecule, or compound capable of inhibiting the activity or expression of F₁F₀-ATP synthase. For instance, an ATP synthase inhibitor decreases or reduces the activity or expression of an ATP synthase enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity. Methods suitable for measuring the activity or expression of an ATP synthase enzyme are well known in the art, such as, for example, total cellular ATP measurement assays, where ATP levels of log phase aerobic and dormant cultures are measured using, for example, an ATP bioluminescence assay kit (Roche Applied Science), and/or measurement of ATP synthesis activity, for example, where ATP synthesis of dormant bacteria grown under Wayne conditions is measured, as described in Koul et al., (2007) Nat. Chem. Biol. 3, 323-324.

F₁F₀-ATP synthase or ATP synthase catalyzes the hydrolysis of ATP to ADP and phosphate. The enzyme comprises five different subunits in the stoichiometry α₃β₃ΓΔε; the three catalytic β-subunits alternate with the three α-subunits around the centrally located single Γ-subunit. Members of the F₁F₀-family of ATP synthases and V-ATPase are present in bacteria, in chloroplast membranes, and in mitochondria. The enzyme is well conserved: the α- and β-subunit polypeptides from different sources show almost 50% sequence identity, while other F₁-subunit polypeptides show more variation (US Patent Publication 2004/0087489, the contents of which are herein incorporated by reference in their entireties).

Accordingly, in some embodiments of the compositions and methods described herein, the ATP synthase inhibitor or ROS target modulator is selected from a group including, but not limited to, IF₁; aurovertins; citreoviridin; citreoviridin acetate; quercetin; oligomycins; peliomycin; diarylquinolines and substituted quinioline derivatives, such as 1-(6-bromo-2-methoxy-quinolin-3-yl)-4-dimethylamino-2-naphthalen-1-yl-1-phenyl-butan-2-ol (also known as R207910 or TMC207), described in WO2004011436; N;N′-Dicyclohexylcarbodiimide; venturicidins; trimethyl tin chloride; triethyl tin chloride; tri-n-propyl tin chloride; tri-n-butyl tin chloride; triphenyl tin chloride; DBCT; ossamycin; leucinostatin; and efrapeptins; siRNA, antisense RNA, or ribozyme molecules that interfere with ATP synthase activity or expression; variants, analogs, or derivatives thereof, or any combination thereof.

In some embodiments of the compositions and methods described herein, an ATP synthase inhibitor is IF₁. Regulation of ATP production is mediated in part by IF₁ (also notated IF₁), which inhibits catalytic activity of the ATP synthase F₁ portion (see, e.g., Pullman et al., 1963 J. Biol. Chem. 238:3762; Tuena et al, 1988 Biochem. Cell Biol. 66:677; Walker et al., 1987 Biochem. 26:8613; Higuti et al., 1993 Biochim. Biophys. Acta 1172:311; U.S. Pat. No. 5,906,923; and references cited therein). Mature IF₁ protein is approximately 84 amino acids in length (9.6 kDa) and is synthesized as an approximately 105 amino acid precursor protein from which the N-terminal signal sequence is cleaved after importation into mitochondria. IF₁ features pH-sensitive, primarily alpha-helical structure that is highly conserved in eukaryotes such as yeast and mammals (Lebowitz et al. 1993 Arch. Biochem. Biophys. 301:64). In the alpha helix conformation IF₁ is inactive as an ATP synthase inhibitor, but at pH<6.7 IF₁ loses its helical structure and is activated to bind to the catalytic portion and inhibit ATP synthase (Jackson et al., 1988 FEBS Lett. 229:224; Mimura et al., 1993 J. Biochem. 113:350). IF₁ inhibition of ATPase activity can also be influenced by mitochondrial membrane potential and/or by IF₁ interactions with phospholipids (see, e.g., Solaini et al., 1997 Biochem J. 327:443 and references cited therein). IF₁ and related proteins are described, for example, in WO98/33909 and references cited therein.

In some embodiments of the compositions and methods described herein, an ATP synthase inhibitor is an efrapeptin. Efrapeptins refer to a family of a polar, hydrophobic peptides isolated from entomopathogenic fungi and are known to be potent inhibitors of mitochondrial F₁F₀-ATPase. With the exception of efrapeptin A and B, efrapeptins are composed of 15 amino acids (usually common amino-acids alanine, glycine, leucine and uncommon amino-acids α-aminobutyric acid, β-alanine, isovaline, and pipecolic acid) with the amino-terminal acetylated and the carboxyl-terminal blocked by N-peptido-1-isobutyl-2[1-pyrrole-(1,2-α)-pyrimidinium,2,3,4,5,6,7,8-,-hexahydro]-ethylamine (Krasnoff, S. B., et al., Antifungal and Insecticidal Properties of the Efrapeptins: Metabolites of the Fungus Tolypocladium niveum, J. Invert. Path., 58: 180-188 (1991)). Efrapeptins inhibit both ATP synthesis and hydrolysis by binding to a unique site in the central cavity of the F₁ catalytic domain of F₁F₀-ATP synthase and inducing a hydrophobic contact with the α-helical structure in the Γ-subunit. It inhibits F₁F₀-ATP synthase activity by blocking the conversion of β-subunit to a nucleotide binding conformation, which is essential for the cyclic interconvertion of the three catalytic sites.

Another family of inhibitors of F₁F₀-ATP synthase activity for use in some embodiments of the compositions and methods described herein is the mytotoxin family. Mycotoxins are secondary metabolites produced by many pathological and food spoilage fungi, including, for example, Aspergillus and Penicillium species. For example, aurovertin B is produced by Calcarisporium Arbuscula, citreoviridin is produced by Penicillium Citreoviride Biourge, while α-zearalenol is produced by Fusarium. Aurovertin B belongs to the aurovertin family. Aurovertin contains an α-pyrone (or 2-pyrone), a six-membered cyclic unsaturated ester. The derivatives of α-pyrone are widely distributed in nature and some of them inhibit ATP synthase by targeting F₁. Known as an ATP synthase inhibitor, aurovertin B acts to prevent the attainment of the tight conformation in the ATPase cycle. Accordingly, in some embodiments of the compositions and methods described herein, an inhibitor of ATP synthase can be selected from aurovertin B, citreoviridin, α-zearalenol, or any other myotoxin.

### Succinate Dehydrogenase Inhibitors

As demonstrated herein, deletion of succinate dehydrogenase in bacteria resulted in increased endogenous ROS production and potentiation of bactericidal antibiotic activity. Accordingly, in some aspects of the compositions and methods described herein, provided herein are inhibitors of succinate dehydrogenase or succinate dehydrogenase inhibitor for use as ROS target modulators. The terms “succinate dehydrogenase inhibitors” or “inhibitors of succinate dehydrogenase,” as used herein, refer to an agent, molecule, or compound capable of inhibiting the activity or expression of succinate dehydrogenase. For instance, a succinate dehydrogenase inhibitor decreases or reduces the activity or expression of a succinate dehydrogenase enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity. Methods suitable for measuring the activity or expression of a succinate dehydrogenase enzyme are well known in the art, such as the assays described herein, and, for example, electrochemical assays, such as non-catalytic voltammetry, and spectrophotometric methods using phenazine ethosulfate as primary electron acceptor coupled to the reduction of 2,6-dichlorophenolindophenol (Biochim Biophys Acta. 2002 Jan. 17; 1553(1-2):140-57).

Succinate dehydrogenase, which is also known as succinate-coenzyme Q reductase (SQR) or respiratory Complex II, is an enzyme complex, bound to the inner mitochondrial membrane of mammalian mitochondria and many bacterial cells. It is the only known enzyme that participates in both the citric acid cycle and the electron transport chain. Succinate dehydrogenase inhibitors are all active substances which have an inhibitory effect on the enzyme succinate dehydrogenase in the mitochondrial or bacterial respiratory chain. There are at least two distinct classes of succinate dehydrogenase inhibitors or inhibitors of complex II: those that bind in the succinate pocket and those that bind in the ubiquinone pocket. Ubiquinone type inhibitors include, for example, carboxin and thenoyltrifluoroacetone. Succinate-analogue inhibitors include the synthetic compound malonate, as well as the TCA cycle intermediates, malate and oxaloacetate.

Accordingly, in some embodiments of the compositions and methods described herein, the succinate dehydrogenase inhibitor or ROS target modulator is selected from a group including, but not limited to, methyl 3-[[(5,6-dihydro-2-methyl-1,4-oxathiin-3-yl)carbonyl]amino]benzoate and ethyl 3-[[(5,6-dihydro-2-methyl-1,4-oxathiin-3-yl)carbonyl]amino]benzoate; malonate; malate; oxaloacetate; 3-nitroproprionic acid; fluopyram or N-{[3-chloro-5-(trifluoromethyl)-2-pyridinyl]ethyl}-2,6-dichlorobenzamide-; isopyrazam, which is a mixture comprising the two syn isomers of 3-(difluoromethyl)-1-methyl-N-[(1RS,4SR,9RS)-1,2,3,4-tetrahydro-9-isopropyl-1,4-methanonaphthalene-5-yl]pyrazole-4-carboxamide and the two anti isomers of 3-(difluoromethyl)-1-methyl-N-[(1RS,4SR,9SR)-1,2,3,4-tetrahydro-9-isopropyl-1,4-methanonaphthalene-5-yl]pyrazole-4-carboxamide; boscalid or 2-chloro-N-(4′-chlorobiphenyl-2-yl)nicotinamide; penthiopyrad or (RS)-N-[2-(1,3-dimethylbutyl)-3-thienyl]-1-methyl-3-(trifluoromethyl)pyr-azole-4-carboxamide; penflufen or N-[2-(1,3-dimethylbutyl)phenyl]-5-fluoro-1,3-dimethyl-1H-pyrazole-4-carbo-xamide; sedaxan, which is a mixture comprising the two cis isomers of 2-[(1RS,2RS)-1,1′-bicycloprop-2-yl]-3-(difluoromethyl)-1-methylpyrazole-4-carboxanilide and the two trans isomers of 2-[(1RS,2SR)-1,1′-bicycloprop-2-yl]-3-(difluoromethyl)-1-methylpyrazole-4-carboxanilide; fluxapyroxad or 3-(difluoromethyl)-1-methyl-N-(3′,4′,5′-trifluoro-biphenyl-2-yl)-1H-pyraz-ole-4-carboxamide; bixafen or N-(3′,4′-dichloro-5-fluoro-1,1′-biphenyl-2-yl)-3-(difluoromethyl)-1-methy-1-1H-pyrazole-4-carboxamide; N-[2-(2,4-dichlorophenyl)-2-methoxy-1-methylethyl]-3-difluoromethyl-1-methyl-1H-pyrazole-4-carboxamide; malonic acid; pentachlorobutadienyl-cysteine (or PCBD-cys); 2-bromohydroquinone; 3-nitropropionic acid; cis-crotonalide fungicides; siRNA, antisense RNA, or ribozyme molecules that interfere with succinate dehydrogenase activity or expression; variants, analogs, or derivatives thereof, or any combination thereof. (White et al., Pesticide Biochemistry and Physiology, 9, 165 (1978); Brouillet et al., Proc. Natl. Acad. Sci. USA 92:7105 (1995); WO 03/070705; WO 03/010149; EP-A-1 389 614; WO 03/074491; WO 2006/015865; WO 2006/015866; WO 2004/035589; EP-A-0 737 682; DE-A 195 31 813; and WO 2005/123690, the contents of each of which are herein incorporated by reference in their entireties).

### Glutamate Dehydrogenase Inhibitors

As demonstrated herein, deletion of glutamate dehydrogenase in bacteria resulted in increased endogenous ROS production and potentiation of bactericidal antibiotic activity. Thus, in some aspects of the compositions and methods described herein, provided herein are “glutamate dehydrogenase inhibitors” or “inhibitors of glutamate dehydrogenase” for use as ROS target modulators. The terms “glutamate dehydrogenase inhibitors” or “inhibitors of glutamate dehydrogenase,” as used herein, refer to an agent, molecule, or compound capable of inhibiting the activity or expression of glutamate dehydrogenase. For instance, a glutamate dehydrogenase inhibitor decreases or reduces the activity or expression of a glutamate dehydrogenase enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a glutamate dehydrogenase enzyme are well known in the art, such as the assays described herein, and, for example, assays measuring the amination reaction catalyzed by glutamate dehydrogenase by measuring decrease in NADH₂ absorption, or assays measuring the deamination reaction catalyzed by glutamate dehydrogenase by measuring the increase in NADH₂ absorption (J Mol Biol. 2010 Jul. 23; 400(4):815-27).

Glutamate dehydrogenase (GLDH) is an enzyme, present in most microbes and the mitochondria of eukaryotes, that converts glutamate to α-ketoglutarate, and vice versa. Glutamate dehydrogenase also has a very high affinity for ammonia (1 mM), and therefore toxic levels of ammonia would have to be present in the body for the reverse reaction to proceed (that is, α-ketoglutarate and ammonia to glutamate and NAD(P)+). In bacteria, the ammonia is assimilated to amino acids via glutamate and amidotransferases.

Accordingly, in some embodiments of the compositions and methods described herein, the glutamate dehydrogenase inhibitor or ROS target modulator is selected from a group including, but not limited to, bromofuroate; 3-carboxy-5-bromofuroic acid; Palmitoyl-Coenzyme-A (Palmitoyl-Co-A); vanadium compounds (including, but not limited to, orthovanadate, vanadyl sulphate, vanadyl acetylacetonate, and combinations thereof), glutarate; 2-oxoglutarate (α.-ketoglutarate); estrogen; estrogen analogues; pyridine-2,6-dicarboxylic acid; (−)-epigallocatechin gailate (EGCG); siRNA, antisense, and ribozyme molecules that interfere with glutamate dehydrogenase activity or expression; variants, analogs, or derivatives thereof, or any combination thereof, such as, but not limited to, 2-oxoglutarate and vanadyl sulphate (U.S. Pat. No. 7,504,321; Cunliffe et al., “The Inhibition of Glutamate Dehydrogenase by Derivatives of Isophthalic Acid,” Phytochemistry 22(6):1357-1360, 1983).

### NADH Dehydrogenase Inhibitors

The inventors have also determined, as demonstrated herein, that inhibition of NADH dehydrogenase, via deletion in bacterial cells, increases endogenous ROS production and potentiation of bactericidal antibiotic activity. Accordingly, in some aspects of the compositions and methods described herein, provided herein are “NADH dehydrogenase inhibitors” or “inhibitors of NADH dehydrogenase” for use as ROS target modulators. The terms “NADH dehydrogenase inhibitors” or “inhibitors of NADH dehydrogenase,” as used herein, refer to an agent, molecule, or compound capable of inhibiting the activity or expression of NADH dehydrogenase. For instance, an NADH dehydrogenase inhibitor decreases or reduces the activity or expression of an NADH dehydrogenase enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of an NADH dehydrogenase enzyme are well known in the art, such as the assays described herein, and, for example, spectrophotometric assays following reduction of 3-(4,5-dimethylthiazol-2-yl)-2,5-diphenyltetrazolium bromide (MTT) as an artificial electron acceptor (Rodriguez-Montelongo et al., Arch Biochem Biophys. 2006 Jul. 1; 451(1):1-7).

Oxidative phosphorylation is the process by which ATP is formed as electrons are transferred from NADH or FADH₂ to O₂ by a series of electron carriers (Stryer, 1988, Biochemistry, Freeman). This process occurs in the mitochondria of eukaryotic cells. More specifically, enzymes that catalyze the electron transport chain reside in the inner membrane of mitochondria, and they are encoded by both nuclear and mitochondrial DNA. These enzymes exist as large protein complexes, and the first complex of the chain is known as NADH dehydrogenase or NADH-Q reductase. It has a molecular weight of 850,000 daltons and consists of over 40 polypeptide subunits, seven of which are encoded by the mitochondrial genome. (Anderson et al., 1981, Nature 290:457; Chomyn et al., 1985, Nature 314:592; Chomyn et al., 1986, Science 234:619). NADH dehydrogenase catalyzes the transfer of electrons from NADH to an electron carrier termed ubiquinone.

Accordingly, in some embodiments of the compositions and methods described herein, the NADH dehydrogenase inhibitor or ROS target modulator is selected from a group including, but not limited to; Amytal; Amytal Sodium; Annonin VI; Aurachin A; Aurachin B; Aureothin; Benzimidazole; Bullactin; calnexin; Capsaicin; Ethoxyformic anhydride; Ethoxyquin; Fenpyroximate; Mofarotene (Ro 40-8757; arotinoids); mofarotene 2-oxoglutarate dehydrogenase; Molvizarin; Myxalamide PI; M2-type pyruvate kinase; Otivarin (annonaceous acetogenins); Pethidine; rhein and other quinone analogs; Phenalamid A₂; Phenoxan; Piericidin A; p-chloromercuribenzoate; Ranolazine (RS-43285); Rolliniasatin-1; Rolliniasatin-2; Rotenone; Squamocin; Thiangazole rotenoids; thiol reagents; Demerol; iron chelators; NAD⁺ (nicotinamide adenine dinucleotide; oxidized form); AMP (adenosine monophosphate); ADP (adenosine diphosphate); ADP-ribosylation factor 3; ATP (adenosine triphosphate); guanidinium salts; NADH; the general class of barbituates; gossypol; polyphenol; dihydroxynaphthoic acids; adenosine diphosphate ribose; rotenoid; acetogenin; nitrosothiols; peroxynitrite; carvedilol; arylazido-beta-alanyl NAD+; adriamycin; 4-hydroxy-2-nonenal; pyridine derivatives; 2-heptyl-4-hydroxyquinoline N-oxide; dicumarol; o-phenanthroline; 2;2′-dipyridyl; other small molecule NADH dehydrogenase inhibitors; siRNA, antisense, and ribozyme molecules that interfere with NADH dehydrogenase subunit gene expression or activity; and analogs, variants, or derivatives thereof, and combinations thereof. (US PAT APPS 20040097409; Uchida et al., 1994 Int. J. Cancer 58:891-897; Singer and Ramsay, 1992, Mol. Mechan. in Bioenergetics Chap. 6, p. 153; Degli Espasti et al., 1994, Biochem. J. 301:161; Friedrich et al., 1994, Eur. J. Biochem, 219:691; Uchida et al., 1994, Int. J. Cancer 58:891; Wyatt et al., 1995, Biochem. Pharmacol. 50:1599; Shimomura et al., 1989, Arch. Biochem Biophy. 270:573).

### Pyruvate Dehydrogenase Inhibitors

The inventors have also determined, as demonstrated herein, that inhibition of pyruvate dehydrogenase, via deletion in bacterial cells, increases endogenous ROS production and potentiation of bactericidal antibiotic activity. Accordingly, in some aspects of the compositions and methods described herein, provided herein are “pyruvate dehydrogenase inhibitors” or “inhibitors of pyruvate dehydrogenase” for use as ROS target modulators. The terms “pyruvate dehydrogenase inhibitors” or “inhibitors of pyruvate dehydrogenase,” as used herein, refer to an agent, molecule, or compound capable of inhibiting the activity or expression of pyruvate dehydrogenase. For instance, a pyruvate dehydrogenase inhibitor decreases or reduces the activity or expression of a pyruvate dehydrogenase enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a pyruvate dehydrogenase enzyme are well known in the art, such as the assays described herein, and, for example, by spectrophotometric monitoring of the reduction of 2,6-dichlorophenolindophenol (2,6-DCIP) (Bioorg Med Chem. 2011 Dec. 15; 19(24):7501-6).

Pyruvate dehydrogenase (E1) is the first component enzyme of pyruvate dehydrogenase complex (PDC). The pyruvate dehydrogenase complex contributes to transforming pyruvate into acetyl-CoA by a process called pyruvate decarboxylation. Acetyl-CoA may then be used in the citric acid cycle to carry out cellular respiration, so pyruvate dehydrogenase contributes to linking the glycolysis metabolic pathway to the citric acid cycle and releasing energy via NADH.

Accordingly, in some embodiments of the compositions and methods described herein, the pyruvate dehydrogenase inhibitor or ROS target modulator is selected from a group including, but not limited to;

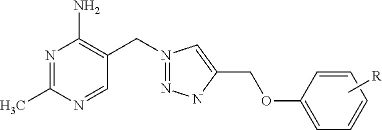

where R is 2-Cl-4-NO₂, 4-NO₂, 4-COOH, or H; secondary amides of (R)-3;3;3-Trifluoro-2-hydroxy-2-methylpropionic acid glyoxylate; (R)-3;3;3-Trifluoro-2-hydroxy-2-methylpropionamides; anilides of (R)-Trifluoro-2-hydroxy-2-methylpropionic Acidhydroxypyruvate; kynurenate; xanthurenate; α-cyano-4-hydroxycinnamic acid; bromopyruvic acid; fluropyruvic acid; AZD-7545; phosphonate and phosphinate analogs of pyruvate; mono- and bifunctional arsenoxides; branched-chain 2-oxo acids; 2-oxo-3-alkynoic acids; tetrahydrothiamin diphosphate (ThDP; 2-thiazolone and 2-thiothiazolone analogs of ThDP; other small molecule pyruvate dehydrogenase inhibitors; siRNA, antisense, and ribozyme molecules that interfere with pyruvate dehydrogenase gene expression or activity; and analogs, variants, or derivatives thereof, or any combination thereof (Bioorg Med Chem. 2011 Dec. 15; 19(24):7501-6; U.S. Pat. Nos. 6,218,435, 7,566,699).

### Cytochrome Bo Terminal Oxidase Inhibitors

As demonstrated herein, deletion of cytochrome bo terminal oxidase in bacteria resulted in increased endogenous ROS production. Accordingly, in some aspects, provided herein are inhibitors of cytochrome oxidases, such as cytochrome bo terminal oxidase, for use as ROS target modulators. The terms “inhibitors of cytochrome oxidases,” “inhibitors of cytochrome oxidase,” inhibitors of cytochrome bo oxidases,” “inhibitors of cytochrome bo oxidase,” as used herein, refer to an agent, molecule, or compound capable of inhibiting the activity or expression of a cytochrome oxidase, such as cytochrome bo oxidase. For instance, a cytochrome oxidase inhibitor decreases or reduces the activity or expression of a cytochrome oxidase enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a cytochrome oxidase enzyme are well known in the art, such as the assays described herein, and, for example, by spectrophotometric monitoring.

Cytochromes bo and bd are two terminal respiratory oxidases found in Escherichia coli and many other bacteria. Both enzymes catalyze the oxidation of ubiquinol by molecular oxygen to produce quinone and water. Cytochrome bd is predominant when the oxygen concentration in the growth medium is low, whereas cyto-chrome bo predominates when the oxygen concentration is high. Cytochrome bo catalyzes the two-electron oxidation of ubiquinol within the membrane and the four-electron reduction of molecular oxygen to water. In the cell the enzyme functions as a proton pump, with a net movement of 2H+/e− across the cytoplasmic membrane, thereby generating a proton-motive force. There are four subunits encoded by the cyoB, cyoA, cyoC and cyoD genes, all of which are necessary for a functional enzyme.

Accordingly, in some embodiments of the compositions and methods described herein, the cytochrome oxidase inhibitor or ROS target modulator is selected from a group including, but not limited to, azide; nitric oxide; cytochrome P450 oxidase inhibitors and uses thereof (described in EP2465855 A1); aurachin A (α-(4,8-Dimethyl-3,7-nonadienyl)-1,2-dihydro-α,4-dimethylfuro[2,3-c]quinoline-2-methanol 5-oxide) and its type II and type III derivatives; Aurachin C (1-Hydroxy-2-methyl-3-(3,7,11-trimethyl-2,6,10-dodecatrienyl)-4(1H)-quinolinone) and its type II and type III derivatives; aurachin D (2-Methyl-3-(3,7,11-trimethyl-2,6,10-dodecatrienyl)-4(1H)-quinolinone) and its type II and type III derivatives; tridecylstigmatelli; stigmatellin; nigericin; hydroxylamine; heptylhydroxyquinoline N-oxide (HQNO); nonylhydroxyquinoline N-oxide (NQNO); dibromothymoquinone (DBMIB); piericidin A; undecylhydroxydioxobenzo-thiazole (UHDBT) (“New inhibitors of the quinol oxidation sites of bacterial cytochromes bo and bd,” Biochemistry, 1995, 34 (3), pp 1076-1083); other small molecule cytochrome oxidase inhibitors; siRNA, antisense, and ribozyme molecules that interfere with cytochrome oxidase gene expression or activity; and analogs, variants, or derivatives thereof, or any combination thereof.

### Triose Phosphate Inhibitors

The inventors have also determined, as demonstrated herein, that inhibition of triose phosphate isomerase, via deletion in bacterial cells, increases endogenous ROS production and potentiation of bactericidal antibiotic activity. Accordingly, in some aspects of the compositions and methods described herein, provided herein are “triose phosphate isomerase inhibitors” or “inhibitors of triose phosphate isomerase” for use as ROS target modulators. The terms “triose phosphate isomerase inhibitors” or “inhibitors of triose phosphate isomerase,” as used herein, refer to an agent, molecule, or compound capable of inhibiting the activity or expression of triose phosphate isomerase. For instance, a triose phosphate isomerase inhibitor decreases or reduces the activity or expression of a triose phosphate isomerase if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a triose phosphate isomerase enzyme are well known in the art, such as the assays described herein, and, for example, by spectrophotometric monitoring of the amount of enzyme that converts one micromole of D-glyceraldehyde-3-phosphate to dihydroxyacetone phosphate per minute at 25° C. and pH 7.6 (Methods of Enzymatic Analysis, Bergmeyer, H.U. ed Vol 1, 515, 1974, Academic Press, New York).

Triose-phosphate isomerase (TPI or TIM) is an enzyme that catalyzes the reversible interconversion of the triose phosphate isomers dihydroxyacetone phosphate and D-glyceraldehyde 3-phosphate. More simply, the enzyme catalyzes the isomerization of a ketose (DHAP) to an aldose (GAP), also referred to as PGAL.

Accordingly, in some embodiments of the compositions and methods described herein, the triose phosphate isomerase inhibitor or ROS target modulator is selected from a group including, but not limited to, 3-haloacetol phosphates; glycidol phosphate; phosphoenol pyruvate; DHAP; GAP; 2-phosphoglycollate; phosphoglycolohydroxamate; 3-phosphoglycerate; glycerol phosphate; phosphoenol pyruvate; 2;9-Dimethyl-β-carbolines and derivatives thereof; 3-(2-benzothiazolylthio)-1-propanesulfonic acid; 2-carboxyethylphosphonic acid; 2-phosphoglyceric acid; N-hydroxy-4-phosphono-butanamide; [2(formyl-hydroxy-amino)-ethyl]-phosphonic acid; other small molecule triose phosphate isomerase inhibitors; siRNA, antisense, and ribozyme molecules that interfere with triose phosphate isomerase gene expression or activity; and analogs, variants, or derivatives thereof, or any combination thereof.

### Glucose-6-Phosphate Dehydrogenase Inhibitors

As demonstrated herein, deletion of glucose-6-phosphate dehydrogenase (G6PD) in bacteria resulted in increased endogenous ROS production. Accordingly, in some aspects, provided herein are inhibitors of glucose-6-phosphate dehydrogenase as ROS target modulators. The terms “glucose-6-phosphate dehydrogenase inhibitors” or “inhibitors of glucose-6-phosphate dehydrogenase,” as used herein, refer to an agent, molecule, or compound capable of inhibiting the activity or expression of glucose-6-phosphate dehydrogenase. In other words, a glucose-6-phosphate dehydrogenase inhibitor decreases or reduces the activity or expression of a glucose-6-phosphate dehydrogenase if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a glucose-6-phosphate dehydrogenase enzyme are well known in the art, such as the assays described herein, and, for example, by spectrophotometric monitoring of the amount of enzyme that will oxidize 1.0 μmole of D-glucose-6-phosphate to 6-phospho-D-gluconate per minute in the presence of β-NADP at pH 7.4 at 25° C.

Glucose-6-phosphate dehydrogenase (G6PD or G6PDH) is a cytosolic enzyme in the pentose phosphate pathway, a metabolic pathway that supplies reducing energy to cells by maintaining the level of the co-enzyme nicotinamide adenine dinucleotide phosphate (NADPH).

Accordingly, in some embodiments of the compositions and methods described herein, the glucose-6-phosphate dehydrogenase inhibitor or ROS target modulator is selected from a group including, but not limited to, dehydroepiandrosterone (DHEA), DHEA-sulfate, 2-deoxyglucose, halogenated DHEA, derivatives of the DHEA 1 described in Hamilton et al., J Med Chem., 2012 May 10; 55(9):4431-45; epiandrosterone; isoflurane; sevoflurane; diazepam; CBF-BS2; cystamine (2,2′-Dithio-bis[ethylamine]); 16α-bromoepiandrosterone (EPI); 16α-hydroxy-5-androsten-17-one; 16α-fluoro-5-androsten-17-one (fluasterone); 16α-fluoro-16β-methyl-5-androsten-17-one; 16α-methyl-5-androsten-17-one; 16β-methyl-5-androsten-17-one; 16α-hydroxy-5α-androstan-17-one; 16α-fluoro-5α-androstan-17-one; 16α-fluoro-160-methyl-5α-androstan-17-one; 16α-methyl-5α-androstan-17-one; or 16β-methyl-5α-androstan-17-one; halogenated (fluorinated), D-hexoses (e.g., 2-Amino-2-deoxy-D-glucose-6-phosphate (D-glucosamine-6-phosphate); other small molecule glucose-6-phosphate dehydrogenase inhibitors; siRNA, antisense, and ribozyme molecules that interfere with glucose-6-phosphate dehydrogenase gene expression or activity; and analogs, variants, or derivatives thereof, or any combination thereof (US Patent Publication 20100298412; U.S. Pat. Nos. 5,001,119 and 5,700,793).

### 6-Phosphogluconate Dehydrogenase Inhibitors

As demonstrated herein, deletion of 6-phosphogluconate dehydrogenase in bacteria resulted in increased endogenous ROS production. Accordingly, in some aspects, provided herein are inhibitors of 6-phosphogluconate dehydrogenase as ROS target modulators. The terms “6-phosphogluconate dehydrogenase inhibitors” or “inhibitors of 6-phosphogluconate dehydrogenase,” as used herein, refer to an agent, molecule, or compound capable of inhibiting the activity or expression of 6-phosphogluconate dehydrogenase. In other words, a 6-phosphogluconate dehydrogenase inhibitor decreases or reduces the activity or expression of a 6-phosphogluconate dehydrogenase if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a 6-phosphogluconate dehydrogenase enzyme are well known in the art, such as the assays described herein, and, for example, by spectrophotometric monitoring of the conversion of nitroblue tetrazolium (NBT) in the presence of phenazine methosulfate (PMS), which reacts with the NADPH produced by dehydrogenases to produce an insoluble blue-purple formazan.

Phosphogluconate dehydrogenase is an enzyme in the pentose phosphate pathway. It forms ribulose 5-phosphate from 6-phosphogluconate. It is an oxidative carboxylase that catalyzes the decarboxylating reduction of 6-phosphogluconate into ribulose 5-phosphate in the presence of NADP. This reaction is a component of the hexose mono-phosphate shunt and pentose phosphate pathways (PPP). Prokaryotic and eukaryotic 6PGD are proteins of about 470 amino acids whose sequences are highly conserved.

Accordingly, in some embodiments of the compositions and methods described herein, the phosphogluconate dehydrogenase inhibitor or ROS target modulator is selected from a group including, but not limited to, 6-aminonicotinamide; aldonate 4-phospho-d-erythronate, 5,6-Dideoxy-6-phosphono-d-arabino-hexonate, 5-deoxy-5-phosphono-d-arabinonate, and other inhibitory 4-carbon and 5-carbon aldonates described in Pasti et al., Bioorg Med Chem. 2003 Apr. 3; 11(7):1207-14; phosphorylated carbohydrate substrates and transition state analogues, non-carbohydrate substrate analogues and triphenylmethane-based compounds described in Hanau et al., Curr. Med. Chem., 11 (2004), p. 2639; other small molecule phosphogluconate dehydrogenase inhibitors; siRNA, antisense, and ribozyme molecules that interfere with phosphogluconate dehydrogenase gene expression or activity; and analogs, variants, or derivatives thereof, or any combination thereof.

### Succinyl Coenzyme A (CoA) Synthetase Inhibitors

As demonstrated herein, deletion of succinyl-CoA synthetase in bacteria resulted in increased endogenous ROS production. Accordingly, in some aspects, provided herein are inhibitors of succinyl-CoA synthetase as ROS target modulators. The terms “succinyl-CoA synthetase inhibitors” or “inhibitors of succinyl-CoA synthetase,” as used herein, refer to an agent, molecule, or compound capable of inhibiting the activity or expression of succinyl-CoA synthetase. In other words, a succinyl-CoA synthetase inhibitor decreases or reduces the activity or expression of a succinyl-CoA synthetase if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a succinyl-CoA synthetase enzyme are well known in the art, such as the assays described herein, and, for example, by the procedure described by Kavanaugh-Black et al., Proc Natl Acad Sci USA. 1994 Jun. 21; 91(13):5883-7.

Succinyl coenzyme A synthetase (also known as succinyl-CoA synthetase or succinate thiokinase or succinate-CoA ligase) is an enzyme that catalyzes the reversible reaction of succinyl-CoA to succinate. The enzyme facilitates the coupling of this reaction to the formation of a nucleoside triphosphate molecule (either GTP or ATP) from an inorganic phosphate molecule and a nucleoside diphosphate molecule (either GDP or ADP). It plays a key role as one of the catalysts involved in the citric acid cycle. Bacterial and mammalian SCSs are made up of α and β subunits. In E. coli two αβ heterodimers link together to form an α2β2 heterotetrameric structure. The E. coli SCS heterotetramer has been crystallized and characterized in great detail. The two a subunits reside on opposite sides of the structure and the two β subunits interact in the middle region of the protein. The two a subunits only interact with a single β unit, whereas the β units interact with a single α unit (to form the αβ dimer) and the β subunit of the other αβ dimer. A short amino acid chain links the two β subunits which gives rise to the tetrameric structure. Mutagenesis experiments have determined that two glutamate residues (one near the catalytic histidine, Glu208α and one near the ATP grasp domain, Glu19713) play a role in the phosphorylation and dephosphorylation of the histidine.

Accordingly, in some embodiments of the compositions and methods described herein, the succinyl-CoA synthetase inhibitor or ROS target modulator is selected from a group including, but not limited to, LY266500; vanadium sulphate; other small molecule succinyl-CoA synthetase inhibitors; siRNA, antisense, and ribozyme molecules that interfere with succinyl-CoA synthetase gene expression or activity; and analogs, variants, or derivatives thereof, or any combination thereof

### Phosphate Acetyltransferase Inhibitors

As demonstrated herein, deletion of phosphate acetyltransferase in bacteria resulted in increased endogenous ROS production. Accordingly, in some aspects, provided herein are inhibitors of phosphate acetyltransferase as ROS target modulators. The terms “phosphate acetyltransferase inhibitors” or “inhibitors of phosphate acetyltransferase,” as used herein, refer to an agent, molecule, or compound capable of inhibiting the activity or expression of phosphate acetyltransferase. In other words, a phosphate acetyltransferase inhibitor decreases or reduces the activity or expression of a phosphate acetyltransferase if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of a phosphate acetyltransferase enzyme are well known in the art, such as the assays described herein, and, for example, transacetylations assays incubating acetyl CoA and glucosamine-1-phosphate followed by mass spectral analysis (Int J Biochem Cell Biol. 2008; 40(11):2560-71).

Phosphate acetyltransferase is an enzyme that catalyzes the chemical reaction acetyl-CoA+phosphate [symbol] CoA+acetyl phosphate

Thus, the two substrates of this enzyme are acetyl-CoA and phosphate, whereas its two products are CoA and acetyl phosphate.

Phosphate acetyltransferase belongs to the family of transferases, specifically those acyltransferases transferring groups other than aminoacyl groups. The systematic name of this enzyme class is acetyl-CoA:phosphate acetyltransferase, but it is also known as phosphotransacetylase, phosphoacylase, and PTA. Phosphate acetyltransferase participates in 3 metabolic pathways, including taurine and hypotaurine metabolism, pyruvate metabolism, and propanoate metabolism.

Accordingly, in some embodiments of the compositions and methods described herein, the phosphate acetyltransferase inhibitor or ROS target modulator is selected from a group including, but not limited to, small molecule phosphate acetyltransferase inhibitors; siRNA, antisense, and ribozyme molecules that interfere with phosphate acetyltransferase gene expression or activity; and analogs, variants, or derivatives thereof, or any combination thereof

### Phosphofructokinase Inhibitors

As demonstrated herein, deletion of phosphofructokinase in bacteria result in increased endogenous ROS production. Accordingly, in some aspects, provided herein are inhibitors of phosphofructokinases as ROS target modulators. The terms “phosphofructokinase inhibitors” or “inhibitors of phosphofructokinase,” as used herein, refer to an agent, molecule, or compound capable of inhibiting the activity or expression of phosphofructokinase. In other words, a phosphofructokinase inhibitor decreases or reduces the activity or expression of a phosphofructokinase if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of phosphofructokinase enzyme are well known in the art, such as the assays described herein, and, for example, colorimetric assays that measure conversion of fructose-6-phosphate and ATP to fructose-diphosphate and ADP, such as the assay manufactured by BIOVISION.

Phosphofructokinase (PFK) is a kinase enzyme that phosphorylates fructose 6-phosphate in glycolysis to fructose-1,6-bisphosphate, a key regulatory step in the glycolytic pathway. PFK exists as a homotetramer in bacteria and mammals (where each monomer possesses 2 similar domains) and as an octomer in yeast (where there are 4 alpha-(PFK1) and 4 beta-chains (PFK2), the latter, like the mammalian monomers, possessing 2 similar domains. PFK is about 300 amino acids in length, and structural studies of the bacterial enzyme have shown it comprises two similar (alpha/beta) lobes: one involved in ATP binding and the other housing both the substrate-binding site and the allosteric site (a regulatory binding site distinct from the active site, but that affects enzyme activity). The identical tetramer subunits adopt 2 different conformations: in a ‘closed’ state, the bound magnesium ion bridges the phosphoryl groups of the enzyme products (ADP and fructose-1,6-bisphosphate); and in an ‘open’ state, the magnesium ion binds only the ADP.

Accordingly, in some embodiments of the compositions and methods described herein, the phosphofructokinase inhibitor or ROS target modulator is selected from a group including, but not limited to, aurintricarboxylic acid; pyruvate; acidosis-inducing agents; 2-deoxy-2-fluoro-D-glucose; citrate and halogenated derivatives of citrate; fructose 2,6-bisphosphate; bromoacetylethanolamine phosphate analogues (e.g., N-(2-methoxyethyl)-bromoacetamide, N-(2-ethoxyethyl)-bromoacetamide, N-(3-methoxypropyl)-bromoacetamide); phosphoglycerate, quinone methides (e.g., taxodone, taxodione), a-methylene lactones (e.g., euparotin acetate eupacunin, vernolepin), argaric acid, quinaldic acid, and 5′-p-flurosuflonylbenzoyl adenosine; small molecule phosphofructokinase inhibitors; siRNA, antisense, and ribozyme molecules that interfere with phosphofructokinase gene expression or activity; and analogs, variants, or derivatives thereof, or any combination thereof.

### Fumarase B Inhibitors

As demonstrated herein, deletion of fumarase B in bacteria result in increased endogenous ROS production. Accordingly, in some aspects, provided herein are inhibitors of fumarase B s as ROS target modulators. The terms “fumarase B inhibitors” or “inhibitors of fumarase B,” as used herein, refer to an agent, molecule, or compound capable of inhibiting the activity or expression of fumarase B. In other words, a fumarase B inhibitor decreases or reduces the activity or expression of a fumarase B enzyme if the compound or agent can reduce the activity or expression of the enzyme by at least 10%, at least 20%, at least 30%, at least 40%, at least 50%, at least 60%, at least 70%, at least 80%, at least 90%, at least 95%, at least 97%, at least 98%, at least 99%, or undetectable, relative to the absence of the inhibitor, using standards assays or methods known in the art for measuring the activity or expression. Methods suitable for measuring the activity or expression of fumarase B enzyme are well known in the art, such as, for example, spectrophotometric monitoring the production of fumarate at 240 nm from L-malate (Bergmeyer H U et al. (1974) In: HU Bergmeyer (ed) Methods of enzymatic analysis. Academic Press, New York).

Fumarase (or fumarate hydratase) is a key enzyme in the TCA cycle that catalyzes the reversible and stereo-specific hydration/dehydration of fumarate to L-malic acide. Fumarase comes in two forms: mitochondrial and cytosolic. Prokaryotes are known to have three different forms of fumarase: Fumarase A, Fumarase B, and Fumarase C. Fumarase C is a part of the class II fumarases, whereas Fumarase A and Fumarase B from Escherichia coli (E. coli) are classified as class I

Accordingly, in some embodiments of the compositions and methods described herein, the fumarase B inhibitor or ROS target modulator is selected from a group including, but not limited to, trans-aconitate; bromomesaconate; citrate; meso-tartaric acid; bismuth; DL-β-fluoromalic acid; S-2,3-Dicarboxyaziridine; small molecule fumarase B inhibitors; siRNA, antisense, and ribozyme molecules that interfere with fumarase B gene expression or activity; and analogs, variants, or derivatives thereof, or any combination thereof.

As demonstrated herein, inhibition of a ROS target potentiates the activity and efficacy of bactericidal antibiotics, such as β-lactams and fluoroquinilones. Accordingly, provided herein in some aspects, are compositions, such as therapeutic compositions, comprising an effective amount of one or more ROS target modulators, as described herein, and an effective amount of an antimicrobial or antibiotic agent.

As used herein, the term “antibiotic” refers to any compound known to one of ordinary skill in the art that will inhibit or reduce the growth of, or kill, one or more bacterial species. Thus, the ability to inhibit or reduce the growth of, or kill, one or more bacterial microorganisms is referred to herein as “antibiotic activity.” In some embodiments, an antibiotic agent for use in the compositions and methods described herein is “bacteriostatic,” meaning that they stop bacteria from reproducing, while not necessarily harming them otherwise. Bacteriostatic antibiotics limit the growth of bacteria by interfering with bacterial protein production, DNA replication, or other aspects of bacterial cellular metabolism, and typically work together with the immune system to remove microorganisms from the body. High concentrations of some bacteriostatic agents are also bactericidal, in some cases, whereas low concentrations of some bactericidal agents are bacteriostatic. In some embodiments, an antibiotic agent (or the effective amount thereof) for use in the compositions and methods described herein is “bactericidal” for the target microbe. That is, the agent kills the target bacterial cells and, ideally, is not substantially toxic to mammalian cells. Bactericidal agents include disinfectants, antiseptics, or antibiotics. Many antibacterial compounds are relatively small molecules with a molecular weight of less than 2000 atomic mass units. The term “antibiotic” includes semi-synthetic modifications of various natural compounds, such as, for example, the beta-lactam antibiotics, which include penicillins (produced by fungi in the genus Penicillium), the cephalosporins, and the carbapenems. Accordingly, the term “antibiotic” includes, but is not limited to, β-lactams (e.g., penicillins and cephalosporins), aminoglycosides (e.g., gentamicin, streptomycin, kanamycin), vancomycins, bacitracins, macrolides (e.g., erythromycins), lincosamides (e.g., clindomycin), chloramphenicols, tetracyclines, amphotericins, cefazolins, clindamycins, mupirocins, sulfonamides and trimethoprim, rifampicins, metronidazoles, quinolones, novobiocins, polymixins, gramicidins, or any salts or variants thereof. The antibiotic used in addition to the ROS target modulator in the various embodiments of the compositions and methods described herein will depend on the type of bacterial infection.

Any of the major classes of antibiotic agents in which bactericidal activity is potentiated or enhanced by inhibiting ROS production can be used with the ROS target modulators described herein. Such classes of antibiotic agents include, for example, nitroimidazole compounds, lincosamides, sulfonamide compounds, dihydrofolate reductase inhibitors, lipopeptide molecules, tetracycline compounds, compounds comprising a beta-lactam moiety, glycopeptides, oxazolidinones, and quinolones. Accordingly, non-limiting examples of antimicrobial and antibiotic agents that are suitable for use with the compositions and methods described herein, provided they can be potentiated by inhibition of a ROS target, include, without limitation, mandelic acid, 2,4-dichlorobenzenemethanol, 4-[bis(ethylthio)methyl]-2-methoxyphenol, 4-epi-tetracycline, 4-hexylresorcinol, 5,12-dihydro-5,7,12,14-tetrazapentacen, 5-chlorocarvacrol, 8-hydroxyquinoline, acetarsol, acetylkitasamycin, acriflavin, alatrofloxacin, ambazon, amfomycin, amikacin, amikacin sulfate, aminoacridine, aminosalicylate calcium, aminosalicylate sodium, aminosalicylic acid, ammoniumsulfobituminat, amorolfin, amoxicillin, amoxicillin sodium, amoxicillin trihydrate, amoxicillin-potassium clavulanate combination, amphotericin B, ampicillin, ampicillin sodium, ampicillin trihydrate, ampicillin-sulbactam, apalcillin, arbekacin, aspoxicillin, astromicin, astromicin sulfate, azanidazole, azidamfenicol, azidocillin, azithromycin, azlocillin, aztreonam, bacampicillin, bacitracin, bacitracin zinc, bekanamycin, benzalkonium, benzethonium chloride, benzoxonium chloride, berberine hydrochloride, biapenem, bibrocathol, biclotymol, bifonazole, bismuth subsalicylate, bleomycin antibiotic complex, bleomycin hydrochloride, bleomycin sulfate, brodimoprim, bromochlorosalicylanilide, bronopol, broxyquinolin, butenafine, butenafine hydrochloride, butoconazol, calcium undecylenate, candicidin antibiotic complex, capreomycin, carbenicillin, carbenicillin disodium, carfecillin, carindacillin, carumonam, carzinophilin, caspofungin acetate, cefacetril, cefaclor, cefadroxil, cefalexin, cefalexin hydrochloride, cefalexin sodium, cefaloglycin, cefaloridine, cefalotin, cefalotin sodium, cefamandole, cefamandole nafate, cefamandole sodium, cefapirin, cefapirin sodium, cefatrizine, cefatrizine propylene glycol, cefazedone, cefazedone sodium salt, cefazolin, cefazolin sodium, cefbuperazone, cefbuperazone sodium, cefcapene, cefcapene pivoxil hydrochloride, cefdinir, cefditoren, cefditoren pivoxil, cefepime, cefepime hydrochloride, cefetamet, cefetamet pivoxil, cefixime, cefmenoxime, cefmetazole, cefmetazole sodium, cefminox, cefminox sodium, cefmolexin, cefodizime, cefodizime sodium, cefonicid, cefonicid sodium, cefoperazone, cefoperazone sodium, ceforanide, cefoselis sulfate, cefotaxime, cefotaxime sodium, cefotetan, cefotetan disodium, cefotiam, cefotiam hexetil hydrochloride, cefotiam hydrochloride, cefoxitin, cefoxitin sodium, cefozopran hydrochloride, cefpiramide, cefpiramide sodium, cefpirome, cefpirome sulfate, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftizoxime sodium, ceftriaxone, ceftriaxone sodium, cefuroxime, cefuroxime axetil, cefuroxime sodium, cetalkonium chloride, cetrimide, cetrimonium, cetylpyridinium, chloramine T, chloramphenicol, chloramphenicol palmitate, chloramphenicol succinate sodium, chlorhexidine, chlormidazole, chlormidazole hydrochloride, chloroxylenol, chlorphenesin, chlorquinaldol, chlortetracycline, chlortetracycline hydrochloride, ciclacillin, ciclopirox, cinoxacin, ciprofloxacin, ciprofloxacin hydrochloride, citric acid, clarithromycin, clavulanate potassium, clavulanate sodium, clavulanic acid, clindamycin, clindamycin hydrochloride, clindamycin palmitate hydrochloride, clindamycin phosphate, clioquinol, cloconazole, cloconazole monohydrochloride, clofazimine, clofoctol, clometocillin, clomocycline, clotrimazol, cloxacillin, cloxacillin sodium, colistin, colistin sodium methanesulfonate, colistin sulfate, cycloserine, dactinomycin, danofloxacin, dapsone, daptomycin, daunorubicin, DDT, demeclocycline, demeclocycline hydrochloride, dequalinium, dibekacin, dibekacin sulfate, dibrompropamidine, dichlorophene, dicloxacillin, dicloxacillin sodium, didecyldimethylammonium chloride, dihydrostreptomycin, dihydrostreptomycin sulfate, diiodohydroxyquinolin, dimetridazole, dipyrithione, dirithromycin, DL-menthol, D-menthol, dodecyltriphenylphosphonium bromide, doxorubicin, doxorubicin hydrochloride, doxycycline, doxycycline hydrochloride, econazole, econazole nitrate, enilconazole, enoxacin, enrofloxacin, eosine, epicillin, ertapenem sodium, erythromycin, erythromycin estolate, erythromycin ethyl succinate, erythromycin lactobionate, erythromycin stearate, ethacridine, ethacridine lactate, ethambutol, ethanoic acid, ethionamide, ethyl alcohol, eugenol, exalamide, faropenem, fenticonazole, fenticonazole nitrate, fezatione, fleroxacin, flomoxef, flomoxef sodium, florfenicol, flucloxacillin, flucloxacillin magnesium, flucloxacillin sodium, fluconazole, flucytosine, flumequine, flurithromycin, flutrimazole, fosfomycin, fosfomycin calcium, fosfomycin sodium, framycetin, framycetin sulphate, furagin, furazolidone, fusafungin, fusidic acid, fusidic acid sodium salt, gatifloxacin, gemifloxacin, gentamicin antibiotic complex, gentamicin c1a, gentamycin sulfate, glutaraldehyde, gramicidin, grepafloxacin, griseofulvin, halazon, haloprogine, hetacillin, hetacillin potassium, hexachlorophene, hexamidine, hexetidine, hydrargaphene, hydroquinone, hygromycin, imipenem, isepamicin, isepamicin sulfate, isoconazole, isoconazole nitrate, isoniazid, isopropanol, itraconazole, josamycin, josamycin propionate, kanamycin, kanamycin sulphate, ketoconazole, kitasamycin, lactic acid, lanoconazole, lenampicillin, leucomycin A1, leucomycin A13, leucomycin A4, leucomycin A5, leucomycin A6, leucomycin A7, leucomycin A8, leucomycin A9, levofloxacin, lincomycin, lincomycin hydrochloride, linezolid, liranaftate, lividomycin, 1-menthol, lomefloxacin, lomefloxacin hydrochloride, loracarbef, lymecyclin, lysozyme, mafenide acetate, magnesium monoperoxophthalate hexahydrate, mecetronium ethylsulfate, mecillinam, meclocycline, meclocycline sulfosalicylate, mepartricin, merbromin, meropenem, metalkonium chloride, metampicillin, methacycline, methenamin, methyl salicylate, methylbenzethonium chloride, methylrosanilinium chloride, meticillin, meticillin sodium, metronidazole, metronidazole benzoate, mezlocillin, mezlocillin sodium, miconazole, miconazole nitrate, micronomicin, micronomicin sulfate, midecamycin, minocycline, minocycline hydrochloride, miocamycin, miristalkonium chloride, mitomycin c, monensin, monensin sodium, morinamide, moxalactam, moxalactam disodium, moxifloxacin, mupirocin, mupirocin calcium, nadifloxacin, nafcillin, nafcillin sodium, naftifine, nalidixic acid, natamycin, neomycin a, neomycin antibiotic complex, neomycin C, neomycin sulfate, neticonazole, netilmicin, netilmicin sulfate, nifuratel, nifuroxazide, nifurtoinol, nifurzide, nimorazole, niridazole, nitrofurantoin, nitrofurazone, nitroxolin, norfloxacin, novobiocin, nystatin antibiotic complex, octenidine, ofloxacin, oleandomycin, omoconazol, orbifloxacin, ornidazole, ortho-phenylphenol, oxacillin, oxacillin sodium, oxiconazole, oxiconazole nitrate, oxoferin, oxolinic acid, oxychlorosene, oxytetracycline, oxytetracycline calcium, oxytetracycline hydrochloride, panipenem, paromomycin, paromomycin sulfate, pazufloxacine, pefloxacin, pefloxacin mesylate, penamecillin, penicillin G, penicillin G potassium, penicillin G sodium, penicillin V, penicillin V calcium, penicillin V potassium, pentamidine, pentamidine diisetionate, pentamidine mesilas, pentamycin, phenethicillin, phenol, phenoxyethanol, phenylmercuriborat, PHMB, phthalylsulfathiazole, picloxydin, pipemidic acid, piperacillin, piperacillin sodium, pipercillin sodium-tazobactam sodium, piromidic acid, pivampicillin, pivcefalexin, pivmecillinam, pivmecillinam hydrochloride, policresulen, polymyxin antibiotic complex, polymyxin B, polymyxin B sulfate, polymyxin B1, polynoxylin, povidone-iodine, propamidin, propenidazole, propicillin, propicillin potassium, propionic acid, prothionamide, protiofate, pyrazinamide, pyrimethamine, pyrithion, pyrrolnitrin, quinoline, quinupristin-dalfopristin, resorcinol, ribostamycin, ribostamycin sulfate, rifabutin, rifampicin, rifamycin, rifapentine, rifaximin, ritiometan, rokitamycin, rolitetracycline, rosoxacin, roxithromycin, rufloxacin, salicylic acid, secnidazol, selenium disulphide, sertaconazole, sertaconazole nitrate, siccanin, sisomicin, sisomicin sulfate, sodium thiosulfate, sparfloxacin, spectinomycin, spectinomycin hydrochloride, spiramycin antibiotic complex, spiramycin b, streptomycin, streptomycin sulphate, succinylsulfathiazole, sulbactam, sulbactam sodium, sulbenicillin disodium, sulbentin, sulconazole, sulconazole nitrate, sulfabenzamide, sulfacarbamide, sulfacetamide, sulfacetamide sodium, sulfachlorpyridazine, sulfadiazine, sulfadiazine silver, sulfadiazine sodium, sulfadicramide, sulfadimethoxine, sulfadoxine, sulfaguanidine, sulfalene, sulfamazone, sulfamerazine, sulfamethazine, sulfamethazine sodium, sulfamethizole, sulfamethoxazole, sulfamethoxazol-trimethoprim, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfonamides, sultamicillin, sultamicillin tosilate, tacrolimus, talampicillin hydrochloride, teicoplanin A2 complex, teicoplanin A2-1, teicoplanin A2-2, teicoplanin A2-3, teicoplanin A2-4, teicoplanin A2-5, teicoplanin A3, teicoplanin antibiotic complex, telithromycin, temafloxacin, temocillin, tenoic acid, terbinafine, terconazole, terizidone, tetracycline, tetracycline hydrochloride, tetracycline metaphosphate, tetramethylthiuram monosulfide, tetroxoprim, thiabendazole, thiamphenicol, thiaphenicol glycinate hydrochloride, thiomersal, thiram, thymol, tibezonium iodide, ticarcillin, ticarcillin-clavulanic acid mixture, ticarcillin disodium, ticarcillin monosodium, tilbroquinol, tilmicosin, tinidazole, tioconazole, tobramycin, tobramycin sulfate, tolciclate, tolindate, tolnaftate, toloconium metilsulfat, toltrazuril, tosufloxacin, triclocarban, triclosan, trimethoprim, trimethoprim sulfate, triphenylstibinsulfide, troleandomycin, trovafloxacin, tylosin, tyrothricin, undecoylium chloride, undecylenic acid, vancomycin, vancomycin hydrochloride, verdamicin, viomycin, virginiamycin antibiotic complex, voriconazol, xantocillin, xibornol and zinc undecylenate.

In some embodiments of the compositions and methods described herein, the antibiotic agent is a β-lactam antibiotic, or an antibiotic comprising a β-lactam moiety. As used herein, “β-lactam antibiotics” (beta-lactam antibiotics) refers to the broad class of antibiotics consisting of all antibiotic agents comprising a β-lactam nucleus in their molecular structures. This class of antibiotics includes a variety of sub-groups, such as, for example, penicillin derivatives (penams), cephalosporins (cephems), monobactams, and penems and carbapenems. Most β-lactam antibiotics act by inhibiting cell wall biosynthesis in the bacterial organism. Bacteria often develop resistance to β-lactam antibiotics by synthesizing a β-lactamase, an enzyme that attacks the β-lactam ring. To overcome this resistance, β-lactam antibiotics are often given with β-lactamase inhibitors such as clavulanic acid.

In some embodiments of the compositions and methods described herein, the β-lactam antibiotic agent is a penam antibiotic or a penicillin antibiotic. As used herein, a “penicillin antibiotic” or “penam antibiotic” refer to a β-lactam antibiotic in which the core ring structure comprises a thiazolidine ring. Non-limiting examples of penicillin β-lactam antibiotics for use in the compositions and methods described herein include amoxicillin, ampicillin, methicillin, oxacillin ((2S,5R,6R)-3,3-dimethyl-6-[(5-methyl-3-phenyl-1,2-oxazole-4-carbonyl)amino]-7-oxo-4-thia-1-azabicyclo[3.2.0]heptane-2-carboxylic acid), nafcillin, cloxacillin, dicloxacillin ((2S,5R,6R)-6-{[3-(2,6-dichlorophenyl)-5-methyl-oxazole-4-carbonyl]amino}-3,3-dimethyl-7-oxo-4-thia-1-azabicyclo[3.2.0]heptane-2-carboxylic acid), flucloxacillin ((2S,5R,6R)-6-({[3-(2-chloro-6-fluorophenyl)-5-methylisoxazole-4-yl]carbonyl}amino)-3,3-dimethyl-7-oxo-4-thia-1-azabicyclo[3.2.0]heptane-2-carboxyl-ic acid), azlocillin, carbenicillin, ticarcillin, mezlocillin, piperacillin ((2S,5R,6R)-6-{[(2R)-2-[(4-ethyl-2,3-dioxo-piperazine-1-carbonyl)amino]-2-phenyl-acetyl]amino}-3,3-dimethyl-7-oxo-4-thia-1-azabicyclo[3.2.0]heptane-2-carboxylic acid); benzathine penicillin (benzathine & benzylpenicillin); benzylpenicillin (penicillin G); phenoxymethylpenicillin (penicillin V); procaine penicillin (procaine & benzylpenicillin); temocillin; co-amoxiclav (amoxicillin & clavulanic acid); and mecillinam,

In some embodiments of the compositions and methods described herein, the β-lactam antibiotic agent is a cephalosporin or cephamycin. As used herein, a “cephalosphorin antibiotic” or “cephamycins antibiotic” refer to a β-lactam antibiotic in which the core ring structure comprises a 3,6-dihydro-2H-1,3-thiazine ring. Non-limiting examples of cephalosporin β-lactam antibiotics for use in the compositions and methods described herein include cefazolin, cefalexin, cefalotin, cefdinir, cefepime, cefotaxime, cefpodoxime proxetil, ceftobiprole, ceftaroline fosamil, cephalosporin C, cephalothin, cefaclor, cefamandole, cefuroxime, cefotetan, cefoxitin, cefixime, ceftazidime, ceftriaxone, and cefpirome.

In some embodiments of the compositions and methods described herein, the β-lactam antibiotic agent is a carbapenem. As used herein, a “carbapenem antibiotic” refers to a β-Lactam antibiotic in which the core ring structure comprises a 2,3-dihydro-1H-pyrrole ring. Non-limiting examples of carbapenem antibiotics for use in the compositions and methods described herein include ertapenem ((4R,5S,6S)-3-[(3S,5S)-5-[(3-carboxyphenyl)carbamoyl]pyrrolidin-3-yl]sulfanyl-6-(1-hydroxyethyl)-4-methyl-7-oxo-1-azabicyclo[3.2.0]hept-2-ene-2-car-boxylic acid); meropenem (3-[5-(dimethylcarbamoyl) pyrrolidin-2-yl]sulfanyl-6-(1-hydroxyethyl)-4-methyl-7-oxo-1-azabicyclo[3-0.2.0]hept-2-ene-2-carbox ylic acid); imipenem, generally given as part of Imipenem/cilastatin; doripenem; panipenem/betamipron; biapenem; razupenem (PZ-601); and tebipenem.

In some embodiments of the compositions and methods described herein, the β-lactam antibiotic agent is a penem. As used herein, a “penem antibiotic” refers to a β-lactam antibiotic in which the core ring structure comprises a 2,3-dihydrothiazole ring. Non-limiting examples of penem antibiotics for use in the compositions and methods described herein include thiopenems, oxypenems, aminopenems, alkylpenems, and arylpenems.

In some embodiments of the compositions and methods described herein, the β-lactam antibiotic agent is a monobactam. As used herein, a “monobactam antibiotic” refers to a β-lactam antibiotic in which the core ring structure is not fused to another ring. Non-limiting examples of monobactam β-lactam antibiotics for use in the compositions and methods described herein include aztreonam, tigemonam, nocardicin A, and tabtoxinine ε-lactam.

In some embodiments of the compositions and methods described herein, when the antibiotic agent is a β-lactam antibiotic agent, the ROS target modulator is selected from a cytochrome oxidase inhibitor, an NADH dehydrogenase inhibitor, a succinate dehydrogenase inhibitor, or any combination thereof.

In some embodiments of the compositions and methods described herein, the antibiotic agent is a fluorquinolone antibiotic. The fluoroquinolones exert their therapeutic effects, in part, by interfering with bacterial DNA replication by inhibiting DNA gyrase. Fluoroquinolones increase the uptake of deoxyuridine, uridine, and thymidine into the DNA of human lymphocytes and decrease pyrimidine production. As used herein, a “fluoroquinolone antibiotic” refers to a compound comprising a polyatomic molecule comprising at least one quinolone moiety having at least one fluorine substituent, and which is capable of providing a bacteriostatic or bactericidal effect.

Non-limiting examples of fluoroquinolones that can be used with the ROS target modulators described herein include ciprofloxacin, moxifloxacin, ofloxacin, balofloxacin, grepafloxacin, levofloxacin ((S)-7-fluoro-6-(4-methylpiperazin-1-yl)-10-oxo-4-thia-1-azatricyclo[7.3.-1.0]trideca-5(13),6,8,11-tetraene-11-carboxylic acid), pazufloxacin, sparfloxacin, temafloxacin, and tosufloxacin.

In some embodiments of the compositions and methods described herein, when the antibiotic agent administered or contacted is a fluoroquinolone, the ROS target modulator is selected from a cytochrome oxidase inhibitor, an NADH dehydrogenase inhibitor, a succinate dehydrogenase inhibitor, a phospho acetyl transferase inhibitor, or any combination thereof.

In some embodiments of the compositions and methods described herein, the antibiotic agent is a nitroimidazole compound antibiotic. As used herein, a “nitroimidazole compound antibiotic” refers to an nitroimidazole (5-Nitro-1H-imidazole) derivative that contains a nitro group, and which is capable of providing a bacteriostatic or bactericidal effect.

Non-limiting examples of nitroimidazole compound antibiotics that can be used with the ROS target modulators described herein include metronidazole (2-(2-methyl-5-nitro-1H-imidazol-1-yl) ethanol), tinidazole, and nimorazole.

In some embodiments of the compositions and methods described herein, the antibiotic agent is a tetracycline antibiotic. Tetracycline antibiotics are named for their four (“tetra-”) hydrocarbon rings (“-cycl-”) derivation (“-ine”). Tetracycline antibiotics are protein synthesis inhibitors, inhibiting the binding of aminoacyl-tRNA to the mRNA-ribosome complex. They do so mainly by binding to the 30S ribosomal subunit in the mRNA translation complex. Tetracyclines also have been found to inhibit matrix metalloproteinases. As used herein, a “tetracycline antibiotic” refers to a subclass of polyketides having an octahydrotetracene-2-carboxamide skeleton, and collectively known as “derivatives of polycyclic naphthacene carboxamide,” and which is capable of providing a bacteriostatic or bactericidal effect.

Non-limiting examples of tetracycline antibiotics that can be used with the ROS target modulators described herein include tetracycline, chlortetracycline, oxytetracycline, demeclocycline, doxycycline, lymecycline, meclocycline, methacycline, minocycline, and rolitetracycline.

In some embodiments of the compositions and methods described herein, the antibiotic agent is an aminoglycoside antibiotic. The term “aminoglycoside antibiotic” refers to any naturally occurring drug, or semi-synthetic or synthetic derivative, comprising a highly-conserved aminocyclitol ring (ring II), which is a central scaffold that is linked to various amino-modified sugar moieties, that has antibiotic activity, as the term is defined herein. Aminoglycosides belong to several subclasses and antibiotics in each subclass show close structural resemblance. Aminoglycosides have several mechanisms of antibiotic activity, including, but not limited to, inhibition of protein synthesis; interfering with proofreading processes during translation, and causing increased rate of error in synthesis with premature termination; inhibition of ribosomal translocation where the peptidyl-tRNA moves from the A-site to the P-site; disruption of bacterial cell membrane integrity; and/or binding to bacterial 30S ribosomal subunit.

Non-limiting examples of aminoglycosides useful in the compositions and methods described herein include streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, and paromomycinlividomycin, and derivatives thereof of each of these aminoglycoside antibiotics, including synthetic and semi-synthetic derivatives.

In some embodiments of the compositions and methods described herein, the ROS target modulator is administered or co-formulated with a bactericidal antibiotic that is subject to efflux from resistant bacterial cells. Because efflux is generally an active process, and requires energy, such resistant bacterial cells must maintain active metabolism, thus rendering them more susceptible to the ROS target modulators described herein.

In those embodiments, where a ROS target modulator is used to potentiate an aminoglycoside antibiotic, one of ordinary skill in the art can first determine whether the increase in basal ROS production by the ROS target modulator negatively impacts proton motive force (PMF) as described in WO2012151474, published Nov. 4, 2012, the contents of which are herein incorporated by reference in their entireties. In such instances, where the ROS target modulator negatively impacts PMF, the ROS target modulator should not be used with the aminoglycoside inhibitor, because PMF is important for aminoglycoside uptake. Accordingly, in some embodiments of the compositions and methods described herein, the antibiotic agent used with the ROS target modulator is not an aminoglycoside antibiotic.

### ROS Target Modulators and Methods of Treatment or Inhibition of Bacterial Infections Thereof

As demonstrated herein, contacting with or administering an effective amount of one or more ROS target modulators with an effective amount of an antibiotic agent that increases ROS production as part of its antibiotic activity can be used in methods of treatment or inhibition of bacterial infections and/or bacterial growth.

Accordingly, in some aspects, provided herein are methods for treating or inhibiting a bacterial infection, the methods comprising administering to a subject having or at risk for a bacterial infection an effective amount of at least one ROS target modulator or inhibitor and an effective amount of an antibiotic agent. The methods described herein can, in some aspects and embodiments, be used to inhibit, delay formation of, treat, and/or prevent or provide prophylactic treatment of bacterial infections in animals, including humans.

As used herein, the terms “inhibit”, “decrease,” “reduce,” “inhibiting” and “inhibition” have their ordinary and customary meanings to generally mean a decrease by a statistically significant amount, and include inhibiting the growth or cell division of a bacterial cell or bacterial cell population, as well as killing such bacteria. Such inhibition is an inhibition of about 1% to about 100% of the growth of the bacteria versus the growth of bacteria in the presence of the antibiotic agent, but in the absence of the effective amount of the one or more ROS target modulators compounds. Preferably, the inhibition is an inhibition of about at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or more, of the growth or survival of the bacteria in comparison to a reference or control level in the absence of the effective amount of the one or more ROS target modulator compounds.

The methods described herein are applicable to the treatment of human and non-human subjects or individuals. The terms “subject” and “individual” are used interchangeably herein, and refer to an animal, for example a human, recipient of the one or more ROS target modulator compounds and antibiotic agent, such as, for example, an NADH hydrogenase inhibitor and a β-lactam antibiotic. For treatment of those disease states which are specific for a specific animal, such as a human subject, the term “subject” refers to that specific animal. The terms ‘non-human animals’ and ‘non-human mammals’ are used interchangeably herein, and include mammals such as rats, mice, rabbits, sheep, cats, dogs, cows, horses, pigs, and non-human primates. In some embodiments, the subject is a veterinary patient such as a dog or cat. The term “subject” can also encompass any vertebrate including but not limited to mammals, reptiles, amphibians and fish.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder, such as a bacterial infection, and include one or more of: ameliorating a symptom of a bacterial infection in a subject; blocking or ameliorating a recurrence of a symptom of a bacterial infection; decreasing in severity and/or frequency a symptom of a bacterial infection in a subject; and stasis, decreasing, or inhibiting growth of a bacterial infection in a subject. Treatment means ameliorating, blocking, reducing, decreasing or inhibiting by about 1% to about 100% versus a subject to whom the effective amount of the one or more ROS target modulator compounds and antibiotic agent has not been administered. Preferably, the ameliorating, blocking, reducing, decreasing or inhibiting is about at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or more, versus a subject to whom the effective amount of the one or more ROS target modulator compounds and antibiotic agent has not been administered. Treatment is generally considered “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein, the phrase “alleviating a symptom of a bacterial infection” is ameliorating any condition or symptom associated with the infection. Alternatively, alleviating a symptom of a bacterial infection can involve reducing the infectious bacterial load in the subject relative to such load in an untreated control. As compared with an equivalent untreated control, such reduction or degree of prevention is at is about at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or more, as measured by any standard technique. Desirably, the bacterial infection is completely cleared as detected by any standard method known in the art, in which case the persistent infection is considered to have been treated. A patient who is being treated, for example, for a persistent infection is one who a medical practitioner has diagnosed as having such a condition. Diagnosis can be by any suitable means. Diagnosis and monitoring can involve, for example, detecting the level of microbial load in a biological sample (for example, a tissue biopsy, blood test, or urine test), detecting the level of a surrogate marker of the microbial infection in a biological sample, detecting symptoms associated with the infection, or detecting immune cells involved in the immune response typical of bacterial infections (for example, detection of antigen specific T cells or antibody production).

As used herein, the terms “preventing” and “prevention” have their ordinary and customary meanings, and include one or more of: preventing an increase in the growth of a population of bacteria in a subject, or on a surface or on a porous material; preventing development of a disease caused by a bacteria in a subject; and preventing symptoms of an infection or disease caused by a bacterial infection in a subject. As used herein, the prevention lasts at least about 0.5 days, 1 day, 2 days, 3 days, 4 days, 5 days, 6 days, 7 days, 10 days, 12 days, 15 days, 20 days, 25 days, 30 days, 35 days, 40 days or more days after administration or application of the effective amount of the one or more ROS target modulator compounds and antibiotic agent, as described herein.

Accordingly, in some aspects, provided herein are methods for inhibiting a bacterial infection, the methods comprising administering to a patient having or at risk for a bacterial infection an effective amount of one or more ROS target modulator compounds and an effective amount of an antibiotic agent.

In some aspects, provided herein are methods for preventing a bacterial infection, the methods comprising administering to a patient having or at risk for a bacterial infection an effective amount of one or more ROS target modulator compounds and an effective amount of an antibiotic agent.

In some aspects, provided herein are methods for inhibiting a bacterial infection, the methods comprising administering to a patient having or at risk for a bacterial infection an effective amount of a pharmaceutical composition comprising one or more ROS target modulator compounds and an antibiotic agent.

In some aspects, provided herein are methods for preventing a bacterial infection, the methods comprising administering to a patient having or at risk for a bacterial infection an effective amount of a pharmaceutical composition comprising one or more ROS target modulator compounds and an antibiotic agent.

Also provided herein, in some aspects, are methods for treating a bacterial infection, comprising: administering to a patient having a bacterial infection and undergoing treatment with an antibiotic, an effective amount of one or more ROS target modulator compounds.

In some embodiments of these methods and all such methods described herein, the ROS target modulator compound is an ATP synthase inhibitor. In some embodiments, the ATP synthase inhibitor is IF₁. In some embodiments, the ATP synthase inhibitor is an efrapeptin. In some embodiments, the ATP synthase inhibitor is selected from aurovertin B, citreoviridin, α-zearalenol, and any other myotoxin.

In some embodiments of these methods and all such methods described herein, the ROS target modulator compound is a succinate dehydrogenase inhibitor. In some embodiments, the succinate dehydrogenase inhibitor is selected from carboxin, thenoyltrifluoroacetone, malonate, malate, and oxaloacetate.

In some embodiments of these methods and all such methods described herein, the ROS target modulator compound is a glutamate dehydrogenase inhibitor. In some embodiments, the glutamate dehydrogenase inhibitor is selected from bromofuroate; 3-carboxy-5-bromofuroic acid; Palmitoyl-Coenzyme-A; orthovanadate; vanadyl sulphate, vanadyl acetylacetonate, glutarate; 2-oxoglutarate; estrogen; pyridine-2,6-dicarboxylic acid; and (−)-epigallocatechin gailate (EGCG).

In some embodiments of these methods and all such methods described herein, the ROS target modulator compound is a NADH dehydrogenase inhibitor. In some embodiments, the NADH dehydrogenase inhibitor is selected from Amytal; Amytal Sodium; Annonin VI; Aurachin A; Aurachin B; Aureothin; Benzimidazole; Bullactin; calnexin; Capsaicin; Ethoxyformic anhydride; Ethoxyquin; Fenpyroximate; Mofarotene; mofarotene 2-oxoglutarate dehydrogenase; Molvizarin; Myxalamide PI; M2-type pyruvate kinase; Otivarin; Pethidine; rhein; Phenalamid A₂; Phenoxan; Piericidin A; p-chloromercuribenzoate; Ranolazine; Rolliniasatin-1; Rolliniasatin-2; Rotenone; Squamocin; Thiangazole rotenoids; thiol reagents; Demerol; iron chelators; NAD⁺ (nicotinamide adenine dinucleotide; oxidized form); AMP (adenosine monophosphate); ADP (adenosine diphosphate); ADP-ribosylation factor 3; ATP (adenosine triphosphate); guanidinium salts; NADH; barbituates; gossypol; polyphenol; dihydroxynaphthoic acids; adenosine diphosphate ribose; rotenoid; acetogenin; nitrosothiols; peroxynitrite; carvedilol; arylazido-beta-alanyl NAD+; adriamycin; 4-hydroxy-2-nonenal; pyridine derivatives; 2-heptyl-4-hydroxyquinoline N-oxide; dicumarol; o-phenanthroline; and 2;2′-dipyridyl.

In some embodiments of these methods and all such methods described herein, the ROS target modulator compound is a pyruvate dehydrogenase inhibitor. In some embodiments, the pyruvate dehydrogenase inhibitor is selected from

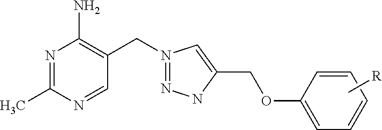

where R is 2-Cl-4-NO₂, 4-NO₂, 4-COOH, or H; secondary amides of (R)-3;3;3-Trifluoro-2-hydroxy-2-methylpropionic acid glyoxylate; (R)-3;3;3-Trifluoro-2-hydroxy-2-methylpropionamides; anilides of (R)-Trifluoro-2-hydroxy-2-methylpropionic acidhydroxypyruvate; kynurenate; xanthurenate; α-cyano-4-hydroxycinnamic acid; bromopyruvic acid; fluropyruvic acid; AZD-7545; phosphonate and phosphinate analogs of pyruvate; mono- and bifunctional arsenoxides; branched-chain 2-oxo acids; 2-oxo-3-alkynoic acids; tetrahydrothiamin diphosphate (ThDP); and 2-thiazolone and 2-thiothiazolone analogs of ThDP.

In some embodiments of these methods and all such methods described herein, the ROS target modulator compound is a cytochrome oxidase inhibitor. In some embodiments, the cytochrome oxidase inhibitor is selected from azide; nitric oxide; cytochrome P450 oxidase inhibitors; aurachin A; Aurachin C; aurachin D; tridecylstigmatelli; stigmatellin; nigericin; hydroxylamine; heptylhydroxyquinoline N-oxide (HQNO); nonylhydroxyquinoline N-oxide (NQNO); dibromothymoquinone (DBMIB); piericidin A; and undecylhydroxydioxobenzo-thiazole (UHDBT).

In some embodiments of these methods and all such methods described herein, the ROS target modulator compound is a triose phosphate isomerase inhibitor. In some embodiments, the triose phosphate isomerase inhibitor is selected from 3-haloacetol phosphate; glycidol phosphate; phosphoenol pyruvate; DHAP; GAP; 2-phosphoglycollate; phosphoglycolohydroxamate; 3-phosphoglycerate; glycerol phosphate; phosphoenol pyruvate; 2;9-Dimethyl-β-carbolines and derivatives thereof; 3-(2-benzothiazolylthio)-1-propanesulfonic acid; 2-carboxyethylphosphonic acid; 2-phosphoglyceric acid; N-hydroxy-4-phosphono-butanamide; and [2(formyl-hydroxy-amino)-ethyl]-phosphonic acid.

In some embodiments of these methods and all such methods described herein, the ROS target modulator compound is a glucose-6-phosphate dehydrogenase inhibitor. In some embodiments, the glucose-6-phosphate dehydrogenase inhibitor is selected from dehydroepiandrosterone (DHEA), DHEA-sulfate; 2-deoxyglucose; halogenated DHEA; epiandrosterone; isoflurane; sevoflurane; diazepam; CBF-BS2; cystamine; 16α-bromoepiandrosterone; 16α-hydroxy-5-androsten-17-one; 16α-fluoro-5-androsten-17-one; 16α-fluoro-16β-methyl-5-androsten-17-one; 16α-methyl-5-androsten-17-one; 16β-methyl-5-androsten-17-one; 16α-hydroxy-5α-androstan-17-one; 16α-fluoro-5α-androstan-17-one; 16α-fluoro-160-methyl-5α-androstan-17-one; 16α-methyl-5α-androstan-17-one; 16β-methyl-5α-androstan-17-one; and 2-amino-2-deoxy-D-glucose-6-phosphate.

In some embodiments of these methods and all such methods described herein, the ROS target modulator compound is a 6-phosphogluconate dehydrogenase inhibitor. In some embodiments, the 6-phosphogluconate dehydrogenase inhibitor is selected from 6-aminonicotinamide; aldonate 4-phospho-d-erythronate; 5,6-Dideoxy-6-phosphono-d-arabino-hexonate; and 5-deoxy-5-phosphono-d-arabinonate.

In some embodiments of these methods and all such methods described herein, the ROS target modulator compound is a succinyl-CoA synthetase inhibitor. In some embodiments, the succinyl-CoA synthetase inhibitor is selected from LY266500 and vanadium sulphate.

In some embodiments of these methods and all such methods described herein, the ROS target modulator compound is a phosphate acetyltransferase inhibitor.

In some embodiments of these methods and all such methods described herein, the ROS target modulator compound is a phosphofructokinase inhibitor. In some embodiments, the phosphofructokinase inhibitor is selected from aurintricarboxylic acid; pyruvate; 2-deoxy-2-fluoro-D-glucose; citrate and halogenated derivatives of citrate; fructose 2,6-bisphosphate; N-(2-methoxyethyl)-bromoacetamide; N-(2-ethoxyethyl)-bromoacetamide; N-(3-methoxypropyl)-bromoacetamide); phosphoglycerate; taxodone; taxodione; euparotin acetate eupacunin; vernolepin; argaric acid, quinaldic acid; and 5′-p-flurosuflonylbenzoyl adenosine.

In some embodiments of these methods and all such methods described herein, the ROS target modulator compound is a fumarase B inhibitor. In some embodiments, the fumarase B inhibitor is selected from trans-aconitate; bromomesaconate; citrate; meso-tartaric acid; bismuth; DL-β-fluoromalic acid; and S-2,3-Dicarboxyaziridine.

In some embodiments of these methods and all such methods described herein, the antibiotic agent is selected from a β-lactams antibiotic; an aminoglycoside antibiotic; vancomycins; bacitracins; macrolides; lincosamides; chloramphenicols; tetracyclines; amphotericins; cefazolins; clindamycins; mupirocins; sulfonamides and trimethoprim; rifampicins; metronidazoles; quinolones; novobiocins; polymixins; gramicidins; or any salts or variants thereof.

In some embodiments of these methods and all such methods described herein, the antibiotic agent is a β-lactam antibiotic or an antibiotic comprising a β-lactam moiety.

In some embodiments of these methods, the β-lactam antibiotic agent is a penam antibiotic or a penicillin antibiotic. In some embodiments of these methods, the penicillin antibiotic is selected from amoxicillin, ampicillin, methicillin, oxacillin, nafcillin, cloxacillin, dicloxacillin, flucloxacillin, azlocillin, carbenicillin, ticarcillin, mezlocillin, piperacillin, benzathine penicillin, benzylpenicillin, phenoxymethylpenicillin, procaine penicillin; temocillin; co-amoxiclav; and mecillinam.

In some embodiments of these methods, the β-lactam antibiotic agent is a cephalosporin or cephamycin. In some embodiments of these methods, the cephalosporin or cephamycin antibiotic is selected from cefazolin, cefalexin, cefalotin, cefdinir, cefepime, cefotaxime, cefpodoxime proxetil, ceftobiprole, ceftaroline fosamil, cephalosporin C, cephalothin, cefaclor, cefamandole, cefuroxime, cefotetan, cefoxitin, cefixime, ceftazidime, ceftriaxone, and cefpirome.

In some embodiments of these methods, the β-lactam antibiotic agent is a carbapenem. In some embodiments of these methods, the carbapenem antibiotic is selected from ertapenem, meropenem, imipenem, doripenem, panipenem/betamipron, biapenem, razupenem, and tebipenem.

In some embodiments of these methods, the β-lactam antibiotic agent is a penem. In some embodiments of these methods, the penem antibiotic is selected from thiopenems, oxypenems, aminopenems, alkylpenems, and arylpenems.

In some embodiments of these methods, the β-lactam antibiotic agent is a monobactam antibiotic. In some embodiments of these methods, the monobactam antibiotic is selected from aztreonam, tigemonam, nocardicin A, and tabtoxinine β-lactam.

In some embodiments of these methods and all such methods described herein, when the antibiotic agent administered or contacted is a β-lactam antibiotic agent, the ROS target modulator is selected from a cytochrome oxidase inhibitor, an NADH dehydrogenase inhibitor, a succinate dehydrogenase inhibitor, or any combination thereof.

In some embodiments of these methods and all such methods described herein, the antibiotic agent is a fluorquinolone antibiotic. In some embodiments of these methods, the fluoroquinolone antibiotic is selected from ciprofloxacin, moxifloxacin, ofloxacin, balofloxacin, grepafloxacin, levofloxacin, pazufloxacin, sparfloxacin, temafloxacin, and tosufloxacin.

In some embodiments of these methods and all such methods described herein, when the antibiotic agent administered or contacted is a fluoroquinolone, the ROS target modulator is selected from a cytochrome oxidase inhibitor, an NADH dehydrogenase inhibitor, a succinate dehydrogenase inhibitor, a phospho acetyl transferase inhibitor, or any combination thereof.

In some embodiments of these methods and all such methods described herein, the antibiotic agent is a nitroimidazole compound antibiotic. In some embodiments of these methods, the nitroimidazole compound antibiotic is selected from metronidazole, tinidazole, and nimorazole.

In some embodiments of these methods and all such methods described herein, the antibiotic agent is a tetracycline antibiotic. In some embodiments of these methods, the tetracycline antibiotic is selected from tetracycline, chlortetracycline, oxytetracycline, demeclocycline, doxycycline, lymecycline, meclocycline, methacycline, minocycline, and rolitetracycline.

In some embodiments of these methods and all such methods described herein, the antibiotic agent is an aminoglycoside antibiotic. In some embodiments of these methods, the aminoglycoside antibiotic is selected from streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, and paromomycinlividomycin.

In other embodiments of the methods described herein, the antibiotic agent administered with the ROS target modulator is not an aminoglycoside antibiotic.

The ROS target modulator compounds described herein that potentiate and improve antibiotic efficacy, as exemplified in E coli, can be effective for increasing ROS production in a variety of bacterial species, including, but not limited to, E. coli, according to the compositions and methods described herein. Accordingly, the ROS target modulator compounds are effective at improving and enhancing the treatment of various disorders and diseases caused by bacterial infections or toxins produced during such infections. Such bacterial infections include those caused by bacteria having a similar metabolic system to E. coli, such as, for example, those comprising one or more of the glycolysis pathway, pentose-phosphate pathway shunt, the EntnerDoudoroff pathway, the TCA cycle, glyoxylate shunt, and acetate metabolism. Other bacterial species for which metabolic constructions are available include Mycobacterium tuberculosis, Staphylococcus aureus, Haemophilus influenzae, and Salmonella typhimurium⁵⁷⁻⁶¹. In some embodiments, bacterial species can be determined to share similar metabolic systems to E. coli, and thus be amenable to the use of the ROS target modulator compounds described herein using the systems-based, genome-scale ROS metabolic models described herein and consequent experimental validation. Accordingly, in some embodiments of the aspects described herein, the bacteria being inhibited by the ROS target modulators and methods thereof is an aerobic bacteria or a facultative anaerobe. As used herein, a “facultative anaerobe” is a bacterium that makes ATP by aerobic respiration if oxygen is present but is also capable of switching to fermentation. In contrast, obligate anaerobes die in the presence of oxygen. In some embodiments, the facultative anaerobe is a bacterial species that uses mixed-acid fermentation in anaerobic conditions and produces one or more of lactate, succinate, ethanol, acetate and/or carbon dioxide. In some embodiments, the bacterial species comprises a metabolic system that comprises one or more of the glycolysis pathway, pentose-phosphate pathway shunt, and/or the EntnerDoudoroff pathway. In some embodiments, the bacterial species comprises a metabolic system that comprises the TCA cycle and/or glyoxylate shunt. In some embodiments, the bacterial species comprises a metabolic system comprising acetate metabolism.

Non-limiting examples of disorders/diseases caused by bacterial infections or toxins produced during bacterial infections, and for which the compositions and methods described herein are applicable in various aspects and embodiments, include, but are not limited to, pneumonia, sepsis or bacteremia, toxic shock syndrome, bacterial meningitis, endocarditis, gastroenteritis, peritonitis, strep throat, osteomyelitis, cholera, diphtheria, tuberculosis, anthrax, botulism, brucellosis, campylobacteriosis, typhus, ear infections (e.g., otitis media), including recurrent ear infections, recurrent pneumonia, gonorrhea, hemolytic-uremic syndrome, listeriosis, lyme disease, mastitis, peritonitis, rheumatic fever, pertussis (Whooping Cough), plague, salmonellosis, scarlet fever, shigellosis, sinusitis, including chronic sinusitis, syphilis, trachoma, tularemia, typhoid fever, and urinary tract infections, including chronic urinary tract infections. In other embodiments, the disorder or disease is an infection of soft tissue or skin, such as acne, cellulitis, abscess, necrotizing fasciitis, impetigo, erysipelas, or an infection of a burn or wound, including surgical wounds and skin ulcer (e.g., diabetic ulcer).

Accordingly, in various embodiments of methods and compositions and methods described herein, the combination of antibiotic and one or more ROS target modulators administered or used is determined based on the nature of the bacterial infection, for example, whether an acute or chronic infection, in the subject.

Non-limiting examples of infectious bacteria causing bacterial infections that are contemplated for use with the combinatorial therapeutic compositions and methods described herein include, but are not limited to: Helicobacterpyloris, Borelia burgdorferi, Legionella pneumophilia, Mycobacteria sps (such as M. tuberculosis, M. avium, M. intracellulare, M. kansaii, M. gordonae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, Bacillus cereus, Bifidobacterium bifidum, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli, Lactobacillus spp.; Nocardia spp.; Rhodococcus equi (coccobacillus); Erysipelothrix rhusiopathiae; Actinomyces spp.; Clostridium botulinum; Clostridium difficile; Mobiluncus spp., Peptostreptococcus spp.; Moraxella catarrhalis; Veillonella spp.; Actinobacillus actinomycetemcomitans; Acinetobacter baumannii; Bordetella pertussis; Brucella spp.; Campylobacter spp.; Capnocytophaga spp.; Cardiobacterium hominis; Eikenella corrodens; Francisella tularensis; Haemophilus ducreyi; Kingella kingae; Pasteurella multocida; Klebsiella granulomatis; Citrobacter spp., Enterobacter spp.; Escherichia coli; Klebsiella pneumoniae; Proteus spp.; Salmonella enteriditis; Salmonella typhi; Shigella spp.; Serratia marcescens; Yersinia enterocolitica; Yersinia pestis; Aeromonas spp.; Plesiomonas shigelloides; Vibrio cholerae; Vibrio parahaemolyticus; Vibrio vulnificus; Acinetobacter spp.; Flavobacterium spp.; Burkholderia cepacia; Burkholderia pseudomallei; Xanthomonas maltophilia or Stenotrophomonas maltophila; Bacteroides fragilis; Bacteroides spp.; Prevotella spp.; Fusobacterium spp.; Spirillum minus; Borrelia burgdorferi; Borrelia recurrentis; Bartonella henselae; Chlamydia trachomatis; Chlamydophila pneumoniae; Chlamydophila psittaci; Coxiella burnetii; Ehrlichia chaffeensis; Anaplasma phagocytophilum; Legionella spp.; Leptospira spp.; Rickettsia rickettsii; Orientia tsutsugamushi; and Treponema pallidum. Mycobacterial infections that can be treated using the methods and compositions described herein include, but are not limited to, those caused by: M. abscessus, M. africanum, M. asiaticum, Mycobacterium avium complex (MAC), M. avium paratuberculosis, M. bovis, M. chelonae, M. fortuitum, M. gordonae, M. haemophilum, M. intracellulare, M. kansasii, M. lentiflavum, M. leprae, M. liflandii, M. malmoense, M. marinum, M. microti, M. phlei, M. pseudoshottsii, M. scrofulaceum, M. shottsii, M. smegmatis, M. triplex, M. tuberculosis, M. ulcerans, M. uvium, and M. xenopi.

Also provided herein in some embodiments and aspects of the compositions and methods described herein, are synergistic combinations of ROS target modulator compounds and antibiotic agents for the treatment of bacterial infections exhibiting antibiotic or drug resistance. In some embodiments of the aspects described herein, the infection is caused by a bacterial species that exhibits antibiotic resistance. In some such embodiments, the infection is caused by a bacterial species that exhibits multi-drug resistance (MDR). In some embodiments, the MDR can be due, at least in-part, to active efflux of the antibiotic drugs from bacterial cells.

As used herein, “multidrug resistance” or “MDR” is a phenomenon in bacteria that occurs via the accumulation of genes, on resistance (R) plasmids or transposons, each coding for resistance to a specific agent, and/or by the action of multidrug efflux pumps, each of which can pump out more than one drug type. One mechanism of multidrug resistance is via mutational alteration of the protein the drug or antibiotic targets. For example, bacteria can become resistant through mutations that make the target protein less susceptible to the agent. Fluoroquinolone resistance is mainly (but not exclusively) due to mutations in the target enzymes, DNA topoisomerases. Ribosomal resistance mutations are often found in the aminoglycoside-resistant clinical strains of Mycobacterium tuberculosis. Erm gene mutations can cause resistance to macrolides (erythromycin and many others), lincosamide, and streptogramin of group B, the MLS phenotype.

Another mechanism of multidrug resistance is via enzymatic inactivation of the drug. For example, aminoglycosides, such as kanamycin, tobramycin, and amikacin, can be inactivated by enzymatic phosphorylation [e.g., by aminoglycoside phosphoryltransferase (APH)], acetylation [by aminoglycoside acetyltransferase (AAC)], or adenylation (by aminoglycoside adenyltransferase or nucleotidyltransferase). β-lactams, such as penicillins, cephalosporins, and carbapenems such as imipenem, can be inactivated by enzymatic hydrolysis by β-lactamases, usually in the periplasm. Genes coding for these inactivating enzymes can produce resistance as additional genetic components on plasmids. Aminoglycosides can be inactivated by modifications that reduce net positive charges on these polycationic antibiotics.

Another mechanism of multidrug resistance is via preventing drug access to targets. Drug access to the molecule targeted by the drug or antibiotic can be reduced locally, or it can be reduced by an active efflux process. For example, Tet(M) or Tet(S) proteins, produced by plasmid-coded genes in gram-positive bacteria, bind to ribosomes with high affinity and change the ribosomal conformation, thereby preventing the association of tetracyclines to ribosomes. Plasmid-coded Qnr proteins protect DNA topoisomerases from (fluoro)quinolones. In terms of drug resistance caused by drug-specific efflux pumps, in gram-negative bacteria, such as E. coli, antibiotic access to a target molecule can be reduced generally by decreasing the influx across the outer membrane barrier, termed herein as “active efflux.” Examples of multidrug efflux pumps that cause MDR via active efflux include, but are not limited to, those belonging to: the Major Facilitator Superfamily or MFS, such as MFS Pumps with 14 transmembrane segments, which actively extrude monocationic biocides and dyes (e.g., QacA and QacB, and EmrB of E. coli), MFS Pumps with 12 TMSs (e.g., NorA of S. aureus; the Small Multidrug Resistance (SMR) family, which extrude cationic compounds such as quaternary ammonium biocides or ethidium, e.g., EmrE of E. coli; Resistance-Nodulation-Division (RND) family, which play an important role in producing multidrug resistance in gram-negative bacteria (e.g., AcrB and AcrD of E. coli and MexB, MexD, MexF, and MexY of P. aeruginosa; other multidrug efflux pumps energized by ionic gradients, such as those belonging to the Multidrug and Toxin Extrusion (MATE) family (e.g., NorM of Vibrio parahaemolyticus), and multidrug efflux pumps of the ATP-Binding Cassette superfamily.

Accordingly, in some embodiments of the compositions and methods described herein, a ROS target modulator compound is administered when the bacterial infection being treated has developed multidrug resistance. In some such embodiments, the multidrug resistance is caused or mediated by active efflux via drug-specific efflux pumps.

In some embodiments of the aspects described herein, the methods of treating a subject having or at increased risk for a bacterial infection, further comprise the step of selecting, diagnosing, or identifying a subject having or at increased risk for a bacterial infection. In such embodiments, a subject is identified as having a bacterial infection by objective determination of the presence of bacterial cells in the subject's body by one of skill in the art. Such objective determinations can be performed through the sole or combined use of tissue analyses, blood analyses, urine analyses, and bacterial cell cultures, in addition to the monitoring of specific symptoms associated with the bacterial infection.

In some embodiments of the methods described herein, the infection is an “acute” or “non-latent infection,” that is, an infection where the bacteria is actively or aggressively proliferating, and typically having a relatively short time course of infection. Such infections can require aggressive antibiotic intervention. Such infections are often termed “acute,” and lead to quickly advancing disease. Acute infections typically begin with an incubation period, during which the bacteria replicate and host innate immune responses are initiated. The cytokines produced early in infection lead to classical symptoms of an acute infection: aches, pains, fever, malaise, and nausea. Once an acute infection is cleared, the infectious agent cannot be detected in the subject. Acute infections, as used herein, do not enter a latent phase where the bacterial agent is present but the subject is non-symptomatic. In some embodiments, an acute infection is one in which the subject has one or more active symptoms of infection, e.g., aches, pains, fever, malaise, nausea, active/proliferating bacterial cells, active/proliferating immune cells, detectable levels of one or more cytokines in the circulation, etc. Non-limiting examples of conditions or disorders mediated by acute infections include diarrheal disorders, toxic shock syndrome, gastroenteritis, peritonitis, strep throat, osteomyelitis, cholera, diphtheria, anthrax, botulism, brucellosis, campylobacteriosis, typhus, ear infections (e.g., otitis media), gonorrhea, hemolytic-uremic syndrome, listeriosis, lyme disease, mastitis, peritonitis, rheumatic fever, pertussis (Whooping Cough), plague, salmonellosis, scarlet fever, shigellosis, sinusitis, primary syphilis, trachoma, tularemia, and urinary tract infections. In other embodiments, the disorder or disease is an infection of soft tissue or skin, such as acne, cellulitis, abscess, necrotizing fasciitis, impetigo, erysipelas, or an infection of a burn or wound, including surgical wounds and skin ulcer (e.g., diabetic ulcer)

Accordingly, in some embodiments of these methods and all such methods described herein, provided herein are methods of inhibiting or preventing an acute infection in a subject before, during, or after an invasive medical treatment, comprising administering to a subject before, during, and/or after an invasive medical treatment an effective amount of one or more ROS target compounds and an effective amount of an antibiotic agent.

Such methods can be used for achieving a systemic and/or local effect against relevant bacteria shortly before or after an invasive medical treatment, such as surgery or insertion of an in-dwelling medical device (e.g. joint replacement (hip, knee, shoulder, etc.)). Treatment can be continued after invasive medical treatment, such as post-operatively or during the in-body time of the device.

In some such embodiments, the one or more ROS target modulator compounds and the antibiotic agent can be administered once, twice, thrice or more, from 1 day, 2 days, 3 days, 4 days, 5 days, 6 days, 7 days or more, to 10 hours, 9 hours, 8 hours, 7 hours, 6 hours, 5 hours, 4 hours, 3 hours, 2 hours, or 1 hour or immediately before surgery for permitting a systemic or local presence of the antibiotic agent in combination with the one or more ROS target modulator compounds. The pharmaceutical composition(s) comprising the antibiotic agent and the one or more ROS target modulator compounds can, in some embodiments, be administered after the invasive medical treatment for a period of time, such as 1 day, 2 days, 3 days, 4 days, 5 days or 6 days, 1 week, 2 weeks, 3 weeks or more, or for the entire time in which the device is present in the body of the subject. As used herein, the term “bi-weekly” refers to a frequency of every 13-15 days, the term “monthly” refers a frequency of every 28-31 days and “bi-monthly” refers a frequency of every 58-62 days.

In some embodiments of these methods, the surface of the in-dwelling device is coated by a solution, such as through bathing or spraying, containing a concentration of about 1 μg/ml to about 500 mg/ml of the antibiotic agent and one or more ROS target modulator compounds described herein. When being applied to an in-dwelling medical device, the surface can be coated by a solution comprising the antibiotic agent and one or more ROS target modulator compounds before its insertion in the body.

In other embodiments of the methods described herein, the bacterial infection is a persistent or a chronic bacterial infection.

As used herein, “persistent infections” refer to those infections that, in contrast to acute infections, are not effectively or completely cleared by a host immune response or by antibiotic administration. Persistent infections include for example, latent, chronic and slow infections. In a “chronic infection,” the infectious agent can be detected in the subject at all times. However, the signs and symptoms of the disease can be present or absent for an extended period of time. Non-limiting examples of chronic infections include a variety of bacterial infections, as described herein below, as well as secondary bacterial infections resulting from or caused by infection with another agent that suppresses or weakens the immune system, such as chronic viral infections, such as, for example, hepatitis B (caused by hepatitis B virus (HBV)) and hepatitis C (caused by hepatitis C virus (HCV)) adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus II, as well as secondary bacterial infections resulting from or caused by infection with a persistent parasitic persistent infection, such as, for example, Leishmania, Toxoplasma, Trypanosoma, Plasmodium, Schistosoma, and Encephalitozoon.

Also provided herein, in some aspects, are methods of inhibiting or preventing growth of or colonization by a persistent, slow growing, stationary-phase or biofilm bacteria in a subject or on a surface. Infections in which bacteria are either slow-growing, persistent, or in a biofilm pose a serious clinical challenge for therapy because cells in these states exhibit tolerance to the activity of antimicrobial agents, such as antibiotics. Osteomyelitis, infective endocarditis, chronic wounds, infections related to in-dwelling devices, infections resulting from second- and third-degree burns, and bacterial infections that are secondary complications of respiratory or mucosal conditions, such as those arising from cystic fibrosis, sinusistis, and viral infections, are non-limiting examples of infections that harbor persistent bacterial cells. Because most antimicrobial agents exert maximal activity against rapidly dividing cells, antimicrobial therapies for these infections are not optimal, requiring protracted treatment times, high and sometimes toxic antibiotic doses, and demonstrating higher failure rates. In contrast, the novel methods and compositions described herein, which combine an effective amount of one or more ROS target modulators to potentiate the efficacy and bactericidal activity of an antibiotic agent, permits increased efficacy of the antibiotic agent and enhanced susceptibility of the bacteria to the agent.

The terms “persistent cell” or “persister bacterial cells” are used interchangeably herein and refer to a metabolically dormant subpopulation of microorganisms, typically bacteria, which are not sensitive to antimicrobial agents such as antibiotics. Persisters typically are not responsive, i.e. are not killed or inhibited by antibiotics, as they have, for example, non-lethally downregulated the pathways on which the antibiotics act. Persisters can develop at non-lethal (or sub-lethal) concentrations of the antibiotic.

Accordingly, in some aspects, provided herein are methods of inhibiting or preventing formation or colonization of a persistent, slow growing, stationary-phase or biofilm bacteria in a subject before, during, or after an invasive medical treatment, comprising administering to a subject before, during, and/or after an invasive medical treatment an effective amount of one or more ROS target compounds and an effective amount of an antibiotic agent.

Such methods can be used for achieving a systemic and/or local effect against relevant bacteria shortly before or after an invasive medical treatment, such as surgery or insertion of an in-dwelling medical device (e.g. joint replacement (hip, knee, shoulder, etc.)). Treatment can be continued after invasive medical treatment, such as post-operatively or during the in-body time of the device.

In some such embodiments, the one or more ROS target modulator compounds and the antibiotic agent can be administered once, twice, thrice or more, from 1 day, 2 days, 3 days, 4 days, 5 days, 6 days, 7 days or more, to 10 hours, 9 hours, 8 hours, 7 hours, 6 hours, 5 hours, 4 hours, 3 hours, 2 hours, or 1 hour or immediately before surgery for permitting a systemic or local presence of the antibiotic agent in combination with the one or more ROS target modulator compounds. The pharmaceutical composition(s) comprising the antibiotic agent and the one or more ROS target modulator compounds can, in some embodiments, be administered after the invasive medical treatment for a period of time, such as 1 day, 2 days, 3 days, 4 days, 5 days or 6 days, 1 week, 2 weeks, 3 weeks or more, or for the entire time in which the device is present in the body of the subject. As used herein, the term “bi-weekly” refers to a frequency of every 13-15 days, the term “monthly” refers a frequency of every 28-31 days and “bi-monthly” refers a frequency of every 58-62 days.

In some embodiments of these methods, the surface of the in-dwelling device is coated by a solution, such as through bathing or spraying, containing a concentration of about 1 μg/ml to about 500 mg/ml of the antibiotic agent and one or more ROS target modulator compounds described herein. When being applied to an in-dwelling medical device, the surface can be coated by a solution comprising the antibiotic agent and one or more ROS target modulator compounds before its insertion in the body.

In some embodiments of the methods described herein, a subject refers to a human subject having a chronic infection or at increased risk for a chronic infection or biofilm formation. A subject that has a chronic infection is a subject having objectively measurable bacterial cells present in the subject's body. A subject that has increased risk for a chronic infection includes subjects with an in-dwelling medical device, for example, or a subject having or having had a surgical intervention.

In some embodiments of the methods described herein, the subject having or at risk for a chronic infection is an immunocompromised subject, such as, for example, HIV-positive patients, who have developed or are at risk for developing pneumonia from either an opportunistic infection or from the reactivation of a suppressed or latent infection; subjects with cystic fibrosis, chronic obstructive pulmonary disease, and other such immunocompromised and/or institutionalized patients.

Also provided herein, in some aspects, are methods of inhibiting or delaying the formation of biofilms, comprising administering to a subject in need thereof or contacting a surface with an effective amount of one or more ROS target modulator compounds and an antibiotic agent in combination.

As used herein, a “biofilm” refers to mass of microorganisms attached to a surface, such as a surface of a medical device, and the associated extracellular substances produced by one or more of the attached microorganisms. The extracellular substances are typically polymeric substances that commonly include a matrix of complex polysaccharides, proteinaceous substances and glycopeptides. The microorganisms can include, but are not limited to, bacteria, fungi and protozoa. In a “bacterial biofilm,” the microorganisms include one or more species of bacteria. The nature of a biofilm, such as its structure and composition, can depend on the particular species of bacteria present in the biofilm. Bacteria present in a biofilm are commonly genetically or phenotypically different than corresponding bacteria not in a biofilm, such as isolated bacteria or bacteria in a colony. “Polymicrobic biofilms” are biofilms that include a plurality of bacterial species.

As used herein, the terms and phrases “delaying”, “delay of formation”, and “delaying formation of” have their ordinary and customary meanings, and are generally directed to increasing the period of time prior to the formation of biofilm, or a slow growing bacterial infection in a subject or on a surface. The delay may be, for example, about 12 hours, about 18 hours, about 24 hours, about 36 hours, about 48 hours, about 60 hours, about 72 hours, about 84 hours, about 96 hours, about 5 days, 6 days, 7 days, 8 days, 9 days, 10 days, or more. Inhibiting formation of a biofilm, as used herein, refers to avoiding the partial or full development or progression of a biofilm, for example, on a surface, such as a surface of an indwelling medical device.

The skilled artisan will understand that the methods of inhibiting and delaying the formation of biofilms can be practiced wherever bacteria, such as persistent, slow-growing, stationary-phase, or biofilm forming bacteria, can be encountered. For example, the methods described herein can be practiced on the surface of or inside of an animal, such as a human; on an inert surface, such as a counter or bench top; on a surface of a piece of medical or laboratory equipment; on a surface of a medical or laboratory tool; or on a surface of an in-dwelling medical device.

Accordingly, in some embodiments, the methods described herein further encompass surfaces coated by one or more ROS target modulator compounds and an antibiotic agent, and/or impregnated with one or more ROS target modulator compounds and an antibiotic agent. Such surfaces include any that can come into contact with a perisistent, slow growing, stationary-phase, biofilm bacteria. In some such embodiments, such surfaces include any surface made of an inert material (although surfaces of a living animal are encompassed within the scope of the methods described herein), including the surface of a counter or bench top, the surface of a piece of medical or laboratory equipment or a tool, the surface of a medical device such as a respirator, and the surface of an in-dwelling medical device. In some such embodiments, such surfaces include those of an in-dwelling medical device, such as surgical implants, orthopedic devices, prosthetic devices and catheters, i.e., devices that are introduced to the body of an individual and remain in position for an extended time. Such devices include, but are not limited to, artificial joints, artificial hearts and implants; valves, such as heart valves; pacemakers; vascular grafts; catheters, such as vascular, urinary and continuous ambulatory peritoneal dialysis (CAPD) catheters; shunts, such as cerebrospinal fluid shunts; hoses and tubing; plates; bolts; valves; patches; wound closures, including sutures and staples; dressings; and bone cement.

As used herein, the term “indwelling medical device,” refers to any device for use in the body of a subject, such as intravascular catheters (for example, intravenous and intra-arterial), right heart flow-directed catheters, Hickman catheters, arteriovenous fistulae, catheters used in hemodialysis and peritoneal dialysis (for example, silastic, central venous, Tenckhoff, and Teflon catheters), vascular access ports, indwelling urinary catheters, urinary catheters, silicone catheters, ventricular catheters, synthetic vascular prostheses (for example, aortofemoral and femoropopliteal), prosthetic heart valves, prosthetic joints, orthopedic implants, penile implants, shunts (for example, Scribner, Torkildsen, central nervous system, portasystemic, ventricular, ventriculoperitoneal), intrauterine devices, tampons, dental implants, stents (for example, ureteral stents), artificial voice prostheses, tympanostomy tubes, gastric feeding tubes, endotracheal tubes, pacemakers, implantable defibrillators, tubing, cannulas, probes, blood monitoring devices, needles, and the like. A subcategory of indwelling medical devices refer to implantable devices that are typically more deeply and/or permanently introduced into the body. Indwelling medical devices can be introduced by any suitable means, for example, by percutaneous, intravascular, intraurethral, intraorbital, intratracheal, intraesophageal, stromal, or other route, or by surgical implantation, for example intraarticular placement of a prosthetic joint.

In some aspects, provided herein are methods of inhibiting the formation of a biofilm on a surface or on a porous material, comprising applying to or contacting a surface or a porous material upon which a biofilm can form one or more ROS target modulator compounds and an antibiotic agent in amounts sufficient to inhibit the formation of a biofilm. In some embodiments of these methods and all such methods described herein, the surface is an inert surface, such as the surface of an in-dwelling medical device.

In some aspects, provided herein are methods of preventing the colonization of a surface by persistent bacteria, comprising applying to or contacting a surface with one or more ROS target modulator compounds and an antibiotic agent in an amount(s) sufficient to prevent colonization of the surface by persistent bacteria.

As used herein, the term “contacting” is meant to broadly refer to bringing a bacterial cell and one or more ROS target modulator compounds and an antibiotic agent into sufficient proximity that the one or more ROS target modulator compounds and the antibiotic agent can exert their effects on any bacterial cell present. The skilled artisan will understand that the term “contacting” includes physical interaction between the one or more ROS target modulator compounds and the antibiotic agent and a bacterial cell, as well as interactions that do not require physical interaction.

In the embodiments of the methods described herein directed to inhibiting or delaying the formation of a biofilm, or preventing the colonization of a surface by persistent bacteria, the material comprising the surface or the porous material can be any material that can be used to form a surface or a porous material. In some such embodiments, the material is selected from: polyethylene, polytetrafluoroethylene, polypropylene, polystyrene, polyacrylamide, polyacrylonitrile, poly(methyl methacrylate), polyamide, polyester, polyurethane, polycarbornate, silicone, polyvinyl chloride, polyvinyl alcohol, polyethylene terephthalate, cobalt, a cobalt-base alloy, titanium, a titanium base alloy, steel, silver, gold, lead, aluminum, silica, alumina, yttria stabilized zirconia polycrystal, calcium phosphate, calcium carbonate, calcium fluoride, carbon, cotton, wool and paper.

In some embodiments of these methods and all such methods described herein, the persistent, slow growing, stationary-phase or biofilm bacteria is any bacterial species or population that comprises persistent cells, can exist in a slow growing or stationary-phase, and/or that can form a biofilm. In some such embodiments, the bacteria is Staphylococcus aureus, Staphylococcus epidermidis, a vancomycin-susceptible enterococci, a vancomycin-resistant enterococci, a Staphylococcus species or a Streptococcus species. In some such embodiments, the bacteria is selected from vancomycin (VAN)-susceptible Enterococcus faecalis (VSE), VAN-resistant E. faecalis (VRE), and Staph. epidermidis.

### Dosing and Modes of Administration

One key advantage of the methods, uses and compositions comprising the one or more ROS target modulator compounds and an antibiotic agent described herein, is the ability of producing marked anti-bacterial effects in a human subject having a bacterial infection and thereby increasing bacterial sensitivity and susceptibility to a variety of antibiotic classes, as well as reducing toxicities and adverse effects. By adding ROS target modulator compounds to a therapeutic regimen or method, the dosage of the antibiotic being administered can, in some embodiments, be reduced relative to the normally administered dosage. The efficacy of the treatments and methods described herein can be measured by various parameters commonly used in evaluating treatment of infections, including but not limited to, reduction in rate of bacterial growth, the presence or number of bacterial cells in a sample obtained from a subject, overall response rate, duration of response, and quality of life.

Accordingly, a “therapeutically effective amount” or “effective amount” of a ROS target modulator compound, formulated alone or in combination with an antibiotic agent, to be administered to a subject is governed by various considerations, and, as used herein, refers to the minimum amount necessary to prevent, ameliorate, or treat, or stabilize, a disorder or condition. An effective amount as used herein also includes an amount sufficient to delay the development of a symptom of a bacterial infection, alter the course of a bacterial infection (for example but not limited to, slow the progression of a symptom of the bacterial infection, such as growth of the bacterial population), or reverse a symptom of the bacterial infection.

Effective amounts, toxicity, and therapeutic efficacy of the ROS target modulator compound, formulated alone or in combination with an antibiotic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the antibiotics and one or more ROS target modulator compounds), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

For example, in some embodiments of the aspects described herein, a given ROS target modulator, including, for example, a variant of the ROS target modulators described herein, is tested for toxicity effects in vivo. For example, single and multiple dose protocols are contemplated for assessing the toxicity to mammals of the ROS target modulators or inhibitors. For the single administration protocol, the inhibitors are administered intravenously, intraperitoneally or subcutaneously to mice at doses ranging from 0 to 1000 mg/kg. The 50% lethal dose (LD50) is calculated based on the mortality rate observed seven days after inhibitor administration. For the multiple administration protocol, the inhibitors are administered intravenously, intraperitoneally or subcutaneously to mice once daily for seven consecutive days at doses ranging from 0 to 1000 mg/kg. The LD50 is calculated based on the mortality rate observed seven days after the final inhibitor administration.

Depending on the type and severity of the infection, about 1 μg/kg to 100 mg/kg (e.g., 0.1-20 mg/kg) of a ROS target modulator is an initial candidate dosage range for administration to the subject, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the infection is treated or cleared, as measured by the methods described above or known in the art. However, other dosage regimens may be useful. The progress of the therapeutic methods described herein is easily monitored by conventional techniques and assays, such as those described herein, or known to one of skill in the art.

The duration of the therapeutic methods described herein can continue for as long as medically indicated or until a desired therapeutic effect (e.g., those described herein) is achieved. In certain embodiments, administration of a combination of an antibiotic agent and one or more ROS target modulator compounds is continued for at least 1 month, at least 2 months, at least 4 months, at least 6 months, at least 8 months, at least 10 months, at least 1 year, at least 2 years, at least 3 years, at least 4 years, at least 5 years, at least 10 years, at least 20 years, or for at least a period of years up to the lifetime of the subject. In those embodiments of the methods described herein relating to chronic infections or biofilm formation, administration is continued for as long as an in-dwelling device is present in the subject.

The ROS target modulators and antibiotic agents described herein, can be administered, individually, but concurrently, in some embodiments, or, in other embodiments, simultaneously, for example in a single formulation comprising both an antibiotic agent and one or more ROS target modulators, to a subject, e.g., a human subject, in accordance with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Exemplary modes of administration of the antibiotics and ROS target modulators, include, but are not limited to, injection, infusion, inhalation (e.g., intranasal or intratracheal), ingestion, rectal, and topical (including buccal and sublingual) administration. Local administration can be used if, for example, extensive side effects or toxicity is associated with the antibiotic agent and/or ROS target modulator compound, and to, for example, permit a high localized concentration of the ROS target modulator compound to the infection site. An ex vivo strategy can also be used for therapeutic applications. Accordingly, any mode of administration that delivers the ROS target modulator with/without the antibiotic agent compounds systemically or to a desired surface or target, and can include, but is not limited to, injection, infusion, instillation, and inhalation administration. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of an antibiotic agent and ROS target modulator compounds other than directly into a target site, tissue, or organ, such as the lung, such that it enters the subject's circulatory system and, thus, is subject to metabolism and other like processes.

The type of antibiotic being used to treat an infection or inhibit biofilm formation in a subject can determine the mode of administration to be used. For example, most aminoglycoside antibiotics are not well-absorbed via the intestine and GI tract, and thus oral administration is ineffective.

### Pharmaceutical Formulations

Therapeutic formulations of one or more ROS target modulator compounds with/without an antibiotic agent can be prepared, in some aspects, by mixing an antibiotic agent and/or ROS target modulator compound having the desired degree of purity with one or more pharmaceutically acceptable carriers, excipients or stabilizers (Remington's Pharmaceutical Sciences 16th edition, Osol, A. Ed. (1980)), in the form of lyophilized formulations or aqueous solutions, either individually in some embodiments, or in combination, e.g., a therapeutic formulation comprising alone an effective amount of an antibiotic agent and an effective amount of one or more ROS target modulator compounds. Such therapeutic formulations of the antibiotics and/or ROS target modulator compounds described herein include formulation into pharmaceutical compositions or pharmaceutical formulations for parenteral administration, e.g., intravenous; mucosal, e.g., intranasal; enteral, e.g., oral; topical, e.g., transdermal; ocular, or other mode of administration.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the activity of, carrying, or transporting the antibiotics and/or ROS target modulator compounds, from one organ, or portion of the body, to another organ, or portion of the body.

Some non-limiting examples of acceptable carriers, excipients, or stabilizers that are nontoxic to recipients at the dosages and concentrations employed, include pH buffered solutions such as phosphate, citrate, and other organic acids; antioxidants, including ascorbic acid and methionine; lubricating agents, such as magnesium stearate, sodium lauryl sulfate and talc; excipients, such as cocoa butter and suppository waxes; oils, such as peanut oil, cottonseed oil, safflower oil, sesame oil, olive oil, corn oil and soybean oil; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, HDL, LDL, or immunoglobulins; hydrophilic polymers, such as polyvinylpyrrolidone; amino acids such as glycine, glutamine, asparagine, histidine, arginine, or lysine; monosaccharides, disaccharides, and other carbohydrates including mannose, starches (corn starch or potato starch), or dextrins; cellulose, and its derivatives, such as sodium carboxymethyl cellulose, methylcellulose, ethyl cellulose, microcrystalline cellulose and cellulose acetate; chelating agents such as EDTA; sugars such as sucrose, glucose, lactose, mannitol, trehalose or sorbitol; salt-forming counter-ions such as sodium; metal complexes (e.g. Zn-protein complexes); glycols, such as propylene glycol; polyols, such as glycerin; esters, such as ethyl oleate and ethyl laurate; agar; buffering agents, such as magnesium hydroxide and aluminum hydroxide; alginic acid; pyrogen-free water; isotonic saline; Ringer's solution; polyesters, polycarbonates and/or polyanhydrides; C2-C12 alcohols, such as ethanol; powdered tragacanth; malt; and/or non-ionic surfactants such as TWEEN™, PLURONICS™ or polyethylene glycol (PEG); and/or other non-toxic compatible substances employed in pharmaceutical formulations. Wetting agents, coloring agents, release agents, coating agents, sweetening agents, flavoring agents, perfuming agents, preservative and antioxidants can also be present in the formulation.

In some embodiments, therapeutic formulations or compositions comprising an antibiotic agent and/or ROS target modulator compound comprises a pharmaceutically acceptable salt, typically, e.g., sodium chloride, and preferably at about physiological concentrations. Optionally, the formulations described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

In some embodiments of the aspects described herein, an antibiotic agent and/or ROS target modulator compound, can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (4) intravaginally or intrarectally, for example, as a pessary, cream or foam; (5) sublingually; (6) ocularly; (7) transdermally; (8) transmucosally; or (9) nasally. Additionally, an antibiotic agent and/or ROS target modulator compound, can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In some embodiments of the compositions and methods described herein, parenteral dosage forms of the compositions comprising an antibiotic agent and/or ROS target modulator compound, can be administered to a subject with a bacterial infection or at risk for bacterial infection by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms described herein are well known to those skilled in the art. Examples of such vehicles include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Topical dosage forms of the ROS target modulators and/or antibiotic agents, are also provided in some embodiments, and include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990). and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer one or more ROS target modulators and/or antibiotic agent, include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms of the ROS target modulators and/or antibiotic agents described herein are well known to those skilled in the pharmaceutical arts, and depend on the particular tissue or organ to which a given pharmaceutical composition or dosage form will be applied. In addition, depending on the specific tissue to be treated, additional components may be used prior to, in conjunction with, or subsequent to treatment with a ROS target modulator and/or antibiotic agent. For example, penetration enhancers can be used to assist in delivering the active ingredients to or across the tissue.

In some embodiments, the compositions comprising an effective amount of one or more ROS target modulators and/or an effective amount of an antibiotic agent, are formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary. In some embodiments, oral dosage forms are not used for the antibiotic agent.

Typical oral dosage forms of the compositions an effective amount of one or more ROS target modulators and/or an effective amount of an antibiotic agent are prepared by combining the pharmaceutically acceptable salt of the one or more ROS target modulators and/or the antibiotic agent, in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Binders suitable for use in the pharmaceutical formulations described herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical formulations described herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions described herein is typically present in from about 50 to about 99 weight percent of the pharmaceutical composition.

Disintegrants are used in the oral pharmaceutical formulations described herein to provide tablets that disintegrate when exposed to an aqueous environment. A sufficient amount of disintegrant that is neither too little nor too much to detrimentally alter the release of the active ingredient(s) should be used to form solid oral dosage forms of the one or more ROS target modulators and/or the antibiotic agent described herein. The amount of disintegrant used varies based upon the type of formulation, and is readily discernible to those of ordinary skill in the art. Disintegrants that can be used to form oral pharmaceutical formulations include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form oral pharmaceutical formulations of the one or more ROS target modulators and/or the antibiotic agent described herein, include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In other embodiments, lactose-free pharmaceutical formulations and dosage forms are provided, wherein such compositions preferably contain little, if any, lactose or other mono- or di-saccharides. As used herein, the term “lactose-free” means that the amount of lactose present, if any, is insufficient to substantially increase the degradation rate of an active ingredient. Lactose-free compositions of the disclosure can comprise excipients which are well known in the art and are listed in the USP (XXI)/NF (XVI), which is incorporated herein by reference.

The oral formulations of the one or more ROS target modulators and/or the antibiotic agent, further encompass, in some embodiments, anhydrous pharmaceutical compositions and dosage forms comprising the one or more ROS target modulators and/or the antibiotic agentdescribed herein as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms described herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

One or more ROS target modulators and/or an antibiotic agent can, in some embodiments of the methods described herein, be administered directly to the airways in the form of an aerosol or by nebulization. Accordingly, for use as aerosols, in some embodiments, one or more ROS target modulators and/or an antibiotic agent, can be packaged in a pressurized aerosol container together with suitable propellants, for example, hydrocarbon propellants like propane, butane, or isobutane with conventional adjuvants. In other embodiments, the one or more ROS target modulators and/or the antibiotic agent can be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means, including by using many nebulizers known and marketed today. As is well known, any suitable gas can be used to apply pressure during the nebulization, with preferred gases being those which are chemically inert to the one or more ROS target modulators and/or the antibiotic agent described herein. Exemplary gases include, but are not limited to, nitrogen, argon or helium.

In other embodiments, one or more ROS target modulators and/or an antibiotic agent, can be administered directly to the airways in the form of a dry powder. For use as a dry powder, the one or more ROS target modulators and/or the antibiotic agent can be administered by use of an inhaler. Exemplary inhalers include metered dose inhalers and dry powdered inhalers.

Suitable powder compositions include, by way of illustration, powdered preparations of one or more ROS target modulators and/or the antibiotic agent, thoroughly intermixed with lactose, or other inert powders acceptable for, e.g., intrabronchial administration. The powder compositions can be administered via an aerosol dispenser or encased in a breakable capsule which may be inserted by the subject into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

In some embodiments, the active ingredients of the formulations comprising the one or more ROS target modulators and/or the antibiotic agent described herein, can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments of these aspects, the one or more ROS target modulators and/or the antibiotic agent, can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control, for example, an aminoglycoside antibiotic's onset of action, duration of action, plasma levels within the therapeutic window, and peak blood levels. In particular, controlled- or extended-release dosage forms or formulations can be used to ensure that the maximum effectiveness of the one or more ROS target modulators and/or the antibiotic agent, is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the compositions comprising one or more ROS target modulators with/without the antibiotic agent described herein Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated ins entirety herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments of the aspects, the one or more ROS target modulators with/without the antibiotic agent for use in the various therapeutic formulations and compositions, and methods thereof described herein, are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred in chronic bacterial conditions, as each pulse dose can be reduced and the total amount of a compound, such as, for example, an antibiotic agent, administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals may be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

In some embodiments, sustained-release preparations comprising the one or more ROS target modulators with/without the antibiotic agent, can be prepared. Suitable examples of sustained-release preparations include semipermeable matrices of solid hydrophobic polymers containing the inhibitor, in which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid.

The formulations comprising the one or more ROS target modulators with/without the antibiotic agent described herein, to be used for in vivo administration are preferably sterile. This is readily accomplished by filtration through, for example, sterile filtration membranes, and other methods known to one of skill in the art.

Embodiments of the various aspects described herein can be illustrated by the following numbered paragraphs:
1. A method for inhibiting a bacterial infection by increasing ROS (reactive oxygen species) production in a bacteria, the method comprising administering to a subject having or at risk for a bacterial infection an effective amount of one or more ROS target modulator compounds and an effective amount of an antibiotic agent.
2. A method for inhibiting a bacterial infection by increasing ROS (reactive oxygen species) production in a bacteria, the method comprising administering to a subject having or at risk for a bacterial infection an effective amount of a pharmaceutical composition comprising one or more ROS target modulator compounds and an antibiotic agent.
3. A method for treating a bacterial infection by increasing ROS (reactive oxygen species) production in a bacteria, comprising administering to a patient having a bacterial infection and undergoing treatment with an antibiotic agent, an effective amount of one or more ROS target modulator compounds.
4. The method of any one of paragraphs 1-3, wherein the ROS target modulator is an inhibitor of an enzyme involved in bacterial glycolysis, pentose-phosphate pathway, EntnerDoudoroff pathway, TCA cycle, glyoxylate shunt, aerobic respiration, or acetate metabolism.
5. The method of any one of paragraphs 1-4, wherein the ROS target modulator is an inhibitor of: ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, or fumarase B.
6. The method of paragraph 5, wherein the inhibitor of ATP synthase is selected from IF1, an efrapeptin, aurovertin B, citreoviridin, α-zearalenol, and any analogs thereof.
7. The method of paragraph 5, wherein the inhibitor of succinate dehydrogenase is selected from carboxin, thenoyltrifluoroacetone, malonate, malate, oxaloacetate, and any analogs thereof.
8. The method of paragraph 5, wherein the inhibitor of glutamate dehydrogenase is selected from bromofuroate; 3-carboxy-5-bromofuroic acid; Palmitoyl-Coenzyme-A; orthovanadate; vanadyl sulphate, vanadyl acetylacetonate, glutarate; 2-oxoglutarate; estrogen; pyridine-2,6-dicarboxylic acid; and (−)-epigallocatechin gailate (EGCG).
9. The method of paragraph 5, wherein the inhibitor of NADH dehydrogenase is selected from Amytal; Amytal Sodium; Annonin VI; Aurachin A; Aurachin B; Aureothin; Benzimidazole; Bullactin; calnexin; Capsaicin; Ethoxyformic anhydride; Ethoxyquin; Fenpyroximate; Mofarotene; mofarotene 2-oxoglutarate dehydrogenase; Molvizarin; Myxalamide PI; M2-type pyruvate kinase; Otivarin; Pethidine; rhein; Phenalamid A2; Phenoxan; Piericidin A; p-chloromercuribenzoate; Ranolazine; Rolliniasatin-1; Rolliniasatin-2; Rotenone; Squamocin; Thiangazole rotenoids; thiol reagents; Demerol; iron chelators; NAD+(nicotinamide adenine dinucleotide; oxidized form); AMP (adenosine monophosphate); ADP (adenosine diphosphate); ADP-ribosylation factor 3; ATP (adenosine triphosphate); guanidinium salts; NADH; barbituates; gossypol; polyphenol; dihydroxynaphthoic acids; acetogenin; adenosine diphosphate ribose; rotenoid; acetogenin; nitrosothiols; peroxynitrite; carvedilol; arylazido-beta-alanyl NAD+; adriamycin; 4-hydroxy-2-nonenal; pyridine derivatives; 2-heptyl-4-hydroxyquinoline N-oxide; dicumarol; o-phenanthroline; and 2;2′-dipyridyl.
10. The method of paragraph 5, wherein the inhibitor of pyruvate dehydrogenase is selected from

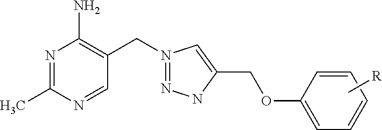

where R is 2-Cl-4-NO₂, 4-NO₂, 4-COOH, or H; secondary amides of (R)-3;3;3-Trifluoro-2-hydroxy-2-methylpropionic acid glyoxylate; (R)-3;3;3-Trifluoro-2-hydroxy-2-methylpropionamides; anilides of (R)-Trifluoro-2-hydroxy-2-methylpropionic acidhydroxypyruvate; kynurenate; xanthurenate; α-cyano-4-hydroxycinnamic acid; bromopyruvic acid; fluropyruvic acid; AZD-7545; phosphonate and phosphinate analogs of pyruvate; mono- and bifunctional arsenoxides; branched-chain 2-oxo acids; 2-oxo-3-alkynoic acids; tetrahydrothiamin diphosphate (ThDP); and 2-thiazolone and 2-thiothiazolone analogs of ThDP.
11. The method of paragraph 5, wherein the inhibitor of cytochrome oxidase is selected from azide; nitric oxide; cytochrome P450 oxidase inhibitors; aurachin A; Aurachin C; aurachin D; tridecylstigmatelli; stigmatellin; nigericin; hydroxylamine; heptylhydroxyquinoline N-oxide (HQNO); nonylhydroxyquinoline N-oxide (NQNO); dibromothymoquinone (DBMIB); piericidin A; and undecylhydroxydioxobenzo-thiazole (UHDBT).
12. The method of paragraph 5, wherein the inhibitor of glucose-6-phosphate dehydrogenase is selected from dehydroepiandrosterone (DHEA), DHEA-sulfate; 2-deoxyglucose; halogenated DHEA; epiandrosterone; isoflurane; sevoflurane; diazepam; CBF-BS2; cystamine; 16α-bromoepiandrosterone; 16α-hydroxy-5-androsten-17-one; 16α-fluoro-5-androsten-17-one; 16α-fluoro-16β-methyl-5-androsten-17-one; 16α-methyl-5-androsten-17-one; 16β-methyl-5-androsten-17-one; 16α-hydroxy-5α-androstan-17-one; 16α-fluoro-5α-androstan-17-one; 16α-fluoro-160-methyl-5α-androstan-17-one; 16α-methyl-5α-androstan-17-one; 16β-methyl-5α-androstan-17-one; and 2-amino-2-deoxy-D-glucose-6-phosphate.
13. The method of paragraph 5, wherein the inhibitor of 6-phosphogluconate dehydrogenase is selected from 6-aminonicotinamide; aldonate 4-phospho-d-erythronate; 5,6-Dideoxy-6-phosphono-d-arabino-hexonate; and 5-deoxy-5-phosphono-d-arabinonate.
14. The method of paragraph 5, wherein the inhibitor of succinyl-CoA synthetase is selected from LY266500 and vanadium sulphate.
15. The method of paragraph 5, wherein the inhibitor of triose phosphate isomerase is selected from 3-haloacetol phosphate; glycidol phosphate; phosphoenol pyruvate; DHAP; GAP; 2-phosphoglycollate; phosphoglycolohydroxamate; 3-phosphoglycerate; glycerol phosphate; phosphoenol pyruvate; 2;9-Dimethyl-β-carbolines and derivatives thereof; 3-(2-benzothiazolylthio)-1-propanesulfonic acid; 2-carboxyethylphosphonic acid; 2-phosphoglyceric acid; N-hydroxy-4-phosphono-butanamide; and [2(formyl-hydroxy-amino)-ethyl]-phosphonic acid.
16. The method of paragraph 5, wherein the inhibitor of phosphofructokinase is selected from aurintricarboxylic acid; pyruvate; 2-deoxy-2-fluoro-D-glucose; citrate and halogenated derivatives of citrate; fructose 2,6-bisphosphate; N-(2-methoxyethyl)-bromoacetamide; N-(2-ethoxyethyl)-bromoacetamide; N-(3-methoxypropyl)-bromoacetamide); phosphoglycerate; taxodone; taxodione; euparotin acetate eupacunin; vernolepin; argaric acid, quinaldic acid; and 5′-p-flurosuflonylbenzoyl adenosine.
17. The method of paragraph 5, wherein the inhibitor of the fumarase B is selected from trans-aconitate; bromomesaconate; citrate; meso-tartaric acid; bismuth; DL-fluoromalic acid; and S-2,3-Dicarboxyaziridine.
18. The method of any one of paragraphs 1-17, wherein the ROS target modulator is an inhibitor of E. coli cyoA, nuoG, or sdhC, or an ortholog thereof.
19. The method of any one of paragraphs 1 to 18, wherein the ROS target modulator is selected for its ability to boost ROS production or increase susceptibility to oxidative stress.
20. The method of paragraph 19, wherein the ROS is O₂⁻, H₂O₂, or O₂⁻ and H₂O₂. 21. The method of any of paragraphs 1-20, wherein the antibiotic is bactericidal or bacteriostatic.
22. The method of any of paragraphs 1-21, wherein the antibiotic agent is a β-lactam, fluoroquinoline, macrolide, nitroimidazole compound, tetracycline, vancomycin, bacitracin, macrolide; lincosamide, chloramphenicol, amphotericin, cefazolins, clindamycins, mupirocins, sulfonamides, trimethoprim, rifampicin, metronidazole, quinolone, novobiocin; polymixin; gramicidin, aminoglycoside, or any salts or variants thereof.
23. The method of any one of paragraphs 1-21, wherein the antibiotic agent is not an aminoglycoside.
24. The method of paragraph 22, wherein the β-lactam antibiotic agent is a penam antibiotic or a penicillin antibiotic.
25. The method of paragraph 24, wherein the penicillin antibiotic is selected from amoxicillin, ampicillin, methicillin, oxacillin, nafcillin, cloxacillin, dicloxacillin, flucloxacillin, azlocillin, carbenicillin, ticarcillin, mezlocillin, piperacillin, penicillin, benzathine penicillin, benzylpenicillin, phenoxymethylpenicillin, procaine penicillin; temocillin; co-amoxiclav; and mecillinam.
26. The method of paragraph 22, wherein the β-lactam antibiotic agent is a cephalosporin or cephamycin.
27. The method of paragraph 26, wherein the cephalosporin or cephamycin is selected from cefazolin, cefalexin, cefalotin, cefdinir, cefepime, cefotaxime, cefpodoxime proxetil, ceftobiprole, ceftaroline fosamil, cephalosporin C, cephalothin, cefaclor, cefamandole, cefuroxime, cefotetan, cefoxitin, cefixime, ceftazidime, ceftriaxone, and cefpirome.
28. The method of paragraph 22, wherein the β-lactam antibiotic agent is a carbapenem.
29. The method of paragraph 28, wherein the carbapenem is selected from ertapenem, meropenem, imipenem, doripenem, panipenem/betamipron, biapenem, razupenem, and tebipenem.
30. The method of paragraph 22, wherein the β-lactam antibiotic agent is a penem.
31. The method of paragraph 30, wherein the penem is selected from thiopenems, oxypenems, aminopenems, alkylpenems, and arylpenems.
32. The method of paragraph 22, wherein the β-lactam antibiotic agent is a monobactam.
33. The method of paragraph 32, wherein the monobactam is selected from aztreonam, tigemonam, nocardicin A, and tabtoxinine β-lactam.
34. The method of paragraph 22, wherein when the antibiotic agent is a β-lactam antibiotic agent, the one or more ROS target modulators is selected from a cytochrome oxidase inhibitor, an NADH dehydrogenase inhibitor, a succinate dehydrogenase inhibitor, or any combination thereof.
35. The method of paragraph 22, wherein the fluorquinolone antibiotic agent is selected from ciprofloxacin, moxifloxacin, ofloxacin, balofloxacin, grepafloxacin, levofloxacin, pazufloxacin, sparfloxacin, temafloxacin, and tosufloxacin.
36. The method of paragraph 22, wherein when the antibiotic agent is a fluorquinolone antibiotic agent, the one or more ROS target modulators is selected from a cytochrome oxidase inhibitor, an NADH dehydrogenase inhibitor, a succinate dehydrogenase inhibitor, a phospho acetyl transferase inhibitor, or any combination thereof.
37. The method of paragraph 22, wherein the nitroimidazole compound antibiotic is selected from metronidazole, tinidazole, and nimorazole.
38. The method of paragraph 22, wherein the tetracycline antibiotic agent is selected from tetracycline, chlortetracycline, oxytetracycline, demeclocycline, doxycycline, lymecycline, meclocycline, methacycline, minocycline, and rolitetracycline.
39. The method of any one of paragraphs 1-38, wherein the bacterial infection involves a gram positive or gram negative bacteria.
40. The method of any one of paragraphs 1-39, wherein the bacterial infection is of an aerobic bacteria or facultative anaerobic bacteria.
41. The method of any one of paragraphs 1-40, wherein the bacterial infection is caused by a bacterial pathogen having an active metabolic system comprising glycolysis, pentose-phosphate pathway, and/or EntnerDoudoroff pathway.
42. The method of any one of paragraphs 1-41, wherein the bacterial infection is caused by a bacterial pathogen having an active metabolic system comprising the TCA cycle, glyoxylate shunt, and/or acetate metabolism.
43. The method of paragraph 39, wherein the bacterial infection is of an enteric or respiratory pathogen.
44. The method of paragraph 39, wherein the bacterial infection is pneumonia, strep throat, bacteremia, sepsis, toxic shock syndrome, endocarditis, abscess, an infection of skin or soft tissue, or is an infected wound or burn.
45. The method of paragraph 39, wherein the bacterial infection is necrotizing fasciitis, osteomyelitis, peritonitis, infected surgical wound, or diabetic ulcer.
46. The method of paragraph 39, wherein the bacterial infection is a chronic or persistent bacterial infection.
47. The method of any one of paragraphs 1-45, wherein the bacterial infection is an acute or non-latent bacterial infection.
48. The method of any one of paragraphs 1-47, wherein the infection is a surface wound, burn, or infection; infection of a mucosal surface; respiratory infection; infections of the eyes, ears, nose, or throat; or infection of an intestinal pathogen.
49. The method of any one of paragraph 1-48, wherein the bacterial infection is resistant to one or more anti-microbial agents.
50. The method of any one of paragraphs 1-49, wherein the bacterial infection involves one or more of E. coli, Mycobacterium sp., Staphylococcus sp., Haemophilus sp., Salmonella sp., Streptococcus sp., Neisseria sp., Pseudomonas sp., Klebsiella sp., Enterobacter sp., Acinetobacter sp., Listeria sp., Campylobacter sp., Enterococcus sp., Bacillus sp., Corynebacterium sp., Clostridium sp., Bacteroides sp., Treponema sp., Lactobacillus sp., Nocardia sp.; Actinomyces sp., Mobiluncus sp., Peptostreptococcus sp., Brucella sp., Campylobacter sp., Proteus sp.; Shigella sp.; Yersinia sp., Aeromonas sp., Vibrio sp., Acinetobacter sp., Flavobacterium sp.; Burkholderia sp., Bacteroides sp., Prevotella sp., Fusobacterium sp., Borrelia sp., Chlamydia sp., Legionella sp., and Leptospira sp.
51. The method of any one of paragraphs 1-49, wherein the bacterial infection involves one or more of E. coli, Klebsiella pneumoniae, Acinetobacter baumanii, Pseudomonas aeruginosa, Streptococcus pneumoniae, Mycobacterium tuberculosis, Staphylococcus aureus, Haemophilus influenzae, and Salmonella typhimurium.
52. The method of any one of paragraphs 1-50, wherein the ROS target modulator and the antibiotic agent are co-formulated.
53. The method of any one of paragraphs 1 and 3-52, wherein the ROS target modulator and the antibiotic agent are administered separately.
54. The method of any one of paragraphs 1-53, wherein the ROS target modulator is administered systemically or locally.
55. The method of any one of paragraphs 1-53, wherein the ROS target modulator is administered intravenously, orally, or topically.
56. The method of any one of paragraphs 1-53, wherein the bacterial infection occurs at or in a surface wound or burn, and the ROS target modulator is administered topically to the affected area.
57. The method of paragraph 56, wherein the ROS target modulator is formulated as a cream, gel, foam, spray, or as a tablet or capsule for oral delivery.
58. A method for inhibiting a bacterial infection by increasing ROS (reactive oxygen species) production in a bacteria, the method comprising administering to a subject having or at risk for a bacterial infection an effective amount of one or more ROS target modulator compounds selected and an effective amount of an antibiotic agent, wherein the ROS target modulator is an inhibitor of ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, or fumarase B, and wherein the antibiotic agent is a β-lactam, fluoroquinoline, macrolide, nitroimidazole compound, tetracycline, vancomycin, bacitracin, macrolide; lincosamide, chloramphenicol, amphotericin, cefazolins, clindamycins, mupirocins, sulfonamides, trimethoprim, rifampicin, metronidazole, quinolone, novobiocin; polymixin; gramicidin, aminoglycoside, or any salts or variants thereof.
59. The method of paragraph 58, wherein the ROS target modulator and the antibiotic agent are co-formulated.
60. The method of paragraph 58, wherein the ROS target modulator and the antibiotic agent are administered separately.
61. The method of any one of paragraphs 58-60, wherein the bacterial infection involves a gram positive or gram negative bacteria.
62. The method of any one of paragraphs 58-61, wherein the bacterial infection is of an aerobic bacteria or facultative anaerobic bacteria.
63. The method of paragraph any one of paragraphs 58-62, wherein the bacterial infection is one or more of sepsis, bacteremia, pneumonia, endocarditis, skin or soft tissue infection, or an infected wound or burn.
64. The method of paragraph 63, wherein the bacterial infection comprises E. coli, P. aeroginusa, K pneumoniae, or A. Baumanii.
65. The method of paragraph 61, wherein the bacterial infection is an acute or non-latent infection.
66. The method of paragraph 61, wherein the bacterial infection is a chronic or persistent bacterial infection.
67. The method of any one of paragraphs 58-66, wherein the antibiotic is bactericidal.
68. The method of paragraph 67, wherein the antibiotic is a β-lactam or fluoroquinolone antibiotic.
69. A ROS target modulator for use in inhibiting or treating a bacterial infection by increasing ROS (reactive oxygen species) production in a bacteria.
70. The use of paragraph 69, wherein the ROS target modulator is an inhibitor of an enzyme involved in bacterial glycolysis, pentose-phosphate pathway, EntnerDoudoroff pathway, TCA cycle, glyoxylate shunt, aerobic respiration, or acetate metabolism.
71. The use of any one of paragraphs 69-70, wherein the ROS target modulator is an inhibitor of: ATP synthase, succinate dehydrogenase, glutamate dehydrogenase, NADH dehydrogenase, pyruvate dehydrogenase, cytochrome oxidase, glucose 6-phosphate dehydrogenase, 6-phosphogluconate dehydrogenase, succinyl-CoA ligase, triose phosphate isomerase, phosphate acetyltransferase, phosphofructokinase, or fumarase B.
72. The use of paragraph 71, wherein the inhibitor of ATP synthase is selected from IF1, an efrapeptin, aurovertin B, citreoviridin, α-zearalenol, and any analogs thereof.
73. The use of paragraph 71, wherein the inhibitor of succinate dehydrogenase is selected from carboxin, thenoyltrifluoroacetone, malonate, malate, oxaloacetate, and any analogs thereof.
74. The use of paragraph 71, wherein the inhibitor of glutamate dehydrogenase is selected from bromofuroate; 3-carboxy-5-bromofuroic acid; Palmitoyl-Coenzyme-A; orthovanadate; vanadyl sulphate, vanadyl acetylacetonate, glutarate; 2-oxoglutarate; estrogen; pyridine-2,6-dicarboxylic acid; and (−)-epigallocatechin gailate (EGCG).
75. The use of paragraph 71, wherein the inhibitor of NADH dehydrogenase is selected from Amytal; Amytal Sodium; Annonin VI; Aurachin A; Aurachin B; Aureothin; Benzimidazole; Bullactin; calnexin; Capsaicin; Ethoxyformic anhydride; Ethoxyquin; Fenpyroximate; Mofarotene; mofarotene 2-oxoglutarate dehydrogenase; Molvizarin; Myxalamide PI; M2-type pyruvate kinase; Otivarin; Pethidine; rhein; Phenalamid A2; Phenoxan; Piericidin A; p-chloromercuribenzoate; Ranolazine; Rolliniasatin-1; Rolliniasatin-2; Rotenone; Squamocin; Thiangazole rotenoids; thiol reagents; Demerol; iron chelators; NAD+(nicotinamide adenine dinucleotide; oxidized form); AMP (adenosine monophosphate); ADP (adenosine diphosphate); ADP-ribosylation factor 3; ATP (adenosine triphosphate); guanidinium salts; NADH; barbituates; gossypol; polyphenol; dihydroxynaphthoic acids; acetogenin; adenosine diphosphate ribose; rotenoid; acetogenin; nitrosothiols; peroxynitrite; carvedilol; arylazido-beta-alanyl NAD+; adriamycin; 4-hydroxy-2-nonenal; pyridine derivatives; 2-heptyl-4-hydroxyquinoline N-oxide; dicumarol; o-phenanthroline; and 2;2′-dipyridyl.
76. The use of paragraph 71, wherein the inhibitor of pyruvate dehydrogenase is selected from

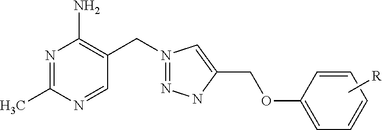

where R is 2-Cl-4-NO₂, 4-NO₂, 4-COOH, or H; secondary amides of (R)-3;3;3-Trifluoro-2-hydroxy-2-methylpropionic acid glyoxylate; (R)-3;3;3-Trifluoro-2-hydroxy-2-methylpropionamides; anilides of (R)-Trifluoro-2-hydroxy-2-methylpropionic acidhydroxypyruvate; kynurenate; xanthurenate; α-cyano-4-hydroxycinnamic acid; bromopyruvic acid; fluropyruvic acid; AZD-7545; phosphonate and phosphinate analogs of pyruvate; mono- and bifunctional arsenoxides; branched-chain 2-oxo acids; 2-oxo-3-alkynoic acids; tetrahydrothiamin diphosphate (ThDP); and 2-thiazolone and 2-thiothiazolone analogs of ThDP.
77. The use of paragraph 71, wherein the inhibitor of cytochrome oxidase is selected from azide; nitric oxide; cytochrome P450 oxidase inhibitors; aurachin A; Aurachin C; aurachin D; tridecylstigmatelli; stigmatellin; nigericin; hydroxylamine; heptylhydroxyquinoline N-oxide (HQNO); nonylhydroxyquinoline N-oxide (NQNO); dibromothymoquinone (DBMIB); piericidin A; and undecylhydroxydioxobenzo-thiazole (UHDBT).
78. The use of paragraph 71, wherein the inhibitor of glucose-6-phosphate dehydrogenase is selected from dehydroepiandrosterone (DHEA), DHEA-sulfate; 2-deoxyglucose; halogenated DHEA; epiandrosterone; isoflurane; sevoflurane; diazepam; CBF-BS2; cystamine; 16α-bromoepiandrosterone; 16α-hydroxy-5-androsten-17-one; 16α-fluoro-5-androsten-17-one; 16α-fluoro-16β-methyl-5-androsten-17-one; 16α-methyl-5-androsten-17-one; 16β-methyl-5-androsten-17-one; 16α-hydroxy-5α-androstan-17-one; 16α-fluoro-5α-androstan-17-one; 16α-fluoro-160-methyl-5α-androstan-17-one; 16α-methyl-5α-androstan-17-one; 16β-methyl-5α-androstan-17-one; and 2-amino-2-deoxy-D-glucose-6-phosphate.
79. The use of paragraph 71, wherein the inhibitor of 6-phosphogluconate dehydrogenase is selected from 6-aminonicotinamide; aldonate 4-phospho-d-erythronate; 5,6-Dideoxy-6-phosphono-d-arabino-hexonate; and 5-deoxy-5-phosphono-d-arabinonate.
80. The use of paragraph 71, wherein the inhibitor of succinyl-CoA synthetase is selected from LY266500 and vanadium sulphate. 81. The use of paragraph 71, wherein the inhibitor of triose phosphate isomerase is selected from 3-haloacetol phosphate; glycidol phosphate; phosphoenol pyruvate; DHAP; GAP; 2-phosphoglycollate; phosphoglycolohydroxamate; 3-phosphoglycerate; glycerol phosphate; phosphoenol pyruvate; 2;9-Dimethyl-β-carbolines and derivatives thereof; 3-(2-benzothiazolylthio)-1-propanesulfonic acid; 2-carboxyethylphosphonic acid; 2-phosphoglyceric acid; N-hydroxy-4-phosphono-butanamide; and [2(formyl-hydroxy-amino)-ethyl]-phosphonic acid.
82. The use of paragraph 71, wherein the inhibitor of phosphofructokinase is selected from aurintricarboxylic acid; pyruvate; 2-deoxy-2-fluoro-D-glucose; citrate and halogenated derivatives of citrate; fructose 2,6-bisphosphate; N-(2-methoxyethyl)-bromoacetamide; N-(2-ethoxyethyl)-bromoacetamide; N-(3-methoxypropyl)-bromoacetamide); phosphoglycerate; taxodone; taxodione; euparotin acetate eupacunin; vernolepin; argaric acid, quinaldic acid; and 5′-p-flurosuflonylbenzoyl adenosine.
83. The use of paragraph 71, wherein the inhibitor of the fumarase B is selected from trans-aconitate; bromomesaconate; citrate; meso-tartaric acid; bismuth; DL-fluoromalic acid; and S-2,3-Dicarboxyaziridine.
84. The use of any one of paragraphs 69-83, wherein the ROS target modulator is an inhibitor of E. coli cyoA, nuoG, or sdhC, or an ortholog thereof.
85. The use of any one of paragraphs 69-84, wherein the ROS target modulator is selected for its ability to boost ROS production or increase susceptibility to oxidative stress.
86. The use of paragraph 85, wherein the ROS is O₂⁻, H₂O₂, or O₂⁻ and H₂O₂.
87. The use of any one of paragraphs 69-86, wherein the bacterial infection involves a gram positive or gram negative bacteria.
88. The use of any one of paragraphs 69-87, wherein the bacterial infection is of an aerobic bacteria or facultative anaerobic bacteria.
89. The use of any one of paragraphs 69-88, wherein the bacterial infection is caused by a bacterial pathogen having an active metabolic system comprising glycolysis, pentose-phosphate pathway, and/or EntnerDoudoroff pathway.
90. The use of any one of paragraphs 69-89, wherein the bacterial infection is caused by a bacterial pathogen having an active metabolic system comprising the TCA cycle, glyoxylate shunt, and/or acetate metabolism.
91. The use of paragraph 87, wherein the bacterial infection is of an enteric or respiratory pathogen.
92. The use of paragraph 87, wherein the bacterial infection is pneumonia, strep throat, bacteremia, sepsis, toxic shock syndrome, endocarditis, abscess, an infection of skin or soft tissue, or is an infected wound or burn.
93. The use of paragraph 87, wherein the bacterial infection is necrotizing fasciitis, osteomyelitis, peritonitis, infected surgical wound, or diabetic ulcer.
94. The use of any one of paragraphs 69-93, wherein the bacterial infection is a chronic or persistent bacterial infection.
95. The use of any one of paragraphs 69-93, wherein the bacterial infection is an acute or non-latent bacterial infection.
96. The use of any one of paragraphs 69-95, wherein the infection is a surface wound, burn, or infection; infection of a mucosal surface; respiratory infection; infections of the eyes, ears, nose, or throat; or infection of an intestinal pathogen.
97. The use of any one of paragraphs 69-96, wherein the bacterial infection is resistant to one or more anti-microbial agents.
98. The use of any one of paragraphs 69-97, wherein the bacterial infection involves one or more of E. coli, Mycobacterium sp., Staphylococcus sp., Haemophilus sp., Salmonella sp., Streptococcus sp., Neisseria sp., Pseudomonas sp., Klebsiella sp., Enterobacter sp., Acinetobacter sp., Listeria sp., Campylobacter sp., Enterococcus sp., Bacillus sp., Corynebacterium sp., Clostridium sp., Bacteroides sp., Treponema sp., Lactobacillus sp., Nocardia sp.; Actinomyces sp., Mobiluncus sp., Peptostreptococcus sp., Brucella sp., Campylobacter sp., Proteus sp.; Shigella sp.; Yersinia sp., Aeromonas sp., Vibrio sp., Acinetobacter sp., Flavobacterium sp.; Burkholderia sp., Bacteroides sp., Prevotella sp., Fusobacterium sp., Borrelia sp., Chlamydia sp., Legionella sp., and Leptospira sp.
99. The use of any one of paragraphs 69-97, wherein the bacterial infection involves one or more of E. coli, Klebsiella pneumoniae, Acinetobacter baumanii, Pseudomonas aeruginosa, Streptococcus pneumoniae, Mycobacterium tuberculosis, Staphylococcus aureus, Haemophilus influenzae, and Salmonella typhimurium.
100. The use of any one of paragraphs 69-99, wherein the ROS target modulator is co-formulated with an antibiotic agent.
101. A method for making an antimicrobial composition, comprising:
   selecting a gene whose deletion increases ROS production or sensitivity to oxidative stress in a bacteria,
   selecting an inhibitor of said gene,
   formulating said inhibitor for administration.
102. The method of paragraph 101, wherein said gene is an enzyme that loses electrons to a flavin, quinone, and/or transition metal center during catalysis, said transition metal center optionally being an iron sulfur protein, aconitase, fumarase, or dihydroxy acid dehydratase.
103. The method of paragraph 101, wherein the gene is a bacterial: ATP synthase, succinate dehydrogenase, glutamate dehydrogenase, NADH dehydrogenase, pyruvate dehydrogenase, cytochrome oxidase, glucose-6-phosphate dehydrogenase, 6-phosphogluconate dehydrogenase, succinyl-CoA ligase, triose phosphate isomerase, phosphate acetyltransferase, phosphofructokinase, or fumerase B.
104. The method of any one of paragraphs 101-103, wherein the inhibitor is co-formulated with a bactericidal antibiotic.
105. The method of paragraph 104, wherein the bactericidal antibiotic is a β-lactam or fluoroquinolone antibiotic.
106. The method of any one of paragraphs 101-105, wherein the bacteria is an aerobe or facultative anaerobe.
107. The method of any one of paragraphs 101-106, wherein the bacteria is a causative agent of sepsis, pneumonia, skin or soft tissue infection, or infected burn or wound.
108. The method of paragraph 107, wherein the bacteria is E. coli, K. pneumoniae, A. baumanii, or P. aeruginosa.
109. The method of any one of paragraphs 101-108, wherein the inhibitor is formulated for intravenous, topical, or oral delivery.
110. A method for identifying metabolic perturbations that increase sensitivity towards oxidative stress in a microorganism, the method comprising the steps of:
   a. generating a genome-scale metabolic model of systems-level ROS production in the microorganism;
   b. systematically deleting genes from the genome-scale metabolic model to identify genes that alter basal ROS production in the microorganism, wherein an increase in the basal ROS production in the microorganism is indicative that deletion of the gene(s) increases sensitivity towards oxidative stress in the microorganism; and
   c. measuring ROS production in a variant of the microorganism genetically modified to lack the genes that alter basal ROS production identified in step (b).
111. The method of paragraph 110, wherein the microorganism is Escherichia coli, Mycobaterium tuberculosis, Staphylococcus aureus, Haemophilus influenzae, Klebsiella pneumoniae, Pseudomonas aeruginosa, Acintebacter baumanii, or Salmonella typhimurium.

This invention is further illustrated by the following examples which should not be construed as limiting. The following exemplary methods were used to demonstrate that inhibiting ROS targets potentiates antibiotic activity and sensitivity of bacterial strains to antibiotics in a ROS-dependent fashion, can be used, for example to identify additional ROS targets and modulators thereof for use in the methods and compositions described herein.

### Examples

### Methods Summary

### Antibiotics and Chemicals

All chemicals and antibiotics were purchased from Sigma or Fisher Scientific. Concentrated stock solutions of menadione, H₂O₂, NaOCl, and ampicillin were prepared fresh daily. H₂O₂, NaOCl, ampicillin, and gentamicin were diluted with or dissolved in sterile deionized water. Ofloxacin and ciprofloxacin were dissolved in 0.1N NaOH. Tetracycline was dissolved in 50% ethanol (v/v). Menadione, carboxin, and chloramphenicol were dissolved in 100% ethanol.

### Strains and Media

Escherichia coli MG1655 was used in this study. Genetic deletions of aceA, appB, atpC, cyoA, edd, fumB, fbaB, gdhA, gltB, gnd, mqo, nuoG, pfkB, pta, pykA, rpiB, sdhC, sucC, talB, tktB and zwf were transduced from the Keio single-gene deletion knockout library⁶³ into MG1655 using the P1 phage method, and confirmed with PCR. The media used for all experiments was M9 minimal media with 10 mM glucose as the sole carbon source or MOPS minimal media with 10 mM glucose (for the HyPer protein experiments).

### Plasmids

The OT response sensor used in this study was constructed previously¹⁰, and utilized the native soxS promoter upstream of the gfpmut3b gene. The H₂O₂ response sensor used the same plasmid backbone and was constructed by PCR-amplifying the native dps promoter and cloning it into the Bam11I and XhoI restriction sites, which formerly contained the soxS promoter. The forward primer for PCR was GCGCCTCGAGCCGCTTCAATGGGGTCTACGCT (SEQ ID NO: 1) and the reverse primer was GGCCGGATCCTCGGAGACATCGTTGCGGGTAT (SEQ ID NO: 2). The H₂O₂ response sensor was confirmed to increase expression of GFP upon addition of H₂O₂.

### GFP Reporter Assays

Fluorescent measurements were performed on a SPECTRAMAX M5 plate reader (Molecular Devices) using Costar black, clear, flat bottom 96-well plates (Fisher). Each well contained 195 μL of M9 minimal glucose media with ampicillin (100 μg/mL) and 5 μL of overnight culture (plasmids carry an AmpR gene for selection). Overnight cultures were grown in M9 minimal glucose media. Strains were grown in the plate reader at 37° C. with shaking. OD₆₀₀ and fluorescence (excitation: 488 nm, emission: 520 nm, bottom read) were monitored every 10 minutes. Fluorescence/OD₆₀₀ values were calculated using ordinary least squares regression for measurements between OD₆₀₀=0.1 and OD₆₀₀=0.4. Values reported are the relative mean and standard error mean for at least three independent biological replicates in Table 2. P-values were calculated using a single-tailed, two-sample t-test, assuming unequal variance.

### HyPer Assays

The HyPer protein is a fluorescent probe that was made by inserting a circularly permuted yellow fluorescent protein into the H₂O₂-sensitive regulatory domain of OxyR⁴⁶. In the presence of increasing concentrations of H₂O₂ the probe's excitation peak shifts ratiometrically from 420 nm to 500 nm, which allows for quantitative measurement of cellular H₂O₂ levels^{46, 47}. HyPer is based on an E. coli H₂O₂-sensing domain, and has been shown to be effective at sensing H₂O₂ within E. coli⁴⁶. HyPer was provided from the manufacturer (Evrogen) as an IPTG-inducible gene in a pQE30 vector (ampicillin selection marker)⁴⁶. Single colonies of strains were inoculated into LB media supplemented with 50 μg/mL ampicillin and grown overnight at 37° C. The ΔatpC and Δzwf strains were run separately with wildtype because those strains grew significantly slower than the other mutant strains. Strains were inoculated 1:100 into MOPS minimal media plus 10 mM Glucose and 50 μg/mL ampicillin, and grown to an OD₆₀₀ of 0.2-0.3. All cultures were then diluted with MOPS minimal media plus 50 μg/mL ampicillin in a black, clear bottom 96-well plate to a final OD₆₀₀ of 0.05, in a final volume of 200 μL per well. 20 μL of mineral oil (Sigma Aldrich) was added to each well to prevent evaporation. Strains were grown with and without 75 μM IPTG in a SpectraMax M5 plate reader (Molecular Devices) at 37° C. with shaking, and OD₆₀₀ and fluorescence (excitation: 420 nm and 500 nm, emission: 530 nm, bottom read) were monitored every 15 minutes for 12 hours. Measurements between OD600=0.2 and OD600=0.6 were corrected for background strain fluorescence by subtracting the fluorescence values for un-induced cultures at the same cell density, as measured by OD₆₀₀. The 420 nm×500 nm curve was linear over this region, and therefore ordinary least squares regression was used to interpolate between time points. The 500 nm excitation fluorescence value that corresponded with 55 fluorescence units from 420 nm excitation was calculated and the 500/420 ratio was obtained for all strains. Values reported in Table 3 are the relative mean and standard error mean for three independent biological replicates. P-values were calculated using a single-tailed, two-sample t-test, assuming unequal variance.

### Antimicrobial Sensitivity Assays

Strains were grown aerobically from an initial inoculation of OD₆₀₀=0.01 to OD₆₀₀=0.16-0.20 in 250 mL baffled flasks filled to 1/10^{th} the total volume and shaken at 300 rpm at 37° C. For menadione, H₂O₂, and ampicillin sensitivity assays, time-zero samples were collected (200-400 μL), then 1 mL aliquots were transferred to 14 mL test tubes, and appropriate volumes of menadione, H₂O₂, or ampicillin stock solutions, not in excess of 15 μL, were added to obtain the final concentrations (1 mM menadione, 5 mM H₂O₂, 7.5 μg/mL ampicillin, 100 ng/mL ofloxacin, 15 ng/mL ciprofloxacin, 500 ng/mL gentamicin, 10 μg/mL tetracycline, and 15 μg/mL chloramphenicol). For NaOCl, due to its reactivity with media components⁶⁴, 10 mL of culture was centrifuged at 3,000 rpm for 10 minutes in a benchtop centrifuge, 9.5 mL of the supernatant was removed and the cell pellet was resuspended with 9.5 mL of sterile phosphate buffered saline (PBS) at pH 7.2. The suspension was spun down again at 3,000 rpm for 10 minutes, and 9.5 mL of the supernatant removed. The cell pellet was resuspended with 4.5 mL of sterile PBS. The cell density was adjusted with sterile PBS to achieve an OD₆₀₀=0.2. Time-zero samples were collected (200-400 μL), 1 mL aliquots were transferred to 14 mL test tubes, and NaOCl stock solutions was added to obtain the final concentration (20 μM NaOCl). At the specified times (1, 2 hours for menadione, H₂O₂, NaOCl; 1, 2, 3, 4 hours for antibiotics), sample aliquots were collected (200-400 μL). All samples were immediately centrifuged at 10 k rpm in a microcentrifuge, 95% of the supernatant was removed, and the cell pellets were resuspended in PBS. Samples were serially-diluted and plated on LB agar plates, which were then incubated overnight at 37° C. Colony forming units were counted approximately 16-18 hours after plating.

### Carboxin Inhibitor Experiments

Strains were grown aerobically from an initial inoculation of OD₆₀₀=0.01 to OD₆₀₀=0.16-0.20 in 250 mL baffled flasks filled to 1/10^{th} the total volume and shaken at 300 rpm at 37° C. Time-zero samples were collected (200-400 μL), then 1 mL aliquots were transferred to 14 mL test tubes. Carboxin solubilized in 100% ethanol or ethanol alone was added to the tubes. Carboxin was added at a final concentration of 500 μM H₂O₂ or ampicillin stock solutions were added to obtain the final concentrations of 5 mM H₂O₂ and 10 μg/mL ampicillin A dose response was also performed of both carboxin (0, 250, 500, 750, and 1000 μM) and ampicillin (0, 5, 7.5, 10, and 15 μg/mL) to determine if the two compounds demonstrate a synergistic interaction. Drug synergism was calculated using the Bliss Independence and Highest Single Agent models^{52, 53}. Specifically, the formula,

BIC_{AB}=A+B−AB (1)

was used to calculate synergism with the Bliss Independence model. A and B are the effects of the two drugs in isolation, whereas, BIC_{AB} is the combined effect of the two drugs as predicted by the Bliss Independence model. If C_{AB}, the experimentally-determined combined effect of the two drugs, is >BIC_{AB}, synergy is observed. In contrast, in the Highest Single Agent model, if C_{AB}>max(A, B) synergy is observed. Since cell death was monitored, the quantitative effect of each compound was defined as the fractional reduction of the population, R=1−CFUₜ/CFU₀, where CFUₜ is the number of CFUs measured after treatment, and CFU₀ is the number of CFUs measured before treatment. R=1 indicates complete loss of the population, R=0 indicates a population in stasis, and R<0 indicates a growing population. Since carboxin was non-lethal and allowed significant growth, even at concentrations as high as 1 mM, the Highest Single Agent model was a much more stringent measure of synergy than the Bliss Independence model. The Bliss Independence model can be written as follows,

BIC_{AB}=A(1−B)+B (2)

If A is a compound that reduces CFUs, such as ampicillin, its effect above the MIC will be 0≦A≦1, whereas if B is a compound that allows growth at all concentrations, its effect will be B<0 regardless of the concentration. Rearrangement of the above yields,

BIC_{AB}/A=1−B+B/A (3)

Since equation 3 yields BIC_{AB}/A<1 for all B<0 and 0<A<1, the Highest Single Agent model requires C_{AB}/A>1 and the Bliss Independence model requires C_{AB}/BIC_{AB}>1 for synergy, the Highest Single Agent model will always be a more strict synergy requirement under these conditions. Synergy can readily be observed from the relative survival curves in FIG. 8 (curves significantly lower than 1) where 7.5 and 10 μg/mL ampicillin synergize with carboxin concentrations from 250-1000 μM.
Modeling Escherichia coli ROS Metabolism

Systems-level metabolic modeling was performed using FBA and the COBRA Toolbox²⁷. Aerobic E. coli metabolism (O₂⁻ uptake=−18.5 mmol/gDW/hr³²) was modeled using iAF1260 with glucose (glucose uptake=−11 mmol/gDW/hr³²) and ammonia as the sole carbon and nitrogen sources. The model was augmented with ROS-generating reactions. Single-gene deletion analysis was performed using the built-in COBRA function.

### Statistical Analysis of Model Performance

Statistical significance was assessed using the null hypothesis that random selection of genes would match experimental results as well as predictions from the modeling approaches described herein. For the GFP reporter systems, where N genes exhibited an increased ROS/BM compared to wildtype (p-value<0.05), and M genes did not (N+M: total number of genes tested), we identified the number of genes, P, our approach predicted to increase ROS/BM. We calculated the (a) total number of ways to pick P genes from N+M, and then calculated the (b) number of ways to pick P genes that would yield C correct predictions, C being defined as the correctly predicted number of genes our approach identified to increase ROS/BM. The ratio of (b)/(a) is the probability that random selection would yield the same frequency of correct predictions as our approach. Agreement was assessed by calculating the number of predictions that agreed with experimental results. For the O2⁻-sensing GFP reporter, 17 of the 21 genes (81%) experimentally tested qualitatively agreed with predictions, whereas for the H2O2-sensing GFP reporter, 19 of the 21 genes (90%) experimentally tested qualitatively agreed with predictions. Identical procedures were used in the analysis of HyPer results, except that a p-value of 0.1 was used to identify genes that exhibited an increased H₂O₂/BM compared to wildtype. For antimicrobial sensitivity assays, statistical significance was assessed similarly, except that N in this case is the number of genes that exhibited a 2-fold increase in susceptibility toward any oxidant after a treatment time of 2 hours.

Modeling Escherichia coli ROS Metabolism

Systems-level metabolic modeling was performed using FBA and the COBRA Toolbox²⁷. Reactions in the current metabolic reconstruction of E. coli (iAF 1260)² involving H₂O₂ and O₂⁻ are presented in Table 4. E. coli has been experimentally shown to generate 14 μM/s H₂O₂³ and 5 μM/s O₂⁻⁴ when grown in glucose media. When aerobic metabolism (O₂ uptake=−18.5 mmol gDW⁻¹ hr⁻¹)² is modeled using iAF 1260 with glucose (glucose uptake=−11 mmol gDW⁻¹ hr⁻¹)², ammonia (unlimited), sulfate (unlimited), and phosphate (unlimited) as the sole carbon, nitrogen, sulfur and phosphorous sources, respectively, while optimizing for biomass, H₂O₂ and O₂⁻ are not produced. Incorporation of transcriptional regulation⁵ does not predict O₂⁻ production, and yields H₂O₂ production at a level ˜600-fold less than experimental measures. This stems from three issues: (1) absence of known ROS-generating reactions, (2) incomplete identification of ROS sources, and (3) optimization of the objective function.

The reactions in Table 4 are involved in ROS detoxification, alternative carbon or nitrogen metabolism, and cofactor or prosthetic biogenesis. Of these reactions, only aspartate oxidase (nadB)⁶ and pyridoxal 5′-phosphate oxidase (pdxH)⁷ are likely to generate endogenous ROS in most environments. The remaining production reactions are involved in degradative pathways that are specific to particular growth environments. With transcriptional regulation incorporated into iAF 1260, pyridoxal 5′-phosphate oxidase generates 2.1×10⁻⁴ mmol H₂O₂ gDW⁻¹ hr⁻¹ when grown aerobically in glucose minimal media. With correction of iAF1260 to reflect recent understanding of the aerobic electron acceptor for aspartate oxidase⁶, O₂, this enzyme generates 23×10⁻⁴ mmol H₂O₂ gDW⁻¹ hr⁻¹ in the same media. Experimentally, E. coli has been measured to generate 14 μM H₂O₂/5, which corresponds to 1233×10⁻⁴ mmol H₂O₂ gDW′ hr⁻¹ using a cell volume of 6.8×10⁻¹⁶ L¹¹ and cell weight of 278×10⁻¹⁵ gDW¹⁵. All other ROS production reactions within iAF 1260 are not utilized in aerobic glucose minimal media, as expected. Therefore, collectively all of the ROS-generating reactions within iAF 1260 produce less than 2% of the H₂O₂ generated by E. coli under similar environmental conditions. This represents a large gap in the metabolic network of E. coli, where 98% of H₂O₂ production and 100% of O2− production are unaccounted for.

Filling the ROS Metabolic Gap in iAF1260

Beyond enzymes within Table 4, experimental evidence exists for only four E. coli enzymes as producers of H₂O₂ and/or O₂⁻ under physiological conditions. These are fumarate reductase (frdABCD)^{6, 9-11}, NADH dehydrogenase II (ndh)^{9, 10}, sulfite reductase (cysIJ)⁹, and succinate dehydrogenase (sdhABCD)¹². iAF1260 includes these enzymes and their intended reactions, but lacks all of their ROS-generating side reactions with the exception of H₂O₂ from aspartate oxidase. This gap in the metabolic network is widened by the absence of yet to be identified ROS sources that account for the majority of ROS in E. coli⁶. Inclusion of all of these reactions into the stoichiometric reconstruction is necessary to model ROS metabolism.

To include all ROS sources in our models described herein, every enzyme with the capacity to lose electrons to O2 was identified using the Ecocyc database⁷. These enzymes use flavins, quinones, and/or transition metal centers during catalysis¹³, and are listed along with their intended, H₂O₂-generating and O₂⁻-generating reactions in Table 1. In total, 133 reactions have the capacity to generate ROS in E. coli and were included in the model. Since electron donors and acceptors varied from one reaction to another, each was dissected separately to identify the ROS-generating side reactions. When ROS-generating reactions were absent from the literature for any particular enzyme, general reactions for electron loss from reduced electron carriers were used. Details of this procedure and the ROS-generating reactions are provided in Table 1. All enzymes were allowed to produce both H₂O₂ and O₂⁻-simultaneously. Enzymes that use flavins or quinones derived both species from O2, while enzymes that only utilize transition metal centers derived O₂⁻ from O₂, and H₂O₂ from O₂⁻. This is in recognition of the fact that enzymes with only transition metal centers (e.g., Fe—S), such as aconitase, fumarase, and dihydroxy acid dehydratase, are readily oxidized by O₂⁻⁷, and that continuous recycling of these enzymes' active sites occurs¹⁴.

Inclusion of ROS-generating reactions is a necessary but insufficient requirement to model ROS production. Consider the following reactions in iAF1260 catalyzed by aspartate oxidase:

L-asp+O₂→α-imsucc+H₂O₂+H⁺

L-asp+UQ→α-imsucc+UQH₂+H⁺

L-asp+MQ→α-imsucc+MQH₂+H⁺

L-asp+fumarate→α-imsucc+succinate+H⁺

where L-asp stands for L-aspartate, α-imsucc for α-iminiosuccinate, MQ for menaquinone, MQH₂ for menaquinol, UQ for ubiquinone, and UQH2 for ubiquinol. When growth is modeled in silico using biomass production as the objective function, electrons flow from L-asp through NadB to an electron acceptor in the following preferential order, UQ>fumarate>MQ>O₂. This yields flux solutions that do not identify NadB as a source of H₂O₂ despite experimental evidence to the contrary⁶. This stems from the optimization reducing potential is lost when electrons flow to O₂ and produce H₂O₂, while electron flow to UQ is favorable because UQH₂ can be used to generate proton motive force (pmf) and drive ATP production. When optimizing for biomass production, the UQ reaction carries flux, subject to material balance and thermodynamic constraints. This “all or none” issue has been addressed previously when evidence for branching of flux exists², and is handled by combining the reactions into one with the proper branching stoichiometries (coupling). For example, if 50% of the electrons from L-asp reduce UQ and 50% reduce MQ, the combined reaction would be:

L-asp+½UQ+½MQ→α-imsucc+½UQH₂+½MQH₂+H⁺

Consider the combination of the UQ and O₂ aspartate oxidase reactions:

L-asp+(1−c_{H202})UQ+(c_{H2O2})O₂→α-imsucc+(1−c_{H2O2})UQH₂+(c_{H2O2})H₂O₂+H⁺

To model H₂O₂ production from NadB, the stoichiometric coefficient that specifies the proportion of electron flow to O2 compared to UQ, c_{H2O2}, needs to be defined. To model whole-cell H₂O₂ metabolism, an analogous constant, c_{i,H2O2}, for every H₂O₂-producing enzyme needs to be defined separately. Differences in the values of these constants reflect the different tendencies to form H₂O₂ between enzymes¹². Analogously, to model endogenous O2− production, separate constants for O2−, c_{i,O2-}, are required. However, whole-cell H₂O₂ production³ and O₂⁻ production⁴ have been measured and can be used to bound the production of H₂O₂ and O₂⁻ from our models.

### Generation of an Ensemble of ROS Models

To allow for uncertainty in the constants, c_{i,H2O2} and c_{I,O2-}, two ensembles of genome-scale metabolic models, each with 1000 different models were employed. The first ensemble drew its constants from an exponential distribution in order to model a centralized ROS production network, while the second drew its constants from a Gaussian distribution to model a distributed ROS production network. Each set of 266 constants was integrated into iAF 1260 and normalized such that simulations of the wildtype model in minimal glucose media matched the best experimental measures of H₂O₂ and O₂⁻ production, and consumption of O₂⁻ was primarily executed by superoxide dismutase (99 9%), instead of damage to transition metal centers (constrained to be <1%). The 99:1 ratio was inspired by the greater than 100-fold difference in rate constants between the reactions of O₂⁻ with superoxide dismutase and aconitase¹⁴. This produced 2,000 different models that generated the exact same quantities of ROS from the wildtype model, but with each using enzymes in a different manner to do so. For each model in the ensemble (wildtype network), it was determined with flux variability analysis (FVA) that at 100% biomass production, the ROS production solution was unique.

It should be noted that coupling the ROS reactions in this manner assumes that ROS production is dependent on and proportional to the intended reaction flux (ROS_{Rxni}αvᵢ). Under balanced growth this assumption is valid, as the initial reaction steps for ROS-generating reactions and their intended counterparts are the same, and it is the promiscuity of the electron carrier for O₂ that generates ROS. For instance, the dehydrogenation of NADH and subsequent electron transfer to the FMN cofactor in the case of NDH-I is dictated by demands for the products of the intended reaction, while the promiscuity of the FMNH2 with O₂ dictates the amount of ROS generated.

### ROS Models Construction Summary

In summary, to model endogenous ROS production in E. coli, iAF 1260 was augmented in the following ways: (1) all possible ROS-generating reactions were included in the metabolic reconstruction, (2) ROS-generating reactions were coupled with the intended reactions of their respective enzymes using ensembles of and c_{i,H2O2} and c_{I,O2-}, (3) experimental measurements of whole-cell H₂O₂ and O₂⁻ production were used to constrain the total electron flow from these reactions to O₂, such that all wildtype models produced the experimentally measured levels of H₂O₂ and O₂⁻, and (4)≧99% of O₂⁻ consumption was required to be performed by superoxide dismutase, as opposed to damage to transition metal centers.

### ROS Model Simulations

The initial media conditions included glucose as the sole carbon source and limiting nutrient, ammonia as the sole nitrogen source, sulfate as the sole sulfur source, phosphate as the sole phosphorous source, and oxygen. Transcriptional regulation from Covert and colleagues⁵ was used to identify gene products that are not present under aerobic glucose growth. The list of genes that were turned off due to transcriptional regulation is presented in Table 5. Reactions contained in iAF1260 that generate ROS stoichiometrically were investigated due to the ease with which miscalculated fluxes for these enzymes could skew results. The quinol monooxygenase, aminoacetone oxidase, and pyridoxamine 5′-phosphate oxidase reactions catalyzed by the ygiN, tynA, and pdxH gene products were omitted; other reactions catalyzed by the gene products of tynA and pdxH were included in the analysis.

Single-gene deletion analysis was used to probe how perturbations to the metabolic network affect ROS production. This was performed with the built-in COBRA function for each of the 2,000 models separately. For each genetic deletion, two distributions of ROS/BM were obtained, one for each ensemble. From these distributions, the mean ROS/BM for that genetic perturbation over the entire ensemble, and the relative mean ROS/BM for that genetic deletion in comparison to wildtype over the entire ensemble were calculated. The values for genetic deletions that altered ROS flux are presented in Table 6. FVA was not performed on each of the mutant networks (2,000 per mutant), because the diversity between networks was hypothesized to be more significant that the diversity in solution space for a single network. Indeed, this was confirmed using FVA for all 2,000 wild-type networks.

Some targets identified by the approaches described herein, such as ΔtpiA, ΔaceE, ΔaceF, Δlpd, could not be grown in minimal glucose media, and thus were not tested experimentally. For example, the inability of dtpiA to grow in minimal glucose media is caused by a requirement to produce methylglyoxal (MG) to provide an outlet for DHAP. The models described herein correctly predicted use of the MG pathway in this deletion, but does not factor in the cytotoxic effects of MG as a potent electrophile^{15, 16}. Deletion of pyruvate dehydrogenase (ΔaceE, ΔaceF, Δlpd) produces acetate auxotrophy, although pyruvate oxidase (poxB) can provide acetate under aerobic conditions to support significantly retarded growth¹⁷⁻¹⁹. The models described herein correctly predicted the use of pyruvate oxidase, but did not factor in its inability to carry sufficient flux to support normal growth. These physiological constraints can be incorporated into iterations of the model as bounds on the reaction fluxes in order to improve the growth/non-growth prediction.

H₂O₂ and O₂⁻ produced in the models described herein were overwhelmingly detoxified by catalase and superoxide dismutase reactions. As stated elsewhere herein, it was not desired to overwhelm the oxidative detoxification and repair capabilities of E. coli with endogenously generated ROS, but instead to increase endogenous production such that the ability of E. coli to cope with exogenous oxidative stress would be compromised. Accordingly, effects of perturbations to oxidant detoxification systems on the models described herein were not studied. Such analyses would require, for example, incorporation of many more reactions accounting for damage and repair of biomolecules and the effects of antioxidant metabolites.

TABLE 1
ROS-generation Reactions within iAF1260

| Rxn Name | Flavin | Quinone | Fe—S | Heme | Reversible | iAF1260 index |
|---|---|---|---|---|---|---|
| ACHBS | 1 | 0 | 0 | 0 | 0 | 166 |
| ACLS | 1 | 0 | 0 | 0 | 0 | 168 |
| ACOAD1f | 1 | 0 | 0 | 0 | 1 | 178 |
| ACOAD2f | 1 | 0 | 0 | 0 | 1 | 179 |
| ACOAD3f | 1 | 0 | 0 | 0 | 1 | 180 |
| ACOAD4f | 1 | 0 | 0 | 0 | 1 | 181 |
| ACOAD5f | 1 | 0 | 0 | 0 | 1 | 182 |
| ACOAD6f | 1 | 0 | 0 | 0 | 1 | 183 |
| ACOAD7f | 1 | 0 | 0 | 0 | 1 | 184 |
| ACOAD8f | 1 | 0 | 0 | 0 | 1 | 185 |
| ACONTa | 0 | 0 | 1 | 0 | 1 | 191 |
| ACONTb | 0 | 0 | 1 | 0 | 1 | 192 |
| AKGDH | 1 | 0 | 0 | 0 | 0 | 256 |
| AMMQLT8 | 0 | 1 | 0 | 0 | 0 | 304 |
| ARBTNR1 | 1 | 0 | 0 | 0 | 0 | 320 |
| ARBTNR2 | 1 | 0 | 0 | 0 | 0 | 321 |
| ASPO3 | 1 | 1 | 0 | 0 | 0 | 359 |
| ASPO4 | 1 | 0 | 0 | 0 | 0 | 360 |
| ASPO5 | 1 | 1 | 0 | 0 | 0 | 361 |
| BTS4 | 0 | 0 | 1 | 0 | 0 | 385 |
| CPGNR1 | 1 | 0 | 0 | 0 | 0 | 459 |
| CPGNR2 | 1 | 0 | 0 | 0 | 0 | 460 |
| CPPPGO2 | 0 | 0 | 1 | 0 | 0 | 468 |
| CYTBD2pp | 0 | 1 | 0 | 1 | 0 | 515 |
| CYTBDpp | 0 | 1 | 0 | 1 | 0 | 516 |
| CYTBO3_4pp | 0 | 1 | 0 | 1 | 0 | 517 |
| DAAD | 1 | 0 | 0 | 0 | 0 | 526 |
| DHAD1 | 0 | 0 | 1 | 0 | 0 | 573 |
| DHAD2 | 0 | 0 | 1 | 0 | 0 | 574 |
| DHNAOT4 | 0 | 1 | 0 | 0 | 0 | 587 |
| DHORD2 | 1 | 1 | 0 | 0 | 0 | 589 |
| DHORD5 | 1 | 1 | 0 | 0 | 0 | 590 |
| DMPPS | 1 | 0 | 1 | 0 | 0 | 608 |
| DMQMT | 0 | 1 | 0 | 0 | 0 | 609 |
| DMSOR1 | 0 | 1 | 0 | 0 | 0 | 610 |
| DMSOR1pp | 0 | 1 | 0 | 0 | 0 | 611 |
| DMSOR2 | 0 | 1 | 0 | 0 | 0 | 612 |
| DMSOR2pp | 0 | 1 | 0 | 0 | 0 | 613 |
| DSBAO1 | 0 | 1 | 0 | 0 | 0 | 628 |
| DSBAO2 | 0 | 1 | 0 | 0 | 0 | 629 |
| FADRx | 1 | 0 | 0 | 0 | 0 | 1037 |
| FADRx2 | 1 | 0 | 1 | 0 | 0 | 1038 |
| FDH4pp | 0 | 1 | 0 | 0 | 0 | 1049 |
| FDH5pp | 0 | 1 | 0 | 0 | 0 | 1050 |
| FDMO | 1 | 0 | 0 | 0 | 0 | 1051 |
| FDMO2 | 1 | 0 | 0 | 0 | 0 | 1052 |
| FDMO3 | 1 | 0 | 0 | 0 | 0 | 1053 |
| FDMO4 | 1 | 0 | 0 | 0 | 0 | 1054 |
| FDMO6 | 1 | 0 | 0 | 0 | 0 | 1055 |
| FE3HOXR1 | 1 | 0 | 0 | 0 | 0 | 1066 |
| FE3HOXR2 | 1 | 0 | 0 | 0 | 0 | 1067 |
| FE3Ri | 1 | 0 | 0 | 0 | 0 | 1074 |
| FECRMR1 | 1 | 0 | 0 | 0 | 0 | 1077 |
| FECRMR2 | 1 | 0 | 0 | 0 | 0 | 1078 |
| FEENTERR1 | 1 | 0 | 0 | 0 | 0 | 1085 |
| FEENTERR2 | 1 | 0 | 0 | 0 | 0 | 1086 |
| FEOXAMR1 | 1 | 0 | 0 | 0 | 0 | 1093 |
| FEOXAMR2 | 1 | 0 | 0 | 0 | 0 | 1094 |
| FLDR | 1 | 0 | 0 | 0 | 0 | 1104 |
| FLVR | 1 | 0 | 1 | 0 | 0 | 1105 |
| FMNRx | 1 | 0 | 0 | 0 | 0 | 1109 |
| FMNRx2 | 1 | 0 | 1 | 0 | 0 | 1110 |
| FRD2 | 1 | 1 | 1 | 0 | 0 | 1114 |
| FRD3 | 1 | 1 | 1 | 0 | 0 | 1115 |
| FUM | 0 | 0 | 1 | 0 | 1 | 1130 |
| G3PD5 | 1 | 1 | 0 | 0 | 0 | 1150 |
| G3PD6 | 1 | 1 | 0 | 0 | 0 | 1151 |
| G3PD7 | 1 | 1 | 0 | 0 | 0 | 1152 |
| GLCDpp | 0 | 1 | 0 | 0 | 0 | 1216 |
| GLUSy | 1 | 0 | 0 | 0 | 0 | 1253 |
| GLXCL | 1 | 0 | 0 | 0 | 0 | 1260 |
| GLYCL | 1 | 0 | 0 | 0 | 0 | 1276 |
| GLYCTO2 | 0 | 1 | 0 | 0 | 0 | 1282 |
| GLYCTO3 | 0 | 1 | 0 | 0 | 0 | 1283 |
| GLYCTO4 | 0 | 1 | 0 | 0 | 0 | 1284 |
| GTHOr | 1 | 0 | 0 | 0 | 1 | 1323 |
| HYD1pp | 0 | 1 | 1 | 0 | 0 | 1411 |
| HYD2pp | 0 | 1 | 1 | 0 | 0 | 1412 |
| HYD3pp | 0 | 1 | 1 | 0 | 0 | 1413 |
| IPDPS | 1 | 0 | 1 | 0 | 0 | 1449 |
| LDH_D | 1 | 0 | 0 | 0 | 1 | 1490 |
| LDH_D2 | 0 | 1 | 0 | 0 | 0 | 1491 |
| L_LACD2 | 1 | 1 | 0 | 0 | 0 | 1570 |
| L_LACD3 | 1 | 1 | 0 | 0 | 0 | 1571 |
| MDH2 | 1 | 1 | 0 | 0 | 0 | 1623 |
| MDH3 | 1 | 1 | 0 | 0 | 0 | 1624 |
| MICITD | 0 | 0 | 1 | 0 | 0 | 1655 |
| MTHFR2 | 1 | 0 | 0 | 0 | 0 | 1702 |
| NADH10 | 1 | 1 | 0 | 0 | 0 | 1712 |
| NADH16pp | 1 | 1 | 1 | 0 | 0 | 1713 |
| NADH17pp | 1 | 1 | 1 | 0 | 0 | 1714 |
| NADH18pp | 1 | 1 | 1 | 0 | 0 | 1715 |
| NADH5 | 1 | 1 | 0 | 0 | 0 | 1716 |
| NADH9 | 1 | 1 | 0 | 0 | 0 | 1717 |
| NADPHQR2 | 1 | 1 | 0 | 0 | 0 | 1720 |
| NADPHQR3 | 1 | 1 | 0 | 0 | 0 | 1721 |
| NADPHQR4 | 1 | 1 | 0 | 0 | 0 | 1722 |
| NADTRHD | 1 | 0 | 0 | 0 | 0 | 1725 |
| NHFRBO | 1 | 0 | 0 | 1 | 0 | 1741 |
| NO3R1bpp | 0 | 1 | 0 | 0 | 0 | 1759 |
| NO3R1pp | 0 | 1 | 1 | 0 | 0 | 1760 |
| NO3R2bpp | 0 | 1 | 0 | 0 | 0 | 1761 |
| NO3R2pp | 0 | 1 | 1 | 0 | 0 | 1762 |
| NODOx | 1 | 0 | 0 | 0 | 0 | 1765 |
| NODOy | 1 | 0 | 0 | 0 | 0 | 1766 |
| NTRIR2x | 1 | 0 | 1 | 1 | 0 | 1814 |
| NTRIR3pp | 0 | 1 | 0 | 0 | 0 | 1815 |
| NTRIR4pp | 0 | 1 | 0 | 0 | 0 | 1816 |
| OBTFL | 0 | 0 | 1 | 0 | 0 | 1831 |
| P5CD | 1 | 0 | 0 | 0 | 0 | 1859 |
| PDH | 1 | 0 | 0 | 0 | 0 | 1890 |
| PFL | 0 | 0 | 1 | 0 | 0 | 1909 |
| POX | 1 | 1 | 0 | 0 | 0 | 2011 |
| PPCDC | 1 | 0 | 0 | 0 | 0 | 2021 |
| PPNCL2 | 1 | 0 | 0 | 0 | 0 | 2029 |
| PPPGO | 1 | 0 | 0 | 0 | 0 | 2032 |
| PPPGO3 | 1 | 0 | 0 | 0 | 0 | 2033 |
| PPPNDO | 1 | 0 | 0 | 0 | 0 | 2034 |
| PROD2 | 1 | 0 | 0 | 0 | 0 | 2048 |
| QULNS | 0 | 0 | 1 | 0 | 0 | 2105 |
| RNTR1c | 1 | 0 | 1 | 0 | 0 | 2134 |
| RNTR2c | 1 | 0 | 1 | 0 | 0 | 2135 |
| RNTR3c | 1 | 0 | 1 | 0 | 0 | 2136 |
| RNTR4c | 1 | 0 | 1 | 0 | 0 | 2137 |
| SERD_L | 0 | 0 | 1 | 0 | 0 | 2155 |
| SUCDi | 1 | 1 | 1 | 0 | 0 | 2192 |
| SULRi | 1 | 0 | 1 | 1 | 0 | 2199 |
| TARTD | 0 | 0 | 1 | 0 | 0 | 2204 |
| THRD_L | 0 | 0 | 1 | 0 | 0 | 2233 |
| TMAOR1 | 0 | 1 | 0 | 0 | 0 | 2247 |
| TMAOR1pp | 0 | 1 | 0 | 0 | 0 | 2248 |
| TMAOR2 | 0 | 1 | 0 | 0 | 0 | 2249 |
| TMAOR2pp | 0 | 1 | 0 | 0 | 0 | 2250 |
| TRDR | 1 | 0 | 0 | 0 | 0 | 2261 |
| UAPGR | 1 | 0 | 0 | 0 | 0 | 2304 |
| UDPGALM | 1 | 0 | 0 | 0 | 0 | 2311 |

| Rxn Name | |
|---|---|
| | Rxn (iAF1260) |
| ACHBS | [c]: 2obut + h + pyr --> 2ahbut + co2 |
| ACLS | [c]: h + (2) pyr --> alac-S + co2 |
| ACOAD1f | [c]: btcoa + fad <==> b2coa + fadh2 |
| ACOAD2f | [c]: fad + hxcoa <==> fadh2 + hx2coa |
| ACOAD3f | [c]: fad + occoa <==> fadh2 + oc2coa |
| ACOAD4f | [c]: dcacoa + fad <==> dc2coa + fadh2 |
| ACOAD5f | [c]: ddcacoa + fad <==> dd2coa + fadh2 |
| ACOAD6f | [c]: fad + tdcoa <==> fadh2 + td2coa |
| ACOAD7f | [c]: fad + pmtcoa <==> fadh2 + hdd2coa |
| ACOAD8f | [c]: fad + stcoa <==> fadh2 + od2coa |
| ACONTa | [c]: cit <==> acon-C + h2o |
| ACONTb | [c]: acon-C + h2o <==> icit |
| AKGDH | [c]: akg + coa + nad --> co2 + nadh + succoa |
| AMMQLT8 | [c]: 2dmmql8 + amet --> ahcys + h + mql8 |
| ARBTNR1 | [c]: (2) arbtn-fe3 + fadh2 --> (2) arbtn + fad + (2) fe2 + (2) h |
| ARBTNR2 | [c]: (2) arbtn-fe3 + fmnh2 --> (2) arbtn + (2) fe2 + fmn + (2) h |
| ASPO3 | [c]: asp-L + q8 --> h + iasp + q8h2 |
| ASPO4 | [c]: asp-L + mqn8 --> h + iasp + mql8 |
| ASPO5 | [c]: asp-L + fum --> h + iasp + succ |
| BTS4 | [c]: amet + dtbt + s --> btn + dad-5 + h + met-L |
| CPGNR1 | [c]: (2) cpgn + fadh2 --> (2) cpgn-un + fad + (2) fe2 + (2) h |
| CPGNR2 | [c]: (2) cpgn + fmnh2 --> (2) cpgn-un + (2) fe2 + fmn + (2) h |
| CPPPGO2 | [c]: (2) amet + cpppg3 --> (2) co2 + (2) dad-5 + (2) met-L + pppg9 |
| CYTBD2pp | (2) h[c] + mql8[c] + (0.5) o2[c] --> (2) h[p] + h2o[c] + mqn8[c] |
| CYTBDpp | (2) h[c] + (0.5) o2[c] + q8h2[c] --> (2) h[p] + h2o[c] + q8[c] |
| CYTBO3_4pp | (4) h[c] + (0.5) o2[c] + q8h2[c] --> (4) h[p] + h2o[c] + q8[c] |
| DAAD | [c]: ala-D + fad + h2o --> fadh2 + nh4 + pyr |
| DHAD1 | [c]: 23dhmb --> 3mob + h2o |
| DHAD2 | [c]: 23dhmp --> 3mop + h2o |
| DHNAOT4 | [c]: dhna + h + octdp --> 2dmmql8 + co2 + ppi |
| DHORD2 | [c]: dhor-S + q8 --> orot + q8h2 |
| DHORD5 | [c]: dhor-S + mqn8 --> mql8 + orot |
| DMPPS | [c]: h + h2mb4p + nadh --> dmpp + h2o + nad |
| DMQMT | [c]: 2omhmbl + amet --> ahcys + h + q8h2 |
| DMSOR1 | [c]: dmso + mql8 --> dms + h2o + mqn8 |
| DMSOR1pp | dmso[p] + mql8[c]: --> dms[p] + h2o[p] + mqn8[c] |
| DMSOR2 | [c]: 2dmmql8 + dmso --> 2dmmq8 + dms + h2o |
| DMSOR2pp | 2dmmql8[c] + dmso[p] --> 2dmmq8[c] + dms[p] + h2o[p] |
| DSBAO1 | dsbard[p] + q8[c] --> dsbaox[p] + q8h2[c] |
| DSBAO2 | dsbard[p] + mqn8[c] --> dsbaox[p] + mql8[c] |
| FADRx | [c]: fad + h + nadh --> fadh2 + nad |
| FADRx2 | [c]: fad + h + nadph --> fadh2 + nadp |
| FDH4pp | for[p] + (2) h[c] + q8[c] --> co2[c] + h[p] + q8h2[c] |
| FDH5pp | for[p] + (2) h[c] + mqn8[c] --> co2[c] + h[p] + mql8[c] |
| FDMO | [c]: fmnh2 + isetac + o2 --> fmn + gcald + h + h2o + so3 |
| FDMO2 | [c]: fmnh2 + mso3 + o2 --> fald + fmn + h + h2o + so3 |
| FDMO3 | [c]: ethso3 + fmnh2 + o2 --> acald + fmn + h + h2o + so3 |
| FDMO4 | [c]: butso3 + fmnh2 + o2 --> btal + fmn + h + h2o + so3 |
| FDMO6 | [c]: fmnh2 + o2 + sulfac --> fmn + glx + h + h2o + so3 |
| FE3HOXR1 | [c]: fadh2 + (2) fe3hox --> fad + (2) fe2 + (2) fe3hox-un + (2) h |
| FE3HOXR2 | [c]: (2) fe3hox + fmnh2 --> (2) fe2 + (2) fe3hox-un + fmn + (2) h |
| FE3Ri | [c]: fadh2 + (2) fe3 --> fad + (2) fe2 + (2) h |
| FECRMR1 | [c]: fadh2 + (2) fecrm --> fad + (2) fe2 + (2) fecrm-un + (2) h |
| FECRMR2 | [c]: (2) fecrm + fmnh2 --> (2) fe2 + (2) fecrm-un + fmn + (2) h |
| FEENTERR1 | [c]: fadh2 + (2) feenter --> (2) enter + fad + (2) fe2 + (2) h |
| FEENTERR2 | [c]: (2) feenter + fmnh2 --> (2) enter + (2) fe2 + fmn + (2) h |
| FEOXAMR1 | [c]: fadh2 + (2) feoxam --> fad + (2) fe2 + (2) feoxam-un + (2) h |
| FEOXAMR2 | [c]: (2) feoxam + fmnh2 --> (2) fe2 + (2) feoxam-un + fmn + (2) h |
| FLDR | [c]: fldox + h + nadph --> fldrd + nadp |
| FLVR | [c]: h + nadph + ribflv --> nadp + rbflvrd |
| FMNRx | [c]: fmn + h + nadh --> fmnh2 + nad |
| FMNRx2 | [c]: fmn + h + nadph --> fmnh2 + nadp |
| FRD2 | [c]: fum + mql8 --> mqn8 + succ |
| FRD3 | [c]: 2dmmql8 + fum --> 2dmmq8 + succ |
| FUM | [c]: fum + h2o <==> mal-L |
| G3PD5 | [c]: glyc3p + q8 --> dhap + q8h2 |
| G3PD6 | [c]: glyc3p + mqn8 --> dhap + mql8 |
| G3PD7 | [c]: 2dmmq8 + glyc3p --> 2dmmql8 + dhap |
| GLCDpp | glc-D[p] + h2o[p] + q8[c] --> glcn[p] + h[p] + q8h2[c] |
| GLUSy | [c]: akg + gln-L + h + nadph --> (2) glu-L + nadp |
| GLXCL | [c]: (2) glx + h --> 2h3oppan + co2 |
| GLYCL | [c]: gly + nad + thf --> co2 + mlthf + nadh + nh4 |
| GLYCTO2 | [c]: glyclt + q8 --> glx + q8h2 |
| GLYCTO3 | [c]: glyclt + mqn8 --> glx + mql8 |
| GLYCTO4 | [c]: 2dmmq8 + glyclt --> 2dmmql8 + glx |
| GTHOr | [c]: gthox + h + nadph <==> (2) gthrd + nadp |
| HYD1pp | (2) h[c] + h2[c] + q8[c] --> (2) h[p] + q8h2[c] |
| HYD2pp | (2) h[c] + h2[c] + mqn8[c] --> (2) h[p] + mql8[c] |
| HYD3pp | 2dmmq8[c] + (2) h[c] + h2[c] --> 2dmmql8[c] + (2) h[p] |
| IPDPS | [c]: h + h2mb4p + nadh --> h2o + ipdp + nad |
| LDH_D | [c]: lac-D + nad <==> h + nadh + pyr |
| LDH_D2 | [c]: lac-D + q8 --> pyr + q8h2 |
| L_LACD2 | [c]: lac-L + q8 --> pyr + q8h2 |
| L_LACD3 | [c]: lac-L + mqn8 --> mql8 + pyr |
| MDH2 | [c]: mal-L + q8 --> oaa + q8h2 |
| MDH3 | [c]: mal-L + mqn8 --> mql8 + oaa |
| MICITD | [c]: 2mcacn + h2o --> micit |
| MTHFR2 | [c]: (2) h + mlthf + nadh --> 5mthf + nad |
| NADH10 | [c]: h + mqn8 + nadh --> mql8 + nad |
| NADH16pp | (4) h[c] + nadh[c] + q8[c] --> (3) h[p] + nad[c] + q8h2[c] |
| NADH17pp | (4) h[c] + mqn8[c] + nadh[c] --> (3) h[p] + mql8[c] + nad[c] |
| NADH18pp | 2dmmq8[c] + (4) h[c] + nadh[c] --> 2dmmql8[c] + (3) h[p] + nad[c] |
| NADH5 | [c]: h + nadh + q8 --> nad + q8h2 |
| NADH9 | [c]: 2dmmq8 + h + nadh --> 2dmmql8 + nad |
| NADPHQR2 | [c]: h + nadph + q8 --> nadp + q8h2 |
| NADPHQR3 | [c]: h + mqn8 + nadph --> mql8 + nadp |
| NADPHQR4 | [c]: 2dmmq8 + h + nadph --> 2dmmql8 + nadp |
| NADTRHD | [c]: nad + nadph --> nadh + nadp |
| NHFRBO | [c]: h + nadh + (2) no --> h2o + n2o + nad |
| NO3R1bpp | no3[p] + q8h2[c] --> h2o[p] + no2[p] + q8[c] |
| NO3R1pp | (2) h[c] + no3[c] + q8h2[c] --> (2) h[p] + h2o[c] + no2[c] + q8[c] |
| NO3R2bpp | mql8[c] + no3[p] --> h2o[p] + mqn8[c] + no2[p] |
| NO3R2pp | (2) h[c] + mql8[c] + no3[c] --> (2) h[p] + h2o[c] + mqn8[c] + no2[c] |
| NODOx | [c]: nadh + (2) no + (2) o2 --> h + nad + (2) no3 |
| NODOy | [c]: nadph + (2) no + (2) o2 --> h + nadp + (2) no3 |
| NTRIR2x | [c]: (5) h + (3) nadh + no2 --> (2) h2o + (3) nad + nh4 |
| NTRIR3pp | (2) h[p] + no2[p] + (3) q8h2[c] --> (2) h2o[p] + nh4[p] + (3) q8[c] |
| NTRIR4pp | (2) h[p] + (3) mql8[c] + no2[p] --> (2) h2o[p] + (3) mqn8[c] + nh4[p] |
| OBTFL | [c]: 2obut + coa --> for + ppcoa |
| P5CD | [c]: 1pyr5c + (2) h2o + nad --> glu-L + h + nadh |
| PDH | [c]: coa + nad + pyr --> accoa + co2 + nadh |
| PFL | [c]: coa + pyr --> accoa + for |
| POX | [c]: h2o + pyr + q8 --> ac + co2 + q8h2 |
| PPCDC | [c]: 4ppcys + h --> co2 + pan4p |
| PPNCL2 | [c]: 4ppan + ctp + cys-L --> 4ppcys + cmp + h + ppi |
| PPPGO | [c]: (3) q8 + pppg9 --> (3) q8h2 + ppp9, change in model |
| PPPGO3 | [c]: (3) fum + pppg9 --> ppp9 + (3) succ |
| PPPNDO | [c]: h + nadh + o2 + pppn --> cechddd + nad |
| PROD2 | [c]: fad + pro-L --> 1pyr5c + fadh2 + h |
| QULNS | [c]: dhap + iasp --> (2) h2o + pi + quln |
| RNTR1c | [c]: atp + fldrd --> datp + fldox + h2o |
| RNTR2c | [c]: fldrd + gtp --> dgtp + fldox + h2o |
| RNTR3c | [c]: ctp + fldrd --> dctp + fldox + h2o |
| RNTR4c | [c]: fldrd + utp --> dutp + fldox + h2o |
| SERD_L | [c]: ser-L --> nh4 + pyr |
| SUCDi | [c]: q8 + succ --> fum + q8h2 |
| SULRi | [c]: (5) h + (3) nadph + so3 --> (3) h2o + h2s + (3) nadp |
| TARTD | [c]: tartr-L --> h2o + oaa |
| THRD_L | [c]: thr-L --> 2obut + nh4 |
| TMAOR1 | [c]: h + mql8 + tmao --> h2o + mqn8 + tma |
| TMAOR1pp | h[p] + mql8[c] + tmao[p] --> h2o[p] + mqn8[c] + tma[p] |
| TMAOR2 | [c]: 2dmmql8 + h + tmao --> 2dmmq8 + h2o + tma |
| TMAOR2pp | 2dmmql8[c] + h[p] + tmao[p] --> 2dmmq8[c] + h2o[p] + tma[p] |
| TRDR | [c]: h + nadph + trdox --> nadp + trdrd |
| UAPGR | [c]: h + nadph + uaccg --> nadp + uamr |
| UDPGALM | [c]: udpgal --> udpgalfur |
| Rxn Name | Putative h2o2 Rxn |
| ACHBS | [c]: pyr + o2 + h2o ---> ac + co2 + h2o2 |
| ACLS | [c]: pyr + o2 + h2o ---> ac + co2 + h2o2 |
| ACOAD1f | [c]: o2 + fadh2 --> fad + h2o2 |
| ACOAD2f | [c]: o2 + fadh2 --> fad + h2o2 |
| ACOAD3f | [c]: o2 + fadh2 --> fad + h2o2 |
| ACOAD4f | [c]: o2 + fadh2 --> fad + h2o2 |
| ACOAD5f | [c]: o2 + fadh2 --> fad + h2o2 |
| ACOAD6f | [c]: o2 + fadh2 --> fad + h2o2 |
| ACOAD7f | [c]: o2 + fadh2 --> fad + h2o2 |
| ACOAD8f | [c]: o2 + fadh2 --> fad + h2o2 |
| ACONTa | [c]: (2) o2s + (3) h + nadh --> (2) h2o2 + nad |
| ACONTb | NA |
| AKGDH | [c]: akg + coa + o2 + h --> co2 + h2o2 + succoa |
| AMMQLT8 | [c]: mql8 + o2 --> h2o2 + mqn8 |
| ARBTNR1 | [c]: o2 + fadh2 --> fad + h2o2 |
| ARBTNR2 | [c]: o2 + fmnh2 --> fmn + h2o2 |
| ASPO3 | [c]: asp-L + o2 --> h + h2o2 + iasp |
| ASPO4 | [c]: asp-L + o2 --> h + h2o2 + iasp |
| ASPO5 | [c]: asp-L + o2 --> h + h2o2 + iasp |
| BTS4 | [c]: (2) o2s + (3) h + nadh --> (2) h2o2 + nad |
| CPGNR1 | [c]: o2 + fadh2 --> fad + h2o2 |
| CPGNR2 | [c]: o2 + fmnh2 --> fmn + h2o2 |
| CPPPGO2 | [c]: (2) o2s + (3) h + nadh --> (2) h2o2 + nad |
| CYTBD2pp | [c]: mql8 + o2 --> h2o2 + mqn8 |
| CYTBDpp | [c]: q8h2 + o2 --> h2o2 + q8 |
| CYTBO3_4pp | [c]: q8h2 + o2 --> h2o2 + q8 |
| DAAD | [c]: ala-D + o2 + h2o --> h2o2 + nh4 + pyr |
| DHAD1 | [c]: (2) o2s + (3) h + nadh --> (2) h2o2 + nad |
| DHAD2 | [c]: (2) o2s + (3) h + nadh --> (2) h2o2 + nad |
| DHNAOT4 | [c]: 2dmmql8 + o2 --> 2dmmq8 + h2o2 |
| DHORD2 | [c]: dhor-S + o2 --> orot + h2o2 |
| DHORD5 | [c]: dhor-S + o2 --> orot + h2o2 |
| DMPPS | [c]: (2) o2s + (3) h + nadh --> (2) h2o2 + nad |
| DMQMT | [c]: q8h2 + o2 --> h2o2 + q8 |
| DMSOR1 | [c]: mql8 + o2 --> h2o2 + mqn8 |
| DMSOR1pp | [c]: mql8 + o2 --> h2o2 + mqn8 |
| DMSOR2 | [c]: 2dmmql8 + o2 --> 2dmmq8 + h2o2 |
| DMSOR2pp | [c]: 2dmmql8 + o2 --> 2dmmq8 + h2o2 |
| DSBAO1 | dsbard[p] + o2[c] --> dsbaox[p] + h2o2[c] |
| DSBAO2 | dsbard[p] + o2[c] --> dsbaox[p] + h2o2[c] |
| FADRx | [c]: o2 + h + nadh --> h2o2 + nad |
| FADRx2 | [c]: o2 + h + nadph --> h2o2 + nadp |
| FDH4pp | for[p] + (2) h[c] + o2[c] --> co2[c] + h[p] + h2o2[c] |
| FDH5pp | for[p] + (2) h[c] + o2[c] --> co2[c] + h[p] + h2o2[c] |
| FDMO | [c]: o2 + fmnh2 --> fmn + h2o2 |
| FDMO2 | [c]: o2 + fmnh2 --> fmn + h2o2 |
| FDMO3 | [c]: o2 + fmnh2 --> fmn + h2o2 |
| FDMO4 | [c]: o2 + fmnh2 --> fmn + h2o2 |
| FDMO6 | [c]: o2 + fmnh2 --> fmn + h2o2 |
| FE3HOXR1 | [c]: o2 + fadh2 --> fad + h2o2 |
| FE3HOXR2 | [c]: o2 + fmnh2 --> fmn + h2o2 |
| FE3Ri | [c]: o2 + fadh2 --> fad + h2o2 |
| FECRMR1 | [c]: o2 + fadh2 --> fad + h2o2 |
| FECRMR2 | [c]: o2 + fmnh2 --> fmn + h2o2 |
| FEENTERR1 | [c]: o2 + fadh2 --> fad + h2o2 |
| FEENTERR2 | [c]: o2 + fmnh2 --> fmn + h2o2 |
| FEOXAMR1 | [c]: o2 + fadh2 --> fad + h2o2 |
| FEOXAMR2 | [c]: o2 + fmnh2 --> fmn + h2o2 |
| FLDR | [c]: o2 + h + nadph --> h2o2 + nadp |
| FLVR | [c]: o2 + h + nadph --> h2o2 + nadp |
| FMNRx | [c]: o2 + h + nadh --> h2o2 + nad |
| FMNRx2 | [c]: o2 + h + nadph --> h2o2 + nadp |
| FRD2 | [c]: mql8 + o2 --> h2o2 + mqn8 |
| FRD3 | [c]: 2dmmql8 + o2 --> 2dmmq8 + h2o2 |
| FUM | [c]: (2) o2s + (3) h + nadh --> (2) h2o2 + nad |
| G3PD5 | [c]: glyc3p + o2 --> dhap + h2o2 |
| G3PD6 | [c]: glyc3p + o2 --> dhap + h2o2 |
| G3PD7 | [c]: glyc3p + o2 --> dhap + h2o2 |
| GLCDpp | glc-D[p] + h2o[p] + o2[c] --> glcn[p] + h[p] + h2o2[c] |
| GLUSy | [c]: nadph + h + o2 --> nadp + h2o2 |
| GLXCL | [c]: glx + o2 + h2o ---> for + co2 + h2o2 |
| GLYCL | [c]: gly + o2 + h + thf --> co2 + mlthf + h2o2 + nh4 |
| GLYCTO2 | [c]: glyclt + o2 --> glx + h2o2 |
| GLYCTO3 | [c]: glyclt + o2 --> glx + h2o2 |
| GLYCTO4 | [c]: glyclt + o2 --> glx + h2o2 |
| GTHOr | [c]: h + nadph + o2 <==> nadp + h2o2 |
| HYD1pp | (2) h[c] + h2[c] + o2[c] --> (2) h[p] + h2o2[c] |
| HYD2pp | (2) h[c] + h2[c] + o2[c] --> (2) h[p] + h2o2[c] |
| HYD3pp | (2) h[c] + h2[c] + o2[c] --> (2) h[p] + h2o2[c] |
| IPDPS | [c]: (2) o2s + (3) h + nadh --> (2) h2o2 + nad |
| LDH_D | [c]: lac-D + o2 --> h2o2 + pyr, |
| | [c]: nadh + h + o2 --> nad + h2o2 |
| LDH_D2 | [c]: lac-D + o2 --> pyr + h2o2 |
| L_LACD2 | [c]: lac-L + o2 --> pyr + h2o2 |
| L_LACD3 | [c]: lac-L + o2 --> pyr + h2o2 |
| MDH2 | [c]: mal-L + o2 --> oaa + h2o2 |
| MDH3 | [c]: mal-L + o2 --> oaa + h2o2 |
| MICITD | [c]: (2) o2s + (3) h + nadh --> (2) h2o2 + nad |
| MTHFR2 | [c]: nadh + h + o2 --> nad + h2o2 |
| NADH10 | [c]: nadh + h + o2 --> nad + h2o2 |
| NADH16pp | [c]: nadh + h + o2 --> nad + h2o2 |
| NADH17pp | [c]: nadh + h + o2 --> nad + h2o2 |
| NADH18pp | [c]: nadh + h + o2 --> nad + h2o2 |
| NADH5 | [c]: nadh + h + o2 --> nad + h2o2 |
| NADH9 | [c]: nadh + h + o2 --> nad + h2o2 |
| NADPHQR2 | [c]: nadph + h + o2 --> nadp + h2o2 |
| NADPHQR3 | [c]: nadph + h + o2 --> nadp + h2o2 |
| NADPHQR4 | [c]: nadph + h + o2 --> nadp + h2o2 |
| NADTRHD | [c]: nadph + h + o2 --> nadp + h2o2 |
| NHFRBO | [c]: nadh + h + o2 --> nad + h2o2 |
| NO3R1bpp | [c]: q8h2 + o2 --> h2o2 + q8 |
| NO3R1pp | [c]: q8h2 + o2 --> h2o2 + q8 |
| NO3R2bpp | [c]: mql8 + o2 --> h2o2 + mqn8 |
| NO3R2pp | [c]: mql8 + o2 --> h2o2 + mqn8 |
| NODOx | [c]: nadh + h + o2 --> nad + h2o2 |
| NODOy | [c]: nadph + h + o2 --> nadp + h2o2 |
| NTRIR2x | [c]: nadh + h + o2 --> nad + h2o2 |
| NTRIR3pp | [c]: q8h2 + o2 --> h2o2 + q8 |
| NTRIR4pp | [c]: mql8 + o2 --> h2o2 + mqn8 |
| OBTFL | [c]: (2) o2s + (3) h + nadh --> (2) h2o2 + nad |
| P5CD | NA |
| PDH | [c]: coa + o2 + h + pyr --> accoa + co2 + h2o2 |
| PFL | [c]: (2) o2s + (3) h + nadh --> (2) h2o2 + nad |
| POX | [c]: h2o + pyr + o2 --> ac + co2 + h2o2 |
| PPCDC | [c]: o2 + fmnh2 --> fmn + h2o2 |
| PPNCL2 | NA |
| PPPGO | [c]: (3) o2 + pppg9 --> (3) h2o2 + ppp9 |
| PPPGO3 | [c]: (3) o2 + pppg9 --> (3) h2o2 + ppp9 |
| PPPNDO | [c]: nadh + h + o2 --> nad + h2o2 |
| PROD2 | [c]: o2 + pro-L --> 1pyr5c + h2o2 + h |
| QULNS | [c]: (2) o2s + (3) h + nadh --> (2) h2o2 + nad |
| RNTR1c | [c]: fldrd + o2 --> fldox + h2o2 |
| RNTR2c | [c]: fldrd + o2 --> fldox + h2o2 |
| RNTR3c | [c]: fldrd + o2 --> fldox + h2o2 |
| RNTR4c | [c]: fldrd + o2 --> fldox + h2o2 |
| SERD_L | [c]: (2) o2s + (3) h + nadh --> (2) h2o2 + nad |
| SUCDi | [c]: o2 + succ --> fum + h2o2 |
| SULRi | [c]: (3) nadph + (3) h + (3) o2 --> (3) nadp + (3) h2o2 |
| TARTD | [c]: (2) o2s + (3) h + nadh --> (2) h2o2 + nad |
| THRD_L | [c]: (2) o2s + (3) h + nadh --> (2) h2o2 + nad |
| TMAOR1 | [c]: mql8 + o2 --> h2o2 + mqn8 |
| TMAOR1pp | [c]: mql8 + o2 --> h2o2 + mqn8 |
| TMAOR2 | [c]: 2dmmql8 + o2 --> 2dmmq8 + h2o2 |
| TMAOR2pp | [c]: 2dmmql8 + o2 --> 2dmmq8 + h2o2 |
| TRDR | [c]: nadph + h + o2 --> nadp + h2o2 |
| UAPGR | [c]: nadph + h + o2 --> nadp + h2o2 |
| UDPGALM | [c]: o2 + fadh2 --> fad + h2o2 |
| | Putative o2s Rxn |
| ACHBS | [c]: pyr + (2) o2 + h2o ---> ac + co2 + (2) o2s + (2) h |
| ACLS | [c]: pyr + (2) o2 + h2o ---> ac + co2 + (2) o2s + (2) h |
| ACOAD1f | [c]: (2) o2 + fadh2 --> fad + (2) o2s + (2) h |
| ACOAD2f | [c]: (2) o2 + fadh2 --> fad + (2) o2s + (2) h |
| ACOAD3f | [c]: (2) o2 + fadh2 --> fad + (2) o2s + (2) h |
| ACOAD4f | [c]: (2) o2 + fadh2 --> fad + (2) o2s + (2) h |
| ACOAD5f | [c]: (2) o2 + fadh2 --> fad + (2) o2s + (2) h |
| ACOAD6f | [c]: (2) o2 + fadh2 --> fad + (2) o2s + (2) h |
| ACOAD7f | [c]: (2) o2 + fadh2 --> fad + (2) o2s + (2) h |
| ACOAD8f | [c]: (2) o2 + fadh2 --> fad + (2) o2s + (2) h |
| ACONTa | [c]: (2) o2 + nadh --> (2) o2s + nad + h |
| ACONTb | NA |
| AKGDH | [c]: akg + coa + (2) o2 --> co2 + (2) o2s + succoa + h |
| AMMQLT8 | [c]: mql8 + (2) o2 --> (2) o2s + mqn8 + (2) h |
| ARBTNR1 | [c]: (2) o2 + fadh2 --> fad + (2) o2s + (2) h |
| ARBTNR2 | [c]: (2) o2 + fmnh2 --> fmn + (2) o2s + (2) h |
| ASPO3 | [c]: asp-L + (2) o2 --> (3) h + (2) o2s + iasp |
| ASPO4 | [c]: asp-L + (2) o2 --> (3) h + (2) o2s + iasp |
| ASPO5 | [c]: asp-L + (2) o2 --> (3) h + (2) o2s + iasp |
| BTS4 | [c]: (2) o2 + nadh --> (2) o2s + nad + h |
| CPGNR1 | [c]: (2) o2 + fadh2 --> fad + (2) o2s + (2) h |
| CPGNR2 | [c]: (2) o2 + fmnh2 --> fmn + (2) o2s + (2) h |
| CPPPGO2 | [c]: (2) o2 + nadh --> (2) o2s + nad + h |
| CYTBD2pp | [c]: mql8 + (2) o2 --> (2) o2s + mqn8 + (2) h |
| CYTBDpp | [c]: q8h2 + (2) o2 --> (2) o2s + q8 + (2) h |
| CYTBO3_4pp | [c]: q8h2 + (2) o2 --> (2) o2s + q8 + (2) h |
| DAAD | [c]: ala-D + (2) o2 + h2o --> (2) o2s + nh4 + pyr + (2) h |
| DHAD1 | [c]: (2) o2 + nadh --> (2) o2s + nad + h |
| DHAD2 | [c]: (2) o2 + nadh --> (2) o2s + nad + h |
| DHNAOT4 | [c]: 2dmmql8 + (2) o2 --> 2dmmq8 + (2)o2s + (2)h |
| DHORD2 | [c]: dhor-S + (2) o2 --> orot + (2) o2s + (2) h |
| DHORD5 | [c]: dhor-S + (2) o2 --> orot + (2) o2s + (2) h |
| DMPPS | [c]: (2) o2 + nadh --> (2) o2s + nad + h |
| DMQMT | [c]: q8h2 + (2) o2 --> (2) o2s + q8 + (2) h |
| DMSOR1 | [c]: mql8 + (2) o2 --> (2) o2s + mqn8 + (2) h |
| DMSOR1pp | [c]: mql8 + (2) o2 --> (2) o2s + mqn8 + (2) h |
| DMSOR2 | [c]: 2dmmql8 + (2) o2 --> 2dmmq8 + (2)o2s + (2)h |
| DMSOR2pp | [c]: 2dmmql8 + (2) o2 --> 2dmmq8 + (2)o2s + (2)h |
| DSBAO1 | dsbard[p] + (2) o2[c] --> dsbaox[p] + (2) o2s[c] + (2) h [c] |
| DSBAO2 | dsbard[p] + (2) o2[c] --> dsbaox[p] + (2) o2s[c] + (2) h [c] |
| FADRx | [c]: (2) o2 + nadh --> (2) o2s + nad + h |
| FADRx2 | [c]: (2) o2 + nadph --> (2) o2s + nadp + h |
| FDH4pp | for[p] + (2) o2[c] --> co2[c] + h[p] + (2) o2s[c] |
| FDH5pp | for[p] + (2) o2[c] --> co2[c] + h[p] + (2) o2s[c] |
| FDMO | [c]: (2) o2 + fmnh2 --> fmn + (2) o2s + (2) h |
| FDMO2 | [c]: (2) o2 + fmnh2 --> fmn + (2) o2s + (2) h |
| FDMO3 | [c]: (2) o2 + fmnh2 --> fmn + (2) o2s + (2) h |
| FDMO4 | [c]: (2) o2 + fmnh2 --> fmn + (2) o2s + (2) h |
| FDMO6 | [c]: (2) o2 + fmnh2 --> fmn + (2) o2s + (2) h |
| FE3HOXR1 | [c]: (2) o2 + fadh2 --> fad + (2) o2s + (2) h |
| FE3HOXR2 | [c]: (2) o2 + fmnh2 --> fmn + (2) o2s + (2) h |
| FE3Ri | [c]: (2) o2 + fadh2 --> fad + (2) o2s + (2) h |
| FECRMR1 | [c]: (2) o2 + fadh2 --> fad + (2) o2s + (2) h |
| FECRMR2 | [c]: (2) o2 + fmnh2 --> fmn + (2) o2s + (2) h |
| FEENTERR1 | [c]: (2) o2 + fadh2 --> fad + (2) o2s + (2) h |
| FEENTERR2 | [c]: (2) o2 + fmnh2 --> fmn + (2) o2s + (2) h |
| FEOXAMR1 | [c]: (2) o2 + fadh2 --> fad + (2) o2s + (2) h |
| FEOXAMR2 | [c]: (2) o2 + fmnh2 --> fmn + (2) o2s + (2) h |
| FLDR | [c]: (2) o2 + nadph --> (2) o2s + nadp + h |
| FLVR | [c]: (2) o2 + nadph --> (2) o2s + nadp + h |
| FMNRx | [c]: (2) o2 + nadh --> (2) o2s + nad + h |
| FMNRx2 | [c]: (2) o2 + nadph --> (2) o2s + nadp + h |
| FRD2 | [c]: mql8 + (2) o2 --> (2) o2s + mqn8 + (2) h |
| FRD3 | [c]: 2dmmql8 + (2) o2 --> 2dmmq8 + (2)o2s + (2)h |
| FUM | [c]: (2) o2 + nadh --> (2) o2s + nad + h |
| G3PD5 | [c]: glyc3p + (2) o2 --> dhap + (2) o2s + (2) h |
| G3PD6 | [c]: glyc3p + (2) o2 --> dhap + (2) o2s + (2) h |
| G3PD7 | [c]: glyc3p + (2) o2 --> dhap + (2) o2s + (2) h |
| GLCDpp | glc-D[p] + h2o[p] + (2) o2[c] --> glcn[p] + h[p] + (2) o2s[c] + (2) h[c] |
| GLUSy | [c]: nadph + (2)o2 --> (2)o2s + nadp + h |
| GLXCL | [c]: glx + (2) o2 + h2o ---> for + co2 + (2) o2s + (2) h |
| GLYCL | [c]: gly + (2) o2 + thf --> co2 + mlthf + (2) o2s + nh4 + h |
| GLYCTO2 | [c]: glyclt + (2) o2 --> glx + (2) o2s + (2) h |
| GLYCTO3 | [c]: glyclt + (2) o2 --> glx + (2) o2s + (2) h |
| GLYCTO4 | [c]: glyclt + (2) o2 --> glx + (2) o2s + (2) h |
| GTHOr | [c]: nadph + (2)o2 --> (2)o2s + nadp + h |
| HYD1pp | h2[c] + (2) o2[c] --> (2) h[p] + (2) o2s[c] |
| HYD2pp | h2[c] + (2) o2[c] --> (2) h[p] + (2) o2s[c] |
| HYD3pp | h2[c] + (2) o2[c] --> (2) h[p] + (2) o2s[c] |
| IPDPS | [c]: (2) o2 + nadh --> (2) o2s + nad + h |
| LDH_D | [c]: lac-D + (2) o2 --> (2) o2s + pyr + (2) h, |
| | [c]: nadh + (2) o2 --> nad + (2) o2s + h |
| LDH_D2 | [c]: lac-D + (2) o2 --> pyr + (2) o2s + (2) h |
| L_LACD2 | [c]: lac-L + (2) o2 --> pyr + (2) o2s + (2) h |
| L_LACD3 | [c]: lac-L + (2) o2 --> pyr + (2) o2s + (2) h |
| MDH2 | [c]: mal-L + (2) o2 --> oaa + (2) o2s + (2) h |
| MDH3 | [c]: mal-L + (2) o2 --> oaa + (2) o2s + (2) h |
| MICITD | [c]: (2) o2 + nadh --> (2) o2s + nad + h |
| MTHFR2 | [c]: nadh + (2)o2 --> (2)o2s + nad + h |
| NADH10 | [c]: nadh + (2)o2 --> (2)o2s + nad + h |
| NADH16pp | [c]: nadh + (2)o2 --> (2)o2s + nad + h |
| NADH17pp | [c]: nadh + (2)o2 --> (2)o2s + nad + h |
| NADH18pp | [c]: nadh + (2)o2 --> (2)o2s + nad + h |
| NADH5 | [c]: nadh + (2)o2 --> (2)o2s + nad + h |
| NADH9 | [c]: nadh + (2)o2 --> (2)o2s + nad + h |
| NADPHQR2 | [c]: nadph + (2)o2 --> (2)o2s + nadp + h |
| NADPHQR3 | [c]: nadph + (2)o2 --> (2)o2s + nadp + h |
| NADPHQR4 | [c]: nadph + (2)o2 --> (2)o2s + nadp + h |
| NADTRHD | [c]: nadph + (2)o2 --> (2)o2s + nadp + h |
| NHFRBO | [c]: nadh + (2)o2 --> (2)o2s + nad + h |
| NO3R1bpp | [c]: q8h2 + (2) o2 --> (2) o2s + q8 + (2) h |
| NO3R1pp | [c]: q8h2 + (2) o2 --> (2) o2s + q8 + (2) h |
| NO3R2bpp | [c]: mql8 + (2) o2 --> (2) o2s + mqn8 + (2) h |
| NO3R2pp | [c]: mql8 + (2) o2 --> (2) o2s + mqn8 + (2) h |
| NODOx | [c]: nadh + (2)o2 --> (2)o2s + nad + h |
| NODOy | [c]: nadph + (2)o2 --> (2)o2s + nadp + h |
| NTRIR2x | [c]: nadh + (2) o2 --> nad + (2) o2s + h |
| NTRIR3pp | [c]: q8h2 + (2) o2 --> (2) o2s + q8 + (2) h |
| NTRIR4pp | [c]: mql8 + (2) o2 --> (2) o2s + mqn8 + (2) h |
| OBTFL | [c]: (2) o2 + nadh --> (2) o2s + nad + h |
| P5CD | NA |
| PDH | [c]: coa + (2) o2 + pyr --> accoa + co2 + (2)o2s + h |
| PFL | [c]: (2) o2 + nadh --> (2) o2s + nad + h |
| POX | [c]: h2o + pyr + (2) o2 --> ac + co2 + (2) o2s + (2) h |
| PPCDC | [c]: (2) o2 + fmnh2 --> fmn + (2) o2s + (2) h |
| PPNCL2 | NA |
| PPPGO | [c]: (6) o2 + pppg9 --> (6) o2s + ppp9 + (6) h |
| PPPGO3 | [c]: (6) o2 + pppg9 --> (6) o2s + ppp9 + (6) h |
| PPPNDO | [c]: nadh + (2)o2 --> (2)o2s + nad + h |
| PROD2 | [c]: (2) o2 + pro-L --> 1pyr5c + (2) o2s + (3) h |
| QULNS | [c]: (2) o2 + nadh --> (2) o2s + nad + h |
| RNTR1c | [c]: fldrd + (2) o2 --> fldox + (2) o2s + (2) h |
| RNTR2c | [c]: fldrd + (2) o2 --> fldox + (2) o2s + (2) h |
| RNTR3c | [c]: fldrd + (2) o2 --> fldox + (2) o2s + (2) h |
| RNTR4c | [c]: fldrd + (2) o2 --> fldox + (2) o2s + (2) h |
| SERD_L | [c]: (2) o2 + nadh --> (2) o2s + nad + h |
| SUCDi | [c]: (2) o2 + succ --> fum + (2) o2s + (2) h |
| SULRi | [c]: (3) nadph + (6) o2 --> (3) nadp + (6) o2s + (3) h |
| TARTD | [c]: (2) o2 + nadh --> (2) o2s + nad + h |
| THRD_L | [c]: (2) o2 + nadh --> (2) o2s + nad + h |
| TMAOR1 | [c]: mql8 + (2) o2 --> (2) o2s + mqn8 + (2) h |
| TMAOR1pp | [c]: mql8 + (2) o2 --> (2) o2s + mqn8 + (2) h |
| TMAOR2 | [c]: 2dmmql8 + (2) o2 --> 2dmmq8 + (2)o2s + (2)h |
| TMAOR2pp | [c]: 2dmmql8 + (2) o2 --> 2dmmq8 + (2)o2s + (2)h |
| TRDR | [c]: nadph + (2)o2 --> (2)o2s + nadp + h |
| UAPGR | [c]: nadph + (2)o2 --> (2)o2s + nadp + h |
| UDPGALM | [c]: (2) o2 + fadh2 --> fad + (2) o2s + (2) h |
| | Combined rxn (c −> h2o2 rxn, k −> o2s rxn) |
| ACHBS | [c]: 2obut + h + (1 + c + k) pyr + (c + 2k) o2 + (c + k) h2o--> 2ahbut + (1 + c + k) co2 + (c + k) ac + |
| | (c) h2o2 + (2k) o2s + (2k) h |
| ACLS | [c]: h + (2 + c + k) pyr + (c + 2k) o2 + (c + k) h2o --> alac-S + (1 + c + k) co2 + (c + k) ac + |
| | (c) h2o2 + (2k) o2s + (2k) h |
| ACOAD1f | [c]: btcoa + (1 − c − k) fad + (c + 2k) o2 --> b2coa + (1 − c − k) fadh2 + |
| | (c) h2o2 + (2k) o2s + (2k) h, OR |
| | [c]: b2coa + (1 + c + k) fadh2 + (c + 2k) o2 --> btcoa + (1 + c + k) fad + (c) h2o2 + (2k) o2s + (2k) h |
| ACOAD2f | [c]: hxcoa + (1 − c − k) fad + (c + 2k) o2 --> hx2coa + (1 − c − k) fadh2 + |
| | (c) h2o2 + (2k) o2s + (2k) h, OR |
| | [c]: hx2coa + (1 + c + k) fadh2 + (c + 2k) o2 --> hxcoa + (1 + c + k) fad + (c) h2o2 + (2k) o2s + (2k) h |
| ACOAD3f | [c]: occoa + (1 − c − k) fad + (c + 2k) o2 --> oc2coa + (1 − c − k) fadh2 + |
| | (c) h2o2 + (2k) o2s + (2k) h, OR |
| | [c]: oc2coa + (1 + c + k) fadh2 + (c + 2k) o2 --> occoa + (1 + c + k) fad + (c) h2o2 + (2k) o2s + (2k) h |
| ACOAD4f | [c]: dcacoa + (1 − c − k) fad + (c + 2k)o2 --> dc2coa + (1 − c − k) fadh2 + |
| | (c) h2o2 + (2k) o2s + (2k) h, OR |
| | [c]: dc2coa + (1 + c + k) fadh2 + (c + 2k) o2 --> dcacoa + (1 + c + k) fad + (c) h2o2 + (2k) o2s + (2k) h |
| ACOAD5f | [c]: ddcacoa + (1 − c − k) fad + (c + 2k) o2 --> dd2coa + (1 − c − k) fadh2 + |
| | (c) h2o2 + (2k) o2s + (2k) h, OR |
| | [c]: dd2coa + (1 + c + k) fadh2 + (c + 2k) o2 --> ddcacoa + (1 + c + k) fad + (c) h2o2 + (2k) o2s + (2k) h |
| ACOAD6f | [c]: tdcoa + (1 − c − k) fad + (c + 2k) o2 --> td2coa + (1 − c − k) fadh2 + |
| | (c) h2o2 + (2k) o2s + (2k) h, OR |
| | [c]: td2coa + (1 + c + k) fadh2 + (c + 2k) o2 --> tdcoa + (1 + c + k) fad + (c) h2o2 + (2k) o2s + (2k) h |
| ACOAD7f | [c]: pmtcoa + (1 − c − k) fad + (c + 2k) o2 --> hdd2coa + (1 − c − k) fadh2 + |
| | (c) h2o2 + (2k) o2s + (2k) h, OR |
| | [c]: hdd2coa + (1 + c + k) fadh2 + (c + 2k) o2 --> pmtcoa + (1 + c + k) fad + (c) h2o2 + (2k) o2s + (2k) h |
| ACOAD8f | [c]: stcoa + (1 − c − k) fad + (c + 2k) o2 --> od2coa + (1 − c − k) fadh2 + |
| | (c) h2o2 + (2k) o2s + (2k) h, OR |
| | [c]: od2coa + (1 + c + k) fadh2 + (c + 2k) o2 --> stcoa + (1 + c + k) fad + (c) h2o2 + (2k) o2s + (2k) h |
| ACONTa | [c]: cit + (2k) o2 + (c + k) nadh + (3c) h + (2c) o2s --> acon-C + h2o + (2c) h2o2 + (2k) o2s + (c + k) nad + (k) h, OR |
| | [c]: acon-C + h2o + (2k) o2 + (c + k) nadh + (2c) o2s + (3c) h --> cit + (2c) h2o2 + (2k) o2s + (c + k) nad + (k) h |
| ACONTb | NA |
| AKGDH | [c]: akg + coa + (1 − c − k) nad + (c + 2k) o2 + (c) h --> co2 + (1 − c − k) nadh + succoa + |
| | (c) h2o2 + (2k) o2s + (k) h |
| AMMQLT8 | [c]: 2dmmql8 + amet + (c + 2k) o2 --> ahcys + (1 + 2k) h + (1 − c − k) mql8 + |
| | (c) h2o2 + (2k) o2s + (c + k) mqn8 |
| ARBTNR1 | [c]: (2) arbtn-fe3 + (1 + c + k) fadh2 + (c + 2k) o2--> (2) arbtn + (1 + c + k) fad + (2) fe2 + (2 + 2k) h + |
| | (c) h2o2 + (2k) o2s |
| ARBTNR2 | [c]: (2) arbtn-fe3 + (1 + c + k) fmnh2 + (c + 2k) o2--> (2) arbtn + (1 + c + k) fmn + (2) fe2 + (2 + 2k) h + |
| | (c) h2o2 + (2k) o2s |
| ASPO3 | [c]: (1 + c + k) asp-L + q8 + (c + 2k) o2 --> (1 + c + 3k) h + (1 + c + k) iasp + q8h2 + |
| | (c) h2o2 + (2k) o2s |
| ASPO4 | [c]: (1 + c + k) asp-L + mqn8 + (c + 2k) o2 --> (1 + c + 3k) h + (1 + c + k) iasp + mql8 + |
| | (c) h2o2 + (2k) o2s |
| ASPO5 | [c]: (1 + c + k) asp-L + fum + (c + 2k) o2 --> (1 + c + 3k) h + (1 + c + k) iasp + succ + |
| | (c) h2o2 + (2k) o2s |
| BTS4 | [c]: amet + dtbt + s + (2k) o2 + (c + k) nadh + (2c) o2s + (3c) h --> btn + dad-5 + h + met-L + (2c) |
| | h2o2 + (2k) o2s + (c + k) nad + (k) h |
| CPGNR1 | [c]: (2) cpgn + (1 + c + k) fadh2 + (c + 2k) o2--> (2) cpgn-un + (1 + c + k) fad + (2) fe2 + (2 + 2k) h + |
| | (c) h2o2 + (2k) o2s |
| CPGNR2 | [c]: (2) cpgn + (1 + c + k) fmnh2 + (c + 2k) o2--> (2) cpgn-un + (1 + c + k) fmn + (2) fe2 + (2 + 2k) h + |
| | (c) h2o2 + (2k) o2s |
| CPPPGO2 | [c]: (2) amet + cpppg3 + (2k) o2 + (c + k) nadh + (2c) o2s + (3c) h --> (2) co2 + (2) dad-5 + (2) |
| | met-L + pppg9 + (2c) h2o2 + (2k) o2s + (c + k) nad + (k) h |
| CYTBD2pp | (2) h[c] + (1 + c + k) mql8[c] + (0.5 + c + 2k) o2[c] --> (2) h[p] + h2o[c] + (1 + c + k) mqn8[c] + |
| | (c) h2o2[c] + (2k) o2s[c] + (2k) h[c] |
| CYTBDpp | (2) h[c] + (0.5 + c + 2k) o2[c] + (1 + c + k) q8h2[c] --> (2) h[p] + h2o[c] + (1 + c + k) q8[c] + |
| | (c) h2o2[c] + (2k) o2s[c] + (2k) h[c] |
| CYTBO3_4pp | (4) h[c] + (0.5 + c + 2k) o2[c] + (1 + c + k) q8h2[c] --> (4) h[p] + h2o[c] + (1 + c + k) q8[c] + |
| | (c) h2o2[c] + (2k) o2s[c] + (2k) h[c] |
| DAAD | [c]: ala-D + (1 − c − k) fad + h2o + (c + 2k) o2 --> nh4 + pyr + (1 − c − k) fadh2 + |
| | (c) h2o2 + (2k) o2s + (2k) h |
| DHAD1 | [c]: 23dhmb + (2k) o2 + (k) nadh + (2c) o2s + (3c) h --> 3mob + h2o + (2c) h2o2 + (2k) o2s + |
| | (c + k) nad + (k) h |
| DHAD2 | [c]: 23dhmp + (2k) o2 + (k) nadh + (2c) h2o2 + (3c) h--> 3mop + h2o + (2c) h2o2 + (2k) o2s + |
| | (c + k) nad + (k) h |
| DHNAOT4 | [c]: dhna + h + octdp + (c + 2k) o2--> (1 − c − k) 2dmmql8 + co2 + ppi + (c + k) 2dmmq8 + |
| | (c) h2o2 + (2k) o2s + (2k) h |
| DHORD2 | [c]: dhor-S + (1 − c − k) q8 + (c + 2k) o2 --> orot + (1 − c − k) q8h2 + |
| | (c) h2o2 + (2k) o2s + (2k) h |
| DHORD5 | [c]: dhor-S + (1 − c − k) mqn8 + (c + 2k) o2 --> (1 − c − k) mql8 + orot + |
| | (c) h2o2 + (2k) o2s + (2k) h |
| DMPPS | [c]: (1 + 3c) h + h2mb4p + (1 + c + k) nadh + (2k) o2 + (2c) o2s--> dmpp + h2o + (1 + c + k) nad + |
| | (2c) h2o2 + (2k) o2s + (k) h |
| DMQMT | [c]: 2omhmbl + amet (c + 2k) o2 --> ahcys + (1 + 2k) h + (1 − c − k) q8h2 + (c + k) q8 + |
| | (c) h2o2 + (2k) o2s |
| DMSOR1 | [c]: dmso + (1 + c + k) mql8 + (c + 2k) o2 --> dms + h2o + (1 + c + k) mqn8 + |
| | (c) h2o2 + (2k) o2s + (2k) h |
| DMSOR1pp | dmso[p] + (1 + c + k) mql8[c] + (c + 2k) o2 [c] --> dms[p] + h2o[p] + (1 + c + k) mqn8[c] + |
| | (c) h2o2[c] + (2k) o2s[c] + (2k) h[c] |
| DMSOR2 | [c]: (1 + c + k) 2dmmql8 + dmso + (c + 2k) o2 --> (1 + c + k) 2dmmq8 + dms + h2o + |
| | (c) h2o2 + (2k) o2s + (2k) h |
| DMSOR2pp | (1 + c + k) 2dmmql8[c] + dmso[p] + (c + 2k) o2 [c] --> (1 + c + k) 2dmmq8[c] + dms[p] + h2o[p] + |
| | (c) h2o2[c] + (2k) o2s[c] + (2k) h[c] |
| DSBAO1 | dsbard[p] + (1 − c − k) q8[c] + (c + 2k) o2[c] --> dsbaox[p] + (1 − c − k) q8h2[c] + |
| | (c) h2o2[c] + (2k) o2s[c] + (2k) h[c] |
| DSBAO2 | dsbard[p] + (1 − c − k) mqn8[c] + (c + 2k) o2[c] --> dsbaox[p] + (1 − c − k) mql8[c] + |
| | (c) h2o2[c] + (2k) o2s[c] + (2k) h[c] |
| FADRx | [c]: (1 − c − k) fad + h + nadh + (c + 2k) o2 --> (1 − c − k) fadh2 + nad + |
| | (c) h2o2 + (2k) o2s + (2k) h |
| FADRx2 | [c]: (1 − c − k) fad + h + nadph --> (1 − c − k) fadh2 + nadp + |
| | (c) h2o2 + (2k) o2s + (2k) h |
| FDH4pp | for[p] + (2) h[c] + (1 − c − k)q8[c] + (c + 2k) o2 [c] --> co2[c] + h[p] + (1 − c − k) q8h2[c] + |
| | (c) h2o2[c] + (2k) o2s[c] + (2k) h[c] |
| FDH5pp | for[p] + (2) h[c] + (1 − c − k) mqn8[c] + (c + 2k) o2[c] --> co2[c] + h[p] + (1 − c − k) mql8[c] + |
| | (c) h2o2[c] + (2k) o2s[c] + (2k) h[c] |
| FDMO | [c]: (1 + c + k) fmnh2 + isetac + (1 + c + 2k) o2 --> (1 + c + k) fmn + gcald + (1 + 2k) h + h2o + so3 + |
| | (c) h2o2 + (2k) o2s |
| FDMO2 | [c]: (1 + c + k) fmnh2 + mso3 + (1 + c + 2k) o2 --> fald + (1 + c + k) fmn + (1 + 2k) h + h2o + so3 + |
| | (c) h2o2 + (2k) o2s |
| FDMO3 | [c]: ethso3 + (1 + c + k) fmnh2 + (1 + c + 2k) o2 --> acald + (1 + c + k) fmn + (1 + 2k) h + h2o + so3 + |
| | (c) h2o2 + (2k) o2s |
| FDMO4 | [c]: butso3 + (1 + c + k) fmnh2 + (1 + c + 2k) o2 --> btal + (1 + c + k) fmn + (1 + 2k) h + h2o + so3 + |
| | (c) h2o2 + (2k) o2s |
| FDMO6 | [c]: (1 + c + k) fmnh2 + (1 + c + 2k) o2 + sulfac --> (1 + c + k) fmn + glx + (1 + 2k) h + h2o + so3 + |
| | (c) h2o2 + (2k) o2s |
| FE3HOXR1 | [c]: (1 + c + k) fadh2 + (2) fe3hox + (c + 2k) o2 --> (1 + c + k) fad + (2) fe2 + (2) fe3hox-un + (2 + 2k) h + |
| | (c) h2o2 + (2k) o2s |
| FE3HOXR2 | [c]: (2) fe3hox + (1 + c + k) fmnh2 + (c + 2k) o2--> (2) fe2 + (2) fe3hox-un + (1 + c + k) fmn + (2 + 2k) h + |
| | (c) h2o2 + (2k) o2s |
| FE3Ri | [c]: (1 + c + k) fadh2 + (2) fe3 + (c + 2k) o2 --> (1 + c + k) fad + (2) fe2 + (2 + 2k) h + |
| | (c) h2o2 + (2k) o2s |
| FECRMR1 | [c]: (1 + c + k) fadh2 + (2) fecrm + (c + 2k) o2 --> (1 + c + k) fad + (2) fe2 + (2) fecrm-un + (2 + 2k) h + |
| | (c) h2o2 + (2k) o2s |
| FECRMR2 | [c]: (2) fecrm + (1 + c + k) fmnh2 + (c + 2k) o2 --> (2) fe2 + (2) fecrm-un + (1 + c + k) fmn + (2 + 2k) h + |
| | (c) h2o2 + (2k) o2s |
| FEENTERR1 | [c]: (1 + c + k) fadh2 + (2) feenter + (c + 2k) o2 --> (2) enter + (1 + c + k) fad + (2) fe2 + (2 + 2k) h + |
| | (c) h2o2 + (2k) o2s |
| FEENTERR2 | [c]: (2) feenter + (1 + c + k) fmnh2 + (c + 2k) o2 --> (2) enter + (2) fe2 + (1 + c + k) fmn + (2 + 2k) h + |
| | (c) h2o2 + (2k) o2s |
| FEOXAMR1 | [c]: (1 + c + k) fadh2 + (2) feoxam + (c + 2k) o2 --> (1 + c + k) fad + (2) fe2 + (2) feoxam-un + (2 + 2k) h + |
| | (c) h2o2 + (2k) o2s |
| FEOXAMR2 | [c]: (2) feoxam + (1 + c + k) fmnh2 + (c + 2k) o2 --> (2) fe2 + (2) feoxam-un + (1 + c + k) fmn + (2 + 2k) h + |
| | (c) h2o2 + (2k) o2s |
| FLDR | [c]: (1 − c − k) fldox + h + nadph (c + 2k) o2 --> (1 − c − k) fldrd + nadp + |
| | (c) h2o2 + (2k)o2s + (2k) h |
| FLVR | [c]: h + nadph + (1 − c − k) ribflv + (c + 2k) o2 --> nadp + (1 − c − k) rbflvrd + |
| | (c) h2o2 + (2k) o2s + (2k) h |
| FMNRx | [c]: (1 − c − k) fmn + h + nadh + (c + 2k) o2--> (1 − c − k) fmnh2 + nad + |
| | (c) h2o2 + (2k) o2s + (2k) h |
| FMNRx2 | [c]: (1 − c − k) fmn + h + nadph + (c + 2k) o2 --> (1 − c − k) fmnh2 + nadp + |
| | (c) h2o2 + (2k) o2s + (2k) h |
| FRD2 | [c]: fum + (1 + c + k) mql8 + (c + 2k) o2 --> (1 + c + k) mqn8 + succ +(c) h2o2 + (2k) o2s + (2k) h |
| FRD3 | [c]: (1 + c + k) 2dmmql8 + fum + (c + 2k) o2 --> (1 + c + k) 2dmmq8 + succ + |
| | (c) h2o2 + (2k) o2s + (2k) h |
| FUM | [c]: fum + h2o + (2k) o2 + (c + k) nadh (2c) o2s + (3c) h --> mal-L + (2c) h2o2 + (2k) o2s + |
| | (c + k) nad + (k) h, OR |
| | [c]: mal-L + (2k) o2 + (c + k) nadh + (2c) h2o2 + (3c) h --> fum + h2o + |
| | (2c) h2o2 + (2k) o2s + (c + k) nad + (k) h |
| G3PD5 | [c]: glyc3p + (1 − c − k) q8 + (c + 2k) o2 --> dhap + (1 − c − k) q8h2 + |
| | (c) h2o2 + (2k) o2s + (2k) h |
| G3PD6 | [c]: glyc3p + (1 − c − k) mqn8 + (c + 2k) o2 --> dhap + (1 − c − k) mql8 + |
| | (c) h2o2 + (2k) o2s + (2k) h |
| G3PD7 | [c]: glyc3p + (1 − c − k) 2dmmq8 + (c + 2k) o2 --> dhap + (1 − c − k) 2dmmql8 + |
| | (c) h2o2 + (2k) o2s + (2k) h |
| GLCDpp | glc-D[p] + h2o[p] + (1 − c − k) q8[c] + (c + 2k) o2 [c] --> glcn[p] + h[p] + (1 − c − k) q8h2[c] + |
| | (c) h2o2[c] + (2k) o2s[c] + (2k) h [c] |
| GLUSy | [c]: akg + gln-L + (1 + c + k) h + (1 + c + k) nadph + (c + 2k) o2 --> (2) glu-L + (1 + c + k) nadp + |
| | (c) h2o2 + (2k) o2s + (2k) h |
| GLXCL | [c]: (2 + c + k) glx + h + (c + 2k) o2 + (c + k) h2o --> 2h3oppan + (1 + c + k) co2 + |
| | (c) h2o2 + (2k) o2s + (2k) h + (c + k) for |
| GLYCL | [c]: (1 + c + k) gly + nad + (1 + c + k) thf + (c + 2k) o2 + |
| | (c) h --> (1 + c + k) co2 + (1 + c + k) mlthf + nadh + (1 + c + k) nh4 + |
| | (c) h2o2 + (2k) o2s + (k) h |
| GLYCTO2 | [c]: glyclt + (1 − c − k) q8 + (c + 2k) o2--> glx + (1 − c − k) q8h2 + |
| | (c) h2o2 + (2k) o2s + (2k) h |
| GLYCTO3 | [c]: glyclt + (1 − c − k) mqn8 + (c + 2k) o2--> glx + (1 − c − k) mql8 + |
| | (c) h2o2 + (2k) o2s + (2k) h |
| GLYCTO4 | [c]: glyclt + (1 − c − k) 2dmmq8 + (c + 2k) o2--> glx + (1 − c − k) 2dmmql8 + |
| | (c) h2o2 + (2k) o2s + (2k) h |
| GTHOr | [c]: gthox + (1 + c) h + (1 + c + k) nadph + (c + 2k) o2 <==> (2) gthrd + (1 + c + k) nadp + |
| | (c) h2o2 + (2k) o2s + (k) h |
| HYD1pp | (2) h[c] + h2[c] + (1 − c − k) q8[c] + (c + 2k) o2[c] --> (2) h[p] + (1 − k − c) q8h2[c] + |
| | (c) h2o2[c] + (2k) o2s[c] + (2k) h[c] |
| HYD2pp | (2) h[c] + h2[c] + (1 − c − k) mqn8[c] + (c + 2k) o2[c] --> (2) h[p] + (1 − k − c) mql8[c] + |
| | (c) h2o2[c] + (2k) o2s[c] + (2k) h[c] |
| HYD3pp | (2) h[c] + h2[c] + (1 − c − k) 2dmmq8[c] + (c + 2k) o2[c] --> (2) h[p] + (1 − k − c) 2dmmql8[c] + |
| | (c) h2o2[c] + (2k) o2s[c] + (2k) h[c] |
| IPDPS | [c]: (1 + 3c) h + h2mb4p + (1 + c + k) nadh + (2k) o2 + (2c) o2s --> h2o + ipdp + (1 + c + k) nad + |
| | (2c) h2o2 + (2k) o2s + (k) h |
| LDH_D | [c]: (1 + c + k) lac-D + nad + (c + 2k) o2 --> (1 + 2k) h + nadh + (1 + c + k) pyr + |
| | (c) h2o2 + (2k) o2s, OR |
| | [c]: (1 + c) h + (1 + c + k) nadh + pyr + (c + 2k) o2 --> lac-D + (1 + c + k) nad + |
| | (c) h2o2 + (2k)o2s + (k)h |
| LDH_D2 | [c]: lac-D + (1 − c − k) q8 + (c + 2k) o2 --> pyr + (1 − c − k) q8h2 + |
| | (c) h2o2 + (2k) o2s + (2k) h |
| L_LACD2 | [c]: lac-L + (1 − c − k) q8 + (c + 2k) o2 --> pyr + (1 − c − k) q8h2 + |
| | (c) h2o2 + (2k) o2s + (2k) h |
| L_LACD3 | [c]: lac-L + (1 − c − k) mqn8 + (c + 2k) o2 --> pyr + (1 − c − k) mql8 + |
| | (c) h2o2 + (2k) o2s + (2k) h |
| MDH2 | [c]: mal-L + (1 − c − k) q8 + (c + 2k) o2 --> oaa + (1 − c − k) q8h2 + |
| | (c) h2o2 + (2k) o2s + (2k) h |
| MDH3 | [c]: mal-L + (1 − c − k) mqn8 + (c + 2k) o2 --> oaa + (1 − c − k) mql8 + |
| | (c) h2o2 + (2k) o2s + (2k) h |
| MICITD | [c]: 2mcacn + h2o + (2k) o2 + (c + k) nadh + (2c) o2s + (3c) h --> micit + |
| | (2c) h2o2 + (2k) o2s + (c + k) nad + (k) h |
| MTHFR2 | [c]: (2 + c) h + mlthf + (1 + c + k) nadh + (c + 2k) o2 --> 5mthf + (1 + c + k) nad + |
| | (c) h2o2 + (2k) o2s + (k)h |
| NADH10 | [c]: (1 + c) h + mqn8 + (1 + c + k) nadh + (c + 2k) o2 --> mql8 + (1 + c + k) nad + |
| | (c) h2o2 + (2k) o2s + (k)h |
| NADH16pp | (4 + c) h[c] + (1 + c + k) nadh[c] + q8[c] + (c + 2k) o2[c] --> (3) h[p] + (1 + c + k) nad[c] + q8h2[c] + |
| | (c) h2o2[c] + (2k) o2s[c] + (k) h[c] |
| NADH17pp | (4 + c) h[c] + mqn8[c] + (1 + c + k) nadh[c] + (c + 2k) o2[c] --> (3) h[p] + mql8[c] + (1 + c + k) nad[c] + |
| | (c) h2o2[c] + (2k) o2s[c] + (k) h[c] |
| NADH18pp | 2dmmq8[c] + (4 + c) h[c] + (1 + c + k) nadh[c] + (c + 2k) o2[c] --> 2dmmql8[c] + (3) h[p] + [1 + c + k) nad[c] + |
| | (c) h2o2[c] + (2k) o2s[c] + (k) h[c] |
| NADH5 | [c]: (1 + c) h + (1 + c + k) nadh + q8 (c + 2k) o2 --> (1 + c + k) nad + q8h2 + |
| | (c) h2o2 + (2k) o2s + (k) h |
| NADH9 | [c]: 2dmmq8 + (1 + c) h + (1 + c + k) nadh + (c + 2k) o2 --> 2dmmql8 + (1 + c + k) nad + |
| | (c) h2o2 + (2k) o2s + (k) h |
| NADPHQR2 | [c]: (1 + c) h + (1 + c + k) nadph + q8 + (c + 2k) o2 --> (1 + c + k) nadp + q8h2 + |
| | (c) h2o2 + (2k) o2s + (k)h |
| NADPHQR3 | [c]: (1 + c) h + mqn8 + (1 + c + k) nadph + (c + 2k) o2--> mql8 + (1 + c + k) nadp + |
| | (c) h2o2 + (2k) o2s + (k) h |
| NADPHQR4 | [c]: 2dmmq8 + (1 + c) h + (1 + c + k) nadph + (c + 2k) o2--> 2dmmql8 + (1 + c + k) nadp + |
| | (c) h2o2 + (2k) o2s + (k) h |
| NADTRHD | [c]: nad + (1 + c + k) nadph + (c) h + (c + 2k) o2 --> nadh + (1 + c + k) nadp + |
| | (c) h2o2 + (2k) o2s + (k)h |
| NHFRBO | [c]: (1 + c) h + (1 + c + k) nadh + (2) no + (c + 2k) o2 --> h2o + n2o + (1 + c + k) nad + |
| | (c) h2o2 + (2k) o2s + (k) h |
| NO3R1bpp | no3[p] + (1 + c + k) q8h2[c] + (c + 2k) o2[c] --> h2o[p] + no2[p] + (1 + c + k) q8[c] + |
| | (c) h2o2[c] + (2k) o2s[c] + (2k) h[c] |
| NO3R1pp | (2) h[c] + no3[c] + (1 + c + k) q8h2[c] + (c + 2k) o2[c] --> (2) h[p] + h2o[c] + no2[c] + (1 + c + k) q8[c] + |
| | (c) h2o2[c] + (2k) o2s[c] + (2k) h[c] |
| NO3R2bpp | (1 + c + k) mql8[c] + no3[p] + (c + 2k) o2[c]--> h2o[p] + (1 + c + k) mqn8[c] + no2[p] + |
| | (c) h2o2[c] + (2k) o2s[c] + (2k) h[c] |
| NO3R2pp | (2) h[c] + (1 + c + k) mql8[c] + no3[c] + (c + 2k) o2 --> (2) h[p] + h2o[c] + (1 + c + k) mqn8[c] + no2[c] + |
| | (c) h2o2[c] + (2k) o2s[c] + (2k) h[c] |
| NODOx | [c]: (c) h + (1 + c + k) nadh + (2) no + (2 + c + 2k) o2 --> (1 + k) h + (1 + c + k) nad + (2) no3 + |
| | (c) h2o2 + (2k) o2s |
| NODOy | [c]: (c) h + (1 + c + k) nadph + (2) no + (2 + c + 2k) o2 --> (1 + k) h + (1 + c + k) nadp + (2) no3 + |
| | (c) h2o2 + (2k) o2s |
| NTRIR2x | [c]: (5 + 3c) h + (3 + 3c + 3k) nadh + no2 + (3c + 6k) o2 --> (2) h2o + (3 + 3c + 3k) nad + nh4 + |
| | (3c) h2o2 + (6k) o2s + (3k) h |
| NTRIR3pp | (2) h[p] + no2[p] + (3 + 3c + 3k) q8h2[c] + (3c + 6k) o2[c] --> (2) h2o[p] + nh4[p] + (3 + 3c + 3k) q8[c] + |
| | (3c) h2o2[c] + (6k) o2s[c] + (6k) h[c] |
| NTRIR4pp | (2) h[p] + (3 + 3c + 3k) mql8[c] + no2[p] +(3c + 6k) o2[c] --> (2) h2o[p] + (3 + 3c + 3k) mqn8[c] + nh4[p] + |
| | (3c) h2o2[c] + (6k) o2s[c] + (6k) h[c] |
| OBTFL | [c]: 2obut + coa + (2k) o2 + (c + k) nadh + (2c) o2s + (3c) h --> for + ppcoa + |
| | (2c) h2o2 + (2k) o2s + (c + k) nad + (k) h |
| P5CD | NA |
| PDH | [c]: (1 + c + k) coa + nad + (1 + c + k) pyr + (c + 2k) o2 + |
| | (c) h--> (1 + c + k) accoa + (1 + c + k) co2 + nadh + |
| | (c) h2o2 + (2k) o2s + (k) h |
| PFL | [c]: coa + pyr + (2k) o2 + (c + k) nadh + (2c) o2s + (3c) h--> accoa + for + |
| | (2c) h2o2 + (2k) o2s + (c + k) nad + (k) h |
| POX | [c]: h2o + pyr + (1 − c − k) q8 + (c + 2k) o2 --> ac + co2 + (1 − c − k) q8h2 + |
| | (c) h2o2 + (2k) o2s + (2k) h |
| PPCDC | [c]: 4ppcys + h + (c + k) fmnh2 + (c + 2k) o2 --> co2 + pan4p + (c + k) fmn + |
| | (c) h2o2 + (2k) o2s + (2k) h |
| PPNCL2 | NA |
| PPPGO | [c]: (3) q8 + (1 + c + k) pppg9 + (3c + 6k) o2--> (1 + c + k) ppp9 + (3) q8h2 + |
| | (3c) h2o2 + (6k) o2s + (6k) h |
| PPPGO3 | [c]: (3) fum + (1 + c + k) pppg9 + (3c + 6k) o2--> (1 + c + k) ppp9 + (3) succ + |
| | (3c) h2o2 + (6k) o2s + (6k) h |
| PPPNDO | [c]: (1 + c) h + (1 + c + k) nadh + (1 + c + 2k) o2 + pppn --> cechddd + (1 + c + k) nad + |
| | (c) h2o2 + (2k) o2s + (k) h |
| PROD2 | [c]: (1 − c − k) fad + pro-L + (c + 2k) o2 --> 1pyr5c + (1 − c − k) fadh2 + |
| | (c) h2o2 + (2k) o2s + (1 + 2k) h |
| QULNS | [c]: dhap + iasp + (2k) o2 + (c + k) nadh + (2c) o2s + (3c) h --> (2) h2o + pi + quln + |
| | (2c) h2o2 + (2k) o2s + (c + k) nad + (k) h |
| RNTR1c | [c]: atp + (1 − c − k) fldrd + (c + 2k) o2 --> datp + (1 − c − k) fldox + h2o + |
| | (c) h2o2 + (2k) o2s + (2k) h |
| RNTR2c | [c]: gtp + (1 − c − k) fldrd + (c + 2k) o2 --> dgtp + (1 − c − k) fldox + h2o + |
| | (c) h2o2 + (2k) o2s + (2k) h |
| RNTR3c | [c]: ctp + (1 − c − k) fldrd + (c + 2k) o2 --> dctp + (1 − c − k) fldox + h2o + |
| | (c) h2o2 + (2k) o2s + (2k) h |
| RNTR4c | [c]: utp + (1 − c − k) fldrd + (c + 2k) o2 --> dutp + (1 − c − k) fldox + h2o + |
| | (c) h2o2 + (2k) o2s + (2k) h |
| SERD_L | [c]: ser-L + (2k) o2 + (c + k) nadh + (2c) o2s + (3c) h --> nh4 + pyr + |
| | (2c) h2o2 + (2k) o2s + (c + k) nad + (k) h |
| SUCDi | [c]: q8 + (1 + c + k) succ + (c + 2k) o2 --> (1 + c + k) fum + q8h2 + |
| | (c) h2o2 + (2k) o2s + (2k) h |
| SULRi | [c]: (5 + 3c) h + (3 + 3c + 3k) nadph + so3 (3c + 6k) o2 --> (3) h2o + h2s + (3 + 3c + 3k) nadp + |
| | (3c) h2o2 + (6k) o2s + (3k) h |
| TARTD | [c]: tartr-L + (2k) o2 + (c + k) nadh (2c) o2s + (3c) h --> h2o + oaa + |
| | (2c) h2o2 + (2k)o2s + (c + k) nad + (k) h |
| THRD_L | [c]: thr-L + (2k) o2 + (c + k) nadh (2c) o2s + (3c) h --> 2obut + nh4 + |
| | (2c) h2o2 + (2k) o2s + (c + k) nad + (k) h |
| TMAOR1 | [c]: h + (1 + c + k) mql8 + tmao + (c + 2k) o2--> h2o + (1 + c + k) mqn8 + tma + |
| | (c) h2o2 + (2k) o2s + (2k) h |
| TMAOR1pp | h[p] + (1 + c + k) mql8[c] + tmao[p] + (c + 2k) o2[c] --> h2o[p] + (1 + c + k) mqn8[c] + tma[p] + |
| | (c) h2o2[c] + (2k) o2s[c] + (2k) h[c] |
| TMAOR2 | [c]: h + (1 + c + k) 2dmmql8 + tmao + (c + 2k) o2--> h2o + (1 + c + k) 2dmmq8 + tma + |
| | (c) h2o2 + (2k) o2s + (2k) h |
| TMAOR2pp | h[p] + (1 + c + k) 2dmmql8[c] + tmao[p] + (c + 2k) o2[c] --> h2o[p] + (1 + c + k) 2dmmq8[c] + tma[p] + |
| | (c) h2o2[c] + (2k) o2s[c] + (2k) h[c] |
| TRDR | [c]: (1 + c) h + (1 + c + k) nadph + trdox + (c + 2k) o2 --> (1 + c + k) nadp + trdrd + |
| | (c) h2o2 + (2k) o2s + (k) h |
| UAPGR | [c]: (1 + c) h + (1 + c + k) nadph + uaccg + (c + 2k) o2--> (1 + c + k) nadp + uamr + |
| | (c) h2o2 + (2k)o2s + (k)h |
| UDPGALM | [c]: udpgal + (c + k) fadh2 + (c + 2k) o2--> udpgalfur + (c + k) fad + |
| | (c) h2o2 + (2k) o2s + (2k) h |
| | Notes |
| ACHBS | PMID: 15236573 |
| ACLS | PMID: 15236573 |
| ACOAD1f | PMID: 14527285, General Rule: reduced flavin donates electrons to oxygen to generate |
| | superoxide and hydrogen peroxide |
| ACOAD2f | PMID: 14527285, General Rule: reduced flavin donates electrons to oxygen to generate |
| | superoxide and hydrogen peroxide |
| ACOAD3f | PMID: 14527285, General Rule: reduced flavin donates electrons to oxygen to generate |
| | superoxide and hydrogen peroxide |
| ACOAD4f | PMID: 14527285, General Rule: reduced flavin donates electrons to oxygen to generate |
| | superoxide and hydrogen peroxide |
| ACOAD5f | PMID: 14527285, General Rule: reduced flavin donates electrons to oxygen to generate |
| | superoxide and hydrogen peroxide |
| ACOAD6f | PMID: 14527285, General Rule: reduced flavin donates electrons to oxygen to generate |
| | superoxide and hydrogen peroxide |
| ACOAD7f | PMID: 14527285, General Rule: reduced flavin donates electrons to oxygen to generate |
| | superoxide and hydrogen peroxide |
| ACOAD8f | PMID: 14527285, General Rule: reduced flavin donates electrons to oxygen to generate |
| | superoxide and hydrogen peroxide |
| ACONTa | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| ACONTb | Aconitase reaction is split in half, therefore only one of the halves was allowed to generate ROS |
| AKGDH | PMID: 15356189, PMID: 12631263 |
| AMMQLT8 | PMID: 14527285, General Rule: reduced quinone donates electrons to oxygen to generate |
| | superoxide and hydrogen peroxide |
| ARBTNR1 | PMID: 14527285, General Rule: reduced flavin donates electrons to oxygen to generate |
| | superoxide and hydrogen peroxide |
| ARBTNR2 | PMID: 14527285, General Rule: reduced flavin donates electrons to oxygen to generate |
| | superoxide and hydrogen peroxide |
| ASPO3 | PMID: 20149100, Due to the physiological relevance of ASPO6 this reaction was inactivated |
| ASPO4 | PMID: 20149100, Due to the physiological relevance of ASPO6 this reaction was inactivated |
| ASPO5 | PMID: 20149100, Due to the physiological relevance of ASPO6 this reaction was inactivated |
| BTS4 | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| CPGNR1 | PMID: 14527285, General Rule: reduced flavin donates electrons to oxygen to generate |
| | superoxide and hydrogen peroxide |
| CPGNR2 | PMID: 14527285, General Rule: reduced flavin donates electrons to oxygen to generate |
| | superoxide and hydrogen peroxide |
| CPPPGO2 | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| CYTBD2pp | PMID: 14527285, General Rule: reduced quinone donates electrons to oxygen to generate |
| | superoxide and hydrogen peroxide |
| CYTBDpp | PMID: 14527285, General Rule: reduced quinone donates electrons to oxygen to generate |
| | superoxide and hydrogen peroxide |
| CYTBO3_4pp | PMID: 14527285, General Rule: reduced quinone donates electrons to oxygen to generate |
| | superoxide and hydrogen peroxide |
| DAAD | PMID: 14527285, General Rule: reduced flavin donates electrons to oxygen to generate |
| | superoxide and hydrogen peroxide |
| DHAD1 | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| DHAD2 | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| DHNAOT4 | PMID: 14527285, General Rule: reduced quinone donates electrons to oxygen to generate |
| | superoxide and hydrogen peroxide |
| DHORD2 | PMID: 14527285, General Rule: reduced flavin donates electrons to oxygen to generate |
| | superoxide and hydrogen peroxide |
| DHORD5 | PMID: 14527285, General Rule: reduced flavin donates electrons to oxygen to generate |
| | superoxide and hydrogen peroxide |
| DMPPS | PMID: 14527285, PMID: 12198182, General Rule: reduced flavin donates electrons to oxygen |
| | to generate superoxide and hydrogen peroxide |
| DMQMT | PMID: 14527285, General Rule: reduced quinone donates electrons to oxygen to generate |
| | superoxide and hydrogen peroxide |
| DMSOR1 | PMID: 14527285, General Rule: reduced quinone donates electrons to oxygen to generate |
| | superoxide and hydrogen peroxide |
| DMSOR1pp | PMID: 14527285, General Rule: reduced quinone donates electrons to oxygen to generate |
| | superoxide and hydrogen peroxide |
| DMSOR2 | PMID: 14527285, General Rule: reduced quinone donates electrons to oxygen to generate |
| | superoxide and hydrogen peroxide |
| DMSOR2pp | PMID: 14527285, General Rule: reduced quinone donates electrons to oxygen to generate |
| | superoxide and hydrogen peroxide |
| DSBAO1 | PMID: 14527285, General Rule: reduced quinone donates electrons to oxygen to generate |
| | superoxide and hydrogen peroxide |
| DSBAO2 | PMID: 14527285, General Rule: reduced quinone donates electrons to oxygen to generate |
| | superoxide and hydrogen peroxide |
| FADRx | PMID: 14527285, General Rule: reduced flavin donates electrons to oxygen to generate |
| | superoxide and hydrogen peroxide |
| FADRx2 | PMID: 14527285, General Rule: reduced flavin donates electrons to oxygen to generate |
| | superoxide and hydrogen peroxide |
| FDH4pp | PMID: 14527285, General Rule: reduced quinone donates electrons to oxygen to generate |
| | superoxide and hydrogen peroxide |
| FDH5pp | PMID: 14527285, General Rule: reduced quinone donates electrons to oxygen to generate |
| | superoxide and hydrogen peroxide |
| FDMO | PMID: 14527285, General Rule: reduced flavin donates electrons to oxygen to generate |
| | superoxide and hydrogen peroxide |
| FDMO2 | PMID: 14527285, General Rule: reduced flavin donates electrons to oxygen to generate |
| | superoxide and hydrogen peroxide |
| FDMO3 | PMID: 14527285, General Rule: reduced flavin donates electrons to oxygen to generate |
| | superoxide and hydrogen peroxide |
| FDMO4 | PMID: 14527285, General Rule: reduced flavin donates electrons to oxygen to generate |
| | superoxide and hydrogen peroxide |
| FDMO6 | PMID: 14527285, General Rule: reduced flavin donates electrons to oxygen to generate |
| | superoxide and hydrogen peroxide |
| FE3HOXR1 | PMID: 14527285, General Rule: reduced flavin donates electrons to oxygen to generate |
| | superoxide and hydrogen peroxide |
| FE3HOXR2 | PMID: 14527285, General Rule: reduced flavin donates electrons to oxygen to generate |
| | superoxide and hydrogen peroxide |
| FE3Ri | PMID: 14527285, General Rule: reduced flavin donates electrons to oxygen to generate |
| | superoxide and hydrogen peroxide |
| FECRMR1 | PMID: 14527285, General Rule: reduced flavin donates electrons to oxygen to generate |
| | superoxide and hydrogen peroxide |
| FECRMR2 | PMID: 14527285, General Rule: reduced flavin donates electrons to oxygen to generate |
| | superoxide and hydrogen peroxide |
| FEENTERR1 | PMID: 14527285, General Rule: reduced flavin donates electrons to oxygen to generate |
| | superoxide and hydrogen peroxide |
| FEENTERR2 | PMID: 14527285, General Rule: reduced flavin donates electrons to oxygen to generate |
| | superoxide and hydrogen peroxide |
| FEOXAMR1 | PMID: 14527285, General Rule: reduced flavin donates electrons to oxygen to generate |
| | superoxide and hydrogen peroxide |
| FEOXAMR2 | PMID: 14527285, General Rule: reduced flavin donates electrons to oxygen to generate |
| | superoxide and hydrogen peroxide |
| FLDR | PMID: 14527285, General Rule: reduced flavin donates electrons to oxygen to generate |
| | superoxide and hydrogen peroxide |
| FLVR | PMID: 14527285, General Rule: reduced flavin donates electrons to oxygen to generate |
| | superoxide and hydrogen peroxide |
| FMNRx | PMID: 14527285, General Rule: reduced flavin donates electrons to oxygen to generate |
| | superoxide and hydrogen peroxide |
| FMNRx2 | PMID: 14527285, General Rule: reduced flavin donates electrons to oxygen to generate |
| | superoxide and hydrogen peroxide |
| FRD2 | PMID: 12200425, PMID: 14527285, General Rule: reduced flavin donates electrons to oxygen |
| | to generate superoxide and hydrogen peroxide |
| FRD3 | PMID: 12200425, PMID: 14527285, General Rule: reduced flavin donates electrons to oxygen |
| | to generate superoxide and hydrogen peroxide |
| FUM | General Rule: ROS generated from continual cycling between repair and damage of Fe—S |
| | cluster (PMID: 1315737, PMID: 15308657). Assume NADH as source of electrons for repair |
| | (PMID: 15308657) |
| G3PD5 | PMID: 14527285, General Rule: reduced flavin donates electrons to oxygen to generate |
| | superoxide and hydrogen peroxide |
| G3PD6 | PMID: 14527285, General Rule: reduced flavin donates electrons to oxygen to generate |
| | superoxide and hydrogen peroxide |
| G3PD7 | PMID: 14527285, General Rule: reduced flavin donates electrons to oxygen to generate |
| | superoxide and hydrogen peroxide |
| GLCDpp | General Rule: reduced quinone donates electrons to oxygen to generate superoxide and |
| | hydrogen peroxide |
| GLUSy | PMID: 8665916, PMID: 15052410 |
| GLXCL | PMID: 15236573, PMID: 8440684 |
| GLYCL | PMID: 12631263, PMID: 15356189 |
| GLYCTO2 | PMID: 14527285, General Rule: reduced quinone donates electrons to oxygen to generate |
| | superoxide and hydrogen peroxide |
| GLYCTO3 | PMID: 14527285, General Rule: reduced quinone donates electrons to oxygen to generate |
| | superoxide and hydrogen peroxide |
| GLYCTO4 | PMID: 14527285, General Rule: reduced quinone donates electrons to oxygen to generate |
| | superoxide and hydrogen peroxide |
| GTHOr | PMID: 7175934 |
| HYD1pp | PMID: 14527285, General Rule: reduced quinone donates electrons to oxygen to generate |
| | superoxide and hydrogen peroxide |
| HYD2pp | PMID: 14527285, General Rule: reduced quinone donates electrons to oxygen to generate |
| | superoxide and hydrogen peroxide |
| HYD3pp | PMID: 14527285, General Rule: reduced quinone donates electrons to oxygen to generate |
| | superoxide and hydrogen peroxide |
| IPDPS | PMID: 14527285, PMID: 12198182, General Rule: reduced flavin donates electrons to oxygen |
| | to generate superoxide and hydrogen peroxide |
| LDH_D | |
| LDH_D2 | PMID: 14527285, General Rule: reduced quinone donates electrons to oxygen to generate |
| | superoxide and hydrogen peroxide |
| L_LACD2 | PMID: 14527285, General Rule: reduced flavin donates electrons to oxygen to generate |
| | superoxide and hydrogen peroxide |
| L_LACD3 | PMID: 14527285, General Rule: reduced flavin donates electrons to oxygen to generate |
| | superoxide and hydrogen peroxide |
| MDH2 | PMID: 14527285, General Rule: reduced flavin donates electrons to oxygen to generate |
| | superoxide and hydrogen peroxide |
| MDH3 | PMID: 14527285, General Rule: reduced flavin donates electrons to oxygen to generate |
| | superoxide and hydrogen peroxide |
| MICITD | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| MTHFR2 | PMID: 14527285, General Rule: reduced flavin donates electrons to oxygen to generate |
| | superoxide and hydrogen peroxide |
| NADH10 | PMID: 14527285, General Rule: reduced flavin donates electrons to oxygen to generate |
| | superoxide and hydrogen peroxide |
| NADH16pp | PMID: 14527285, General Rule: reduced flavin donates electrons to oxygen to generate |
| | superoxide and hydrogen peroxide |
| NADH17pp | PMID: 14527285, General Rule: reduced flavin donates electrons to oxygen to generate |
| | superoxide and hydrogen peroxide |
| NADH18pp | PMID: 14527285, General Rule: reduced flavin donates electrons to oxygen to generate |
| | superoxide and hydrogen peroxide |
| NADH5 | PMID: 14527285, General Rule: reduced flavin donates electrons to oxygen to generate |
| | superoxide and hydrogen peroxide |
| NADH9 | PMID: 14527285, General Rule: reduced flavin donates electrons to oxygen to generate |
| | superoxide and hydrogen peroxide |
| NADPHQR2 | PMID: 14527285, General Rule: reduced flavin donates electrons to oxygen to generate |
| | superoxide and hydrogen peroxide |
| NADPHQR3 | PMID: 14527285, General Rule: reduced flavin donates electrons to oxygen to generate |
| | superoxide and hydrogen peroxide |
| NADPHQR4 | PMID: 14527285, General Rule: reduced flavin donates electrons to oxygen to generate |
| | superoxide and hydrogen peroxide |
| NADTRHD | PMID: 14527285, General Rule: reduced flavin donates electrons to oxygen to generate |
| | superoxide and hydrogen peroxide |
| NHFRBO | PMID: 14527285, General Rule: reduced flavin donates electrons to oxygen to generate |
| | superoxide and hydrogen peroxide |
| NO3R1bpp | PMID: 14527285, General Rule: reduced quinone donates electrons to oxygen to generate |
| | superoxide and hydrogen peroxide |
| NO3R1pp | PMID: 14527285, General Rule: reduced quinone donates electrons to oxygen to generate |
| | superoxide and hydrogen peroxide |
| NO3R2bpp | PMID: 14527285, General Rule: reduced quinone donates electrons to oxygen to generate |
| | superoxide and hydrogen peroxide |
| NO3R2pp | PMID: 14527285, General Rule: reduced quinone donates electrons to oxygen to generate |
| | superoxide and hydrogen peroxide |
| NODOx | PMID: 14527285, General Rule: reduced flavin donates electrons to oxygen to generate |
| | superoxide and hydrogen peroxide |
| NODOy | PMID: 14527285, General Rule: reduced flavin donates electrons to oxygen to generate |
| | superoxide and hydrogen peroxide |
| NTRIR2x | PMID: 14527285, General Rule: reduced flavin donates electrons to oxygen to generate |
| | superoxide and hydrogen peroxide |
| NTRIR3pp | PMID: 14527285, General Rule: reduced quinone donates electrons to oxygen to generate |
| | superoxide and hydrogen peroxide |
| NTRIR4pp | PMID: 14527285, General Rule: reduced quinone donates electrons to oxygen to generate |
| | superoxide and hydrogen peroxide |
| OBTFL | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| P5CD | PMID: 7966312 |
| PDH | PMID: 14527285, General Rule: reduced flavin donates electrons to oxygen to generate |
| | superoxide and hydrogen peroxide |
| PFL | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| POX | PMID: 14527285, General Rule: reduced flavin donates electrons to oxygen to generate |
| | superoxide and hydrogen peroxide |
| PPCDC | PMID: 10922366 . . . |
| PPNCL2 | PMID: 10922366, Bifunctional enzyme, FMN participates in only |
| | 4′-phosphopantothenoylcysteine decarboxylase activity |
| PPPGO | PMID: 14527285, General Rule: reduced flavin donates electrons to oxygen to generate |
| | superoxide and hydrogen peroxide |
| PPPGO3 | PMID: 14527285, General Rule: reduced flavin donates electrons to oxygen to generate |
| | superoxide and hydrogen peroxide |
| PPPNDO | PMID: 14527285, General Rule: reduced flavin donates electrons to oxygen to generate |
| | superoxide and hydrogen peroxide |
| PROD2 | PMID: 14527285, General Rule: reduced flavin donates electrons to oxygen to generate |
| | superoxide and hydrogen peroxide |
| QULNS | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| RNTR1c | PMID: 14527285, General Rule: reduced flavin donates electrons to oxygen to generate |
| | superoxide and hydrogen peroxide |
| RNTR2c | PMID: 14527285, General Rule: reduced flavin donates electrons to oxygen to generate |
| | superoxide and hydrogen peroxide |
| RNTR3c | PMID: 14527285, General Rule: reduced flavin donates electrons to oxygen to generate |
| | superoxide and hydrogen peroxide |
| RNTR4c | PMID: 14527285, General Rule: reduced flavin donates electrons to oxygen to generate |
| | superoxide and hydrogen peroxide |
| SERD_L | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| SUCDi | PMID: 14527285, General Rule: reduced flavin donates electrons to oxygen to generate |
| | superoxide and hydrogen peroxide |
| SULRi | PMID: 14527285, General Rule: reduced flavin donates electrons to oxygen to generate |
| | superoxide and hydrogen peroxide |
| TARTD | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| THRD_L | PMID: 1315737, PMID: 15308657, PMID: 15308657, General Rule: ROS generated from |
| | continual cycling between repair and damage of Fe—S cluster (PMID: 1315737, PMID: |
| | 15308657). Assume NADH as source of electrons for repair (PMID: 15308657) |
| TMAOR1 | PMID: 14527285, General Rule: reduced quinone donates electrons to oxygen to generate |
| | superoxide and hydrogen peroxide |
| TMAOR1pp | PMID: 14527285, General Rule: reduced quinone donates electrons to oxygen to generate |
| | superoxide and hydrogen peroxide |
| TMAOR2 | PMID: 14527285, General Rule: reduced quinone donates electrons to oxygen to generate |
| | superoxide and hydrogen peroxide |
| TMAOR2pp | PMID: 14527285, General Rule: reduced quinone donates electrons to oxygen to generate |
| | superoxide and hydrogen peroxide |
| TRDR | PMID: 14527285, General Rule: reduced flavin donates electrons to oxygen to generate |
| | superoxide and hydrogen peroxide |
| UAPGR | PMID: 14527285, General Rule: reduced flavin donates electrons to oxygen to generate |
| | superoxide and hydrogen peroxide |
| UDPGALM | PMID: 14527285, General Rule: reduced flavin donates electrons to oxygen to generate |
| | superoxide and hydrogen peroxide |

Enzyme usage of flavin, quinone, or transition metal was identified using Ecocyc database (PMID: 18974181)
ROS-generating side reactions were often ill-defined, when necessary, approximations were made from enzyme homologues in other species, ROS -generating reactions from enzymes in the same family, and general rules for electron transfer from promiscuous electron carriers
Catalase has heme, superoxide dismutase has transition metal, and alkyl hydroperoxide reductase has flavin, but all were not considered to be ROS sources as they are detoxification enzymes

TABLE 2
GFP/BM Measurements for Deletion Strains with Reporter Plasmids

| | Relative GFP/OD | Standard Error | P-value |
|---|---|---|---|
| ΔaceA (soxS-gfp) | 0.870 | 0.028 | 1.00 |
| ΔappB (soxS-gfp) | 1.001 | 0.029 | 0.58 |
| ΔatpC (soxS-gfp) | 1.943 | 0.035 | 0.00 |
| ΔcyoA (soxS-gfp) | 1.160 | 0.028 | 0.01 |
| Δedd (soxS-gfp) | 0.998 | 0.023 | 0.77 |
| ΔfbaB (soxS-gfp) | 1.020 | 0.026 | 0.30 |
| ΔfumB (soxS-gfp) | 1.101 | 0.031 | 0.01 |
| ΔgdhA (soxS-gfp) | 0.969 | 0.019 | 0.85 |
| ΔglsB (soxS-gfp) | 0.923 | 0.092 | 0.71 |
| Δgnd (soxS-gfp) | 1.611 | 0.082 | 0.00 |
| Δmqo (soxS-gfp) | 0.938 | 0.099 | 0.65 |
| ΔnuoG (soxS-gfp) | 1.055 | 0.019 | 0.02 |
| ΔpfkB (soxS-gfp) | 1.186 | 0.033 | 0.00 |
| Δpta (soxS-gfp) | 1.241 | 0.042 | 0.00 |
| ΔpykA (soxS-gfp) | 0.975 | 0.027 | 0.75 |
| ΔrplB (soxS-gfp) | 1.037 | 0.043 | 0.07 |
| ΔsdhC (soxS-gfp) | 0.959 | 0.017 | 0.75 |
| ΔsucC (soxS-gfp) | 0.950 | 0.016 | 0.98 |
| ΔtalB (soxS-gfp) | 1.048 | 0.019 | 0.05 |
| ΔtkrB (soxS-gfp) | 1.006 | 0.025 | 0.12 |
| Δzwf (soxS-gfp) | 1.391 | 0.074 | 0.01 |
| ΔaceA (dps-gfp) | 0.922 | 0.017 | 0.99 |
| ΔappB (dps-gfp) | 0.980 | 0.018 | 0.82 |
| ΔatpC (dps-gfp) | 2.212 | 0.038 | 0.00 |
| ΔcyoA (dps-gfp) | 1.106 | 0.029 | 0.01 |
| Δedd (dps-gfp) | 0.970 | 0.016 | 0.95 |
| ΔfbaB (dps-gfp) | 1.026 | 0.015 | 0.05 |
| ΔfumB (dps-gfp) | 0.977 | 0.022 | 0.66 |
| ΔgdhA (dps-gfp) | 1.009 | 0.031 | 0.39 |
| ΔgltB (dps-gfp) | 0.990 | 0.048 | 0.77 |
| Δgnd (dps-gfp) | 1.107 | 0.025 | 0.03 |
| Δmqo (dps-gfp) | 1.010 | 0.047 | 0.48 |
| ΔnuoG (dps-gfp) | 1.181 | 0.031 | 0.00 |
| ΔpfkB (dps-gfp) | 1.251 | 0.020 | 0.00 |
| Δpta (dps-gfp) | 1.243 | 0.019 | 0.00 |
| ΔpykA (dps-gfp) | 1.024 | 0.017 | 0.11 |
| ΔrplB (dps-gfp) | 0.988 | 0.009 | 0.37 |
| ΔsdhC (dps-gfp) | 1.126 | 0.021 | 0.00 |
| ΔsucC (dps-gfp) | 1.181 | 0.021 | 0.00 |
| ΔtalB (dps-gfp) | 0.994 | 0.012 | 0.10 |
| ΔtktB (dps-gfp) | 1.021 | 0.016 | 0.36 |
| Δzwf (dps-gfp) | 1.439 | 0.054 | 0.02 |

Note:
dps promoter is the hydrogen peroxide sensitive promoter
Note:
soxS is the superoxide sensitive reporter

TABLE 3
Relative (500 nm/420 nm) Fluorescence Ratio of Multiple Strains

| Strain | Average Ratio (500/420) | STD | p-value |
|---|---|---|---|
| wildtype | 47.51 | 8.74 | |
| ΔaceA | 57.36 | 11.86 | 0.16 |
| ΔappB | 51.74 | 9.76 | 0.30 |
| ΔcyoA | 59.34 | 7.31 | 0.07 |
| Δedd | 51.94 | 8.17 | 0.28 |
| ΔfbaB | 43.00 | 6.84 | 0.26 |
| ΔfumB | 50.75 | 7.88 | 0.33 |
| ΔgdhA | 49.68 | 9.71 | 0.39 |
| ΔgltB | 51.99 | 9.16 | 0.29 |
| Δgnd | 56.86 | 10.23 | 0.15 |
| Δmqo | 53.66 | 5.71 | 0.19 |
| ΔnuoG | 67.76 | 13.37 | 0.05 |
| ΔpfkB | 53.48 | 7.44 | 0.21 |
| Δpta | 42.90 | 9.24 | 0.28 |
| ΔpykA | 53.32 | 8.75 | 0.23 |
| ΔrplB | 52.24 | 7.80 | 0.26 |
| ΔsdhC | 61.48 | 8.17 | 0.06 |
| ΔsucC | 45.77 | 0.21 | 0.38 |
| ΔtalB | 52.73 | 9.66 | 0.26 |
| ΔtktB | 53.40 | 10.21 | 0.25 |
| wildtype | 50.14 | 5.48 | |
| ΔatpC | 65.60 | 2.18 | 0.02 |
| Δzwf | 63.24 | 5.96 | 0.03 |

Note:
ΔatpC and Δzwf were run separately with wildtype due to growth issues in the 96-well plate format

TABLE 4
ROS generation within iAF2260

| Rxn Name | Flavin | Quinone | Fe—S | Heme | Reversible | iAF1260 index |
|---|---|---|---|---|---|---|
| 42A12BOOXpp | 0 | 1 | 0 | 0 | 0 | 114 |
| AACTOOR | 1 | 1 | 0 | 0 | 0 | 135 |
| ASPO6 | 1 | 0 | 0 | 0 | 0 | 362 |
| GGPTRCO | 0 | 0 | 0 | 0 | 0 | 1210 |
| MTRPOX | 1 | 0 | 0 | 0 | 0 | 1705 |
| PDX5POi | 1 | 0 | 0 | 0 | 0 | 1892 |
| PEAMNOpp | 0 | 1 | 0 | 0 | 0 | 1902 |
| PYAM5PO | 1 | 0 | 0 | 0 | 0 | 2094 |
| QMO2 | 0 | 1 | 0 | 0 | 0 | 2103 |
| QMO3 | 0 | 1 | 0 | 0 | 0 | 2104 |
| SARCOX | 1 | 0 | 0 | 0 | 0 | 2144 |
| TYROXDApp | 0 | 1 | 0 | 0 | 0 | 2284 |
| URIC | 0 | 0 | 0 | 0 | 1 | 2345 |

| Rxn Name | Rxn (iAF1260) | Notes |
|---|---|---|
| 42A12BOOXpp | [p]: dopa + h2o + o2 --> 34dhpac + h2o2 + nh4 | PMID: 17593909 |
| AACTOOR | [c]: aact + h2o + o2 --> h2o2 + mthgxl + nh4 | PMID: 17593909 |
| ASPO6 | [c]: asp-L + o2 --> h + h2o2 + iasp | PMID: 17593909 |
| GGPTRCO | [c]: ggptrc + h2o + o2 --> ggbutal + h2o2 + nh4 | PMID: 17593909 |
| MTRPOX | [c]: Nmtrp + h2o + o2 --> fald + h2o2 + trp-L | PMID: 17593909 |
| PDX5POi | [c]: o2 + pdx5p --> h2o2 + pydsx5p | PMID: 17593909 |
| PEAMNOpp | [p]: h2o + o2 + peamn --> h2o2 + nh4 +pacald | PMID: 17593909 |
| PYAM5PO | [c]: h2o + o2 + pyam5p --> h2o2 + nh4 + pydx5p | PMID: 17593909 |
| QMO2 | [c]: (2) o2 + q8h2 --> (2) h + (2) o2s + q8 | PMID: 17593909 |
| QMO3 | [c]: mql8 + (2) o2 --> (2) h + mqn8 + (2) o2s | PMID: 17593909 |
| SARCOX | [c]: h2o + o2 + sarcs --> fald + gly + h2o2 | PMID: 17593909 |
| TYROXDApp | [p]: h2o + o2 + tym --> 4hoxpacd + h2o2 + nh4 | PMID: 17593909 |
| URIC | [c]: (2) h2o + o2 + urate --> alltn + co2 + h2o2 | PMID: 17593909 |

TABLE 5
Enzymes and Regulators Turned Off Due to Transcriptional Regulation in Minimal glt

| B number | Gene name | Regulatory Rule | Function |
|---|---|---|---|
| b0034 | calF | (Fnr AND Crp AND NOT NarL) | Regulation |
| b0036 | calD | (Crp AND CalF) | Enzymatic activity |
| b0038 | calB | (Crp AND CalF) | Enzymatic activity |
| b0040 | calT | (Crp AND CalF) | Enzymatic activity |
| b0061 | araD | (AraC OR (AraC AND Crp)) | Enzymatic activity |
| b0062 | araA | (AraC OR (AraC AND Crp)) | Enzymatic activity |
| b0063 | araB | (AraC OR (AraC AND Crp)) | Enzymatic activity |
| b0064 | araC | (arab-L(e) > 0) | Regulation |
| b0125 | hpt | (Crp) | Enzymatic activity |
| b0162 | sdaR | ((glcr(e) > 0) OR (galct-D(e) > 0)) | Regulation |
| b0313 | betl | (chol(e) > 0) | Regulation |
| b0331 | prpB | (ppa(e) > 0) | Enzymatic activity |
| b0333 | prpC | (ppa(e) > 0) | Enzymatic activity |
| b0334 | prpD | (ppa(e) > 0) | Enzymatic activity |
| b0335 | prpE | (ppa(e) > 0) | Enzymatic activity |
| b0338 | cynR | (cynt(e) > 0) | Regulation |
| b0340 | cynS | (CynR) | Enzymatic activity |
| b0341 | cynX | (cynt(e) > 0) | Enzymatic activity |
| b0343 | lacY | (“CRP noGLC” AND NOT(Lacl)) | Enzymatic activity |
| b0344 | lacZ | (“CRP noGLC” AND NOT(Lacl)) | Enzymatic activity |
| b0346 | mhpR | (3hoppn(e) > 0) | Regulation |
| b0347 | mhpA | (MhpR) | Enzymatic activity |
| b0348 | mhpB | (MhpR) | Enzymatic activity |
| b0349 | mhpC | (MhpR) | Enzymatic activity |
| b0350 | mhpD | (MhpR) | Enzymatic activity |
| b0351 | mhpF | (MhpR) | Enzymatic activity |
| b0352 | mhpE | (MhpR) | Enzymatic activity |
| b0365 | tauA | (Cbl AND CysB) | Enzymatic activity |
| b0366 | tauB | (Cbl AND CysB) | Enzymatic activity |
| b0367 | tauC | (Cbl AND CysB) | Enzymatic activity |
| b0368 | tauD | (Cbl AND CysB) | Enzymatic activity |
| b0403 | malZ | (MalT) | Enzymatic activity |
| b0504 | ybbS/AIIS | (NOT(o2(e) > 0) AND NOT AIIR AND | Regulation |
| | | NOT (nh4(e) > 0)) | |
| b0505 | aIIA | (NOT AIIR) | Enzymatic activity |
| b0507 | gcl | (NOT AIIR) | Enzymatic activity |
| b0509 | glxR | (NOT AIIR) | Enzymatic activity |
| b0512 | aIIB | (NOT AIIR) | Enzymatic activity |
| b0514 | glxK | (NOT AIIR) | Enzymatic activity |
| b0516 | aIIC | (AIIS) | Enzymatic activity |
| b0612 | citT | (CitB AND (NOT (o2(e) > 0))) | Enzymatic activity |
| b0615 | citF | (CitB) | Enzymatic activity |
| b0616 | citE | (CitB) | Enzymatic activity |
| b0617 | citD | (CitB) | Enzymatic activity |
| b0619 | dpiB/citA | (cit(e) > 0) | Regulation |
| b0620 | dpiA/citB | (CitA) | Regulation |
| b0621 | dcuC | (Fnr OR ArcA) | Enzymatic activity |
| b0652 | gltL | (NOT (glc-D(e) > 0)) | Enzymatic activity |
| b0653 | gltK | (NOT (glc-D(e) > 0)) | Enzymatic activity |
| b0654 | gltJ | (NOT (glc-D(e) > 0)) | Enzymatic activity |
| b0655 | gltI | (NOT (glc-D(e) > 0)) | Enzymatic activity |
| b0677 | nagA | (NOT (NagC)) | Enzymatic activity |
| b0678 | nagB | (NOT (NagC) OR (gam(e) > 0)) | Enzymatic activity |
| b0679 | nagE | (NOT (NagC)) | Enzymatic activity |
| b0683 | fur | ((fe2(e) > 0) AND (OxyR OR SoxS)) | Regulation |
| b0694 | kdpE | (KdpD) | Regulation |
| b0695 | kdpD | (NOT (k(e) > 1)) | Regulation |
| b0696 | kdpC | (KdpE) | Enzymatic activity |
| b0697 | kdpB | (KdpE) | Enzymatic activity |
| b0698 | kdpA | (KdpE) | Enzymatic activity |
| b0774 | bioA | (NOT (BirA)) | Enzymatic activity |
| b0775 | bioB | (NOT (BirA)) | Enzymatic activity |
| b0776 | bioF | (NOT (BirA)) | Enzymatic activity |
| b0778 | bioD | (NOT (BirA)) | Enzymatic activity |
| b0840 | deoR | (NOT((PPM2 > 0) OR (PPM2 > 0))) | Regulation |
| b0871 | poxB | ((NOT (Growth > 0)) AND (RpoS)) | Enzymatic activity |
| b0894 | dmsA | (Fnr AND NOT NarL) | Enzymatic activity |
| b0895 | dmsB | (Fnr AND NOT NarL) | Enzymatic activity |
| b0896 | dmsC | (Fnr AND NOT NarL) | Enzymatic activity |
| b0902 | pflA | (ArcA OR Fnr AND (Crp OR | Enzymatic activity |
| | | NOT(NarL))) | |
| b0903 | pflB | (ArcA OR fnr AND (Crp OR | Enzymatic activity |
| | | NOT(NarL))) | |
| b0904 | focA | (ArcA OR Fnr AND (Crp OR NOT | Enzymatic activity |
| | | (NarL))) | |
| b0972 | hyaA | ((ArcA OR Fnr) AND (AppY)) | Enzymatic activity |
| b0973 | hyaB | ((ArcA OR Fnr) AND (AppY)) | Enzymatic activity |
| b0974 | hyaC | ((ArcA OR Fnr) AND (AppY)) | Enzymatic activity |
| b0993 | torS | (tmao(e) > 0) | Regulation |
| b0995 | torR | (TorS) | Regulation |
| b1014 | putA | ((pro-L(e) > 0) OR Crp OR Nac) | Enzymatic activity |
| b1015 | putP | ((pro-L(e) > 0) OR Crp OR Nac) | Enzymatic activity |
| b1189 | dadA | (ala-L(e) > 0 AND NOT Crp) | Enzymatic activity |
| b1190 | dadX | (((ala-L(e) > 0) OR (ala-D(e) > 0)) AND | Enzymatic activity |
| | | Crp) | |
| b1197 | treA | (RpoS) | Enzymatic activity |
| b1221 | narL | ((no3(e) > 0) OR (no2(e) > 0)) | Regulation |
| b1223 | narK | (Fnr OR NarL) | Enzymatic activity |
| b1224 | narG | (Fnr AND NarL) | Enzymatic activity |
| b1225 | narH | (Fnr AND NarL) | Enzymatic activity |
| b1226 | narJ | (Fnr AND NarL) | Enzymatic activity |
| b1227 | narI | (Fnr AND NarL) | Enzymatic activity |
| b1276 | acnA | (SoxS) | Enzymatic activity |
| b1323 | tyrR | ((trp-L(e) > 0) OR (tyr-L(e) > 0) OR (phe- | Regulation |
| | | L(e) > 0)) | |
| b1334 | fnr | (NOT (o2(e) > 0)) | Regulation |
| b1384 | feaR | (Crp) | Regulation |
| b1385 | feaB | (FeaR) | Enzymatic activity |
| b1386 | tynA | (FeaB) | Enzymatic activity |
| b1474 | fdnG | (Fnr OR NarL) | Enzymatic activity |
| b1475 | fdnH | (Fnr OR NarL) | Enzymatic activity |
| b1476 | fdnI | (Fnr OR NarL) | Enzymatic activity |
| b1493 | gadB | ((NOT (Growth > 0)) OR (pH < 4)) | Enzymatic activity |
| b1519 | tam | (NOT (Growth > 0)) | Enzymatic activity |
| b1521 | uxaB | (NOT ExuR) | Enzymatic activity |
| b1531 | marA | (“Salicylate”) | Regulation |
| b1594 | mlc | (NOT (glc-D(e) > 0)) | Regulation |
| b1611 | fumC | (MarA OR Rob OR SoxS AND | Enzymatic activity |
| | | (NOT(ArcA))) | |
| b1621 | malX | ((MalT AND Crp) OR MalT) | Enzymatic activity |
| b1622 | malY | (NOT (Mall)) | Enzymatic activity |
| b1623 | add | ((ade(e) > 0) OR (hxan(e) > 0)) | Enzymatic activity |
| b1646 | sodC | (NOT(Growth > 0) AND NOT Fnr) | Enzymatic activity |
| b1658 | purR | ((hxan(e) > 0) OR (gua(e) > 0)) | Regulation |
| b1702 | pps | (Cra) | Enzymatic activity |
| b1732 | katE | (NOT(Growth > 0)) | Enzymatic activity |
| b1744 | astE | ((NOT(Growth > 0) AND RpoS) OR | Enzymatic activity |
| | | (NRI_hi AND RpoN)) | |
| b1745 | astB | ((NOT(Growth > 0) AND RpoS) OR | Enzymatic activity |
| | | (NRI_hi AND RpoN)) | |
| b1746 | astD | ((NOT(Growth > 0) AND RpoS) OR | Enzymatic activity |
| | | (NRI_hi AND RpoN)) | |
| b1747 | astA | ((NOT(Growth > 0) AND RpoS) OR | Enzymatic activity |
| | | (NRI_hi AND RpoN)) | |
| b1748 | astC | ((NOT(Growth > 0) AND RpoS) OR | Enzymatic activity |
| | | (NRI_hi AND RpoN)) | |
| b1814 | sdaA | ((gly(e) > 0 OR leu-L(e) > 0 OR NOT | Enzymatic activity |
| | | (o2(e) > 0)) AND ((NOT Lrp) OR (Lrp | |
| | | AND leu-L(e) > 0))) | |
| b1896 | otsA | (RpoS) | Enzymatic activity |
| b1897 | otsB | (RpoS) | Enzymatic activity |
| b1898 | araH_2 | (AraC OR (AraC AND Crp)) | Regulation |
| b1899 | araH_1 | (AraC OR (AraC AND Crp)) | Regulation |
| b1900 | araG | (AraC OR (AraC AND Crp)) | Enzymatic activity |
| b1901 | araF | (AraC OR (AraC AND Crp)) | Enzymatic activity |
| b1987 | cbl | (NOT ((so4(e) > 0) OR (cys-L(e) > 0)) | Regulation |
| | | AND CysB) | |
| b1988 | nac | (NRI_low AND RpoN) | Regulation |
| b1991 | cobT | (cbi(e) > 0) | Enzymatic activity |
| b1992 | cobS | (cbi(e) > 0) | Enzymatic activity |
| b1933 | cobU | (cbi(e) > 0) | Enzymatic activity |
| b2087 | gatR_1 | (NOT (galt(e) > 0)) | Regulation |
| b2090 | gatR_2 | (NOT (galt(e) > 0)) | Regulation |
| b2097 | fbaB | ((pyr(e) > 0) OR (lac-D(e) > 0) AND | Enzymatic activity |
| | | NOT(glc-D(e) > 0)) | |
| b2143 | cdd | (Crp AND NOT (CytR)) | Enzymatic activity |
| b2148 | mglC | (Crp AND NOT (GalS)) | Enzymatic activity |
| b2149 | mglA | (Crp AND NOT (GalS)) | Enzymatic activity |
| b2150 | mglB | (Crp AND NOT (GalS)) | Enzymatic activity |
| b2219 | atoS | (acac(e) > 0) | Regulation |
| b2220 | atoC | (AtoS) | Regulation |
| b2221 | atoD | (AtoC) | Enzymatic activity |
| b2222 | atoA | (AtoC) | Enzymatic activity |
| b2223 | atoE | (AtoC) | Enzymatic activity |
| b2224 | atoB | (AtoC) | Enzymatic activity |
| b2232 | ublG | ((o(e) > 0) AND Crp) | Enzymatic activity |
| b2239 | glpQ | ((NOT GlpR OR Fnr) AND Crp) | Enzymatic activity |
| b2240 | glpT | (NOT (GlpR) AND Crp) | Enzymatic activity |
| b2241 | glpA | (“CRP noRIB” AND (Fnr OR ArcA) AND | Enzymatic activity |
| | | (NOT(GlpR))) | |
| b2242 | glpB | (“CRP noRIB” AND (Fnr OR ArcA) AND | Enzymatic activity |
| | | (NOT(GlpR))) | |
| b2243 | glpC | (“CRP noRIB” AND (Fnr OR ArcA) AND | Enzymatic activity |
| | | (NOT(GlpR))) | |
| b2364 | dsdC | (ser-D(e) > 0) | Regulation |
| b2366 | dsdA | ((Crp AND DsdC) OR DsdC) | Enzymatic activity |
| b2405 | xapR | (xtsn(e) > 0) | Regulation |
| b2406 | xapB | (XapR) | Enzymatic activity |
| b2407 | xapA | (XapR) | Enzymatic activity |
| b2458 | eutD | (ethanolamine(e) > 0) | Regulation |
| b2492 | focB | (ArcA OR Fnr AND (Crp OR NOT | Enzymatic activity |
| | | (NarL))) | |
| b2537 | hcaR | (pppn(e) > 0) | Regulation |
| b2538 | hcaE | (HcaR AND (NOT(“LBMedia”))) | Enzymatic activity |
| b2539 | hcaF | (HcaR AND (NOT(“LBMedia”))) | Enzymatic activity |
| b2540 | hcaC | (HcaR AND (NOT(“LBMedia”))) | Enzymatic activity |
| b2541 | hcaB | (HcaR AND (NOT(“LBMedia”))) | Enzymatic activity |
| b2542 | hcaD | (HcaR AND (NOT(“LBMedia”))) | Enzymatic activity |
| b2564 | pdxJ | (RpoE) | Regulation |
| b2573 | rpoE | (“heat shock”) | Regulation |
| b2600 | tyrA | (NOT(((phe-L(e) > 10) OR (tyr-L(e) > 0)) | Regulation |
| | | AND TyR)) | |
| b2601 | aroF | (NOT(((phe-L(e) > 10) OR (tyr-L(e) > 0)) | Enzymatic activity |
| | | AND TyrR)) | |
| b2702 | srlA | ((NOT GutR) AND “CRP noGL”) | Enzymatic activity |
| b2703 | srlE | ((NOT GutR) AND “CRP noGL”) | Enzymatic activity |
| b2704 | srlB | ((NOT GutR) AND “CRP noGL”) | Enzymatic activity |
| b2705 | srlD | (GutM AND (NOT GutR) AND “CRP | Enzymatic activity |
| | | noGL”) | |
| b2719 | hycG | (FhlA AND RpoN AND (NOT (o2(e) > | Enzymatic activity |
| | | 0))) | |
| b2720 | hycF | (FhlA AND RpoN AND (NOT (o2(e) > | Enzymatic activity |
| | | 0))) | |
| b2721 | hycE | (FhlA AND RpoN AND (NOT (o2(e) > | Enzymatic activity |
| | | 0))) | |
| b2722 | hycD | (FhlA AND RpoN AND (NOT (o2(e) > | Enzymatic activity |
| | | 0))) | |
| b2723 | hycC | (FhlA AND RpoN AND (NOT (o2(e) > | Enzymatic activity |
| | | 0))) | |
| b2724 | hycB | (FhlA AND RpoN AND (NOT (o2(e) > | Enzymatic activity |
| | | 0))) | |
| b2731 | fhlA | ((NOT (o2(e) > 0)) AND (NOT (no3(e) > | Regulation |
| | | 0)) AND (NOT (no2(e) > 0)) AND | |
| | | (NOT (tmao(e) > 0)) AND (NOT | |
| | | (dmso(e) > 0)) AND (for(e) > 0)) | |
| b2741 | rpoS | (NOT (Growth > 0)) | Regulation |
| b2787 | gudD | (SdaR) | Enzymatic activity |
| b2796 | sdaC | (Crp OR (NOT (Lrp) AND (leu-L(e) > 0) | Enzymatic activity |
| | | AND Crp)) | |
| b2799 | fucO | (((((FucR) OR (rmn(e) > 0)) AND (NOT | Enzymatic activity |
| | | (o2(e) > 0))) AND Crp) OR (((FucR) OR | |
| | | (rmn(e) > 0)) AND (NOT (o2(e) > 0)))) | |
| b2800 | fucA | ((FucR AND Crp) OR FucR) | Enzymatic activity |
| b2801 | fucP | ((FucR AND Crp) OR FucR) | Enzymatic activity |
| b2802 | fucI | ((FucR AND Crp) OR FucR) | Enzymatic activity |
| b2803 | fucK | ((FucR AND Crp) OR FucR) | Enzymatic activity |
| b2805 | fucR | (fuc-L(e) > 0) | Regulation |
| b2808 | gcvA | (NOT GcvR) | Regulation |
| b2841 | araE | (Crp) | Enzymatic activity |
| b2927 | epd | (Crp) | Regulation |
| b2957 | ansB | (Fnr AND Crp) | Enzymatic activity |
| b2975 | glcA | (NOT ArcA AND GlcC) | Enzymatic activity |
| b2976 | glcB | (NOT (ArcA) AND (GlcC)) | Enzymatic activity |
| b2980 | glcC | ((ac(e) > 0) OR (glyclt(e) > 0)) | Regulation |
| b2994 | hybC | (FhlA AND RpoN AND (NOT (o2(e) > | Enzymatic activity |
| | | 0))) | |
| b2997 | hybO | (FhlA AND RpoN AND (NOT (o2(e) > | Enzymatic activity |
| | | 0))) | |
| b3061 | ttdA | (NOT(o2(e) > 0) AND (tartr-L(e) > 0)) | Enzymatic activity |
| b3062 | ttdB | (NOT(o2(e) > 0) AND (tartr-L(e) > 0)) | Enzymatic activity |
| b3091 | uxaA | (NOT ExuR) | Enzymatic activity |
| b3092 | uxaC | (NOT ExuR) | Enzymatic activity |
| b3093 | exuT | (NOT ExuR) | Enzymatic activity |
| b3094 | exuR | (NOT(GUI1 > 0) AND NOT(GUI2 > 0) | Regulation |
| | | AND NOT(MANAO > 0) AND | |
| | | NOT(TAGURr > 0) AND NOT(GUI1 < 0) | |
| | | AND NOT(GUI2 < 0) AND | |
| | | NOT(MANAO < 0) AND | |
| | | NOT(TAGURr < 0) | |
| b3111 | tdcGa | (Crp OR NOT(o2(e) > 0)) | Regulation |
| b3112 | tdcGb | (Crp OR NOT(o2(e) > 0)) | Regulation |
| b3118 | tdcA | ((thr-L(e) > 0) AND (ser-L(e) > 0) AND | Regulation |
| | | (val-L(e) > 0) AND (Ile-L(e) > 0) AND | |
| | | NOT (o2(e) > 0)) | |
| b3119 | tdcR | ((thr-L(e) > 0) AND (ser-L(e) > 0) AND | Regulation |
| | | (val-L(e) > 0) AND (Ile-L(e) > 0) AND | |
| | | NOT (o2(e) > 0)) | |
| b3124 | garK | (SdaR) | Enzymatic activity |
| b3125 | garR | (SdaR) | Enzymatic activity |
| b3126 | garL | (SdaR) | Enzymatic activity |
| b3128 | garD | (SdaR) | Enzymatic activity |
| b3237 | argR | (arg-L(e) > 0) | Regulation |
| b3357 | crp | (“CRP noGLC”) | Regulation |
| b3365 | nirB | (Fnr AND NarL) | Enzymatic activity |
| b3366 | nirD | (Fnr AND NarL) | Enzymatic activity |
| b3367 | nirC | (Fnr AND NarL) | Enzymatic activity |
| b3368 | cysG | (Fnr OR NarL) | Regulation |
| b3405 | ompR | (“high osmolarity”) | Regulation |
| b3415 | gntT | (NOT (GntR) AND “CRP GLCN”) | Enzymatic activity |
| b3416 | malQ | (MalT) | Enzymatic activity |
| b3417 | malP | (MAlT) | Enzymatic activity |
| b3418 | malT | ((malt(e) > 0) OR (malttr(e) > 0) OR | Regulation |
| | | (maltttr(e) > 0) OR (malthx(e) > 0) OR | |
| | | (maltpt(e) > 0)) | |
| b3425 | glpE | (Crp) | Enzymatic activity |
| b3426 | glpD | (“CRP noMAL” AND (NOT(ArcA OR | Enzymatic activity |
| | | GlpR))) | |
| b3428 | glgP | (Crp) | Enzymatic activity |
| b3437 | gntK | (NOT (GntR) AND “CRP GLCN”) | Enzymatic activity |
| b3450 | ugpC | (Crp OR PhoB) | Enzymatic activity |
| b3451 | ugpE | (Crp OR PhoB) | Enzymatic activity |
| b3452 | ugpA | (Crp OR PhoB) | Enzymatic activity |
| b3453 | ugpB | (Crp OR PhoB) | Enzymatic activity |
| b3454 | livF | (NOT(leu-L(e) > 0) OR Lrp) | Enzymatic activity |
| b3455 | livG | (NOT(leu-L(e) > 0) OR Lrp | Enzymatic activity |
| b3456 | livM | (NOT(leu-L(e) > 0) OR Lrp) | Enzymatic activity |
| b3457 | livH | (NOT(leu-L(e) > 0) OR Lrp) | Enzymatic activity |
| b3458 | livK | (NOT(leu-L(e) > 0) OR Lrp) | Enzymatic activity |
| b3460 | livJ | (NOT(leu-L(e) > 0) OR Lrp) | Enzymatic activity |
| b3500 | gor | (OxyR OR RpoS) | Enzymatic activity |
| b3517 | gadA | ((NOT (Growth > 0) AND NOT Crp) | Enzymatic activity |
| | | OR (pH < 4)) | |
| b3519 | treF | (RpoS) | Enzymatic activity |
| b3526 | kdgK | (NOT KdgR) | Enzymatic activity |
| b3528 | dctA | (((“CRP noMAN”) AND NOT(ArcA) | Enzymatic activity |
| | | AND (DcuR)) AND RpoN) | |
| b3564 | xylB | ((XylR AND Crp) OR XylR) | Enzymatic activity |
| b3565 | xylA | ((XylR AND Crp) OR XylR) | Enzymatic activity |
| b3566 | xylF | ((XylR AND Crp) OR XylR) | Enzymatic activity |
| b3567 | xylG | ((XylR AND Crp) OR XylR) | Enzymatic activity |
| b3568 | xylH | ((XylR AND Crp) OR XylR) | Enzymatic activity |
| b3569 | xylR | (xyl-D(e) > 0) | Regulation |
| b3575 | yiaK | ((NOT YiaJ) AND Crp) | Enzymatic activity |
| b3581 | sgbH | (NOT YiaJ) | Enzymatic activity |
| b3583 | sgbE | (NOT YiaJ) | Enzymatic activity |
| b3588 | aldB | (RpoS AND (Crp)) | Enzymatic activity |
| b3599 | mtlA | (NOT (MtlR)) | Enzymatic activity |
| b3600 | mtlD | (NOT MtlR) | Enzymatic activity |
| b3605 | lidD | (lac-L(e) > 0 AND o2(e) > 0) | Enzymatic activity |
| b3616 | tdh | (NOT (Lrp) AND (leu-L(e) > 0)) | Enzymatic activity |
| b3617 | kbl | (NOT (Lrp) AND (leu-L(e) > 0)) | Enzymatic activity |
| b3653 | gltS | (asp-L(e) > 0)) | Enzymatic activity |
| b3666 | uhpT | (Crp OR UhpA | Enzymatic activity |
| b3668 | uhpB | (g6p(e) > 0) | Regulation |
| b3669 | uhpA | (UhpB) | Regulation |
| b3670 | livN | (NOT(leu-L(e) > 0 OR val-L(e) > 0) | Enzymatic activity |
| | | AND Crp) | |
| b3671 | livB | (NOT(leu-L(e) > 0 OR val-L(e) > 0) | Enzymatic activity |
| | | AND Crp) | |
| b3691 | dgoT | (galctn-D(e) > 0) | Enzymatic activity |
| b3692 | dgoA | (galctn-D(e) > 0) | Enzymatic activity |
| b3693 | dgoK | (galctn-D(e) > 0) | Enzymatic activity |
| b3708 | tnaA | (Crp AND (trp-L(e) > 0 OR cys-L(e) > 0)) | Enzymatic activity |
| b3709 | tnaB | (Crp AND (trp-L(e) > 0)) | Enzymatic activity |
| b3725 | pstB | (PhoB) | Enzymatic activity |
| b3726 | pstA | (PhoB) | Enzymatic activity |
| b3727 | pstC | (PhoB) | Enzymatic activity |
| b3728 | pstS | (PhoB) | Enzymatic activity |
| b3748 | rbsD | (“CRP noXYL” AND NOT(RbsR)) | Enzymatic activity |
| b3749 | rbsA | (“CRP noXYL” AND NOT(RbsR)) | Enzymatic activity |
| b3750 | rbsC | (“CRP noXYL” AND NOT(RbsR)) | Enzymatic activity |
| b3751 | rbsB | (“CRP noXYL” AND NOT(RbsR)) | Enzymatic activity |
| b3752 | rbsK | (“CRP noXYL” AND NOT(RbsR)) | Enzymatic activity |
| b3753 | rbsR | (NOT (rib-D(e) > 0)) | Regulation |
| b3767 | ilvG_1 | (NOT(leu-L(e) > 0 OR ile-L(e) > 0 OR | Regulation |
| | | val-L(e) > 0) AND Lrp) | |
| b3768 | ilvG_2 | (NOT(leu-L(e) > 0 OR ile-L(e) > 0 OR | Regulation |
| | | val-L(e) > 0) AND Lrp) | |
| b3870 | glnA | (Crp AND RpoN) | Enzymatic activity |
| b3902 | rhaD | (RhaS OR (RhaS AND Crp)) | Enzymatic activity |
| b3903 | rhaA | (RhaS OR (RhaS AND Crp)) | Enzymatic activity |
| b3904 | rhaB | (RhaS OR (RhaS AND Crp)) | Enzymatic activity |
| b3905 | rhaS | (RhaR) | Regulation |
| b3906 | rhaR | (rmn(e) > 0) | Regulation |
| b3907 | rhaT | (RhaS OR (RhaS AND Crp)) | Enzymatic activity |
| b3908 | sodA | (NOT (ArcA OR Fur) OR (MarA OR Rob | Enzymatic activity |
| | | OR SoxS)) | |
| b3909 | kdgT | (NOT KdgR) | Enzymatic activity |
| b3912 | cpxR | (“Stress”) | Regulation |
| b3926 | glpK | (“CRP noMAL” AND (NOT(GlpR))) | Enzymatic activity |
| b3934 | cytR | (cytd(e) > 0) | Regulation |
| b3938 | metI | (met-L(e) > 0) | Regulation |
| b3951 | pflD | (ArcA OR Fnr) | Enzymatic activity |
| b3952 | pflC | (ArcA OR Fnr) | Enzymatic activity |
| b3961 | oxyR | (h2o2(e) > 0) | Regulation |
| b3973 | birA | (btn(e) > 0) | Regulation |
| b4014 | aceB | (NOT (IclR) AND (NOT (ArcA) OR NOT | Enzymatic activity |
| | | (Cra))) | |
| b4015 | aceA | (NOT (IclR) AND (NOT (ArcA) OR NOT | Enzymatic activity |
| | | (Cra))) | |
| b4031 | xylE | (xylR) | Enzymatic activity |
| b4032 | malG | ((MalT AND Crp) OR MalT) | Enzymatic activity |
| b4033 | malF | ((MalT AND Crp) OR MalT) | Enzymatic activity |
| b4034 | malE | ((MalT AND Crp) OR MalT) | Enzymatic activity |
| b4035 | malK | ((MalT AND Crp) OR MalT) | Enzymatic activity |
| b4036 | lamB | ((MalT AND Crp) OR MalT) | Enzymatic activity |
| b4039 | ubiC | ((NOT Fnr) AND Crp) | Regulation |
| b4040 | ubiA | ((NOT Fnr) AND Crp) | Regulation |
| b4054 | tyrB | (NOT(((phe-L(e) > 0) OR (tyr-L(e) > 0)) | Enzymatic activity |
| | | AND TyrR)) | |
| b4062 | soxS | (SoxR) | Regulation |
| b4063 | soxR | ((h2o2(e) > 0) OR (“Oxidative | Regulation |
| | | Stress”)) | |
| b4069 | acs | (RpoS OR Fnr OR ((NOT IclR) AND | Enzymatic activity |
| | | (“CRP noSUCC”))) | |
| b4079 | fdhF | (FhlA AND RpoN AND (NOT (o2(e) > | Enzymatic activity |
| | | 0))) | |
| b4090 | rpiB | (NOT(RpiR)) | Enzymatic activity |
| b4111 | proP | (NOT (Crp) AND Fis AND RpoS) | Enzymatic activity |
| b4116 | adiY | ((pH < 7) AND NOT (o2(e) > 0)) AND | Regulation |
| | | NOT (“Rich Medium”)) | |
| b4117 | adiA | (AdiY) | Enzymatic activity |
| b4118 | melR | ((melib(e) > 0) OR (melib(e) > 0 AND | Regulation |
| | | Crp)) | |
| b4119 | melA | ((MelR) OR (MelR AND Crp)) | Enzymatic activity |
| b4120 | melB | ((MelR) OR (MelR AND Crp)) | Enzymatic activity |
| b4123 | dcuB | (((“CRP noMAN”) AND (Fnr) AND | Enzymatic activity |
| | | (DcuR)) AND NOT (NarL)) | |
| b4124 | dcuR | (DcuS) | Regulation |
| b4125 | dcuS | ((succ(e) > 0) OR (asp-L(e) > 0) OR | Regulation |
| | | (fum(e) > 0) OR (mal-L(e) > 0)) | |
| b4131 | cadA | (ArcA OR CadC) | Enzymatic activity |
| b4132 | cadB | (ArcA OR CadC) | Enzymatic activity |
| b4133 | cadC | (lys-L(e) > 0) | Regulation |
| b4139 | aspA | ((Crp AND NOT (Fnr)) OR Fnr) | Enzymatic activity |
| b4151 | frdD | (Fnr OR DcuR OR NOT (NarL)) | Enzymatic activity |
| b4152 | frdC | (Fnr OR DcuR OR NOT (NarL)) | Enzymatic activity |
| b4153 | frdB | (Fnr OR DcuR OR NOT (NarL)) | Enzymatic activity |
| b4154 | frdA | (Fnr OR DcuR OR NOT (NarL)) | Enzymatic activity |
| b4238 | nrdD | (Fnr) | Enzymatic activity |
| b4264 | idnR | ((idon-L(e) > 0) OR (Sdglcn(e) > 0)) | Regulation |
| b4265 | idnT | (IdnR) | Enzymatic activity |
| b4266 | idnO | (IdnR) | Enzymatic activity |
| b4267 | idnD | (IdnR) | Enzymatic activity |
| b4321 | gntP | (Crp AND NOT (glcn(e) > 0)) | Enzymatic activity |
| b4322 | uxuA | (NOT ExuR AND NOT UxuR) | Enzymatic activity |
| b4323 | uxuB | (NOT ExuR AND NOT UxuR) | Enzymatic activity |
| b4324 | uxuR | (NOT(MANAO > 0) AND NOT(GUI1 > 0) | Regulation |
| | | AND NOT(MANAO < 0) AND | |
| | | NOT(GUI1 > 0)) | |
| b4382 | deoA | (NOT (DeoR OR CytR) AND Crp) | Enzymatic activity |
| b4390 | nadR | (“high NAD”) | Enzymatic activity |
| b4393 | trpR | (trp-L(e) > 0) | Regulation |
| b4396 | rob | (“dipyridyl”) | Regulation |
| b4401 | arcA | (NOT (o2(e) > 0)) | Regulation |

Note:
regulatory rules adapted from Covert and colleagues, except where indicated below
Note:
b2458 regulation was added (PMID: 21046341)
Note:
b3942 regulation was changed to allow catalase activity under normal growth, original regulatory rules prevent KatG expression under all conditions, glucose media
Note:
b0506 regulation was changed to (NOT(glyoxalate(e) > 0)) (PMID: 10978349)
Note:
b2087 insertional mutation renders regulator inactive

TABLE 6
ROS/BM for genetic selections that altered ROS flux

| | wild-type | b0114 | b0116 | b0171 | b0242 | b0243 |
|---|---|---|---|---|---|---|
| Exponential Superoxide | | | | | | |
| Mean | 0.048 | 0.063 | 0.064 | 0.048 | 0.048 | 0.048 |
| Relative Mean | 1.00 | 1.32 | 1.34 | 1.00 | 1.00 | 1.00 |
| Exponential Peroxide | | | | | | |
| Mean | 0.133 | 0.179 | 0.181 | 0.133 | 0.134 | 0.134 |
| Relative Mean | 1.00 | 1.34 | 1.36 | 1.00 | 1.00 | 1.00 |
| Gaussian Superoxide | | | | | | |
| Mean | 0.048 | 0.063 | 0.063 | 0.048 | 0.048 | 0.048 |
| Relative Mean | 1.00 | 1.32 | 1.33 | 1.00 | 1.00 | 1.00 |
| Gaussian Peroxide | | | | | | |
| Mean | 0.133 | 0.175 | 0.177 | 0.133 | 0.134 | 0.134 |
| Relative Mean | 1.00 | 1.31 | 1.33 | 1.00 | 1.00 | 1.00 |

| | wild-type | b0429 | b0721 | b0726 | b0728 | b0767 | b0910 | b1091 | b1761 |
|---|---|---|---|---|---|---|---|---|---|
| Exponential Superoxide | | | | | | | | | |
| Mean | 0.048 | 0.064 | 0.050 | 0.050 | 0.049 | 0.053 | 0.048 | 0.048 | 0.057 |
| Relative Mean | 1.00 | 1.34 | 1.05 | 1.05 | 1.03 | 1.11 | 1.00 | 1.00 | 1.19 |
| Exponential Peroxide | | | | | | | | | |
| Mean | 0.133 | 0.146 | 0.143 | 0.143 | 0.141 | 0.145 | 0.134 | 0.134 | 0.158 |
| Relative Mean | 1.00 | 1.09 | 1.08 | 1.08 | 1.05 | 1.09 | 1.00 | 1.00 | 1.18 |
| Gaussian Superoxide | | | | | | | | | |
| Mean | 0.048 | 0.059 | 0.051 | 0.051 | 0.050 | 0.051 | 0.048 | 0.048 | 0.056 |
| Relative Mean | 1.00 | 1.23 | 1.07 | 1.07 | 1.05 | 1.08 | 1.00 | 1.00 | 1.17 |
| Gaussian Peroxide | | | | | | | | | |
| Mean | 0.133 | 0.154 | 0.142 | 0.142 | 0.140 | 0.144 | 0.134 | 0.134 | 0.156 |
| Relative Mean | 1.00 | 1.15 | 1.07 | 1.07 | 1.05 | 1.08 | 1.00 | 1.00 | 1.17 |
| | wild-type | b1852 | b2029 | b2276 | b2297 | b2415 | b2436 | b2500 | b2501 |
| Exponential Superoxide | | | | | | | | | |
| Mean | 0.048 | 0.053 | 0.053 | 0.051 | 0.051 | 0.049 | 0.048 | 0.048 | 0.049 |
| Relative Mean | 1.00 | 1.11 | 1.11 | 1.29 | 1.08 | 1.03 | 1.00 | 1.00 | 1.03 |
| Exponential Peroxide | | | | | | | | | |
| Mean | 0.133 | 0.145 | 0.145 | 0.150 | 0.142 | 0.137 | 0.133 | 0.134 | 0.137 |
| Relative Mean | 1.00 | 1.09 | 1.09 | 1.12 | 1.06 | 1.03 | 1.00 | 1.00 | 1.03 |
| Gaussian Superoxide | | | | | | | | | |
| Mean | 0.018 | 0.051 | 0.051 | 0.064 | 0.050 | 0.049 | 0.048 | 0.048 | 0.049 |
| Relative Mean | 1.00 | 1.08 | 1.08 | 1.33 | 1.06 | 1.03 | 1.00 | 1.00 | 1.03 |
| Gaussian Peroxide | | | | | | | | | |
| Mean | 0.133 | 0.144 | 0.144 | 0.164 | 0.141 | 0.137 | 0.133 | 0.134 | 0.137 |
| Relative Mean | 1.00 | 1.08 | 1.08 | 1.23 | 1.06 | 1.03 | 1.00 | 1.00 | 1.03 |

| | wild-type | b3919 | b4025 | b4208 | b4388 |
|---|---|---|---|---|---|
| Exponential Superoxide | | | | | |
| Mean | 0.048 | 0.056 | 0.049 | 0.048 | 0.052 |
| Relative Mean | 1.00 | 1.19 | 1.04 | 1.00 | 1.08 |
| Exponential Peroxide | | | | | |
| Mean | 0.133 | 0.156 | 0.141 | 0.133 | 0.145 |
| Relative Mean | 1.00 | 1.17 | 1.06 | 1.00 | 1.08 |
| Gaussian Superoxide | | | | | |
| Mean | 0.048 | 0.055 | 0.050 | 0.048 | 0.052 |
| Relative Mean | 1.00 | 1.16 | 1.05 | 1.00 | 1.09 |
| Gaussian Peroxide | | | | | |
| Mean | 0.133 | 0.155 | 0.140 | 0.133 | 0.145 |
| Relative Mean | 1.00 | 1.16 | 1.05 | 1.00 | 1.08 |

Notes:
b0114, b0115 deletions result in identical metabolic networks
b0429, b0430, b0431, b0432 deletions result in identical metabolic networks
b0721, b0722, b0723, b0724 deletions result in identical metabolic networks
b0726, b0727 deletions result in identical metabolic networks
b0728, b0729 deletions result in identical metabolic networks
b2276, b2277, b2278, b2279, b2280, b2281, b2282, b2283, b2284, b2285, b2286, b2287, b2288 deletions result in identical metabolic networks
b2415, b2416 deletions result in identical metabolic networks
b2903, b2904, b2905 deletions result in identical metabolic networks
b2913, b2914 deletions result in identical metabolic networks
b3731, b3732, b3733, b3734, b3735, b3736, b3737, b3738, b3739 deletions result in identical metabolic networks

### REFERENCES

1. Arias, C. A. & Murray, B. E. Antibiotic-resistant bugs in the 21st century—a clinical super-challenge. N Engl J Med 360, 439-443 (2009).
2. Boucher, H. W. et al. Bad bugs, no drugs: no ESKAPE! An update from the Infectious Diseases Society of America. Clin Infect Dis 48, 1-12 (2009).
3. Cottarel, G. & Wierzbowski, J. Combination drugs, an emerging option for antibacterial therapy. Trends Biotechnol 25, 547-555 (2007).
4. Council, N. R. Treating Infectious Diseases in a Microbial World: Report of Two Workshops on Novel Antimicrobial Therapeutics. (National Academy Press, Washington, D.C.; 2006).
5. Escaich, S. Antivirulence as a new antibacterial approach for chemotherapy. Curr Opin Chem Biol 12, 400-408 (2008).
6. Kohanski, M. A., Dwyer, D. J., Hayete, B., Lawrence, C. A. & Collins, J. J. A common mechanism of cellular death induced by bactericidal antibiotics. Cell 130, 797-810 (2007).
7. Imlay, J. A. Pathways of oxidative damage. Annu Rev Microbiol 57, 395-418 (2003).
8. Carlioz, A. & Touati, D. Isolation of superoxide dismutase mutants in Escherichia coli: is superoxide dismutase necessary for aerobic life? EMBO J 5, 623-630 (1986).
9. Loewen, P. C. Isolation of catalase-deficient Escherichia coli mutants and genetic mapping of katE, a locus that affects catalase activity. J Bacteriol 157, 622-626 (1984).
10. Dwyer, D. J., Kohanski, M. A., Hayete, B. & Collins, J. J. Gyrase inhibitors induce an oxidative damage cellular death pathway in Escherichia coli. Mol Syst Biol 3, 91 (2007).
11. Wang, X. & Zhao, X. Contribution of oxidative damage to antimicrobial lethality. Antimicrob Agents Chemother 53, 1395-1402 (2009).
12. Barczak, A. K. & Hung, D. T. Productive steps toward an antimicrobial targeting virulence. Curr Opin Microbiol 12, 490-496 (2009).
13. Hebrard, M., Viala, J. P., Meresse, S., Barras, F. & Aussel, L. Redundant hydrogen peroxide scavengers contribute to Salmonella virulence and oxidative stress resistance. J Bacteriol 191, 4605-4614 (2009).
14. Liu, C. I. et al. A cholesterol biosynthesis inhibitor blocks Staphylococcus aureus virulence. Science 319, 1391-1394 (2008).
15. Gonzalez-Flecha, B. & Demple, B. Metabolic sources of hydrogen peroxide in aerobically growing Escherichia coli. J Biol Chem 270, 13681-13687 (1995).
16. Imlay, J. A. & Fridovich, I. Assay of metabolic superoxide production in Escherichia coli. J Biol Chem 266, 6957-6965 (1991).
17. Messner, K. R. & Imlay, J. A. The identification of primary sites of superoxide and hydrogen peroxide formation in the aerobic respiratory chain and sulfite reductase complex of Escherichia coli. J Biol Chem 274, 10119-10128 (1999).
18. Korshunov, S. & Imlay, J. A. Two sources of endogenous hydrogen peroxide in Escherichia coli. Mol Microbiol 75, 1389-1401 (2010).
19. Korshunov, S. & Imlay, J. A. Detection and quantification of superoxide formed within the periplasm of Escherichia coli. J Bacteriol 188, 6326-6334 (2006).
20. Massey, V. Activation of molecular oxygen by flavins and flavoproteins. J Biol Chem 269, 22459-22462 (1994).
21. Seaver, L. C. & Imlay, J. A. Are respiratory enzymes the primary sources of intracellular hydrogen peroxide? J Biol Chem 279, 48742-48750 (2004).
22. Feist, A. M. & Palsson, B. O. The growing scope of applications of genome-scale metabolic reconstructions using Escherichia coli. Nat Biotechnol 26, 659-667 (2008).
24. Mahadevan, R., Palsson, B. O. & Lovley, D. R. In situ to in silico and back: elucidating the physiology and ecology of Geobacter spp. using genome-scale modelling. Nat Rev Microbiol 9, 39-50 (2011).
25. Oberhardt, M. A., Palsson, B. O. & Papin, J. A. Applications of genome-scale metabolic reconstructions. Mol Syst Biol 5, 320 (2009).
26. Karr, J. R. et al. A whole-cell computational model predicts phenotype from genotype. Cell 150, 389-401 (2012).
27. Lerman, J. A. et al. In silico method for modelling metabolism and gene product expression at genome scale. Nat Commun 3, 929 (2012).
28. Becker, S. A. et al. Quantitative prediction of cellular metabolism with constraint-based models: the COBRA Toolbox. Nat Protoc 2, 727-738 (2007).
29. Orth, J. D., Thiele, I. & Palsson, B. O. What is flux balance analysis? Nat Biotechnol 28, 245-248 (2010).
30. Becker, S. A. & Palsson, B. O. Context-specific metabolic networks are consistent with experiments. PLoS Comput Biol 4, e1000082 (2008).
31. Covert, M. W., Knight, E M., Reed, J. L., Herrgard, M. J. & Palsson, B. O. Integrating high-throughput and computational data elucidates bacterial networks. Nature 429, 92-96 (2004).
32. Feist, A. M. et al. A genome-scale metabolic reconstruction for Escherichia coli K-12 MG1655 that accounts for 1260 ORFs and thermodynamic information. Mol Syst Biol 3, 121 (2007).
33. Feist, A. et al. A genome-scale metabolic reconstruction for Escherichia coli K-12 MG1655 that accounts for 1260 ORFs and thermodynamic information. Molecular systems biology [electronic resource] 3, 121 (2007).
34. Imlay, J. A. A metabolic enzyme that rapidly produces superoxide, fumarate reductase of Escherichia coli. J Biol Chem 270, 19767-19777 (1995).
35. Messner, K. R. & Imlay, J. A. Mechanism of superoxide and hydrogen peroxide formation by fumarate reductase, succinate dehydrogenase, and aspartate oxidase. J Biol Chem 277, 42563-42571 (2002).
36. Messner, K. R. & Imlay, J. A. In vitro quantitation of biological superoxide and hydrogen peroxide generation. Methods Enzymol 349, 354-361 (2002).
37. Edwards, J. S., Ibarra, R. U. & Palsson, B. O. In silico predictions of Escherichia coli metabolic capabilities are consistent with experimental data. Nat Biotechnol 19, 125-130 (2001).
38. Seaver, L. C. & Imlay, J. A. Alkyl hydroperoxide reductase is the primary scavenger of endogenous hydrogen peroxide in Escherichia coli. J Bacteriol 183, 7173-7181 (2001).
39. Mahadevan, R. & Schilling, C. H. The effects of alternate optimal solutions in constraint-based genome-scale metabolic models. Metab Eng 5, 264-276 (2003).
40. Keseler, I. M. et al. EcoCyc: a comprehensive view of Escherichia coli biology. Nucleic Acids Res 37, D464-470 (2009).
41. Abdel-Hamid, A. M., Attwood, M. M. & Guest, J. R. Pyruvate oxidase contributes to the aerobic growth efficiency of Escherichia coli. Microbiology 147, 1483-1498 (2001).
42. Anderson, A. & Cooper, R. A. Gluconeogenesis in Escherichia coli The role of triose phosphate isomerase. FEBS Lett 4, 19-20 (1969).
43. Chang, Y. Y. & Cronan, J. E., Jr. Genetic and biochemical analyses of Escherichia coli strains having a mutation in the structural gene (poxB) for pyruvate oxidase. J Bacteriol 154, 756-762 (1983).
44. Henning, U., Dennert, G., Hertel, R. & Shipp, W. S. Translation of the structural genes of the E. coli pyruvate dehydrogenase complex. Cold Spring Harb Symp Quant Biol 31, 227-234 (1966).
45. Irani, M. H. & Maitra, P. K. Properties of Escherichia coli mutants deficient in enzymes of glycolysis. J Bacteriol 132, 398-410 (1977).
46. Ravnikar, P. D. & Somerville, R. L. Genetic characterization of a highly efficient alternate pathway of serine biosynthesis in Escherichia coli. J Bacteriol 169, 2611-2617 (1987).
47. Belousov, V. V. et al. Genetically encoded fluorescent indicator for intracellular hydrogen peroxide. Nat Methods 3, 281-286 (2006).
48. Malinouski, M., Zhou, Y., Belousov, V. V., Hatfield, D. L. & Gladyshev, V. N. Hydrogen peroxide probes directed to different cellular compartments. PLoS One 6, e14564 (2011).
48. Imlay, J. A. Cellular defenses against superoxide and hydrogen peroxide. Annu Rev Biochem 77, 755-776 (2008).
49. Winter, J., Ilbert, M., Graf, P. C., Ozcelik, D. & Jakob, U. Bleach activates a redox-regulated chaperone by oxidative protein unfolding. Cell 135, 691-701 (2008).
50. Taber, H. W., Mueller, J. P., Miller, P. F. & Arrow, A. S. Bacterial uptake of aminoglycoside antibiotics. Microbiol Rev 51, 439-457 (1987).
51. Jensen, P. R. & Michelsen, 0. Carbon and energy metabolism of atp mutants of Escherichia coli. J Bacteriol 174, 7635-7641 (1992).
52. Yeh, P. & Kishony, R. Networks from drug-drug surfaces. Mol Syst Biol 3, 85 (2007).
53. Hegreness, M., Shoresh, N., Damian, D., Hartl, D. & Kishony, R. Accelerated evolution of resistance in multidrug environments. Proc Natl Acad Sci USA 105, 13977-13981 (2008).
54. US EPA: R.E.D. Facts-Carboxin, Vol. EPA 738-F-04-0102004).
55. Boshoff, H. I. et al. The transcriptional responses of Mycobacterium tuberculosis to inhibitors of metabolism: novel insights into drug mechanisms of action. J Biol Chem 279, 40174-40184 (2004).
56. Weber, W. et al. A synthetic mammalian gene circuit reveals antituberculosis compounds. Proc Natl Acad Sci USA 105, 9994-9998 (2008).
57. Schilling, C. H. & Palsson, B. O. Assessment of the metabolic capabilities of Haemophilus influenzae Rd through a genome-scale pathway analysis. J Theor Biol 203, 249-283 (2000).
58. AbuOun, M. et al. Genome scale reconstruction of a Salmonella metabolic model: comparison of similarity and differences with a commensal Escherichia coli strain. J Biol Chem 284, 29480-29488 (2009).
59. Jamshidi, N. & Palsson, B. O. Investigating the metabolic capabilities of Mycobacterium tuberculosis H37Rv using the in silico strain iNJ661 and proposing alternative drug targets. BMC Syst Biol 1, 26 (2007).
60. Becker, S. A. & Palsson, B. O. Genome-scale reconstruction of the metabolic network in Staphylococcus aureus N315: an initial draft to the two-dimensional annotation. BMC Microbiol 5, 8 (2005).
61. Papin, J. A., Price, N. D. & Palsson, B. O. Extreme pathway lengths and reaction participation in genome-scale metabolic networks. Genome Res 12, 1889-1900 (2002).
62. Henry, C. S. et al. High-throughput generation, optimization and analysis of genome-scale metabolic models. Nat Biotechnol 28, 977-982 (2010).
63. Baba, T. et al. Construction of Escherichia coli K-12 in-frame, single-gene knockout mutants: the Keio collection. Mol Syst Biol 2, 2006 0008 (2006).
64. Dukan, S. & Touati, D. Hypochlorous acid stress in Escherichia coli: resistance, DNA damage, and comparison with hydrogen peroxide stress. J Bacteriol 178, 6145-6150 (1996).

### SUPPORTING REFERENCES

1. Becker, S. A. et al. Quantitative prediction of cellular metabolism with constraint-based models: the COBRA Toolbox. Nat Protoc 2, 727-738 (2007).
2. Feist, A. M. et al. A genome-scale metabolic reconstruction for Escherichia coli K-12 MG1655 that accounts for 1260 ORFs and thermodynamic information. Mol Syst Biol 3, 121 (2007).
3. Seaver, L. C. & Imlay, J. A. Alkyl hydroperoxide reductase is the primary scavenger of endogenous hydrogen peroxide in Escherichia coli. J Bacteriol 183, 7173-7181 (2001).
4. Imlay, J. A. & Fridovich, I. Assay of metabolic superoxide production in

Escherichia coli. J Biol Chem 266, 6957-6965 (1991).
5. Covert, M. W., Knight, E. M., Reed, J. L., Herrgard, M. J. & Palsson, B. O. Integrating high-throughput and computational data elucidates bacterial networks. Nature 429, 92-96 (2004).
6. Korshunov, S. & Imlay, J. A. Two sources of endogenous hydrogen peroxide in Escherichia coli. Mol Microbiol 75, 1389-1401 (2010).
7. Keseler, I. M. et al. EcoCyc: a comprehensive view of Escherichia coli biology. Nucleic Acids Res 37, D464-470 (2009).
8. Heldal, M., Norland, S. & Tumyr, 0. X-ray microanalytic method for measurement of dry matter and elemental content of individual bacteria. Appl Environ Microbiol 50, 1251-1257 (1985).
9. Messner, K. R. & Imlay, J. A. The identification of primary sites of superoxide and hydrogen peroxide formation in the aerobic respiratory chain and sulfite reductase complex of Escherichia coli. J Biol Chem 274, 10119-10128 (1999).
10. Seaver, L. C. & Imlay, J. A. Are respiratory enzymes the primary sources of intracellular hydrogen peroxide? J Biol Chem 279, 48742-48750 (2004).
11. Imlay, J. A. A metabolic enzyme that rapidly produces superoxide, fumarate reductase of Escherichia coli. J Biol Chem 270, 19767-19777 (1995).
12. Messner, K. R. & Imlay, J. A. In vitro quantitation of biological superoxide and hydrogen peroxide generation. Methods Enzymol 349, 354-361 (2002).
13. Imlay, J. A. Pathways of oxidative damage. Annu Rev Microbiol 57, 395-418 (2003).
14. Gardner, P. R. & Fridovich, I. Inactivation-reactivation of aconitase in Escherichia coli. A sensitive measure of superoxide radical. J Biol Chem 267, 8757-8763 (1992).
15. Anderson, A. & Cooper, R. A. Gluconeogenesis in Escherichia coli The role of triose phosphate isomerase. FEBS Lett 4, 19-20 (1969).
16. Irani, M. H. & Maitra, P. K. Properties of Escherichia coli mutants deficient in enzymes of glycolysis. J Bacteriol 132, 398-410 (1977).
17. Abdel-Hamid, A. M., Attwood, M. M. & Guest, J. R. Pyruvate oxidase contributes to the aerobic growth efficiency of Escherichia coli. Microbiology 147, 1483-1498 (2001).
18. Chang, Y. Y. & Cronan, J. E., Jr. Genetic and biochemical analyses of Escherichia coli strains having a mutation in the structural gene (poxB) for pyruvate oxidase. J Bacteriol 154, 756-762 (1983).
19. Henning, U., Dennert, G., Hertel, R. & Shipp, W. S. Translation of the structural genes of the E. coli pyruvate dehydrogenase complex. Cold Spring Harb Symp Quant Biol 31, 227-234 (1966).
20. Jensen, P. R. & Michelsen, 0. Carbon and energy metabolism of atp mutants of Escherichia coli. J Bacteriol 174, 7635-7641 (1992).

1. A method for inhibiting a bacterial infection by increasing ROS (reactive oxygen species) production in a bacteria, the method comprising administering to a subject having or at risk for a bacterial infection an effective amount of one or more ROS target modulator compounds and an effective amount of an antibiotic agent.

2-111. (canceled)
